(19) United States
(12) Patent Application Publication
(10) Pub. No.: US 2022/0228147 A1
(43) Pub. Date: Jul. 21, 2022

(54) USES OF TREM COMPOSITIONS TO MODULATE TRNA POOLS

(71) Applicant: FLAGSHIP PIONEERING, INC., Cambridge, MA (US)

(72) Inventors: David Arthur Berry, Newton, MA (US); Theonie Anastassiadis, Boston, MA (US); Christine Elizabeth Hajdin, Boston, MA (US); Noubar Boghos Afeyan, Lexington, MA (US)

(21) Appl. No.: 17/615,427

(22) PCT Filed: May 29, 2020
(86) PCT No.: PCT/US2020/035306

§ 371 Date: Nov. 30, 2021

Related U.S. Application Data
(60) Provisional application No. 62/855,561, filed on May 31, 2019.

(51) Int. Cl. C12N 15/113

(52) U.S. Cl. CPC C12N 15/113; C12N 2310/531

(57) ABSTRACT

The invention relates generally to uses of tRNA-based effector molecules (TREMs) and methods of making the same.

### CROSS-REFERENCE TO RELATED APPLICATIONS

This application claims priority to U.S. Provisional Application 62/855,561 filed on May 31, 2019, the entire contents of which are hereby incorporated by reference.

### SEQUENCE LISTING

The instant application contains a Sequence Listing which has been submitted electronically in ASCII format and is hereby incorporated by reference in its entirety. Said ASCII copy, created on May 19, 2020, is named F2099-7001WO_SL.txt and is 105,520 bytes in size.

### BACKGROUND

tRNA-based effector molecules (TREMs) are complex molecules which possess a number of functions including the initiation and elongation of proteins. Compositions comprising a TREM can be used to modulate said functions to treat or prevent disease.

### SUMMARY

In one aspect, the invention features a method of modulating a tRNA pool in a cell comprising an endogenous open reading frame (ORF), which ORF comprises a codon having a first sequence, comprising:

optionally, acquiring knowledge of the abundance of one or both of (i) and (ii), e.g., acquiring knowledge of the relative amounts of (i) and (ii) in the cell wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;

contacting the cell with a composition comprising a TREM, wherein the TREM has an anticodon that pairs with: (a) the codon having the first sequence; or (b) the codon other than the codon having the first sequence, in an amount and for a time sufficient to modulate the relative amount of the first tRNA moiety and the second tRNA moiety in the cell,

thereby modulating the tRNA pool in the cell.

In an embodiment, the composition comprising a TREM is a pharmaceutically acceptable composition.

In an embodiment, the TREM does not comprise an anticodon that pairs with a stop codon.

In an embodiment, the method comprises acquiring knowledge of (i). In an embodiment, the method comprises acquiring knowledge of (ii). In an embodiment, the method comprises acquiring knowledge of (i) and (ii).

In one aspect, the invention features a method of modulating a tRNA pool in a subject having an endogenous ORF, which ORF comprises a codon having a first sequence, comprising:

optionally, acquiring knowledge of the abundance of one or both of (i) and (ii), e.g., acquiring knowledge of the relative amounts of: (i) and (ii) in the subject wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;

contacting the subject with a composition comprising a TREM, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety in the subject,

thereby modulating the tRNA pool in the subject.

In an embodiment, the composition comprising a TREM is a pharmaceutically acceptable composition.

In an embodiment, the TREM does not comprise an anticodon that pairs with a stop codon.

In an embodiment, the method comprises acquiring knowledge of (i). In an embodiment, the method comprises acquiring knowledge of (ii). In an embodiment, the method comprises acquiring knowledge of (i) and (ii).

In another aspect, the disclosure provides a method of evaluating a tRNA pool in a cell having an endogenous ORF, which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the cell wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the cell.

In an embodiment, the method comprises acquiring knowledge of (i). In an embodiment, the method comprises acquiring knowledge of (ii). In an embodiment, the method comprises acquiring knowledge of (i) and (ii).

In an embodiment, acquiring knowledge of (i) comprises acquiring a value for the abundance, e.g., relative amount, of (i).

In an embodiment, acquiring knowledge of (ii) comprises acquiring a value for the abundance, e.g., relative amount, of (ii).

In an embodiment, responsive to said value, the method comprises administering a composition comprising a TREM, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety.

In another aspect, the disclosure provides a method of evaluating a tRNA pool in a subject having an endogenous ORF, which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the subject wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the subject.

In an embodiment, the method comprises acquiring knowledge of (i). In an embodiment, the method comprises acquiring knowledge of (ii). In an embodiment, the method comprises acquiring knowledge of (i) and (ii).

In an embodiment, acquiring knowledge of (i) comprises acquiring a value for the abundance, e.g., relative amount, of (i).

In an embodiment, acquiring knowledge of (ii) comprises acquiring a value for the abundance, e.g., relative amount, of (ii).

In an embodiment, responsive to said value, the method comprises administering a composition comprising a TREM, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety.

In another aspect, the invention features a method of modulating a tRNA pool in a subject, or a cell, comprising an endogenous ORF comprising a codon comprising a synonymous mutation (a synonymous mutation codon or SMC), comprising:

providing a composition comprising a TREM, wherein the TREM comprises an isoacceptor tRNA moiety comprising an anticodon sequence that pairs with the SMC (the TREM);

contacting the subject with a composition comprising a TREM, or in the case of a cell, contacting the cell with the TREM from a composition comprising a TREM, in an amount and for a time sufficient to modulate the tRNA pool in the subject, or in the cell,

thereby modulating the tRNA pool in the subject or the cell.

In an embodiment, prior to contacting with the composition comprising a TREM, the subject or the cell comprises a first tRNA moiety having an anticodon that pairs with the SMC (the first tRNA moiety), and a second tRNA moiety having an anticodon that pairs with a codon other than the SMC (the second tRNA moiety).

In another aspect, the invention features a method of treating a subject having an endogenous ORF which comprises a codon having a first sequence, comprising:

providing a composition comprising a TREM, wherein the TREM comprises an isoacceptor tRNA moiety having: (a) an anticodon that pairs with the codon of the ORF having the first sequence; or (b) an anticodon that pairs with a codon other than the codon having the first sequence,

contacting the subject with the composition comprising a TREM in an amount and for a time sufficient to treat the subject,

thereby treating the subject.

In another aspect, the disclosure provides a method of treating a subject having an endogenous ORF comprising a codon comprising a synonymous mutation (a synonymous mutation codon or SMC), comprising:

providing a composition comprising a TREM, wherein the TREM comprises an isoacceptor tRNA moiety having an anticodon that pairs with the SMC (the TREM);

contacting the subject with the composition comprising a TREM in an amount and for a time sufficient to treat the subject,

thereby treating the subject.

In another aspect, the invention provides a method of treating a subject having an endogenous ORF comprising a codon having a first sequence, comprising:

(i) acquiring, e.g., directly or indirectly acquiring, a value for the status of the codon having the first sequence in the subject, wherein said value comprises a measure of the presence or absence of the codon having the first sequence in a sample from the subject; and identifying the subject as having the codon having the first sequence; and

(ii) responsive to said value, administering a composition comprising a TREM, wherein the TREM comprises an isoacceptor tRNA moiety having an anticodon that pairs with the codon having the first sequence, to the subject,

thereby treating the subject.

In another aspect, the invention features a method of treating a subject having an endogenous ORF comprising a codon comprising a synonymous mutation (a synonymous mutation codon or SMC), comprising:

(i) acquiring, e.g., directly or indirectly acquiring, a value for the SMC status of the subject, wherein said value comprises a measure of the presence or absence of SMC in a sample from the subject, and identifying the subject as having a SMC; and

(ii) responsive to said value, administering a composition comprising a TREM, wherein the TREM comprises an isoacceptor tRNA moiety having an anticodon that pairs with the SMC, to the subject,

thereby treating the subject.

In an aspect, the invention features a method of selecting a therapy for a subject having an endogenous ORF which comprises a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

wherein the presence of the codon having the first sequence is indicative that said subject is likely to be a responder to the therapy, or said subject will respond, or will likely respond, to the therapy,

thereby selecting the therapy.

In an aspect, the invention features a method of selecting a therapy for a subject having an endogenous ORF comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject, and

wherein the presence of SMC is indicative that said subject is likely to be a responder to the therapy, or said subject will respond, or will likely respond, to the therapy,

thereby selecting the therapy.

In an aspect, the invention provides a method of evaluating a subject having an endogenous ORF comprising a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

identifying the subject as having a codon having the first sequence,

thereby evaluating the subject.

In an aspect, the invention features a method of evaluating a subject having an endogenous ORF comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject; and

identifying the subject as having a SMC,

thereby evaluating the subject.

As disclosed herein, tRNA-based effector molecules (TREMs) are complex molecules which can mediate a variety of cellular processes. Compositions comprising a TREM or pharmaceutical compositions comprising a TREM can be administered to cells, tissues or subjects to modulate tRNA pools in a subject, tissue or a cell, e.g., in vitro or in vivo. Also disclosed herein are methods of treating or preventing a disorder or a symptom of a disorder by administering compositions comprising a TREM or pharmaceutical compositions comprising a TREM. Further disclosed herein are compositions comprising a TREM, or pharmaceutical compositions comprising a TREM, preparations, and methods of making the same.

Additional features of any of the aforesaid compositions (e.g., composition comprising a TREM or pharmaceutical composition comprising a TREM); methods of using said compositions and/or methods of making the same include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. A method of evaluating a tRNA pool in a cell having an endogenous ORF, which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the cell wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the cell.
2. A method of evaluating a tRNA pool in a subject having an endogenous ORF, which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the subject wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the subject.
3. The method of embodiment 1 or 2, comprising acquiring knowledge of (i).
4. The method of embodiment 1 or 2, comprising acquiring knowledge of (ii).
5. The method of embodiment 1 or 2, comprising acquiring knowledge of (i) and (ii).
6. The method of any one of embodiments 1-3 or 5, wherein acquiring knowledge of (i) comprises acquiring a value for the abundance, e.g., relative amount, of (i).
7. The method of any one of embodiments 1-2 or 4-5, wherein acquiring knowledge of (ii) comprises acquiring a value for the abundance, e.g., relative amount, of (ii).
8. The method of embodiment 6 or 7, wherein responsive to said value, the method comprises administering a composition comprising a TREM, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety.
9. A method of modulating a tRNA pool in a cell comprising an endogenous open reading frame (ORF), which ORF comprises a codon having a first sequence, comprising:

optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;

contacting the cell with a composition comprising a TREM, wherein the TREM has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the cell,

thereby modulating the tRNA pool in the cell.

10. The method of embodiment 9, wherein the TREM comprises an anticodon that pairs with (a).
11. The method of embodiment 9, wherein the TREM comprises an anticodon that pairs with (b).
12. A method of modulating a tRNA pool in a subject having an endogenous open reading frame (ORF), which ORF comprises a codon having a first sequence, comprising:

optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;

contacting the subject with a composition comprising a TREM, wherein the TREM has an anticodon that pairs with: (a) the codon having the first sequence; or (b) the codon other than the codon having the first sequence, in an amount and/or for a time sufficient to modulate the relative amounts of the first tRNA moiety and the second tRNA moiety in the subject,

thereby modulating the tRNA pool in the subject.

13. The method of embodiment 12, wherein the TREM comprises an anticodon that pairs with (a).
14. The method of embodiment 12, wherein the TREM comprises an anticodon that pairs with (b).
15. The method of any one of embodiments 9-14, comprising acquiring knowledge of (i).
16. The method of any one of embodiments 9-14, comprising acquiring knowledge of (ii).
17. The method of any one of embodiments 9-14, comprising acquiring knowledge of (i) and (ii).
18. The method of any one of embodiments 9-14, wherein acquiring knowledge of (i) comprises acquiring a value for the abundance, e.g., relative amounts, of (i).
19. The method of any one of embodiments 9-14, wherein acquiring knowledge of (ii) comprises acquiring a value for the abundance, e.g., relative amounts, of (ii).
20. The method of embodiment 18 or 19, wherein responsive to said value, the cell or subject is contacted with the composition comprising the TREM having an anticodon that pairs with (a) or (b).
21. A method of modulating a tRNA pool in a subject having an endogenous open reading frame (ORF) comprising a codon comprising a synonymous mutation (a synonymous mutation codon or SMC), comprising:

providing a composition comprising a TREM, wherein the TREM comprises an isoacceptor tRNA moiety comprising an anticodon sequence that pairs with the SMC (the TREM);

contacting the subject with the composition comprising a TREM in an amount and/or for a time sufficient to modulate the tRNA pool in the subject,

thereby modulating the tRNA pool in the subject.

22. A method of modulating a tRNA pool in a cell comprising an endogenous open reading frame (ORF) comprising a codon comprising a synonymous mutation (a synonymous mutation codon or SMC), comprising:

providing a composition comprising a TREM, wherein the TREM comprises an isoacceptor tRNA moiety comprising an anticodon sequence that pairs with the SMC (the TREM);

contacting the cell with the composition comprising a TREM in an amount and/or for a time sufficient to modulate the tRNA pool in the cell,

thereby modulating the tRNA pool in the cell.

23. The method of embodiment 21 or 22, comprising acquiring knowledge of the abundance of one or both of (i) and (ii) e.g., acquiring knowledge of the relative amounts of (i) and (ii) wherein (i) is a tRNA moiety having an anticodon that pairs with the SMC (the first tRNA moiety) and (ii) is an isoacceptor tRNA moiety having an anticodon that pairs with a codon other than the SMC (the second tRNA moiety), in the subject or cell.
24. The method of embodiment 23, comprising acquiring knowledge of (i).
25. The method of embodiment 23, comprising acquiring knowledge of (ii).
26. The method of embodiment 23, comprising acquiring knowledge of (i) and (ii).
27. The method of embodiment 23, wherein acquiring knowledge of (i) comprises acquiring a value for the abundance, e.g., relative amounts, of (i).
28. The method of embodiment 23, wherein acquiring knowledge of (ii) comprises acquiring a value for the abundance, e.g., relative amounts, of (ii).
29. The method of embodiment 27 or 28, wherein responsive to said value, the cell or subject is contacted with the TREM.
30. A method of treating a subject having an endogenous open reading frame (ORF) which comprises a codon having a first sequence, comprising:

providing a composition comprising a TREM, wherein the TREM comprises an isoacceptor tRNA moiety having: an anticodon that pairs with the codon of the ORF having the first sequence; or an anticodon that pairs with a codon other than the codon having the first sequence,

contacting the subject with the composition comprising a TREM in an amount and/or for a time sufficient to treat the subject,

thereby treating the subject.

31. A method of treating a subject having an endogenous open reading frame (ORF) comprising a codon comprising a synonymous mutation (a synonymous mutation codon or SMC), comprising:

providing a composition comprising a TREM, wherein the TREM comprises an isoacceptor tRNA moiety having an anticodon that pairs with the SMC (the TREM);

contacting the subject with the composition comprising a TREM in an amount and/or for a time sufficient to treat the subject,

thereby treating the subject.

32. The method of embodiment 30 or 31, comprising acquiring knowledge of the abundance of one or both (i) and (ii), e.g., acquiring knowledge of the relative amounts of:

(i) a tRNA moiety having an anticodon that pairs with the codon having a first sequence or the SMC (the first tRNA moiety); and/or

(ii) an isoacceptor tRNA moiety having an anticodon that pairs with a codon other than a codon having the first sequence, or pairs with a codon other than the SMC (the second tRNA moiety).

33. The method of embodiment 32, comprising acquiring knowledge of (i).
34. The method of embodiment 32, comprising acquiring knowledge of (ii).
35. The method of embodiment 32, comprising acquiring knowledge of (i) and (ii).
36. The method of embodiment 32, wherein acquiring knowledge of (i) comprises acquiring a value for the abundance, e.g., relative amounts, of (i).
37. The method of embodiment 32, wherein acquiring knowledge of (ii) comprises acquiring a value for the abundance, e.g., relative amounts, of (ii).
38. The method of embodiment 27 or 28, wherein responsive to said value, the subject is contacted with the TREM.
39. A method of treating a subject having an endogenous open reading frame (ORF) comprising a codon having a first sequence, comprising:

(i) acquiring, e.g., directly or indirectly acquiring, a value for the status of the codon having the first sequence in the subject, wherein said value comprises a measure of the presence or absence of the codon having the first sequence in a sample from the subject; and identifying the subject as having the codon having the first sequence; and

(ii) responsive to said value, administering a composition comprising a TREM, wherein the TREM comprises an isoacceptor tRNA moiety having an anticodon that pairs with the codon having the first sequence, to the subject,

thereby treating the subject.

40. A method of treating a subject having an endogenous open reading frame (ORF) comprising a codon comprising a synonymous mutation (a synonymous mutation codon or SMC), comprising:

(i) acquiring, e.g., directly or indirectly acquiring, a value for the SMC status of the subject, wherein said value comprises a measure of the presence or absence of SMC in a sample from the subject, and identifying the subject as having a SMC; and

(ii) responsive to said value, administering a composition comprising a TREM, wherein the TREM comprises comprising an isoacceptor tRNA moiety having an anticodon that pairs with the SMC, to the subject,

thereby treating the subject.

41. A method of selecting a therapy for a subject having an endogenous open reading frame (ORF) which comprises a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

wherein the presence of the codon having the first sequence is indicative that said subject is likely to be a responder to the therapy, or said subject will respond, or will likely respond, to the therapy,

thereby selecting the therapy.

42. A method of selecting a therapy for a subject having an endogenous open reading frame (ORF) comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject, and

wherein the presence of SMC is indicative that said subject is likely to be a responder to the therapy, or said subject will respond, or will likely respond, to the therapy,

thereby selecting the therapy.

43. A method of evaluating a subject having an endogenous open reading frame (ORF) comprising a codon having a first sequence, comprising:

acquiring, e.g., directly or indirectly acquiring, a value for the status of the codon having the first sequence in the subject, wherein said value comprises a measure of the presence or absence of the codon having the first sequence in a sample from the subject; and

identifying the subject as having a codon having the first sequence,

thereby evaluating the subject.

44. A method of evaluating a subject having an endogenous open reading frame (ORF) comprising a codon comprising a synonymous mutation (a synonymous mutation codon or SMC), comprising:

acquiring, e.g., directly or indirectly acquiring, a value for the SMC status of the subject, wherein said value comprises a measure of the presence or absence of SMC in a sample from the subject; and

identifying the subject as having a SMC,

thereby evaluating the subject.

45. The method of any one of embodiments 8-44, wherein the TREM does not comprise an anticodon that pairs with a stop codon.
46. The method of any one of embodiments 1-45, wherein: (a) the ORF codon having the first sequence; or (b) the SMC; is other than a stop codon, e.g., TAA, TGA or TAG.
47. The method of any one of embodiments 8-46, wherein the TREM comprises a canonical anti-codon/charging site combination.
48. The method of any one of embodiments 1-47, wherein: (a) the ORF codon having the first sequence; or (b) the SMC; has a mutation, e.g., a SNP, in the first position of said codon.
49. The method of any one of embodiments 1-48, wherein: (a) the ORF codon having the first sequence; or (b) the SMC; has a mutation, e.g., a SNP, in the second position of said codon. 50. The method of any one of embodiments 1-49, wherein: (a) the ORF codon having the first sequence; or (b) the SMC; has a mutation, e.g., a SNP, in the third position of said codon.
51. The method of any one of embodiments 1-20, 23-29, 32-38 or 45-50, wherein the first tRNA moiety comprises an endogenous tRNA, and the second tRNA moiety comprises an endogenous tRNA, e.g., wherein the cell or subject has not been contacted with a composition comprising a TREM.
52. The method of any one of embodiments 1-20, 23-29, 32-38, or 45-51 wherein one of the first tRNA moiety and the second tRNA moiety comprises an endogenous tRNA and a TREM.
53. The method of any one of the preceding embodiments wherein: (a) the ORF codon having the first sequence; or (b) the SMC; in the absence of contact with the composition comprising a TREM, is associated with a phenotype, e.g., an unwanted phenotype, e.g., a disorder or symptom, e.g., a disorder or symptom chosen from Table 1.
54. The method of embodiment 53, wherein the disorder or symptom is chosen from a disease group provided in Table 1, e.g., cardiovascular, dermatology, endocrine, immunology, neurology, oncology, ophthalmology, or respiratory.
55. The method of embodiment 53 or 54, wherein the disorder is cardiac hypertrophy.
56. The method of embodiment 53 or 54, wherein the disorder is coronary artery disease.
57. The method of embodiment 53 or 54, wherein the disorder is hypertension.
58. The method of embodiment 53 or 54, wherein the disorder or symptom is an obesity-related trait.
59. The method of embodiment 53 or 54, wherein the disorder is type-1 diabetes.
60. The method of embodiment 53 or 54, wherein the disorder is type-2 diabetes.
61. The method of embodiment 53 or 54, wherein the disorder is psoriasis.
62. The method of embodiment 53 or 54, wherein the disorder is endometriosis.
63. The method of embodiment 53 or 54, wherein the disorder is a chronic inflammatory disease, e.g., ankylosing spondylitis, Crohn's disease, psoriasis, primary sclerosing cholangitis, ulcerative colitis, or pleiotropy.
64. The method of embodiment 53 or 54, wherein the disorder is Crohn's disease.
65. The method of embodiment 53 or 54, wherein the disorder is Grave's disease.
66. The method of embodiment 53 or 54, wherein the disorder is Alzheimer's disease, e.g., age onset Alzheimer's disease or familial Alzheimer's disease.
67. The method of embodiment 53 or 54, wherein the disorder is a major depressive disorder.
68. The method of embodiment 53 or 54, wherein the disorder is migraine.
69. The method of embodiment 53 or 54, wherein the disorder is Parkinson's disease.
70. The method of embodiment 53 or 54, wherein the disorder is schizophrenia.
71. The method of embodiment 53 or 54, wherein the disorder or symptom is adverse response to chemotherapy, e.g., neutropenia or leukopenia.
72. The method of embodiment 53 or 54, wherein the disorder is breast cancer, e.g., early onset breast cancer.
73. The method of embodiment 53 or 54, wherein the disorder is ovarian cancer.
74. The method of embodiment 53 or 54, wherein the disorder is colorectal cancer.
75. The method of embodiment 53 or 54, wherein the disorder is carboplatin disposition in epithelial ovarian cancer.
76. The method of embodiment 53 or 54, wherein the disorder is Clostridium difficile infection in multiple myeloma.
77. The method of embodiment 53 or 54, wherein the disorder is endometrial cancer, e.g., with endometrioid histology.
78. The method of embodiment 53 or 54, wherein the disorder is esophageal squamous cell cancer.
79. The method of embodiment 53 or 54, wherein the disorder is glioblastoma.
80. The method of embodiment 53 or 54, wherein the disorder is lung cancer.
81. The method of embodiment 53 or 54, wherein the disorder or symptom is Macrophage Migration Inhibitory Factor levels.
82. The method of embodiment 53 or 54, wherein the disorder is oral cavity and pharyngeal cancer.
83. The method of embodiment 53 or 54, wherein the disorder is pancreatic cancer.
84. The method of embodiment 53 or 54, wherein the disorder is myopia.
85. The method of embodiment 53 or 54, wherein the disorder is COPD.
86. The method of embodiment 53 or 54, wherein the disorder is asthma.
87. The method of any one of embodiments 1-86, wherein the ORF codon having the first sequence; or the SMC is disposed in a gene, e.g., transcript, provided in Table 1.
88. The method of any one of embodiments 1-87, wherein the ORF codon having the first sequence; or the SMC comprises a codon provided in Table 1, e.g., a codon listed in the “Codon From/To” column of Table 1, e.g., the second codon listed in said column in Table 1.
89. The method of any one of embodiments 1-88, wherein contacting with the composition comprising a TREM, is associated with a second phenotype, e.g., an amelioration of an unwanted phenotype, e.g., amelioration of a disorder or symptom, e.g., amelioration of a disorder or symptom chosen from Table 1.
90. The method of embodiment 89, wherein the disorder or symptom is chosen from a disease group provided in Table 1, e.g., cardiovascular, dermatology, endocrine, immunology, neurology, oncology, ophthalmology, or respiratory.
91. The method of any one of the preceding embodiments, wherein the subject has a disorder or a symptom chosen from Table 1 or the cell from the subject is associated with a disorder or symptom listed in Table 1, e.g., a disease group chosen from cardiovascular, dermatology, endocrine, immunology, neurology, oncology, ophthalmology, or respiratory disease.
92. The method of embodiment 91, wherein the disorder is cardiac hypertrophy.
93. The method of embodiment 91, wherein the disorder is coronary artery disease.
94. The method of embodiment 91, wherein the disorder is hypertension.
95. The method of embodiment 91, wherein the disorder or symptom is an obesity-related trait.
96. The method of embodiment 91, wherein the disorder is type-1 diabetes.
97. The method of embodiment 91, wherein the disorder is type-2 diabetes.
98. The method of embodiment 91, wherein the disorder is psoriasis.
99. The method of embodiment 91, wherein the disorder is endometriosis.
100. The method of embodiment 91, wherein the disorder is a chronic inflammatory disease, e.g., ankylosing spondylitis, Crohn's disease, psoriasis, primary sclerosing cholangitis, ulcerative colitis, or pleiotropy.
101. The method of embodiment 91, wherein the disorder is Crohn's disease.
102. The method of embodiment 91, wherein the disorder is Grave's disease.
103. The method of embodiment 91, wherein the disorder is Alzheimer's disease, e.g., age onset Alzheimer's disease or familial Alzheimer's disease.
104. The method of embodiment 91, wherein the disorder is a major depressive disorder.
105. The method of embodiment 91, wherein the disorder is migraine.
106. The method of embodiment 91, wherein the disorder is Parkinson's disease.
107. The method of embodiment 91, wherein the disorder is schizophrenia.
108. The method of embodiment 91, wherein the disorder or symptom is adverse response to chemotherapy, e.g., neutropenia or leukopenia.
109. The method of embodiment 91, wherein the disorder is breast cancer, e.g., early onset breast cancer.
110. The method of embodiment 91, wherein the disorder is ovarian cancer.
111. The method of embodiment 91, wherein the disorder is colorectal cancer.
112. The method of embodiment 91, wherein the disorder is carboplatin disposition in epithelial ovarian cancer.
113. The method of embodiment 91, wherein the disorder is Clostridium difficile infection in multiple myeloma.
114. The method of embodiment 91, wherein the disorder is endometrial cancer, e.g., with endometrioid histology.
115. The method of embodiment 91, wherein the disorder is esophageal squamous cell cancer.
116. The method of embodiment 91, wherein the disorder is glioblastoma.
117. The method of embodiment 91, wherein the disorder is lung cancer.
118. The method of embodiment 91, wherein the disorder or symptom is Macrophage Migration Inhibitory Factor levels.
119. The method of embodiment 91, wherein the disorder is oral cavity and pharyngeal cancer.
120. The method of embodiment 91, wherein the disorder is pancreatic cancer.
121. The method of embodiment 91, wherein the disorder is myopia.
122. The method of embodiment 91, wherein the disorder is COPD.
123. The method of embodiment 91, wherein the disorder is asthma.
124. The method of any one of the preceding embodiments, wherein acquiring knowledge comprises obtaining a value for the relative amounts of the first tRNA moiety and the second tRNA moiety.
125. The method of embodiment 124, wherein responsive to said value, the method comprises contacting the subject or cell, with a composition comprising a TREM.
126. The method of any one of the preceding embodiments, wherein the first tRNA moiety comprises an endogenous tRNA, and the second tRNA moiety comprises an endogenous tRNA, e.g., where the cell or subject has not been contacted with a composition comprising a TREM.
127. The method of any one of the preceding embodiments, wherein one of the first tRNA moiety and the second tRNA moiety comprises an endogenous tRNA and a TREM.
128. The method of any one of embodiments 8-127, wherein, prior to contact with the subject or cell, the first tRNA moiety is more abundant than the second tRNA moiety.
129. The method of any one of the preceding embodiments, wherein first tRNA moiety is:

at least 0.5%, 1%, 2%, 3%, 4%, 5%, 6%, 7%, 8%, 9%, 10%; 15%, 20%, 25%, 30%, 40%, 50%, 60%, 70%, 80%, 85%, 90%, 95% or 99% more abundant; or

between 0.5-99%, 1-99%, 2-99%, 3-99%, 4-99%, 5-99%, 6-99%, 7-99%, 8-99%, 9-99%, 10-99%, 15-99%, 20-99%, 25-99%, 30-99%, 40-99%, 50-99%, 60-99%, 70-99%, 80-99%, 95-99%, 0.5-95%, 0.5-90%, 0.5-85%, 0.5-80%, 0.5-70%, 0.5-60%, 0.5-50%, 0.5-40%, 0.5-30%, 0.5-25%, 0.5-20%, 0.5-15%, 0.5-10%, 0.5-9%, 0.5-8%, 0.5-7%, 0.5-6%, 0.5-5%, 0.5-4%, 0.5-3%, 0.5-2%, or 0.5-1% more abundant;

than the second tRNA moiety, e.g., wherein abundance is determined by an assay described in any of Examples 29-32.

130. The method of any one of embodiments 8-129, wherein prior to contact with the subject or cell, the second tRNA moiety is more abundant than the first tRNA moiety.
131. The method of any one of the preceding embodiments, wherein the second tRNA moiety is:

at least 0.5%, 1%, 2%, 3%, 4%, 5%, 6%, 7%, 8%, 9%, 10%; 15%, 20%, 25%, 30%, 40%, 50%, 60%, 70%, 80%, 85%, 90%, 95% or 99% more abundant; or

between 0.5-99%, 1-99%, 2-99%, 3-99%, 4-99%, 5-99%, 6-99%, 7-99%, 8-99%, 9-99%, 10-99%, 15-99%, 20-99%, 25-99%, 30-99%, 40-99%, 50-99%, 60-99%, 70-99%, 80-99%, 95-99%, 0.5-95%, 0.5-90%, 0.5-85%, 0.5-80%, 0.5-70%, 0.5-60%, 0.5-50%, 0.5-40%, 0.5-30%, 0.5-25%, 0.5-20%, 0.5-15%, 0.5-10%, 0.5-9%, 0.5-8%, 0.5-7%, 0.5-6%, 0.5-5%, 0.5-4%, 0.5-3%, 0.5-2%, or 0.5-1% more abundant;

than the first tRNA moiety, e.g., wherein abundance is determined by an assay described in any of Examples 29-32.

132. The method of any one of embodiments 8-131, wherein contacting or treating a cell or subject with a composition comprising a TREM comprises modulating a tRNA pool in the cell or subject.
133. The method of any one of embodiments 9-29 or 45-132, wherein modulating comprises increasing the amount of the first tRNA moiety as compared to the second tRNA moiety.
134. The method of embodiment 133, wherein the increase is in the amount, e.g., absolute amount, of the first tRNA moiety in subject or a treated cell.
135. The method of embodiment 133 or 134, wherein the increase is relative to a reference, e.g., a component of the cell treated, e.g., the baseline level of the first tRNA moiety or of the second tRNA moiety.
136. The method of any one of embodiments 133-135, wherein the amount of the first tRNA moiety is increased by at least 1.5, 2, 3, 4, 5, 10, 15, 20, 25, 50 or 100 fold, or between 1-100 fold, 1-50 fold, 1-25 fold, 1-20 fold, 1-15 fold, 1-10 fold, 1-5 fold, 1-4 fold, 1-3 fold, 1-2 fold, 2-100 fold, 3-100 fold, 4-100 fold, 5-100 fold, 10-100 fold, 15-100 fold, 20-100 fold, 25-100 fold, or 50-100 fold, relative to the reference.
137. The method of any one of embodiments 9-29 or 45-136, wherein modulating comprises increasing the relative amount of the second tRNA moiety as compared to the first tRNA moiety.
138. The method of embodiment 137, wherein the increase is in the amount, e.g., absolute amount, of the second tRNA moiety in subject or a treated cell.
139. The method of embodiment 137 or 138, wherein the increase is relative to a reference, e.g., a component of the cell treated, e.g., the baseline level of the second tRNA moiety or of the first tRNA moiety.
140. The method of any one of embodiments 137-139, wherein the amount of the first tRNA moiety is increased by at least 1.5, 2, 3, 4, 5, 10, 15, 20, 25, 50 or 100 fold relative to the reference.
141. The method of any one of embodiments 9-29 or 45-140, wherein modulating comprises modulating a ratio of the first tRNA moiety to the second tRNA moiety.
142. The method of embodiment 141, wherein the ratio of the first tRNA moiety to the second tRNA moiety is 1:10,000, 1:5000, 1:1000, 1:900, 1:800, 1:700, 1:600, 1:500, 1:400, 1:300, 1:200, 1:100, 1:50, 1:40, 1:30, 1:20, 1:10, 1:9, 1:8, 1:7, 1:6, 1:5, 1:4, 1:3, 1:2, 1:1.
143. The method of embodiment 141, wherein the ratio of the second tRNA moiety to the first tRNA moiety is 1:10,000, 1:5000, 1:1000, 1:900, 1:800, 1:700, 1:600, 1:500, 1:400, 1:300, 1:200, 1:100, 1:50, 1:40, 1:30, 1:20, 1:10, 1:9, 1:8, 1:7, 1:6, 1:5, 1:4, 1:3, 1:2, 1:1.
144. The method of any one of embodiments 141-143, wherein the ratio of the first tRNA moiety to the second tRNA moiety is increased. 145. The method of any one of embodiments 141-143, wherein the ratio of the first tRNA moiety to the second tRNA moiety is decreased.
146. The method of any one of embodiments 141-145, wherein the ratio of the first tRNA moiety to the second tRNA moiety is compared to a ratio in a reference, e.g., an otherwise similar cell not contacted with a composition comprising a TREM.
147. The method of any one of the preceding embodiments, wherein the cell is a human cell or the subject is a human.
148. The method of any one of embodiments 8-147, wherein a mutant copy of the ORF is not introduced to the cell or the subject.
149. The method of any one of embodiments 8-147, wherein contacting or treating with the composition comprising a TREM increases production and/or function of a translation product of the ORF, e.g., as evaluated by an assay described in any one of Examples 33-38.
150. The method of any one of the preceding embodiments, wherein the ORF or a SMC containing ORF encodes a polypeptide.
151. The method of any one of the preceding embodiments, wherein the ORF or a SMC containing ORF is a chromosomal ORF.
152. The method of any one of embodiments 1-150, wherein the ORF or a SMC containing ORF is a mitochondrial ORF.
153. The method of any one of embodiments 8-152, wherein contacting with the composition comprising a TREM ameliorates a symptom or disorder, e.g., a symptom or disorder associated with the codon having the first sequence or the SMC.
154. The method of embodiment 153, wherein the symptom or disorder is chosen from Table 1.
155. The method of embodiment 153 or 154, wherein the disorder is cardiac hypertrophy.
156. The method of embodiment 153 or 154, wherein the disorder is coronary artery disease.
157. The method of embodiment 153 or 154, wherein the disorder is hypertension.
158. The method of embodiment 153 or 154, wherein the disorder or symptom is an obesity-related trait.
159. The method of embodiment 153 or 154, wherein the disorder is type-1 diabetes.
160. The method of embodiment 153 or 154, wherein the disorder is type-2 diabetes.
161. The method of embodiment 153 or 154, wherein the disorder is psoriasis.
162. The method of embodiment 153 or 154, wherein the disorder is endometriosis.
163. The method of embodiment 153 or 154, wherein the disorder is a chronic inflammatory disease, e.g., ankylosing spondylitis, Crohn's disease, psoriasis, primary sclerosing cholangitis, ulcerative colitis, or pleiotropy.
164. The method of embodiment 153 or 154, wherein the disorder is Crohn's disease.
165. The method of embodiment 153 or 154, wherein the disorder is Grave's disease.
166. The method of embodiment 153 or 154, wherein the disorder is Alzheimer's disease, e.g., age onset Alzheimer's disease or familial Alzheimer's disease.
167. The method of embodiment 153 or 154, wherein the disorder is a major depressive disorder.
168. The method of embodiment 153 or 154, wherein the disorder is migraine.
169. The method of embodiment 153 or 154, wherein the disorder is Parkinson's disease.
170. The method of embodiment 153 or 154, wherein the disorder is schizophrenia.
171. The method of embodiment 153 or 154, wherein the disorder or symptom is adverse response to chemotherapy, e.g., neutropenia or leukopenia.
172. The method of embodiment 153 or 154, wherein the disorder is breast cancer, e.g., early onset breast cancer.
173. The method of embodiment 153 or 154, wherein the disorder is ovarian cancer.
174. The method of embodiment 153 or 154, wherein the disorder is colorectal cancer.
175. The method of embodiment 153 or 154, wherein the disorder is carboplatin disposition in epithelial ovarian cancer.
176. The method of embodiment 153 or 154, wherein the disorder is Clostridium difficile infection in multiple myeloma.
177. The method of embodiment 153 or 154, wherein the disorder is endometrial cancer, e.g., with endometrioid histology.
178. The method of embodiment 153 or 154, wherein the disorder is esophageal squamous cell cancer.
179. The method of embodiment 153 or 154, wherein the disorder is glioblastoma.
180. The method of embodiment 153 or 154, wherein the disorder is lung cancer.
181. The method of embodiment 153 or 154, wherein the disorder or symptom is Macrophage Migration Inhibitory Factor levels.
182. The method of embodiment 153 or 154, wherein the disorder is oral cavity and pharyngeal cancer.
183. The method of embodiment 153 or 154, wherein the disorder is pancreatic cancer.
184. The method of embodiment 153 or 154, wherein the disorder is myopia.
185. The method of embodiment 153 or 154, wherein the disorder is COPD.
186. The method of embodiment 153 or 154, wherein the disorder is asthma.
187. The method of any one of the preceding embodiments, wherein the ORF codon having the first sequence or the SMC is present in one allele, e.g., the subject or the cell is heterozygous for the codon having the first sequence or the SMC.
188. The method of any one of the preceding embodiments, wherein the ORF codon having the first sequence or the SMC is present in both alleles, e.g., the subject or the cell is homozygous for the codon having the first sequence or the SMC.
189. The method of any one of embodiments 8-188, comprising modulating protein production in a subject or in a cell.
190. The method of any one of embodiments 8-189, comprising modulating the translation product profile, e.g., the amount, rate of amino acid incorporation, rate of production, conformation, activity, cellular location, rate of modification, or co-translational interaction with a binding partner, of a polypeptide, in a subject or in a cell.
191. The method of any one of embodiments 8-190, comprising modulating initiation or elongation of a polypeptide translated from an mRNA comprising the ORF codon having the first sequence or the SMC.
192. The method of any one of embodiments 8-191, wherein the composition comprising a TREM is made by a method described herein, e.g., using a synthetic method (e.g., synthesized using solid state synthesis or liquid phase synthesis); using in vitro transcription (IVT), or by expressing a vector encoding a TREM in a cell.
193. The method of embodiment 192, wherein the method comprises:

(a) providing a host cell, comprising exogenous nucleic acid, e.g., a DNA or RNA, encoding a TREM under conditions sufficient to express the TREM; and

(b) purifying the expressed TREM from the host cell culture to produce a composition comprising a TREM, thereby making a composition comprising a TREM.

194. The method of embodiment 8-193, wherein the composition comprising a TREM is a pharmaceutical composition comprising a TREM.
195. The method of any of embodiments 8-194, wherein the composition comprising a TREM comprises a pharmaceutical excipient.
196. The method of any of embodiments 193-195, comprising introducing the exogenous DNA or RNA into the mammalian host cell.
197. The method of any of embodiments 193-196, wherein the nucleic acid comprises a DNA, which upon transcription, expresses a TREM.
198. The method of any of embodiments 193-197, wherein the nucleic acid comprises an RNA, which upon reverse transcription, results in a DNA which can be transcribed to provide the TREM.
199. The method of any of embodiments 8-198 wherein the composition comprising a TREM comprises a TREM fragment, e.g., as described herein.
200. The method of any of embodiments 193-199, wherein the host cell is a mammalian cell.
201. The method of any of embodiments 193-200, wherein the host cell comprises a cell selected from a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, a Chinese Hamster Ovary (CHO) cell, or a MCF7 cell.
202. The method of any of embodiments 193-201, wherein the host cell is a non-mammalian cell, e.g., a bacterial cell, a yeast cell or an insect cell.
203. The method of any of embodiments 8-202, wherein the TREM is a GMP-grade composition comprising a recombinant TREM (e.g., a composition comprising a TREM made in compliance with cGMP, and/or in accordance with similar requirements) comprising an RNA sequence at least 80% identical to an RNA sequence encoded by a DNA sequence listed in Table 2, or a fragment or functional fragment thereof.
204. The method of any of embodiments 8-203, wherein the TREM comprises one or more post-transcriptional modifications listed in Table 3.
205. The method of embodiment 203 or 204, wherein the composition comprising a recombinant TREM is at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.
206. The method of embodiment 203 or 204, wherein the composition comprising a recombinant TREM is between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to 500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.
207. The method of any one of embodiments 8-206, wherein the composition comprising a TREM comprises one or more, e.g., a plurality, of TREMs.
208. The composition of any one of embodiments 8-207, wherein the composition comprising a TREM (or an intermediate in the production of a composition comprising a TREM) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the composition comprising a TREM;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) Fragments of less than 0.1%, 1%, 2%, 3%, 4%, 5%, 6%, 7%, 8%, 9% or 10%;
(vi) low levels or absence of endotoxins, e.g., as measured by the Limulus amebocyte lysate (LAL) test;
(vii) in-vitro translation activity, e.g., as measured by an assay described in Example 14;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of or an undetectable level of viral contamination.
   209. The method of any of embodiments 8-208, wherein the contacting is an in vitro method, e.g., a cell or tissue, is contacted with the composition comprising a TREM in vitro.
   210. The method of any of embodiments 8-209, wherein the contacting is an ex vivo method, e.g., a cell or tissue, is contacted with the composition comprising a TREM ex vivo, and optionally, the contacted cell or tissue is introduced, e.g., administered, into a subject, e.g., the subject from which the cell or tissue came, or a different subject.
   211. The method of any of embodiments 8-209, wherein the method is an in vivo method, e.g., a subject, or a tissue or cell of a subject, is contacted with the composition comprising a TREM in vivo.
   212. The method of any of embodiments 8-211, wherein the composition comprising a TREM is administered with a delivery agent, e.g., a liposome, a polymer (e.g., a polymer conjugate), a particle, a microsphere, microparticle, or a nanoparticle.
   213. The method of any of embodiments 8-211, wherein the composition comprising a TREM is administered without a carrier, e.g., via naked delivery of the TREM.
   214. The method of any of embodiments 8-213, wherein the TREM enhances:

(a) the stability of a product, e.g., a protein, and/or

(b) ribosome occupancy of a product.

215. The method of any of embodiments 8-214, wherein the TREM:
modulates ribosome occupancy;
modulates protein translation or stability;
modulates mRNA stability;
modulates protein folding or structure;
modulates protein transduction or compartmentalization;
modulates codon usage;
modulates cell fate; or
modulates a signaling pathway, e.g., a cellular signaling pathway.
   216. The method of any of embodiments 8-215, wherein the TREM comprises a post-transcriptional modification from Table 3.
   217. The method of any of embodiments 8-216, wherein the TREM comprises cognate adaptor function, and wherein the TREM mediates acceptance and incorporation of an amino acid associated in nature with the anti-codon of the TREM in the initiation or elongation of a peptide chain.
   218. The method of any of embodiments 8-217, wherein the TREM comprises an RNA sequence at least 80% identical to an RNA sequence of a tRNA which occurs naturally.
   219. The method of any of embodiments 8-218, wherein the TREM comprises an RNA sequence at least 80% identical to an RNA encoded by a DNA sequence listed in Table 2, or a fragment or functional fragment thereof.
   220. The method of any of embodiments 8-219, wherein the TREM comprises an RNA sequence encoded by a DNA sequence listed in Table 2, or a fragment thereof.
   221. The method of any of embodiments 8-220, wherein the TREM comprises an RNA sequence at least XX % identical to an RNA sequence encoded by a DNA sequence listed in Table 2, or a fragment thereof, wherein XX is selected from 80, 85, 90, 95, 96, 97, 98, or 99.
   222. The method of embodiment 221, wherein XX is 80.
   223. The method of embodiment 221, wherein XX is 85.
   224. The method of embodiment 221, wherein XX is 90.
   225. The method of embodiment 221, wherein XX is 95.
   226. The method of embodiment 221, wherein XX is 97.
   227. The method of embodiment 221, wherein XX is 98.
   228. The method of embodiment 221, wherein XX is 99.
   229. The method of any of embodiments 219-228, wherein the DNA sequence is SEQ ID NO:1 or a fragment thereof, or SEQ ID NO:2 or a fragment thereof, or SEQ ID NO: 3 or a fragment thereof, or SEQ ID NO:4 or a fragment thereof, or SEQ ID NO: 5 or a fragment thereof, or SEQ ID NO: 6 or a fragment thereof, or SEQ ID NO: 7 or a fragment thereof, or SEQ ID NO:8 or a fragment thereof, or SEQ ID NO: 9 or a fragment thereof, or SEQ ID NO:10 or a fragment thereof, or SEQ ID NO: 11 or a fragment thereof, or SEQ ID NO:12 or a fragment thereof, or SEQ ID NO: 13 or a fragment thereof, or SEQ ID NO: 14 or a fragment thereof, or SEQ ID NO: 15 or a fragment thereof, or SEQ ID NO: 16 or a fragment thereof, or SEQ ID NO: 17 or a fragment thereof, or SEQ ID NO: 18 or a fragment thereof, or SEQ ID NO: 19 or a fragment thereof, or SEQ ID NO: 20 or a fragment thereof, or SEQ ID NO: 21 or a fragment thereof, or SEQ ID NO: 22 or a fragment thereof, or SEQ ID NO: 23 or a fragment thereof, or SEQ ID NO: 24 or a fragment thereof, or SEQ ID NO: 25 or a fragment thereof, or SEQ ID NO: 26 or a fragment thereof, or SEQ ID NO: 27 or a fragment thereof, or SEQ ID NO: 28 or a fragment thereof, or SEQ ID NO: 29 or a fragment thereof, or SEQ ID NO: 30 or a fragment thereof, or SEQ ID NO: 31 or a fragment thereof, or SEQ ID NO: 32 or a fragment thereof, or SEQ ID NO: 33 or a fragment thereof, or SEQ ID NO: 34 or a fragment thereof, or SEQ ID NO: 35 or a fragment thereof, or SEQ ID NO: 36 or a fragment thereof, or SEQ ID NO: 37 or a fragment thereof, or SEQ ID NO: 38 or a fragment thereof, or SEQ ID NO: 39 or a fragment thereof, or SEQ ID NO: 40 or a fragment thereof, or SEQ ID NO: 41 or a fragment thereof, or SEQ ID NO: 42 or a fragment thereof, or SEQ ID NO: 43 or a fragment thereof, or SEQ ID NO: 44 or a fragment thereof, or SEQ ID NO: 45 or a fragment thereof, or SEQ ID NO: 46 or a fragment thereof, or SEQ ID NO: 47 or a fragment thereof, or SEQ ID NO: 48 or a fragment thereof, or SEQ ID NO: 49 or a fragment thereof, or SEQ ID NO: 50 or a fragment thereof, or SEQ ID NO: 51 or a fragment thereof, or SEQ ID NO: 52 or a fragment thereof, or SEQ ID NO: 53 or a fragment thereof, or SEQ ID NO: 54 or a fragment thereof, or SEQ ID NO: 55 or a fragment thereof, or SEQ ID NO: 56 or a fragment thereof, or SEQ ID NO: 57 or a fragment thereof, or SEQ ID NO: 58 or a fragment thereof, or SEQ ID NO: 59 or a fragment thereof, or SEQ ID NO: 60 or a fragment thereof, or SEQ ID NO: 61 or a fragment thereof, or SEQ ID NO: 62 or a fragment thereof, or SEQ ID NO: 63 or a fragment thereof, or SEQ ID NO: 64 or a fragment thereof, or SEQ ID NO: 65 or a fragment thereof, or SEQ ID NO: 66 or a fragment thereof, or SEQ ID NO: 67 or a fragment thereof, or SEQ ID NO: 68 or a fragment thereof, or SEQ ID NO: 69 or a fragment thereof, or SEQ ID NO: 70 or a fragment thereof,} or SEQ ID NO: 71 or a fragment thereof, or SEQ ID NO: 72 or a fragment thereof, or SEQ ID NO: 73 or a fragment thereof, or SEQ ID NO: 74 or a fragment thereof, or SEQ ID NO: 75 or a fragment thereof, or SEQ ID NO: 76 or a fragment thereof, or SEQ ID NO: 77 or a fragment thereof, or SEQ ID NO: 78 or a fragment thereof, or SEQ ID NO: 79 or a fragment thereof, or SEQ ID NO: 80 or a fragment thereof, or SEQ ID NO: 81 or a fragment thereof, or SEQ ID NO: 82 or a fragment thereof, or SEQ ID NO: 83 or a fragment thereof, or SEQ ID NO: 84 or a fragment thereof, or SEQ ID NO: 85 or a fragment thereof, or SEQ ID NO: 86 or a fragment thereof, or SEQ ID NO: 87 or a fragment thereof, or SEQ ID NO: 88 or a fragment thereof, or SEQ ID NO: 89 or a fragment thereof, or SEQ ID NO: 90 or a fragment thereof, or SEQ ID NO: 91 or a fragment thereof, or SEQ ID NO: 92 or a fragment thereof, or SEQ ID NO: 93 or a fragment thereof, or SEQ ID NO: 94 or a fragment thereof, or SEQ ID NO: 95 or a fragment thereof, or SEQ ID NO: 96 or a fragment thereof, or SEQ ID NO: 97 or a fragment thereof, or SEQ ID NO: 98 or a fragment thereof, or SEQ ID NO: 99 or a fragment thereof, or SEQ ID NO: 100 or a fragment thereof, or SEQ ID NO: 101 or a fragment thereof, or SEQ ID NO: 102 or a fragment thereof, or SEQ ID NO: 103 or a fragment thereof, or SEQ ID NO: 104 or a fragment thereof, or SEQ ID NO: 105 or a fragment thereof, or SEQ ID NO: 106 or a fragment thereof, or SEQ ID NO: 107 or a fragment thereof, or SEQ ID NO: 108 or a fragment thereof, or SEQ ID NO:109 or a fragment thereof, or SEQ ID NO: 110 or a fragment thereof, or SEQ ID NO: 111 or a fragment thereof, or SEQ ID NO: 112 or a fragment thereof, or SEQ ID NO: 113 or a fragment thereof, or SEQ ID NO: 114 or a fragment thereof, or SEQ ID NO: 115 or a fragment thereof, or SEQ ID NO: 116 or a fragment thereof, or SEQ ID NO: 117 or a fragment thereof, or SEQ ID NO: 118 or a fragment thereof, or SEQ ID NO: 119 or a fragment thereof, or SEQ ID NO: 120 or a fragment thereof, or SEQ ID NO: 121 or a fragment thereof, or SEQ ID NO: 122 or a fragment thereof, or SEQ ID NO: 123 or a fragment thereof, or SEQ ID NO: 124 or a fragment thereof, or SEQ ID NO: 125 or a fragment thereof, or SEQ ID NO: 126 or a fragment thereof, or SEQ ID NO: 127 or a fragment thereof, or SEQ ID NO: 128 or a fragment thereof, or SEQ ID NO: 129 or a fragment thereof, or SEQ ID NO: 130 or a fragment thereof, or SEQ ID NO: 131 or a fragment thereof, or SEQ ID NO: 132 or a fragment thereof, or SEQ ID NO: 133 or a fragment thereof, or SEQ ID NO: 134 or a fragment thereof, or SEQ ID NO: 135 or a fragment thereof, or SEQ ID NO:136 or a fragment thereof, or SEQ ID NO: 137 or a fragment thereof, or SEQ ID NO: 138 or a fragment thereof, or SEQ ID NO: 139 or a fragment thereof, or SEQ ID NO: 140 or a fragment thereof, or SEQ ID NO: 141 or a fragment thereof, or SEQ ID NO: 142 or a fragment thereof, or SEQ ID NO: 143 or a fragment thereof, or SEQ ID NO: 144 or a fragment thereof, or SEQ ID NO: 145 or a fragment thereof, or SEQ ID NO: 146 or a fragment thereof, or SEQ ID NO: 147 or a fragment thereof, or SEQ ID NO: 148 or a fragment thereof, or SEQ ID NO: 149 or a fragment thereof, or SEQ ID NO: 150 or a fragment thereof, or SEQ ID NO: 151 or a fragment thereof, or SEQ ID NO: 152 or a fragment thereof, or SEQ ID NO: 153 or a fragment thereof, or SEQ ID NO: 154 or a fragment thereof, or SEQ ID NO: 155 or a fragment thereof, or SEQ ID NO: 156 or a fragment thereof, or SEQ ID NO: 157 or a fragment thereof, or SEQ ID NO: 158 or a fragment thereof, or SEQ ID NO: 159 or a fragment thereof, or SEQ ID NO: 160 or a fragment thereof, or SEQ ID NO: 161 or a fragment thereof, or SEQ ID NO: 162 or a fragment thereof, or SEQ ID NO: 163 or a fragment thereof, or SEQ ID NO: 164 or a fragment thereof, or SEQ ID NO: 165 or a fragment thereof, or SEQ ID NO: 166 or a fragment thereof, or SEQ ID NO: 167 or a fragment thereof, or SEQ ID NO: 168 or a fragment thereof, or SEQ ID NO: 169 or a fragment thereof, or SEQ ID NO: 170 or a fragment thereof, or SEQ ID NO: 171 or a fragment thereof, or SEQ ID NO: 172 or a fragment thereof, or SEQ ID NO: 173 or a fragment thereof, or SEQ ID NO: 174 or a fragment thereof, or SEQ ID NO: 175 or a fragment thereof, or SEQ ID NO: 176 or a fragment thereof, or SEQ ID NO: 177 or a fragment thereof, or SEQ ID NO: 178 or a fragment thereof, or SEQ ID NO: 179 or a fragment thereof, or SEQ ID NO: 180 or a fragment thereof, or SEQ ID NO: 181 or a fragment thereof, or SEQ ID NO: 182 or a fragment thereof, or SEQ ID NO: 183 or a fragment thereof, or SEQ ID NO: 184 or a fragment thereof, or SEQ ID NO: 185 or a fragment thereof, or SEQ ID NO: 186 or a fragment thereof, or SEQ ID NO: 187 or a fragment thereof, or SEQ ID NO: 188 or a fragment thereof, or SEQ ID NO: 189 or a fragment thereof, or SEQ ID NO: 190 or a fragment thereof, or SEQ ID NO: 191 or a fragment thereof, or SEQ ID NO: 192 or a fragment thereof, or SEQ ID NO: 193 or a fragment thereof, or SEQ ID NO: 194 or a fragment thereof, or SEQ ID NO: 195 or a fragment thereof, or SEQ ID NO: 196 or a fragment thereof, or SEQ ID NO: 197 or a fragment thereof, or SEQ ID NO: 198 or a fragment thereof, or SEQ ID NO: 199 or a fragment thereof, or SEQ ID NO: 200 or a fragment thereof, or SEQ ID NO: 201 or a fragment thereof, or SEQ ID NO: 202 or a fragment thereof, or SEQ ID NO: 203 or a fragment thereof, or SEQ ID NO: 204 or a fragment thereof, or SEQ ID NO: 205 or a fragment thereof, or SEQ ID NO: 206 or a fragment thereof, or SEQ ID NO: 207 or a fragment thereof, or SEQ ID NO: 208 or a fragment thereof, or SEQ ID NO: 209 or a fragment thereof, or SEQ ID NO: 210 or a fragment thereof, or SEQ ID NO: 211 or a fragment thereof, or SEQ ID NO: 212 or a fragment thereof, or SEQ ID NO: 213 or a fragment thereof, or SEQ ID NO: 214 or a fragment thereof, or SEQ ID NO: 215 or a fragment thereof, or SEQ ID NO: 216 or a fragment thereof, or SEQ ID NO: 217 or a fragment thereof, or SEQ ID NO: 218 or a fragment thereof, or SEQ ID NO: 219 or a fragment thereof, or SEQ ID NO: 220 or a fragment thereof, or SEQ ID NO: 221 or a fragment thereof, or SEQ ID NO: 222 or a fragment thereof, or SEQ ID NO: 223 or a fragment thereof, or SEQ ID NO: 224 or a fragment thereof, or SEQ ID NO: 225 or a fragment thereof, or SEQ ID NO: 226 or a fragment thereof, or SEQ ID NO: 227 or a fragment thereof, or SEQ ID NO: 228 or a fragment thereof, or SEQ ID NO: 229 or a fragment thereof, or SEQ ID NO: 230 or a fragment thereof, or SEQ ID NO: 231 or a fragment thereof, or SEQ ID NO: 232 or a fragment thereof, or SEQ ID NO: 233 or a fragment thereof, or SEQ ID NO: 234 or a fragment thereof, or SEQ ID NO: 235 or a fragment thereof, or SEQ ID NO: 236 or a fragment thereof, or SEQ ID NO: 237 or a fragment thereof, or SEQ ID NO: 238 or a fragment thereof, or SEQ ID NO: 239 or a fragment thereof, or SEQ ID NO: 240 or a fragment thereof, or SEQ ID NO: 241 or a fragment thereof, or SEQ ID NO: 242 or a fragment thereof, or SEQ ID NO: 243 or a fragment thereof, or SEQ ID NO: 244 or a fragment thereof, or SEQ ID NO: 245 or a fragment thereof, or SEQ ID NO: 246 or a fragment thereof, or SEQ ID NO: 247 or a fragment thereof, or SEQ ID NO: 248 or a fragment thereof, or SEQ ID NO: 249 or a fragment thereof, or SEQ ID NO: 250 or a fragment thereof, or SEQ ID NO: 251 or a fragment thereof, or SEQ ID NO: 252 or a fragment thereof, or SEQ ID NO: 253 or a fragment thereof, or SEQ ID NO: 254 or a fragment thereof, or SEQ ID NO: 255 or a fragment thereof, or SEQ ID NO: 256 or a fragment thereof, or SEQ ID NO: 257 or a fragment thereof, or SEQ ID NO: 258 or a fragment thereof, or SEQ ID NO: 259 or a fragment thereof, or SEQ ID NO: 260 or a fragment thereof, or SEQ ID NO: 261 or a fragment thereof, or SEQ ID NO: 262 or a fragment thereof, or SEQ ID NO: 263 or a fragment thereof, or SEQ ID NO: 264 or a fragment thereof, or SEQ ID NO: 265 or a fragment thereof, or SEQ ID NO: 266 or a fragment thereof, or SEQ ID NO: 267 or a fragment thereof, or SEQ ID NO: 268 or a fragment thereof, or SEQ ID NO: 269 or a fragment thereof, or SEQ ID NO: 270 or a fragment thereof, or SEQ ID NO: 271 or a fragment thereof, or SEQ ID NO: 272 or a fragment thereof, or SEQ ID NO: 273 or a fragment thereof, or SEQ ID NO: 274 or a fragment thereof, or SEQ ID NO: 275 or a fragment thereof, or SEQ ID NO: 276 or a fragment thereof, or SEQ ID NO: 277 or a fragment thereof, or SEQ ID NO: 278 or a fragment thereof, or SEQ ID NO: 279 or a fragment thereof, or SEQ ID NO: 280 or a fragment thereof, or SEQ ID NO: 281 or a fragment thereof, or SEQ ID NO: 282 or a fragment thereof, or SEQ ID NO: 283 or a fragment thereof, or SEQ ID NO: 284 or a fragment thereof, or SEQ ID NO: 285 or a fragment thereof, or SEQ ID NO: 286 or a fragment thereof, or SEQ ID NO: 287 or a fragment thereof, or SEQ ID NO: 288 or a fragment thereof, or SEQ ID NO: 289 or a fragment thereof, or SEQ ID NO: 290 or a fragment thereof, or SEQ ID NO: 291 or a fragment thereof, or SEQ ID NO: 292 or a fragment thereof, or SEQ ID NO: 293 or a fragment thereof, or SEQ ID NO: 294 or a fragment thereof, or SEQ ID NO: 295 or a fragment thereof, or SEQ ID NO: 296 or a fragment thereof, or SEQ ID NO: 297 or a fragment thereof, or SEQ ID NO: 298 or a fragment thereof, or SEQ ID NO: 299 or a fragment thereof, or SEQ ID NO: 300 or a fragment thereof, or SEQ ID NO: 301 or a fragment thereof, or SEQ ID NO: 302 or a fragment thereof, or SEQ ID NO: 303 or a fragment thereof, or SEQ ID NO: 304 or a fragment thereof, or SEQ ID NO: 305 or a fragment thereof, or SEQ ID NO: 306 or a fragment thereof, or SEQ ID NO: 307 or a fragment thereof, or SEQ ID NO: 308 or a fragment thereof, or SEQ ID NO: 309 or a fragment thereof, or SEQ ID NO: 310 or a fragment thereof, or SEQ ID NO: 311 or a fragment thereof, or SEQ ID NO: 312 or a fragment thereof, or SEQ ID NO: 313 or a fragment thereof, or SEQ ID NO: 314 or a fragment thereof, or SEQ ID NO: 315 or a fragment thereof, or SEQ ID NO: 316 or a fragment thereof, or SEQ ID NO: 317 or a fragment thereof, or SEQ ID NO: 318 or a fragment thereof, or SEQ ID NO: 319 or a fragment thereof, or SEQ ID NO: 320 or a fragment thereof, or SEQ ID NO: 321 or a fragment thereof, or SEQ ID NO: 322 or a fragment thereof, or SEQ ID NO: 323 or a fragment thereof, or SEQ ID NO: 324 or a fragment thereof, or SEQ ID NO: 325 or a fragment thereof, or SEQ ID NO: 326 or a fragment thereof, or SEQ ID NO: 327 or a fragment thereof, or SEQ ID NO: 328 or a fragment thereof, or SEQ ID NO: 329 or a fragment thereof, or SEQ ID NO: 330 or a fragment thereof, or SEQ ID NO: 331 or a fragment thereof, or SEQ ID NO: 332 or a fragment thereof, or SEQ ID NO: 333 or a fragment thereof, or SEQ ID NO: 334 or a fragment thereof, or SEQ ID NO: 335 or a fragment thereof, or SEQ ID NO: 336 or a fragment thereof, or SEQ ID NO: 337 or a fragment thereof, or SEQ ID NO: 338 or a fragment thereof, or SEQ ID NO: 339 or a fragment thereof, or SEQ ID NO: 340 or a fragment thereof, or SEQ ID NO: 341 or a fragment thereof, or SEQ ID NO: 342 or a fragment thereof, or SEQ ID NO: 343 or a fragment thereof, or SEQ ID NO: 344 or a fragment thereof, or SEQ ID NO: 345 or a fragment thereof, or SEQ ID NO: 346 or a fragment thereof, or SEQ ID NO: 347 or a fragment thereof, or SEQ ID NO: 348 or a fragment thereof, or SEQ ID NO: 349 or a fragment thereof, or SEQ ID NO: 350 or a fragment thereof, or SEQ ID NO: 351 or a fragment thereof, or SEQ ID NO: 352 or a fragment thereof, or SEQ ID NO: 353 or a fragment thereof, or SEQ ID NO: 354 or a fragment thereof, or SEQ ID NO: 355 or a fragment thereof, or SEQ ID NO: 356 or a fragment thereof, or SEQ ID NO: 357 or a fragment thereof, or SEQ ID NO: 358 or a fragment thereof, or SEQ ID NO: 359 or a fragment thereof, or SEQ ID NO: 360 or a fragment thereof, or SEQ ID NO: 361 or a fragment thereof, or SEQ ID NO: 362 or a fragment thereof, or SEQ ID NO: 363 or a fragment thereof, or SEQ ID NO: 364 or a fragment thereof, or SEQ ID NO: 365 or a fragment thereof, or SEQ ID NO: 366 or a fragment thereof, or SEQ ID NO: 367 or a fragment thereof, or SEQ ID NO: 368 or a fragment thereof, or SEQ ID NO: 369 or a fragment thereof, or SEQ ID NO: 370 or a fragment thereof, or SEQ ID NO: 371 or a fragment thereof, or SEQ ID NO: 372 or a fragment thereof, or SEQ ID NO: 373 or a fragment thereof, or SEQ ID NO: 374 or a fragment thereof, or SEQ ID NO: 375 or a fragment thereof, or SEQ ID NO: 376 or a fragment thereof, or SEQ ID NO: 377 or a fragment thereof, or SEQ ID NO: 378 or a fragment thereof, or SEQ ID NO: 379 or a fragment thereof, or SEQ ID NO: 380 or a fragment thereof, or SEQ ID NO: 381 or a fragment thereof, or SEQ ID NO: 382 or a fragment thereof, or SEQ ID NO: 383 or a fragment thereof, or SEQ ID NO: 384 or a fragment thereof, or SEQ ID NO: 385 or a fragment thereof, or SEQ ID NO: 386 or a fragment thereof, or SEQ ID NO: 387 or a fragment thereof, or SEQ ID NO: 388 or a fragment thereof, or SEQ ID NO: 389 or a fragment thereof, or SEQ ID NO: 390 or a fragment thereof, or SEQ ID NO: 391 or a fragment thereof, or SEQ ID NO: 392 or a fragment thereof, or SEQ ID NO: 393 or a fragment thereof, or SEQ ID NO: 394 or a fragment thereof, or SEQ ID NO: 395 or a fragment thereof, or SEQ ID NO: 396 or a fragment thereof, or SEQ ID NO: 397 or a fragment thereof, or SEQ ID NO: 398 or a fragment thereof, or SEQ ID NO: 399 or a fragment thereof, or SEQ ID NO: 400 or a fragment thereof, or SEQ ID NO: 401 or a fragment thereof, or SEQ ID NO: 402 or a fragment thereof, or SEQ ID NO: 403 or a fragment thereof, or SEQ ID NO: 404 or a fragment thereof, or SEQ ID NO: 405 or a fragment thereof, or SEQ ID NO: 406 or a fragment thereof, or SEQ ID NO: 407 or a fragment thereof, or SEQ ID NO: 408 or a fragment thereof, or SEQ ID NO: 409 or a fragment thereof, or SEQ ID NO: 410 or a fragment thereof, or SEQ ID NO: 411 or a fragment thereof, or SEQ ID NO: 412 or a fragment thereof, or SEQ ID NO: 413 or a fragment thereof, or SEQ ID NO: 414 or a fragment thereof, or SEQ ID NO: 415 or a fragment thereof, or SEQ ID NO: 416 or a fragment thereof, or SEQ ID NO: 417 or a fragment thereof, or SEQ ID NO: 418 or a fragment thereof, or SEQ ID NO: 419 or a fragment thereof, or SEQ ID NO: 420 or a fragment thereof, or SEQ ID NO: 421 or a fragment thereof, or SEQ ID NO: 422 or a fragment thereof, or SEQ ID NO: 423 or a fragment thereof, or SEQ ID NO: 424 or a fragment thereof, or SEQ ID NO: 425 or a fragment thereof, or SEQ ID NO: 426 or a fragment thereof, or SEQ ID NO: 427 or a fragment thereof, or SEQ ID NO:428 or a fragment thereof, or SEQ ID NO: 429 or a fragment thereof, or SEQ ID NO: 430 or a fragment thereof, or SEQ ID NO: 431 or a fragment thereof, or SEQ ID NO: 432 or a fragment thereof, or SEQ ID NO: 433 or a fragment thereof, or SEQ ID NO: 434 or a fragment thereof, or SEQ ID NO: 435 or a fragment thereof, or SEQ ID NO: 436 or a fragment thereof, or SEQ ID NO: 437 or a fragment thereof, or SEQ ID NO: 438 or a fragment thereof, or SEQ ID NO: 439 or a fragment thereof, or SEQ ID NO: 440 or a fragment thereof, or SEQ ID NO: 441 or a fragment thereof, or SEQ ID NO: 442 or a fragment thereof, or SEQ ID NO: 443 or a fragment thereof, or SEQ ID NO: 444 or a fragment thereof, or SEQ ID NO: 445 or a fragment thereof, or SEQ ID NO: 446 or a fragment thereof, or SEQ ID NO: 447 or a fragment thereof, or SEQ ID NO: 448 or a fragment thereof, or SEQ ID NO: 449 or a fragment thereof, or SEQ ID NO: 450 or a fragment thereof, or SEQ ID NO: 451 or a fragment thereof.
   230. A method of making a tRNA effector molecule (TREM), comprising a synthetic method (e.g., synthesized using solid state synthesis or liquid phase synthesis); or an in vitro transcription (IVT) method.
   231. A method of making a tRNA effector molecule (TREM), comprising:

(a) providing a host cell, comprising exogenous nucleic acid, e.g., a DNA or RNA, encoding a TREM under conditions sufficient to express the TREM, and

(b) purifying the expressed TREM from the host cell culture to produce a composition comprising a TREM, thereby making a composition comprising a TREM.

232. The method of embodiment 230 or 231 wherein, wherein the composition comprising a TREM comprises a TREM fragment, e.g., as described herein.
233. The method of embodiment 232, wherein the TREM fragment is produced in vivo, in the host cell.
234. The method of embodiment 232 or 233, wherein the TREM fragment is produced by fragmenting an expressed TREM after production of the TREM by the cell, e.g., a TREM produced by the host cell is fragmented after release or purification from the host cell, e.g., the TREM is fragmented ex vivo.
235. The method of any of embodiments 230-234, wherein the method results in an increase, e.g., at least a 2.2, 2.5, 3, 4, 5, 6, 7, 8, 9, 10, or 20-fold increase in the production of total of endogenous tRNA and TREM in the host cell (e.g., as measured by an assay described in any of Examples 1-3 or 7-11), e.g., as compared with a reference cell, e.g., a similar cell but not engineered or modified to express a TREM.
236. The method of embodiment 235, wherein method results in an increase in TREM production and/or tRNA production between 2.2 to 20-fold, between 2.2 to 15-fold, between 2.2 to 10-fold, between 2.2 to 9-fold, between 2.2 to 8-fold, between 2.2 to 7-fold, between 2.2 to 6-fold, between 2.2 to 5-fold, between 2.2 to 4-fold, between 2.2 to 3-fold, between 2.2 to 2.5-fold, between 2.5 to 20-fold, between 3 to 20-fold, between 4 to 20-fold, between 5 to 20-fold, between 6 to 20-fold, between 7 to 20-fold, between 8 to 20-fold, between 9 to 20-fold, between 10 to 20-fold, or between 15 to 20-fold.
237 The method of any of embodiments 230-236, wherein the method results in a detectable level of TREM in the host cell, e.g., as measured by an assay described in any of Examples 1-3 or 7-11.
238. The method of any of embodiments 230-237, wherein the host cell is capable of a post-transcriptional modification, of the TREM.
239. The method of any of embodiments 230-238, wherein the host cell is capable of a post-transcriptional modification, of the TREM, e.g., a post-transcriptional modification selected from Table 3.
240. The method of any of embodiments 230-239, wherein the host cell has been modified to modulate, e.g., increase, its ability to provide a post-transcriptional modification, of the TREM, e.g., a post-transcriptional modification selected from Table 3, e.g., the host cell has been modified to provide for, an increase, or decrease in, the expression of a gene, e.g., a gene encoding an enzyme from Table 3, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC.
241. The method of any one of embodiments 230-240, wherein the host cell is a mammalian cell capable of a post-transcriptional modification, of the TREM, e.g., a post-transcriptional modification selected from Table 3.
242. The method of any of embodiments 230-241, wherein the host cell comprises a HeLa cell, a HEK293 cell, a HT-1080 cell, a PER.C6 cell, a HKB-11 cell, a CAP cell or a HuH-7 cell.
243. The method of any of embodiments 230-242, wherein the host cell has increased expression of an oncogene, e.g., Ras, c-myc or c-jun.
244. The method of any of embodiments 230-243, wherein the host cell has decreased expression of a tumor suppressor, e.g., p53 or Rb.
245. The method of any of embodiments 230-244, wherein the host cell has increased expression of RNA Polymerase III (RNA Pol III).
246. The method of any of embodiments 230-245, wherein the host cell is a non-mammalian host cell.
247. The method of any of embodiments 230-246, wherein the host cell is a bacterial cell, e.g., an E. coli cell, or a yeast cell.
248. The method of any of embodiments 230-247, further comprising measuring one or more of the following characteristics of the composition comprising a TREM (or an intermediate in the production of a composition comprising a TREM):
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the composition comprising a TREM;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) fragments of less than 0.1%, 1%, 2%, 3%, 4%, 5%, 6%, 7%, 8%, 9% or 10%;
(vi) low levels or absence of endotoxins, e.g., as measured by the Limulus amebocyte lysate (LAL) test;
(vii) in-vitro translation activity, e.g., as measured by an assay described in Example 14;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP <71>, and/or the composition or preparation meets the standard of USP <85>; or
(x) viral contamination, e.g., the composition or preparation has an absence of or an undetectable level of viral contamination.
   249. The method of embodiment 248, further comprising, comparing the measured value with a reference value or a standard.
   250. The method of embodiment 249, further comprising, in response to the comparison, modulating the composition comprising a TREM to:
(i) increase the purity of the composition;
(ii) decrease the amount of HCP in the composition;
(iii) decrease the amount of DNA in the composition;
(iv) decrease the amount of fragments in the composition;
(v) decrease the amount of endotoxins in the composition;
(vi) increase the in vitro translation activity of the composition;
(vii) increase the TREM concentration of the composition; or
(viii) increase the sterility of the composition.
   251. The TREM of any of embodiments 230-250, wherein the TREM was purified from host cells cultured in a bioreactor.
   252. The bioreactor of embodiment 251,
(i) comprising at least 1×10⁷, 1×10⁸, 1×10⁹, 1×10¹⁰, 1×10¹¹, 1×10¹², 1×10¹³, or 1×10¹⁴ host cells;
(ii) comprising between 100 mL and 100 liters of culture medium, e.g., at least 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, or 100 liters of culture medium;
(iii) wherein the bioreactor is selected from a continuous flow bioreactor, a batch process bioreactor, a perfusion bioreactor, and a fed batch bioreactor; or
(iv) wherein the bioreactor is held under conditions sufficient to express the TREM.
   253. The method of any of embodiments 230-252, wherein the TREM is encoded by, or expressed from, a nucleic acid sequence comprising:
(i) a control region sequence;
(ii) a sequence encoding a modified TREM;
(iii) a sequence encoding more than one TREM; or
(iv) a sequence other than a tRNA^{MET} sequence.
   254. The method of embodiment 253, wherein the nucleic acid sequence comprises a promoter sequence.
   255. The method of embodiment 253 or 254, wherein the nucleic acid sequence comprises a promoter sequence that comprises an RNA polymerase III (Pol III) recognition site, e.g., a Pol III binding site, e.g., a U6 promoter sequence or fragment thereof.

Other features, objects, and advantages of the invention will be apparent from the description and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTIONS OF THE DRAWINGS

FIG. 1 Panel A depicts the mRNA and protein sequence, and the endogenous tRNA pool for a non-SNP subject. The sequence of the second codon is GTG (depicted by the open triangle) encoding for the amino acid valine. Two Valine isoacceptor tRNA species are shown. Each of the two tRNA species recognize different Valine codons. The two species have different abundances. The species that recognize the wildtype codon, GTG, are not shaded and are in higher abundance. The shaded species, which has a lower abundance, does not pair with the wildtype codon. Thus, the Valine isoacceptor tRNA species that corresponds to the codon used (GTG) is abundant.

FIG. 1 Panel B depicts the mRNA and protein sequence, and the endogenous tRNA pool for a subject having a single nucleotide polymorphism (SNP) at the third position of the second codon (shown with a closed triangle) in the depicted mRNA sequence. The composition of the endogenous tRNA pool is the same as described for FIG. 1 Panel A. However, incorporation of Valine at the second codon now depends on the use of a less abundant tRNA species (the shaded species). As a consequence, as shown in FIG. 1 Panel B, translation is compromised. Other consequences of using a less abundant tRNA species may also be, e.g., interruption of the elongation of the peptide chain, lower protein production, protein misfolding, protein mislocalization, altered protein function, or altered mRNA transcript stability.

FIG. 1 Panel C depicts the same mRNA sequence as in FIG. 1 Panel B, which includes a SNP at the third position of the second codon. The endogenous tRNAs of the pool are the same as in Panels A and B, but the pool is supplemented with exogenous TREMs which increase the abundance of species that will pair with the SNP codon. This can result in an improvement in the translation of the mRNA.

FIG. 2 Panel A depicts the mRNA and protein sequence, and the endogenous tRNA pool for a non-SNP subject. The sequence of the second codon is GTG (depicted by the open triangle) encoding for the amino acid Valine. Two Valine isoacceptor tRNA species are shown. Each of the two tRNA species recognize different Valine codons. The two species have different abundances. The species that recognize the wildtype codon, GTG, are not shaded and are in higher abundance. The shaded species, which has a lower abundance, does not pair with the wildtype codon. Thus, the Valine isoacceptor tRNA species that corresponds to the codon used (GTG) is abundant. This results in translation of the mRNA sequence into the corresponding protein as depicted.

FIG. 2 Panel B depicts the mRNA and protein sequence, and the endogenous tRNA pool for a subject having a single nucleotide polymorphism (SNP) at the third position of the second codon (shown with a closed triangle) in the depicted mRNA sequence. The composition of the endogenous tRNA pool is the same as described for FIG. 2 Panel A. However, incorporation of Valine at the second codon now depends on the use of a less abundant tRNA species (the shaded species). As a consequence, as shown in FIG. 2 Panel B, translation of the mRNA sequence into the corresponding protein is compromised.

FIG. 2 Panel C depicts the same mRNA sequence as in FIG. 2 Panel B, which includes a SNP at the third position of the second codon (shown with a closed triangle). The endogenous tRNAs of the pool are the same as in Panels A and B, but the pool is supplemented with exogenous TREMs which increase the abundance of species that will pair with the SNP codon. As a consequence, translation of the mRNA sequence into the corresponding protein is not compromised and is similar to that of a non-SNP subject.

FIG. 3 The top row depicts the endogenous tRNA pool and, moving to the right, the mRNA and protein sequence, for a non-SNP subject. The sequence of the second codon is GTG (depicted by the open triangle) encoding for the amino acid Valine. Two Valine isoacceptor tRNA species are shown. Each of the two tRNA species recognize different Valine codons. The two species have different abundances. The species that recognize the wildtype codon, GTG, are not shaded and are in higher abundance. The shaded species, which has a lower abundance, does not pair with the wildtype codon. Thus, the Valine isoacceptor tRNA species that corresponds to the codon used (GTG) is abundant. This results in translation of the mRNA sequence into the corresponding protein as depicted. Using a more abundant tRNA species may also have an impact on transcript stability, protein expression, protein function, protein folding, or protein localization.

FIG. 3 The middle row depicts the endogenous tRNA pool and the mRNA and protein sequence for a subject having a single nucleotide polymorphism (SNP) at the third position of the second codon (shown with a closed triangle) in the depicted mRNA sequence. The composition of the endogenous tRNA pool is the same as described for FIG. 3 top row. However, incorporation of Valine at the second codon now depends on the use of a less abundant tRNA species (the shaded species). As a consequence, as shown in FIG. 3 middle row, translation of the mRNA sequence into the corresponding protein is compromised. Using a less abundant tRNA species may also reduce transcript stability, reduce protein expression, change protein function, change protein folding, or change protein localization.

FIG. 3 The bottom row depicts the same mRNA sequence as in FIG. 3 middle row, which includes a SNP at the third position of the second codon (shown with a closed triangle). The endogenous tRNAs of the pool are the same as in the top row and the middle row, but the pool is supplemented with exogenous TREMs which increase the abundance of species that can pair with the SNP codon. As a consequence, translation of the mRNA sequence into the corresponding protein is not compromised.

FIGS. 4A-4C are graphs showing an increase in cell growth in three cells lines after transfection with a TREM corresponding to the initiator methionine (iMet). FIG. 4A is a graph showing increased % cellular confluency (a measure of cell growth) of U2OS cells transfected with Cy3-labeled iMet-CAT-TREM or transfected with a Cy3-labeled non-targeted control. FIG. 4B is a graph showing increased % cellular confluency (a measure of cell growth) of H1299 cells transfected with Cy3-labeled iMet-CAT-TREM or transfected with a Cy3-labeled non-targeted control. FIG. 4C is a graph showing increased % cellular confluency (a measure of cell growth) of Hela cells transfected with Cy3-labeled iMet-CAT-TREM or transfected with a Cy3-labeled non-targeted control.

FIG. 5 is a graph depicting an increase in NanoLuc reporter expression upon addition of iMET-TREM to a translational reaction with cell free lysate. As a control, a translational reaction with buffer was performed.

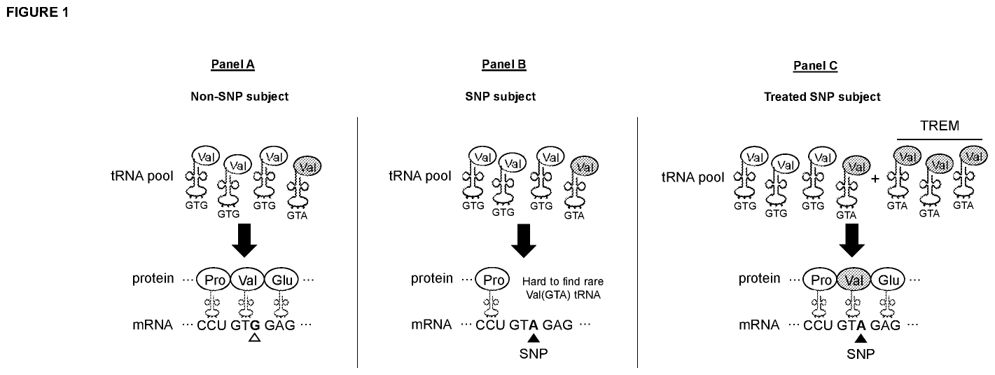

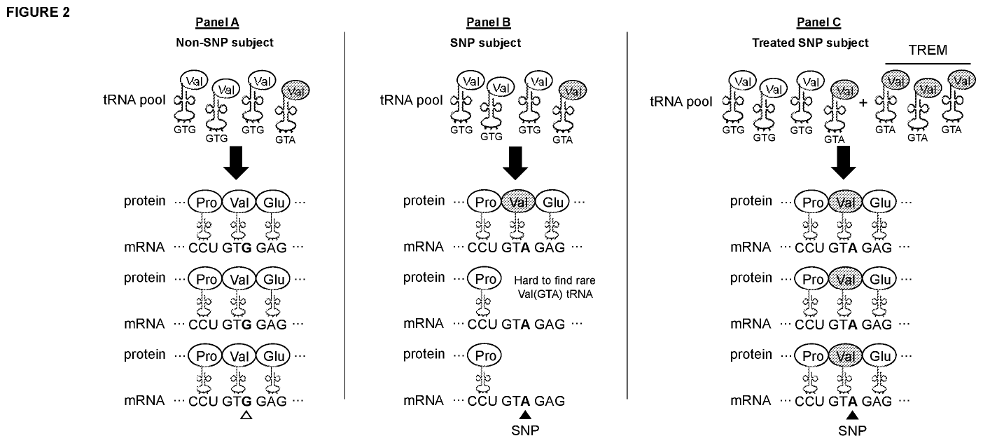

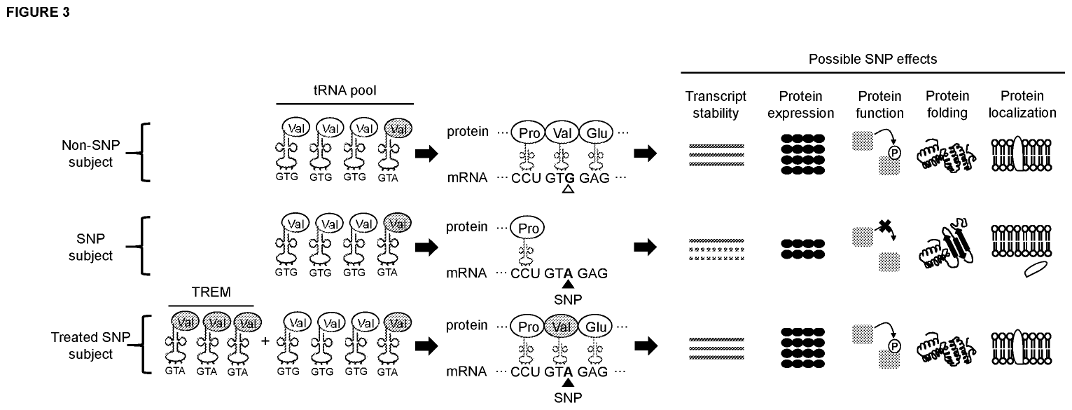

### DETAILED DESCRIPTION OF CERTAIN EMBODIMENTS

The present disclosure features, inter alia, methods of using tRNA-based effector molecules (TREMs) to modulate tRNA pools in a cell or a subject. Also disclosed herein are methods of treating a disorder or ameliorating a symptom of a disorder by administering a composition comprising a TREM or a pharmaceutical composition comprising a TREM. As disclosed herein tRNA-based effector molecules (TREMs) are complex molecules which can mediate a variety of cellular processes. Pharmaceutical compositions comprising a TREM can be administered to a cell, a tissue, or to a subject to modulate these functions.

### Definitions

“Acquire” or “acquiring” as the terms are used herein, refer to obtaining possession of a value, e.g., a numerical value, by “directly acquiring” or “indirectly acquiring” the physical entity or value. “Directly acquiring” refers to performing a process (e.g., performing an analytical method) to obtain the value. “Indirectly acquiring” refers to receiving the value from another party or source (e.g., a third party laboratory that directly acquired the or value).

A “cognate adaptor function TREM,” as that term is used herein, refers to a TREM which mediates initiation or elongation with the AA (the cognate AA) associated in nature with the anti-codon of the TREM.

A “decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulation that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

An “increased expression,” as that term is used herein, refers to an increase in comparison to a reference, e.g., in the case where altered control region, or addition of an agent, results in an increased expression of the subject product, it is increased relative to an otherwise similar cell without the alteration or addition.

An “isoacceptor,” as that term is used herein, refers to a plurality of tRNA molecule or TREMs wherein each molecule of the plurality comprises a different naturally occurring anticodon sequence and each molecule of the plurality mediates the incorporation of the same amino acid and that amino acid is the amino acid that naturally corresponds to the anticodons of the plurality.

A “non-cognate adaptor function TREM,” as that term is used herein, refers to a TREM which mediates initiation or elongation with an AA (a non-cognate AA) other than the AA associated in nature with the anticodon of the TREM. In an embodiment, a non-cognate adaptor function TREM is also referred to as a mischarged TREM (mTREM).

An “oncogene,” as that term is used herein, refers to a gene that modulates one or more cellular processes including: cell fate determination, cell survival and genome maintenance. In an embodiment, an oncogene provides a selective growth advantage to the cell in which it is present, e.g., deregulated, e.g., genetically deregulated (e.g., mutated or amplified) or epigenetically deregulated. Exemplary oncogenes include, Myc (e.g., c-Myc, N-Myc or L-Myc), c-Jun, Wnt, or RAS.

A “pharmaceutical composition,” as that term is used herein, refers to a composition that is suitable for pharmaceutical use. Typically, a pharmaceutical composition comprises a pharmaceutical excipient. In an embodiment, a pharmaceutical composition can comprise a TREM (a pharmaceutical composition comprising a TREM). In an embodiment the TREM will be the only active ingredient in a pharmaceutical composition comprising a TREM. In embodiments a pharmaceutical composition, e.g., a pharmaceutical composition comprising a TREM, is free, substantially free, or has less than a pharmaceutically acceptable amount, of host cell proteins, DNA, e.g., host cell DNA, endotoxins, and bacteria. In an embodiment, a pharmaceutical composition, e.g., a pharmaceutical composition comprising a TREM, is a GMP-grade composition in compliance with current good manufacturing practice (cGMP) guidelines, or other similar requirements. In an embodiment, a pharmaceutical composition, e.g., a pharmaceutical composition comprising a TREM is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 3.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “synthetic TREM,” as that term is used herein, refers to a TREM which was synthesized other than in a cell having an endogenous nucleic acid encoding the TREM, e.g., by cell-free solid phase synthesis. A synthetic TREM can have the same, or a different, sequence, set of post-transcriptional modifications, or tertiary structure, as a native tRNA.

A “TREM expressed in a heterologous cell,” as that term is used herein, refers to a TREM made under non-native conditions. E.g., a TREM, i) made in a cell that, differs, e.g., genetically, metabolically (e.g., has a different profile of gene expression or has a different level of a cellular component, e.g., an absorbed nutrient), or epigenetically, from a naturally occurring cell; ii) made in a cell that, is cultured under conditions, e.g., nutrition, pH, temperature, cell density, or stress conditions, that are different from native conditions (native conditions are the conditions under which a cell makes a tRNA in nature); or iii) was made in a cell at a level, at a rate, or at a concentration, or was localized in a compartment or location, that differs from a reference, e.g., at a level, at a rate, or at a concentration, or was localized in a compartment or location, that differs from that which occurs under native conditions. A TREM expressed in a heterologous cell can have the same, or a different, sequence, set of post-transcriptional modifications, or tertiary structure, as a native tRNA.

A “tRNA”, as that term is used herein, refers to a naturally occurring transfer ribonucleic acid in its native state.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. A TREM can have a plurality (e.g., 2, 3, 4, 5, 6, 7, 8, 9) of the structures and functions of (a)-(v).

In an embodiment, the TREM comprises an anticodon and can accept an amino acid and mediate the incorporation of the amino acid into a polypeptide chain, e.g., a naturally occurring tRNA or a tRNA described herein.

In an embodiment, a TREM is non-native, as evaluated by structure or the way in which it was made.

In an embodiment, a TREM comprises one or more of the following structures or properties:

(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 2. In an embodiment, the TREM can comprise a fragment or analog of an AStD, e.g., an AStD encoded by a nucleic acid in Table 2, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 2. E.g., one of ordinary skill can determine the sequence which corresponds to an AStD from a tRNA sequence encoded by a nucleic acid in Table 2);

(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 2. In an embodiment, the TREM can comprise a fragment or analog of a DHD, e.g., a DHD encoded by a nucleic acid in Table 2, which fragment in embodiments has DHD activity and in other embodiments does not have DHD activity;

(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 2. In an embodiment, the TREM can comprise a fragment or analog of an ACHD, e.g., an ACHD encoded by a nucleic acid in Table 2, which fragment in embodiments has ACHD activity and in other embodiments does not have ACHD activity;

(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 2. In an embodiment, the TREM can comprise a fragment or analog of a VLD, e.g., a VLD encoded by a nucleic acid in Table 2, which fragment in embodiments has VLD activity and in other embodiments does not have VLD activity;

(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 2. In an embodiment, the TREM can comprise a fragment or analog of a THD, e.g., a THD encoded by a nucleic acid in Table 2, which fragment in embodiments has THD activity and in other embodiments does not have THD activity;

(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 2. In an embodiment, the TREM can comprise a fragment or analog of a stem or loop structure, e.g., a stem or loop structure encoded by a nucleic acid in Table 2, which fragment in embodiments has activity of a stem or loop structure, and in other embodiments does not have activity of a stem or loop structure;

(g) a tertiary structure, e.g., an L-shaped tertiary structure;

(h) adaptor function, i.e., the TREM mediates acceptance of an amino acid, e.g., its cognate amino acid and transfer of the AA in the initiation or elongation of a polypeptide chain;

(i) cognate adaptor function wherein the TREM mediates acceptance and incorporation of an amino acid (e.g., cognate amino acid) associated in nature with the anti-codon of the TREM to initiate or elongate a polypeptide chain;

(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;

(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;

(l) a structure which allows for ribosome binding;

(m) a post-transcriptional modification, e.g., it comprises one or more modifications from Table 3, e.g., 1, 2, 3, 4, 5, 6, 7, 8, 9, 10, 11, 12, 13, 14, or 15 modifications listed in Table 3;

(n) the ability to inhibit a functional property of a tRNA, e.g., any of properties (h)-(k) possessed by a tRNA;

(o) the ability to modulate cell fate;

(p) the ability to modulate ribosome occupancy;

(q) the ability to modulate protein translation;

(r) the ability to modulate mRNA stability;

(s) the ability to modulate protein folding and structure;

(t) the ability to modulate protein transduction or compartmentalization;

(u) the ability to modulate protein stability;

(v) the ability to modulate a signaling pathway, e.g., a cellular signaling pathway;

(w) the anticodon does not pair with a stop codon, e.g., is an anticodon that pairs with other than UAG, UAA or UGA; or

(x) comprises an anticodon, can accept an amino acid and mediate the incorporation of the amino acid into a polypeptide chain, e.g., a naturally occurring tRNA or a tRNA described herein.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g)

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:

(i) an amino acid attachment domain that binds an amino acid (e.g., an AStD, as described in (a) herein; and

(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 2, or a fragment or functional fragment thereof. In an embodiment, a TREM comprises an RNA sequence encoded by a DNA sequence listed in Table 2, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 2, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 2, or a fragment or a functional fragment thereof. In an embodiment, a TREM comprises a TREM domain, e.g., a domain described herein, comprising an RNA sequence encoded by DNA sequence listed in Table 2, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 2, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “composition comprising a TREM” as that term is used herein, refers to a composition comprising a TREM described herein. A composition comprising a TREM can comprise one or more species of TREMs. In an embodiment, the composition comprises only a single species of TREM. In an embodiment, the composition comprises a first TREM species and a second TREM species. By way of example, in an embodiment, the first species and the second species are isoacceptors but have different sequences from one another. In an embodiment, the composition can comprise a first species that mediates the incorporation of a first amino acid, e.g., alanine, and a second species that mediates the incorporation of a second amino acid, e.g., lysine. In an embodiment, the composition comprises X TREM species, wherein X=2, 3, 4, 5, 6, 7, 8, 9, or 10. In an embodiment, the TREM has at least 70, 75, 80, 85, 90, or 95, or has 100%, identity with a sequence encoded by a nucleic acid in Table 2. In an embodiment, the TREM is purified from cell culture. In an embodiment, the cell culture from which the TREM is purified comprises at least 1×10⁷ host cells, 1×10⁸ host cells, 1×10⁹ host cells, 1×10¹⁰ host cells, 1×10¹¹ host cells, 1×10¹² host cells, 1×10¹³ host cells, or 1×10¹⁴ host cells. In an embodiment, the composition comprising the TREM is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid.

In an embodiment, the composition is dry, e.g., a lyophilized material. In an embodiment, the composition is a frozen composition. In an embodiment, the composition is sterile, e.g., the composition supports the growth of fewer than 100 viable microorganisms as tested under aseptic conditions, the composition meets the standard of USP <71>, and/or the composition meets the standard of USP <85>. In an embodiment, the composition comprises at least 0.5 g, 1.0 g, 50 g, 10 g, 15 g, 25 g, 50 g, 100 g, 200 g, 400 g, or 500 g (e.g., as determined by dry weight) of TREM.

A “tRNA pool,” as that term is used herein, refers to the pool of all species, e.g., endogenous tRNAs and TREMS, which can function as tRNAs. The endogenous tRNA pool for a cell or subject that has not been administered a TREM includes only endogenous tRNAs. A TREM can be added to modulate a tRNA pool comprising only endogenous tRNAs, but can also be administered to a cell or subject that has a tRNA pool that includes TREMs that have been administered previously. In an embodiment, the TREM which is administered to a cell or a subject, mediates initiation or elongation by incorporating the amino acid (the cognate amino acid) associated in nature with a particular anticodon. In an embodiment, the TREM which is administered has an anticodon other than a stop codon.

A “tumor suppressor,” as that term is used herein, refers to a gene that modulates one or more cellular processes including: cell fate determination, cell survival and genome maintenance. In an embodiment, a tumor suppressor provides a selective growth advantage to the cell in which it is deregulated, e.g., genetically deregulated (e.g., mutated or deleted) or epigenetically deregulated. Exemplary tumor suppressors include p53 or Rb.

“Pairs with” or “pairing,” as those terms are used herein, refer to the correspondence of a codon with an anticodon and includes fully complementary codon:anticodon pairs as well as “wobble” pairing, in which the third position need not be complementary. Fully complementary pairing refers to pairing of all three positions of the codon with the corresponding anticodon according to Watson-Crick base pairing. Wobble pairing refers to complementary pairing of the first and second positions of the codon with the corresponding anticodon according to Watson-Crick base pairing, and flexible pairing at the third position of the codon with the corresponding anticodon.

The terms modified, replace, derived and similar terms, when used or applied in reference to a product, refer only to the end product or structure of the end product, and are not restricted by any method of making or manufacturing the product, unless expressly provided as such in this disclosure.

Headings, titles, subtitles, numbering or other alpha/numeric hierarchies are included merely for ease of reading and absent explicit language to the contrary do not indicate order of performance, order of importance, magnitude or other value.

Synonymous SNPs and Method of Modulating tRNA Pool

A single nucleotide polymorphism (SNP) is a mutation that is found in the genome. A SNP can occur anywhere in the genome, e.g., in a coding sequence (e.g., an exon), or in a regulatory region (e.g., in an intron, a promoter element, an enhancer), or in a non-coding sequence.

A SNP that occurs in a coding sequence, e.g., an exon, can affect the corresponding polypeptide by altering a codon to specify a different amino acid, e.g., a different amino acid compared to that specified by the non-mutated codon.

A SNP that occurs in a coding sequence which alters a codon but does not change the amino acid specified by said mutated codon will not change the amino acid that is incorporated into the corresponding polypeptide at that position. This is possible due to the degeneracy of the genetic codon (i.e. more than one codon specifying one amino acid). Codon degeneracy is supported by “wobble” base pairing at the first base of the tRNA anticodon. For example, if a wildtype CTT codon which specifies the amino acid leucine is mutated to a CTC codon which specifies the same amino acid Leucine, no change to the corresponding protein with respect to its composition at that particular position is expected. Both codons CTT and CTC are recognized by tRNAs that specify the amino acid Leucine. These different species of tRNAs are referred to as isoacceptor tRNAs.

A mutation which changes a codon but does not change the corresponding amino acid specified by the mutated codon is called a synonymous SNP. Synonymous SNPs are also known as silent SNPs.

Synonymous SNPs found in the human population are linked to certain diseases. Since synonymous SNPs are not expected to alter the composition of the polypeptide chain, without wishing to be bound by theory, is it believed that the effect of a synonymous SNP is linked to bias in codon usage. For example, a synonymous SNP may result in reduced protein translation, altered protein folding, altered protein localization or altered protein function. The relationship between codon usage and tRNA abundance is currently being investigated.

In an embodiment, the amount of a tRNA in a cell is correlated with codon usage. In an embodiment, a tRNA which pairs with a codon that is highly used is more abundant than a tRNA which pairs with a codon that is not highly used. In an embodiment, a tRNA which pairs with a codon that is not highly used is less abundant than a tRNA which pairs with a codon that is highly used.

As defined herein, the tRNA pool in a cell is the tRNA pool of all species, e.g., endogenous tRNAs and TREMS, which can function as tRNAs. The endogenous tRNA pool for a cell or subject that has not been administered a TREM includes only endogenous tRNAs. The tRNA pool for a cell or subject that has been administered a TREM includes endogenous tRNAs and the TREM.

Without wishing to be bound by theory, it is believed that the tRNA pool in a cell or subject can be altered by administering a composition comprising a TREM to the cell or subject. In an embodiment, the tRNA pool in a cell or subject that has been administered a Composition comprising a TREM comprises endogenous tRNAs and the administered TREM.

In an embodiment, a subject or a cell having a synonymous SNP has a tRNA pool which has a lower abundance of the tRNA that pairs with the SNP codon. In an embodiment, administration of a TREM that pairs with the SNP codon to the subject or cell, increases the amount of the isoaccepting tRNA pool in the subject or cell, e.g., increase the amount of amino acid specifying molecule that can pair with the SNP codon.

Exemplary synonymous SNPs are provided in Table 1. The column with the heading “codon from/to” describes a wildtype codon for a particular transcript and the mutated codon. In an embodiment, a cell or subject described in a method of treatment, a method of modulating a tRNA pool, or a method of evaluation disclosed herein has a SNP provided in Table 1. In an embodiment, a cell or subject described in a method of treatment, a method of modulating a tRNA pool, or a method of evaluation disclosed herein has a disease listed in Table 1. In an embodiment, a cell or subject described in a method of treatment, a method of modulating a tRNA pool, or a method of evaluation disclosed herein has a SNP and the corresponding disease listed in Table 1.

TABLE 1
Exemplary SNPs and correlated diseases

| | | | | | Ratio of |
|---|---|---|---|---|---|
| | | | | | tRNA |
| | | | Codon | Anticodon | frequency |
| Disease Group | Disease | Transcript | from/To | from/to | (from/to) |
| Cardiovascular | Cardiac hypertrophy | ENST00000357596 | CTA/CTC | TAG/GAG | 19.3 |
| Cardiovascular | Cardiac hypertrophy | ENST00000357596 | CTA/CTG | TAG/CAG | 4.8 |
| Cardiovascular | Cardiac hypertrophy | ENST00000378319 | CTA/CTC | TAG/GAG | 19.3 |
| Cardiovascular | Cardiac hypertrophy | ENST00000378319 | CTA/CTG | TAG/CAG | 4.8 |
| Cardiovascular | Cardiac hypertrophy | ENST00000442195 | CTA/CTC | TAG/GAG | 19.3 |
| Cardiovascular | Cardiac hypertrophy | ENST00000442195 | CTA/CTG | TAG/CAG | 4.8 |
| Cardiovascular | Cardiac hypertrophy | ENST00000465045 | CTA/CTC | TAG/GAG | 19.3 |
| Cardiovascular | Cardiac hypertrophy | ENST00000465045 | CTA/CTG | TAG/CAG | 4.8 |
| Cardiovascular | Cardiac hypertrophy | ENST00000485919 | CTA/CTC | TAG/GAG | 19.3 |
| Cardiovascular | Cardiac hypertrophy | ENST00000485919 | CTA/CTG | TAG/CAG | 4.8 |
| Cardiovascular | Cardiac hypertrophy | ENST00000488958 | CTA/CTC | TAG/GAG | 19.3 |
| Cardiovascular | Cardiac hypertrophy | ENST00000488958 | CTA/CTG | TAG/CAG | 4.8 |
| Cardiovascular | Cardiac hypertrophy | ENST00000496623 | CTA/CTC | TAG/GAG | 19.3 |
| Cardiovascular | Cardiac hypertrophy | ENST00000496623 | CTA/CTG | TAG/CAG | 4.8 |
| Cardiovascular | Coronary artery disease | ENST00000375377 | CAC/CAT | GTG/ATG | 4.8 |
| Cardiovascular | Coronary artery disease | ENST00000375377 | CAC/CAT | GTG/ATG | 4.8 |
| Cardiovascular | Coronary artery disease | ENST00000413988 | GGC/GGT | GCC/ACC | 2235.1 |
| Cardiovascular | Hypertension | ENST00000229768 | GGA/GGG | TCC/CCC | 1.2 |
| Cardiovascular | Hypertension | ENST00000563726 | CCA/CCT | TGG/AGG | 16.7 |
| Cardiovascular | Hypertension | ENST00000563726 | CCA/CCG | TGG/CGG | 2.2 |
| Cardiovascular | Obesity-related traits | ENST00000318130 | ATC/ATT | GAT/AAT | 1443.7 |
| Cardiovascular | Obesity-related traits | ENST00000367512 | ATC/ATT | GAT/AAT | 1443.7 |
| Cardiovascular | Obesity-related traits | ENST00000439962 | ATC/ATT | GAT/AAT | 1443.7 |
| Cardiovascular | Type 1 diabetes | ENST00000261340 | AGC/AGT | GCT/ACT | 400.5 |
| Cardiovascular | Type 1 diabetes | ENST00000290855 | AGC/AGT | GCT/ACT | 400.5 |
| Cardiovascular | Type 1 diabetes | ENST00000325960 | AGC/AGT | GCT/ACT | 400.5 |
| Cardiovascular | Type 1 diabetes | ENST00000430909 | AGC/AGT | GCT/ACT | 400.5 |
| Cardiovascular | Type 1 diabetes | ENST00000444971 | AGC/AGT | GCT/ACT | 400.5 |
| Cardiovascular | Type 1 diabetes | ENST00000476198 | AGC/AGT | GCT/ACT | 400.5 |
| Cardiovascular | Type 1 diabetes | ENST00000479877 | AGC/AGT | GCT/ACT | 400.5 |
| Cardiovascular | Type 1 diabetes | ENST00000536355 | AGC/AGT | GCT/ACT | 400.5 |
| Cardiovascular | Type 1 diabetes | ENST00000543300 | AGC/AGT | GCT/ACT | 400.5 |
| Cardiovascular | Type 1 diabetes | ENST00000544322 | AGC/AGT | GCT/ACT | 400.5 |
| Cardiovascular | Type 1 diabetes | ENST00000263125 | CGA/AGA | TCG/TCT | 0.9 |
| Cardiovascular | Type 1 diabetes | ENST00000397176 | CGA/AGA | TCG/TCT | 0.9 |
| Cardiovascular | Type 1 diabetes | ENST00000539722 | CGA/AGA | TCG/TCT | 0.9 |
| Cardiovascular | Type 1 diabetes | ENST00000610727 | CGA/AGA | TCG/TCT | 0.9 |
| Cardiovascular | Type 2 diabetes | ENST00000226760 | AAC/AAT | GTT/ATT | 655.6 |
| Cardiovascular | Type 2 diabetes | ENST00000503569 | AAC/AAT | GTT/ATT | 655.6 |
| Dermatology | Psoriasis | ENST00000289431 | TGC/TGT | GCA/ACA | 111.3 |
| Dermatology | Psoriasis | ENST00000422556 | TGC/TGT | GCA/ACA | 111.3 |
| Endocrine | Endometriosis | ENST00000381434 | TAC/TAT | GTA/ATA | 268.1 |
| Endocrine | Endometriosis | ENST00000417746 | TAC/TAT | GTA/ATA | 268.1 |
| Endocrine | Endometriosis | ENST00000456383 | TAC/TAT | GTA/ATA | 268.1 |
| Endocrine | Endometriosis | ENST00000611532 | TAC/TAT | GTA/ATA | 268.1 |
| Immunology | Chronic inflammatory diseases | ENST00000171111 | GAC/GAT | GTC/ATC | 238.2 |
| | (ankylosing spondylitis, Crohn's | | | | |
| | disease, psoriasis, primary | | | | |
| | sclerosing_cholangitis, ulcerative | | | | |
| | colitis)(pleiotropy) | | | | |
| Immunology | Chronic inflammatory diseases | ENST00000393623 | GAC/GAT | GTC/ATC | 238.2 |
| | (ankylosing spondylitis, Crohn's | | | | |
| | disease, psoriasis, primary | | | | |
| | sclerosing_cholangitis, ulcerative | | | | |
| | colitis)(pleiotropy) | | | | |
| Immunology | Chronic inflammatory diseases | ENST00000592478 | GAC/GAT | GTC/ATC | 238.2 |
| | (ankylosing spondylitis, Crohn's | | | | |
| | disease, psoriasis, primary | | | | |
| | sclerosing_cholangitis, ulcerative | | | | |
| | colitis)(pleiotropy) | | | | |
| Immunology | Crohn's disease | ENST00000300590 | ATC/ATT | GAT/AAT | 1443.7 |
| Immunology | Crohn's disease | ENST00000330943 | ATC/ATT | GAT/AAT | 1443.7 |
| Immunology | Crohn's disease | ENST00000423026 | ATC/ATT | GAT/AAT | 1443.7 |
| Immunology | Crohn's disease | ENST00000568993 | ATC/ATT | GAT/AAT | 1443.7 |
| Immunology | Crohn's disease | ENST00000610485 | ATC/ATT | GAT/AAT | 1443.7 |
| Immunology | Graves' disease | ENST00000561890 | CAC/CAT | GTG/ATG | 4.8 |
| Neurology | Alzheimer disease and age of | ENST00000356711 | AAG/AAA | CTT/TTT | 2.1 |
| | onset | | | | |
| Neurology | Alzheimer disease and age of | ENST00000357550 | AAG/AAA | CTT/TTT | 2.1 |
| | onset | | | | |
| Neurology | Alzheimer disease and age of | ENST00000457511 | AAG/AAA | CTT/TTT | 2.1 |
| | onset | | | | |
| Neurology | Alzheimer's disease or family | ENST00000198536 | AGG/CGG | CCT/CCG | 1.3 |
| | history of Alzheimer's disease | | | | |
| Neurology | Alzheimer's disease or family | ENST00000350573 | AGG/CGG | CCT/CCG | 1.3 |
| | history of Alzheimer's disease | | | | |
| Neurology | Alzheimer's disease or family | ENST00000394000 | AGG/CGG | CCT/CCG | 1.3 |
| | history of Alzheimer's disease | | | | |
| Neurology | Alzheimer's disease or family | ENST00000432297 | AGG/CGG | CCT/CCG | 1.3 |
| | history of Alzheimer's disease | | | | |
| Neurology | Alzheimer's disease or family | ENST00000453419 | AGG/CGG | CCT/CCG | 1.3 |
| | history of Alzheimer's disease | | | | |
| Neurology | Major depressive disorder | ENST00000292591 | CTG/TTG | CAG/CAA | 1.4 |
| Neurology | Major depressive disorder | ENST00000337755 | CTG/TTG | CAG/CAA | 1.4 |
| Neurology | Major depressive disorder | ENST00000519836 | CTG/TTG | CAG/CAA | 1.4 |
| Neurology | Major depressive disorder | ENST00000520918 | CTG/TTG | CAG/CAA | 1.4 |
| Neurology | Major depressive disorder | ENST00000373352 | TGC/TGT | GCA/ACA | 111.3 |
| Neurology | Major depressive disorder | ENST00000373371 | TGC/TGT | GCA/ACA | 111.3 |
| Neurology | Major depressive disorder | ENST00000430147 | TGC/TGT | GCA/ACA | 111.3 |
| Neurology | Major depressive disorder | ENST00000451404 | TGC/TGT | GCA/ACA | 111.3 |
| Neurology | Major depressive disorder | ENST00000610552 | TGC/TGT | GCA/ACA | 111.3 |
| Neurology | Migraine | ENST00000348159 | AAC/AAT | GTT/ATT | 655.6 |
| Neurology | Migraine | ENST00000360595 | AAC/AAT | GTT/ATT | 655.6 |
| Neurology | Migraine | ENST00000368240 | AAC/AAT | GTT/ATT | 655.6 |
| Neurology | Migraine | ENST00000475587 | AAC/AAT | GTT/ATT | 655.6 |
| Neurology | Parkinson's disease | ENST00000262407 | GTC/GTT | GAC/AAC | 725.5 |
| Neurology | Parkinson's disease | ENST00000587295 | GTC/GTT | GAC/AAC | 725.5 |
| Neurology | Parkinson's disease | ENST00000648408 | GTC/GTT | GAC/AAC | 725.5 |
| Neurology | Parkinson's disease | ENST00000398238 | AAG/AAA | CTT/TTT | 2.1 |
| Neurology | Parkinson's disease | ENST00000465370 | AAG/AAA | CTT/TTT | 2.1 |
| Neurology | Parkinson's disease | ENST00000575068 | AAG/AAA | CTT/TTT | 2.1 |
| Neurology | Parkinson's disease | ENST00000576346 | AAG/AAA | CTT/TTT | 2.1 |
| Neurology | Schizophrenia | ENST00000304743 | CTG/CTC | CAG/GAG | 4.0 |
| Neurology | Schizophrenia | ENST00000439984 | CTG/CTC | CAG/GAG | 4.0 |
| Oncology | Adverse response to | ENST00000282849 | GTG/GTA | CAC/TAC | 5.2 |
| | chemotherapy | | | | |
| | (neutropenia/leucopenia)(all | | | | |
| | antimicrotubule drugs) | | | | |
| Oncology | Breast cancer | ENST00000360962 | GTG/GTC | CAC/GAC | 1.2 |
| Oncology | Breast cancer | ENST00000525449 | GTG/GTC | CAC/GAC | 1.2 |
| Oncology | Breast cancer | ENST00000525908 | GTG/GTC | CAC/GAC | 1.2 |
| Oncology | Breast cancer | ENST00000527634 | GTG/GTC | CAC/GAC | 1.2 |
| Oncology | Breast cancer | ENST00000532565 | GTG/GTC | CAC/GAC | 1.2 |
| Oncology | Breast cancer | ENST00000540737 | GTG/GTC | CAC/GAC | 1.2 |
| Oncology | Breast cancer | ENST00000642265 | GTG/GTC | CAC/GAC | 1.2 |
| Oncology | Breast cancer | ENST00000359435 | AAG/AAA | CTT/TTT | 2.1 |
| Oncology | Breast cancer | ENST00000447614 | AAG/AAA | CTT/TTT | 2.1 |
| Oncology | Breast cancer | ENST00000595632 | AAG/AAA | CTT/TTT | 2.1 |
| Oncology | Breast cancer | ENST00000598188 | AAG/AAA | CTT/TTT | 2.1 |
| Oncology | Breast cancer | ENST00000601043 | AAG/AAA | CTT/TTT | 2.1 |
| Oncology | Breast cancer (early onset) | ENST00000399503 | ACC/ACT | GGT/AGT | 1099.1 |
| Oncology | Breast cancer (early onset) | ENST00000399503 | ACC/ACT | GGT/AGT | 1099.1 |
| Oncology | Carboplatin disposition in | ENST00000370449 | CAC/CAT | GTG/ATG | 4.8 |
| | epthelial ovarian cancer | | | | |
| Oncology | Carboplatin disposition in | ENST00000647814 | CAC/CAT | GTG/ATG | 4.8 |
| | epthelial ovarian cancer | | | | |
| Oncology | Clostridium difficile infection in | ENST00000335385 | CTC/CTT | GAG/AAG | 90.2 |
| | multiple myeloma | | | | |
| Oncology | Colorectal cancer | ENST00000349830 | CCC/CCG | GGG/CGG | 3.4 |
| Oncology | Colorectal cancer | ENST00000349830 | CCC/CCT | GGG/AGG | 25.7 |
| Oncology | Colorectal cancer | ENST00000398564 | CCC/CCG | GGG/CGG | 3.4 |
| Oncology | Colorectal cancer | ENST00000398564 | CCC/CCT | GGG/AGG | 25.7 |
| Oncology | Colorectal cancer | ENST00000518288 | CCC/CCG | GGG/CGG | 3.4 |
| Oncology | Colorectal cancer | ENST00000518288 | CCC/CCT | GGG/AGG | 25.7 |
| Oncology | Colorectal cancer | ENST00000520359 | CCC/CCG | GGG/CGG | 3.4 |
| Oncology | Colorectal cancer | ENST00000520359 | CCC/CCT | GGG/AGG | 25.7 |
| Oncology | Colorectal cancer | ENST00000522435 | CCC/CCG | GGG/CGG | 3.4 |
| Oncology | Colorectal cancer | ENST00000522435 | CCC/CCT | GGG/AGG | 25.7 |
| Oncology | Endometrial cancer | ENST00000247270 | AGC/AGT | GCT/ACT | 400.5 |
| | ((endometrioid histology) | | | | |
| Oncology | Esophageal squamous cell | ENST00000269187 | CTG/TTG | CAG/CAA | 1.4 |
| | cancer (length of survival) | | | | |
| Oncology | Esophageal squamous cell | ENST00000440549 | CTG/TTG | CAG/CAA | 1.4 |
| | cancer (length of survival) | | | | |
| Oncology | Esophageal squamous cell | ENST00000586829 | CTG/TTG | CAG/CAA | 1.4 |
| | cancer (length of survival) | | | | |
| Oncology | Esophageal squamous cell | ENST00000590986 | CTG/TTG | CAG/CAA | 1.4 |
| | cancer (length of survival) | | | | |
| Oncology | Esophageal squamous cell | ENST00000330794 | GTG/GTC | CAC/GAC | 1.2 |
| | carcinoma | | | | |
| Oncology | Esophageal squamous cell | ENST00000330794 | GTG/GTA | CAC/TAC | 5.2 |
| | carcinoma | | | | |
| Oncology | Esophageal squamous cell | ENST00000510817 | GTG/GTC | CAC/GAC | 1.2 |
| | carcinoma | | | | |
| Oncology | Esophageal squamous cell | ENST00000510817 | GTG/GTA | CAC/TAC | 5.2 |
| | carcinoma | | | | |
| Oncology | Glioblastoma | ENST00000320521 | CTC/CTT | GAG/AAG | 90.2 |
| Oncology | Lung cancer in ever smokers | ENST00000326828 | TAC/TAT | GTA/ATA | 268.1 |
| Oncology | Lung cancer in ever smokers | ENST00000348639 | TAC/TAT | GTA/ATA | 268.1 |
| Oncology | Lung cancer in ever smokers | ENST00000559658 | TAC/TAT | GTA/ATA | 268.1 |
| Oncology | Macrophage Migration | ENST00000344836 | GTG/GTC | CAC/GAC | 1.2 |
| | Inhibitory Factor levels | | | | |
| Oncology | Macrophage Migration | ENST00000381886 | GTG/GTC | CAC/GAC | 1.2 |
| | Inhibitory Factor levels | | | | |
| Oncology | Oralcavity and pharyngeal | ENST00000209665 | AGG/AGA | CCT/TCT | 1.1 |
| | cancer | | | | |
| Oncology | Oralcavity and pharyngeal | ENST00000437033 | AGG/AGA | CCT/TCT | 1.1 |
| | cancer | | | | |
| Oncology | Oralcavity and pharyngeal | ENST00000476959 | AGG/AGA | CCT/TCT | 1.1 |
| | cancer | | | | |
| Oncology | Oralcavity and pharyngeal | ENST00000482593 | AGG/AGA | CCT/TCT | 1.1 |
| | cancer | | | | |
| Oncology | Pancreatic cancer | ENST00000162330 | CTC/CTT | GAG/AAG | 90.2 |
| Oncology | Pancreatic cancer | ENST00000393420 | CTC/CTT | GAG/AAG | 90.2 |
| Oncology | Pancreatic cancer | ENST00000393422 | CTC/CTT | GAG/AAG | 90.2 |
| Oncology | Pancreatic cancer | ENST00000418647 | CTC/CTT | GAG/AAG | 90.2 |
| Oncology | Pancreatic cancer | ENST00000420641 | CTC/CTT | GAG/AAG | 90.2 |
| Oncology | Pancreatic cancer | ENST00000535626 | CTC/CTT | GAG/AAG | 90.2 |
| Oncology | Pancreatic cancer | ENST00000538440 | CTC/CTT | GAG/AAG | 90.2 |
| Oncology | Pancreatic cancer | ENST00000542031 | CTC/CTT | GAG/AAG | 90.2 |
| Ophthalmology | Myopia | ENST00000358540 | GTG/GTA | CAC/TAC | 5.2 |
| Ophthalmology | Myopia | ENST00000396166 | GTG/GTA | CAC/TAC | 5.2 |
| Ophthalmology | Myopia | ENST00000396168 | GTG/GTA | CAC/TAC | 5.2 |
| Ophthalmology | Myopia | ENST00000396170 | GTG/GTA | CAC/TAC | 5.2 |
| Ophthalmology | Myopia | ENST00000477854 | GTG/GTA | CAC/TAC | 5.2 |
| Ophthalmology | Myopia (pathological) | ENST00000336812 | ACG/ACT | CGT/AGT | 190.9 |
| Ophthalmology | Myopia (pathological) | ENST00000336812 | ACG/ACT | CGT/AGT | 190.9 |
| Respiratory | Asthma | ENST00000427077 | CTA/TTA | TAG/TAA | 2.0 |
| Respiratory | Asthma | ENST00000346243 | TCC/TCT | GGA/AGA | 5.7 |
| Respiratory | Asthma | ENST00000346872 | TCC/TCT | GGA/AGA | 5.7 |
| Respiratory | Asthma | ENST00000350532 | TCC/TCT | GGA/AGA | 5.7 |
| Respiratory | Asthma | ENST00000351680 | TCC/TCT | GGA/AGA | 5.7 |
| Respiratory | Asthma | ENST00000377944 | TCC/TCT | GGA/AGA | 5.7 |
| Respiratory | Asthma | ENST00000377945 | TCC/TCT | GGA/AGA | 5.7 |
| Respiratory | Asthma | ENST00000377952 | TCC/TCT | GGA/AGA | 5.7 |
| Respiratory | Asthma | ENST00000377958 | TCC/TCT | GGA/AGA | 5.7 |
| Respiratory | Asthma | ENST00000394189 | TCC/TCT | GGA/AGA | 5.7 |
| Respiratory | Asthma | ENST00000439016 | TCC/TCT | GGA/AGA | 5.7 |
| Respiratory | Asthma | ENST00000439167 | TCC/TCT | GGA/AGA | 5.7 |
| Respiratory | Asthma | ENST00000467757 | TCC/TCT | GGA/AGA | 5.7 |
| Respiratory | Asthma | ENST00000535189 | TCC/TCT | GGA/AGA | 5.7 |
| Respiratory | Asthma | ENST00000583368 | TCC/TCT | GGA/AGA | 5.7 |
| Respiratory | Asthma | ENST00000623724 | TCC/TCT | GGA/AGA | 5.7 |
| Respiratory | Post bronchodilator FEV1/FVC | ENST00000225275 | AAC/AAT | GTT/ATT | 655.6 |
| | ratio in COPD | | | | |
| Respiratory | Post bronchodilator FEV1/FVC | ENST00000240335 | CCC/CCA | GGG/TGG | 1.5 |
| | ratio in COPD | | | | |
| Respiratory | Post bronchodilator FEV1/FVC | ENST00000240335 | CCC/CCG | GGG/CGG | 3.4 |
| | ratio in COPD | | | | |
| Respiratory | Post bronchodilator FEV1/FVC | ENST00000240335 | CCC/CCT | GGG/AGG | 25.7 |
| | ratio in COPD | | | | |
| Respiratory | Post bronchodilator FEV1/FVC | ENST00000393853 | CCC/CCA | GGG/TGG | 1.5 |
| | ratio in COPD | | | | |
| Respiratory | Post bronchodilator FEV1/FVC | ENST00000393853 | CCC/CCG | GGG/CGG | 3.4 |
| | ratio in COPD | | | | |
| Respiratory | Post bronchodilator FEV1/FVC | ENST00000393853 | CCC/CCT | GGG/AGG | 25.7 |
| | ratio in COPD | | | | |
| Respiratory | Post bronchodilator FEV1/FVC | ENST00000642491 | CCC/CCA | GGG/TGG | 1.5 |
| | ratio in COPD | | | | |
| Respiratory | Post bronchodilator FEV1/FVC | ENST00000642491 | CCC/CCG | GGG/CGG | 3.4 |
| | ratio in COPD | | | | |
| Respiratory | Post bronchodilator FEV1/FVC | ENST00000642491 | CCC/CCT | GGG/AGG | 25.7 |
| | ratio in COPD | | | | |
| Respiratory | Post bronchodilator FEV1/FVC | ENST00000644296 | CCC/CCA | GGG/TGG | 1.5 |
| | ratio in COPD | | | | |
| Respiratory | Post bronchodilator FEV1/FVC | ENST00000644296 | CCC/CCG | GGG/CGG | 3.4 |
| | ratio in COPD | | | | |
| Respiratory | Post bronchodilator FEV1/FVC | ENST00000644296 | CCC/CCT | GGG/AGG | 25.7 |
| | ratio in COPD | | | | |

### Host Cells

A host cell is a cell (e.g., a cultured cell) that can be used for expression and/or purification of a TREM. In an embodiment, a host cell comprises a mammalian cell or a non-mammalian cell. In an embodiment, a host cell comprises a mammalian cell, e.g., a human cell, or a rodent cell. In an embodiment, a host cell comprises a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell. In an embodiment, a host cell comprises a cancer cell, e.g., a solid tumor cell (e.g., a breast cancer cell (e.g., a MCF7 cell), a pancreatic cell line (e.g. a MIA PaCa-2 cell), a lung cancer cell, or a prostate cancer cell, or a hematological cancer cell). In an embodiment, a host cell is a primary cell, e.g., a cell that has not been immortalized or a cell with a finite proliferation capacity. In an embodiment, a host cell is a cell derived from a subject, e.g., a patient.

In an embodiment, a host cell comprises a non-mammalian cell, e.g., a bacterial cell, a yeast cell or an insect cell. In an embodiment, a host cell comprises a bacterial cell, e.g., an E. coli cell. In an embodiment, a host cell comprises a yeast cell, e.g., a S. cerevisiae cell. In an embodiment, a host cell comprises an insect cell, e.g., a Sf-9 cell or a Hi5 cell.

In an embodiment, a host cell comprises a cell that expresses one or more tissue specific tRNAs. For example, a host cell can comprise a cell derived from a tissue associated with expression of a tRNA, e.g., a tissue specific tRNA. In an embodiment, a host cell that expresses a tissue specific tRNA is modified to express a TREM, or a fragment thereof.

In an embodiment, a host cell is a cell that can be maintained under conditions that allow for expression of a TREM.

In an embodiment, a host cell is capable of post-transcriptionally modifying the TREM, e.g., adding a post-transcriptional modification selected from Table 3. In an embodiment, a host cell expresses (e.g., naturally or heterologously) an enzyme listed in Table 3. In an embodiment, a host cell expresses (e.g., naturally or heterologously) an enzyme, e.g., an enzyme having nuclease activity (e.g., endonuclease activity or ribonuclease activity), e.g., or one or more of Dicer, Angiogenin, RNaseA, RNaseP, RNaseZ, Rny1 or PrrC.

### Method of Culturing Host Cell

A host cell can be cultured in a medium that promotes growth, e.g., proliferation or hyperproliferation of the host cell. A host cell can be cultured in a suitable media, e.g., any of the following media: DMEM, MEM, MEM alpha, RPMI, F-10 media, F-12 media, DMEM/F-12 media, IMDM, Medium 199, Leibovitz L-15, McCoys's 5A, MDCB media, or CMRL media. In an embodiment the media is supplemented with glutamine. In an embodiment, the media is not supplemented with glutamine. In an embodiment, a host cell is cultured in media that has an excess of nutrients, e.g., is not nutrient limiting.

A host cell can be cultured in a medium comprising or supplemented with one or a combination of growth factors, cytokines or hormones, e.g., one or a combination of serum (e.g., fetal bovine serum (FBS)), HEPES, fibroblast growth factor (FGFs), epidermal growth factors (EGFs), insulin-like growth factors (IGFs), transforming growth factor beta (TGFb), platelet derived growth factor (PDGFs), hepatocyte growth factor (HGFs), or tumor necrosis factor (TNFs).

A host cell, e.g., a non-mammalian host cell, can be cultured in any of the following media: Luria Broth, YPD media or Grace's Medium.

A host cell can also be cultured under conditions that induce stress, e.g., cellular stress, osmotic stress, translational stress, or oncogenic stress. In an embodiment, a host cell expressing a TREM, cultured under conditions that induce stress (e.g., as described herein) results in a fragment of the TREM, e.g., as described herein.

A host cell can be cultured under nutrient limiting conditions, e.g., the host cell is cultured in media that has a limited amount of one or more nutrients. Examples of nutrients that can be limiting are amino acids, lipids, carbohydrates, hormones, growth factors or vitamins. In an embodiment, a host cell expressing a TREM, cultured in media that has a limited amount of one or more nutrients, e.g., the media is nutrient starved, results in a fragment of the TREM, e.g., as described herein. In an embodiment, a host cell expressing a TREM, cultured in media that has a limited amount of one or more nutrients, e.g., the media is nutrient starved, results in a TREM that is uncharged (e.g. a uTREM).

A host cell can comprise an immortalized cell, e.g., a cell which expresses one or more enzymes involved in immortalization, e.g., TERT. In an embodiment, a host cell can be propagated indefinitely.

A host cell can be cultured in suspension or as a monolayer. Host cell cultures can be performed in a cell culture vessel or a bioreactor. Cell culture vessels include a cell culture dish, plate or flask. Exemplary cell culture vessels include 35 mm, 60 mm, 100 mm, or 150 mm dishes, multi-well plates (e.g., 6-well, 12-well, 24-well, 48-well or 96 well plates), or T-25, T-75 or T-160 flasks.

In an embodiment, a host cell can be cultured in a bioreactor. A bioreactor can be, e.g., a continuous flow batch bioreactor, a perfusion bioreactor, a batch process bioreactor or a fed batch bioreactor. A bioreactor can be maintained under conditions sufficient to express the TREM. The culture conditions can be modulated to optimize yield, purity or structure of the TREM. In an embodiment, a bioreactor comprises at least 1×10⁷, 1×10⁸, 1×10⁹, 1×10¹⁰, 1×10¹¹, 1×10¹², 1×10¹³, or 1×10¹⁴ host cells. In an embodiment, a bioreactor comprises between 1×10⁷ to 1×10¹⁴ host cells; between 1×10⁷ to 0.5×10¹⁴ host cells; between 1×10⁷ to 1×10¹³ host cells; between 1×10⁷ to 0.5×10¹³ host cells; between 1×10⁷ to 1×10¹² host cells; between 1×10⁷ to 0.5×10¹² host cells; between 1×10⁷ to 1×10¹¹ host cells; between 1×10⁷ to 0.5×10¹¹ host cells; between 1×10⁷ to 1×10¹⁰ host cells; between 1×10⁷ to 0.5×10¹⁰ host cells; between 1×10⁷ to 1×10⁹ host cells; between 1×10⁷ to 0.5×10⁹ host cells; between 1×10⁷ to 1×10⁸ host cells; between 1×10⁷ to 0.5×10⁸ host cells; between 0.5×10⁸ to 1×10¹⁴ host cells; between 1×10⁸ to 1×10¹⁴ host cells; between 0.5×10⁹ to 1×10¹⁴ host cells; between 1×10⁹ to 1×10¹⁴ host cells; between 0.5×10¹⁰ to 1×10¹⁴ host cells; between 1×10¹⁰ to 1×10¹⁴ host cells; between 0.5×10¹¹ to 1×10¹⁴ host cells; between 1×10¹¹ to 1×10¹⁴ host cells; between 0.5×10¹² to 1×10¹⁴ host cells; between 1×10¹² to 1×10¹⁴ host cells; between 0.5×10¹³ to 1×10¹⁴ host cells; between 1×10¹³ to 1×10¹⁴ host cells; or between 0.5×10¹³ to 1×10¹⁴ host cells.

In an embodiment, a bioreactor comprises at least 1×10⁵ host cells/mL, 2×10⁵ host cells/mL, 3×10⁵ host cells/mL, 4×10⁵ host cells/mL, 5×10⁵ host cells/mL, 6×10⁵ host cells/mL, 7×10⁵ host cells/mL, 8×10⁵ host cells/mL, 9×10⁵ host cells/mL, 1×10⁶ host cells/mL, 2×10⁶ host cells/mL, 3×10⁶ host cells/mL, 4×10⁶ host cells/mL, 5×10⁶ host cells/mL, 6×10⁶ host cells/mL, 7×10⁶ host cells/mL, 8×10⁶ host cells/mL, 9×10⁶ host cells/mL, 1×10⁷ host cells/mL, 2×10⁷ host cells/mL, 3×10⁷ host cells/mL, 4×10⁷ host cells/mL, 5×10⁷ host cells/mL, 6×10⁷ host cells/mL, 7×10⁷ host cells/mL, 8×10⁷ host cells/mL, 9×10⁷ host cells/mL, 1×10⁸ host cell/mL, 2×10⁸ host cells/mL, 3×10⁸ host cells/mL, 4×10⁸ host cells/mL, 5×10⁸ host cells/mL, 6×10⁸ host cells/mL, 7×10⁸ host cells/mL, 8×10⁸ host cells/mL, 9×10⁸ host cells/mL, or 1×10⁹ host cells/mL. In an embodiment, a bioreactor comprises between 1×10⁵ host cells/mL to 1×10⁹ host cells/mL, between 5×10⁵ host cells/mL to 1×10⁹ host cells/mL, between 1×10⁶ host cells/mL to 1×10⁹ host cells/mL; between 5×10⁶ host cells/mL to 1×10⁹ host cells/mL, between 1×10⁷ host cells/mL to 1×10⁹ host cells/mL, between 5×10⁷ host cells/mL to 1×10⁹ host cells/mL, between 1×10⁸ host cells/mL to 1×10⁹ host cells/mL, between 5×10⁸ host cells/mL to 1×10⁹ host cells/mL, between 1×10⁵ host cells/mL to 5×10⁸ host cells/mL, between 1×10⁵ host cells/mL to 1×10⁸ host cells/mL, between 1×10⁵ host cells/mL to 5×10⁷ host cells/mL, between 1×10⁵ host cells/mL to 1×10⁷ host cells/mL, between 1×10⁵ host cells/mL to 5×10⁶ host cells/mL, between 1×10⁵ host cells/mL to 1×10⁶ host cells/mL, or between 1×10⁵ host cells/mL to 5×10⁵ host cells/mL.

In an embodiment, a batch process bioreactor comprises 1×10⁶ to 1×10⁷ host cells/ml. In an embodiment, a batch process bioreactor with a 100 mL volume comprises 1×10⁸ to 1×10⁹ host cells.

In an embodiment, a batch process bioreactor with a 100 L volume comprises 1×10¹¹ to 1×10¹² host cells.

In an embodiment, a fed batch bioreactor comprises 1×10⁷ to 3×10⁷ host cells/ml.

In an embodiment, a fed batch bioreactor with a 100 mL volume comprises 1×10⁹ to 3×10⁹ host cells.

In an embodiment, a fed batch bioreactor with a 100 L volume comprises 1×10¹² to 3×10¹² host cells.

In an embodiment, a perfusion bioreactor comprises 1×10⁸ host cells/ml.

In an embodiment, a perfusion bioreactor with a 100 mL volume comprises 1×10¹⁰ host cells.

In an embodiment, a perfusion bioreactor with a 100 L volume comprises 1×10¹³ host cells.

In an embodiment, a bioreactor is maintained under conditions that promote growth of the host cell, e.g., at a temperature (e.g., 37° C.) and gas concentration (e.g., 5% CO₂) that is permissive for growth of the host cell.

For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Exemplary bioreactor units, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. Any suitable bioreactor diameter can be used.

In an embodiment, the bioreactor can have a volume between about 100 mL and about 100 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters. Additionally, suitable reactors can be multi-use, single-use, disposable, or non-disposable and can be formed of any suitable material including metal alloys such as stainless steel (e.g., 316 L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

### Method of Modifying Host Cells

A host cell can be modified to optimize the production of a TREM, e.g., to have optimized TREM yield, purity, structure (e.g., folding), or stability. In an embodiment, a host cell can be modified (e.g., using a method described herein), to increase or decrease the expression of a desired molecule, e.g., gene, which optimizes production of the TREM, e.g., optimizes yield, purity, structure or stability of the TREM. In an embodiment, a host cell can be epigenetically modified, e.g., using a method described herein, to increase or decrease the expression of a desired gene, which optimizes production.

In an embodiment, a host cell can be modified to increase or decrease the expression of an oncogene (e.g., as described herein), a tumor suppressor (e.g., as described herein) or a molecule involved in tRNA or TREM modulation (e.g., a gene involved in tRNA or TREM transcription, processing, modification, stability or folding). Exemplary oncogenes include Myc (e.g., c-Myc, N-Myc or L-Myc), c-Jun, Wnt, or RAS. Exemplary tumor suppressors include p53 or Rb. Exemplary molecules involved in tRNA or TREM modulation include: RNA Polymerase III (Pol III) and Pol III accessory molecules (e.g., TFIIIB); Maf1, Trm1, Mck1 or Kns 1; enzymes involved in tRNA or TREM modification, e.g., genes listed in Table 3; or molecules with nuclease activity, e.g., or one or more of Dicer, Angiogenin, RNaseA, RNaseP, RNaseZ, Rny1 or PrrC.

In an embodiment, a host cell can be modified by: transfection (e.g., transient transfection or stable transfection); transduction (e.g., viral transduction, e.g., lentiviral, adenoviral or retroviral transduction); electroporation; lipid-based delivery of an agent (e.g., liposomes), nanoparticle based delivery of an agent; or other methods known in the art.

In an embodiment, a host cell can be modified to increase the expression of, e.g., overexpress, a desired molecule, e.g., a gene (e.g., an oncogene, or a gene involved in tRNA or TREM modulation (e.g., a gene encoding an enzyme listed in Table 3, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC. Exemplary methods of increasing the expression of a gene include: (a) contacting the host cell with a nucleic acid (e.g., DNA, or RNA) encoding the gene; (b) contacting the host cell with a peptide that expresses the target protein; (c) contacting the host cell with a molecule (e.g., a small RNA (e.g., a micro RNA, or a small interfering RNA) or a low molecular weight compound) that modulates, e.g., increases the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of a negative regulator of the target gene. In an embodiment, a nucleic acid encoding the gene, or a plasmid containing a nucleic acid encoding the gene can be introduced into the host cell by transfection or electroporation. In an embodiment, a nucleic acid encoding a gene can be introduced into the host cell by contacting the host cell with a virus (e.g., a lentivirus, adenovirus or retrovirus) expressing the gene.

In an embodiment, a host cell can be modified to decrease the expression of, e.g., minimize the expression, of a desired molecule, e.g., a gene (e.g., a tumor suppressor, or a gene involved in tRNA or TREM modulation). Exemplary methods of decreasing the expression of a gene include: (a) contacting the host cell with a nucleic acid (e.g., DNA, or RNA) encoding an inhibitor of the gene (e.g., a dominant negative variant or a negative regulator of the gene or protein encoded by the gene); (b) contacting the host cell with a peptide that inhibits the target protein; (c) contacting the host cell with a molecule (e.g., a small RNA (e.g., a micro RNA, or a small interfering RNA) or a low molecular weight compound) that modulates, e.g., inhibits the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of the target gene. In an embodiment, a nucleic acid encoding an inhibitor of the gene, or a plasmid containing a nucleic acid encoding an inhibitor of the gene can be introduced into the host cell by transfection or electroporation. In an embodiment, a nucleic acid encoding an inhibitor of the gene can be introduced into the host cell by contacting the host cell with a virus (e.g., a lentivirus, adenovirus or retrovirus) expressing the inhibitor of the gene.

In an embodiment, a host cell (e.g., a host cell described herein) is modified (e.g., by transfection with a nucleic acid), to express, e.g., overexpress, an oncogene, e.g., an oncogene described herein, e.g., c-Myc.

In an embodiment, a host cell (e.g., a host cell described herein) is modified (e.g., by transfection with a nucleic acid), to repress, e.g., downregulate, expression of a tumor suppressor, e.g., a tumor suppressor described herein, e.g., p53 or Rb.

In an embodiment, a host cell (e.g., a HEK293T cell) is modified (e.g., using a CRISPR/Cas9 molecule) to inhibit, e.g., knockout, expression of a gene that modulates a tRNA or TREM, e.g., Maf1. In an embodiment, a host cell (e.g., a HEK293T cell) is modified to overexpress a gene that modulates a tRNA or TREM, e.g., Trm1.

In an embodiment, a host cell (e.g., a HEK293T cell) is modified to overexpress a gene that modulates a tRNA or TREM, e.g., Trm1, and to overexpress an oncogene, e.g., an oncogene described herein, e.g., c-Myc.

### TREM

A “tRNA-based effector molecule” or “TREM” refers to an RNA molecule comprising one or more of the properties described herein. A TREM can be charged with an amino acid, e.g., a cognate amino acid; charged with a non-cognate amino acid (e.g., a mischarged TREM (mTREM); or not charged with an amino acid, e.g., an uncharged TREM (uTREM).

In an embodiment, a TREM comprises a ribonucleic acid (RNA) sequence encoded by a deoxyribonucleic acid (DNA) sequence disclosed in Table 2, e.g., any one of SEQ ID NOs: 1-451 disclosed in Table 2. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 2, e.g., any one of SEQ ID NOs: 1-451 disclosed in Table 2. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 2, e.g., any one of SEQ ID NOs: 1-451 disclosed in Table 2.

In an embodiment, a TREM comprises at least 30 consecutive nucleotides of an RNA sequence encoded by a DNA sequence disclosed in Table 2, e.g., at least 30 consecutive nucleotides of an RNA sequence encoded by any one of SEQ ID NOs: 1-451 disclosed in Table 2. In an embodiment, a TREM comprises at least 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 2, e.g., any one of SEQ ID NOs: 1-451 disclosed in Table 2. In an embodiment, a TREM comprises at least 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 2, e.g., any one of SEQ ID NOs: 1-451 disclosed in Table 2.

TABLE 2
List of tRNA sequences

| SEQ | | |
|---|---|---|
| ID | tRNA name | tRNA sequence |
| 1 | Ala_AGC_chr6: 287637 | GGGGGTATAGCTCAGTGGTAGAGCGCGTGCTTAGCATGCACGAGGTCCTGGGTTCGAT |
| | 41-28763812 (−) | CCCCAGTACCTCCA |
| 2 | Ala_AGC_chr6: 266874 | GGGGAATTAGCTCAAGTGGTAGAGCGCTTGCTTAGCACGCAAGAGGTAGTGGGATCG |
| | 85-26687557 (+) | ATGCCCACATTCTCCA |
| 3 | Ala_AGC_chr6: 265720 | GGGGAATTAGCTCAAATGGTAGAGCGCTCGCTTAGCATGCGAGAGGTAGCGGGATCG |
| | 92-26572164 (−) | ATGCCCGCATTCTCCA |
| 4 | Ala_AGC_chr6: 266827 | GGGGAATTAGCTCAAGTGGTAGAGCGCTTGCTTAGCATGCAAGAGGTAGTGGGATCG |
| | 15-26682787 (+) | ATGCCCACATTCTCCA |
| 5 | Ala_AGC_chr6: 267056 | GGGGAATTAGCTCAAGCGGTAGAGCGCTTGCTTAGCATGCAAGAGGTAGTGGGATCG |
| | 06-26705678 (+) | ATGCCCACATTCTCCA |
| 6 | Ala_AGC_chr6: 266735 | GGGGAATTAGCTCAAGTGGTAGAGCGCTTGCTTAGCATGCAAGAGGTAGTGGGATCA |
| | 90-26673662 (+) | ATGCCCACATTCTCCA |
| 7 | Ala_AGC_chr14: 89445 | GGGGAATTAGCTCAAGTGGTAGAGCGCTCGCTTAGCATGCGAGAGGTAGTGGGATCG |
| | 442-89445514 (+) | ATGCCCGCATTCTCCA |
| 8 | Ala_AGC_chr6: 581966 | GGGGAATTAGCCCAAGTGGTAGAGCGCTTGCTTAGCATGCAAGAGGTAGTGGGATCG |
| | 23-58196695 (−) | ATGCCCACATTCTCCA |
| 9 | Ala_AGC_chr6: 288062 | GGGGGTGTAGCTCAGTGGTAGAGCGCGTGCTTAGCATGCACGAGGCCCCGGGTTCAAT |
| | 21-28806292 (−) | CCCCGGCACCTCCA |
| 10 | Ala_AGC_chr6: 285749 | GGGGGTGTAGCTCAGTGGTAGAGCGCGTGCTTAGCATGTACGAGGTCCCGGGTTCAAT |
| | 33-28575004 (+) | CCCCGGCACCTCCA |
| 11 | Ala_AGC_chr6: 286260 | GGGGATGTAGCTCAGTGGTAGAGCGCATGCTTAGCATGCATGAGGTCCCGGGTTCGAT |
| | 14-28626085 (−) | CCCCAGCATCTCCA |
| 12 | Ala_AGC_chr6: 286783 | GGGGGTGTAGCTCAGTGGTAGAGCGCGTGCTTAGCATGCACGAGGCCCTGGGTTCAAT |
| | 66-28678437 (+) | CCCCAGCACCTCCA |
| 13 | Ala_AGC_chr6: 287798 | GGGGGTATAGCTCAGCGGTAGAGCGCGTGCTTAGCATGCACGAGGTCCTGGGTTCAAT |
| | 49-28779920 (−) | CCCCAATACCTCCA |
| 14 | Ala_AGC_chr6: 286874 | GGGGGTGTAGCTCAGTGGTAGAGCGCGTGCTTAGCATGCACGAGGCCCCGGGTTCAAT |
| | 81-28687552 (+) | CCCTGGCACCTCCA |
| 15 | Ala_AGC_chr2: 272740 | GGGGGATTAGCTCAAATGGTAGAGCGCTCGCTTAGCATGCGAGAGGTAGCGGGATCG |
| | 82-27274154 (+) | ATGCCCGCATCCTCCA |
| 16 | Ala_AGC_chr6: 267307 | GGGGAATTAGCTCAGGCGGTAGAGCGCTCGCTTAGCATGCGAGAGGTAGCGGGATCG |
| | 37-26730809 (+) | ACGCCCGCATTCTCCA |
| 17 | Ala_CGC_chr6: 265537 | GGGGATGTAGCTCAGTGGTAGAGCGCATGCTTCGCATGTATGAGGTCCCGGGTTCGAT |
| | 31-26553802 (+) | CCCCGGCATCTCCA |
| 18 | Ala_CGC_chr6: 286416 | GGGGATGTAGCTCAGTGGTAGAGCGCATGCTTCGCATGTATGAGGCCCCGGGTTCGAT |
| | 13-28641684 (−) | CCCCGGCATCTCCA |
| 19 | Ala_CGC_chr2: 157257 | GGGGATGTAGCTCAGTGGTAGAGCGCGCGCTTCGCATGTGTGAGGTCCCGGGTTCAAT |
| | 281-157257352 (+) | CCCCGGCATCTCCA |
| 20 | Ala_CGC_chr6: 286970 | GGGGGTGTAGCTCAGTGGTAGAGCGCGTGCTTCGCATGTACGAGGCCCCGGGTTCGAC |
| | 92-28697163 (+) | CCCCGGCTCCTCCA |
| 21 | Ala_TGC_chr6: 287575 | GGGGGTGTAGCTCAGTGGTAGAGCGCATGCTTTGCATGTATGAGGTCCCGGGTTCGAT |
| | 47-28757618 (−) | CCCCGGCACCTCCA |
| 22 | Ala_TGC_chr6: 286112 | GGGGATGTAGCTCAGTGGTAGAGCGCATGCTTTGCATGTATGAGGTCCCGGGTTCGAT |
| | 22-28611293 (+) | CCCCGGCATCTCCA |
| 23 | Ala_TGC_chr5: 180633 | GGGGATGTAGCTCAGTGGTAGAGCGCATGCTTTGCATGTATGAGGCCCCGGGTTCGAT |
| | 868-180633939 (+) | CCCCGGCATCTCCA |
| 24 | Ala_TGC_chr12: 12542 | GGGGATGTAGCTCAGTGGTAGAGCGCATGCTTTGCACGTATGAGGCCCCGGGTTCAAT |
| | 4512-125424583 (+) | CCCCGGCATCTCCA |
| 25 | Ala_TGC_chr6: 287850 | GGGGGTGTAGCTCAGTGGTAGAGCGCATGCTTTGCATGTATGAGGCCTCGGGTTCGAT |
| | 12-28785083 (−) | CCCCGACACCTCCA |
| 26 | Ala_TGC_chr6: 287261 | GGGGGTGTAGCTCAGTGGTAGAGCACATGCTTTGCATGTGTGAGGCCCCGGGTTCGAT |
| | 41-28726212 (−) | CCCCGGCACCTCCA |
| 27 | Ala_TGC_chr6: 287705 | GGGGGTGTAGCTCAGTGGTAGAGCGCATGCTTTGCATGTATGAGGCCTCGGTTCGATC |
| | 77-28770647 (−) | CCCGACACCTCCA |
| 28 | Arg_ACG_chr6: 263283 | GGGCCAGTGGCGCAATGGATAACGCGTCTGACTACGGATCAGAAGATTCCAGGTTCGA |
| | 68-26328440 (+) | CTCCTGGCTGGCTCG |
| 29 | Arg_ACG_chr3: 457304 | GGGCCAGTGGCGCAATGGATAACGCGTCTGACTACGGATCAGAAGATTCTAGGTTCGA |
| | 91-45730563 (−) | CTCCTGGCTGGCTCG |
| 30 | Arg_CCG_chr6: 287107 | GGCCGCGTGGCCTAATGGATAAGGCGTCTGATTCCGGATCAGAAGATTGAGGGTTCGA |
| | 29-28710801 (−) | GTCCCTTCGTGGTCG |
| 31 | Arg_CCG_chr17: 66016 | GACCCAGTGGCCTAATGGATAAGGCATCAGCCTCCGGAGCTGGGGATTGTGGGTTCGA |
| | 013-66016085 (−) | GTCCCATCTGGGTCG |
| 32 | Arg_CCT_chr17: 73030 | GCCCCAGTGGCCTAATGGATAAGGCACTGGCCTCCTAAGCCAGGGATTGTGGGTTCGA |
| | 001-73030073 (+) | GTCCCACCTGGGGTA |
| 33 | Arg_CCT_chr17: 73030 | GCCCCAGTGGCCTAATGGATAAGGCACTGGCCTCCTAAGCCAGGGATTGTGGGTTCGA |
| | 526-73030598 (−) | GTCCCACCTGGGGTG |
| 34 | Arg_CCT_chr16: 32029 | GCCCCGGTGGCCTAATGGATAAGGCATTGGCCTCCTAAGCCAGGGATTGTGGGTTCGA |
| | 01-3202973 (+) | GTCCCACCCGGGGTA |
| 35 | Arg_CCT_chr7: 139025 | GCCCCAGTGGCCTAATGGATAAGGCATTGGCCTCCTAAGCCAGGGATTGTGGGTTCGA |
| | 446-139025518 (+) | GTCCCATCTGGGGTG |
| 36 | Arg_CCT_chr16: 32439 | GCCCCAGTGGCCTGATGGATAAGGTACTGGCCTCCTAAGCCAGGGATTGTGGGTTCGA |
| | 18-3243990 (+) | GTTCCACCTGGGGTA |
| 37 | Arg_TCG_chr15: 89878 | GGCCGCGTGGCCTAATGGATAAGGCGTCTGACTTCGGATCAGAAGATTGCAGGTTCGA |
| | 304-89878376 (+) | GTCCTGCCGCGGTCG |
| 38 | Arg_TCG_chr6: 263230 | GACCACGTGGCCTAATGGATAAGGCGTCTGACTTCGGATCAGAAGATTGAGGGTTCGA |
| | 46-26323118 (+) | ATCCCTCCGTGGTTA |
| 39 | Arg_TCG_chr17: 73031 | GACCGCGTGGCCTAATGGATAAGGCGTCTGACTTCGGATCAGAAGATTGAGGGTTCGA |
| | 208-73031280 (+) | GTCCCTTCGTGGTCG |
| 40 | Arg_TCG_chr6: 262999 | GACCACGTGGCCTAATGGATAAGGCGTCTGACTTCGGATCAGAAGATTGAGGGTTCGA |
| | 05-26299977 (+) | ATCCCTTCGTGGTTA |
| 41 | Arg_TCG_chr6: 285108 | GACCACGTGGCCTAATGGATAAGGCGTCTGACTTCGGATCAGAAGATTGAGGGTTCGA |
| | 91-28510963 (−) | ATCCCTTCGTGGTTG |
| 42 | Arg_TCG_chr9: 112960 | GGCCGTGTGGCCTAATGGATAAGGCGTCTGACTTCGGATCAAAAGATTGCAGGTTTGA |
| | 803-112960875 (+) | GTTCTGCCACGGTCG |
| 43 | Arg_TCT_chr1: 943131 | GGCTCCGTGGCGCAATGGATAGCGCATTGGACTTCTAGAGGCTGAAGGCATTCAAAGG |
| | 29-94313213 (+) | TTCCGGGTTCGAGTCCCGGCGGAGTCG |
| 44 | Arg_TCT_chr17: 80242 | GGCTCTGTGGCGCAATGGATAGCGCATTGGACTTCTAGTGACGAATAGAGCAATTCAA |
| | 43-8024330 (+) | AGGTTGTGGGTTCGAATCCCACCAGAGTCG |
| 45 | Arg_TCT_chr9: 131102 | GGCTCTGTGGCGCAATGGATAGCGCATTGGACTTCTAGCTGAGCCTAGTGTGGTCATT |
| | 355-131102445 (−) | CAAAGGTTGTGGGTTCGAGTCCCACCAGAGTCG |
| 46 | Arg_TCT_chr11: 59318 | GGCTCTGTGGCGCAATGGATAGCGCATTGGACTTCTAGATAGTTAGAGAAATTCAAAG |
| | 767-59318852 (+) | GTTGTGGGTTCGAGTCCCACCAGAGTCG |
| 47 | Arg_TCT_chr1: 159111 | GTCTCTGTGGCGCAATGGACGAGCGCGCTGGACTTCTAATCCAGAGGTTCCGGGTTCG |
| | 401-159111474 (−) | AGTCCCGGCAGAGATG |
| 48 | Arg_TCT_chr6: 275299 | GGCTCTGTGGCGCAATGGATAGCGCATTGGACTTCTAGCCTAAATCAAGAGATTCAAA |
| | 63-27530049 (+) | GGTTGCGGGTTCGAGTCCCTCCAGAGTCG |
| 49 | Asn_GTT_chr1: 161510 | GTCTCTGTGGCGCAATCGGTTAGCGCGTTCGGCTGTTAACCGAAAGGTTGGTGGTTCG |
| | 031-161510104 (+) | ATCCCACCCAGGGACG |
| 50 | Asn_GTT_chr1: 143879 | GTCTCTGTGGCGCAATCGGCTAGCGCGTTTGGCTGTTAACTAAAAGGTTGGCGGTTCG |
| | 832-143879905 (−) | AACCCACCCAGAGGCG |
| 51 | Asn_GTT_chr1: 144301 | GTCTCTGTGGTGCAATCGGTTAGCGCGTTCCGCTGTTAACCGAAAGCTTGGTGGTTCGA |
| | 611-144301684 (+) | GCCCACCCAGGGATG |
| 52 | Asn_GTT_chr1: 149326 | GTCTCTGTGGCGCAATCGGCTAGCGCGTTTGGCTGTTAACTAAAAAGTTGGTGGTTCG |
| | 272-149326345 (−) | AACACACCCAGAGGCG |
| 53 | Asn_GTT_chr1: 148248 | GTCTCTGTGGCGCAATCGGTTAGCGCGTTCGGCTGTTAACCGAAAGGTTGGTGGTTCG |
| | 115-148248188 (+) | AGCCCACCCAGGGACG |
| 54 | Asn_GTT_chr1: 148598 | GTCTCTGTGGCGCAATCGGTTAGCGCATTCGGCTGTTAACCGAAAGGTTGGTGGTTCG |
| | 314-148598387 (−) | AGCCCACCCAGGGACG |
| 55 | Asn_GTT_chr1: 172161 | GTCTCTGTGGCGCAATCGGTTAGCGCGTTCGGCTGTTAACCGAAAGATTGGTGGTTCG |
| | 72-17216245 (+) | AGCCCACCCAGGGACG |
| 56 | Asn_GTT_chr1: 168470 | GTCTCTGTGGCGCAATCGGTTAGCGCGTTCGGCTGTTAACTGAAAGGTTGGTGGTTCG |
| | 80-16847153 (−) | AGCCCACCCAGGGACG |
| 57 | Asn_GTT_chr1: 149230 | GTCTCTGTGGCGCAATGGGTTAGCGCGTTCGGCTGTTAACCGAAAGGTTGGTGGTTCG |
| | 570-149230643 (−) | AGCCCATCCAGGGACG |
| 58 | Asn_GTT_chr1: 148000 | GTCTCTGTGGCGTAGTCGGTTAGCGCGTTCGGCTGTTAACCGAAAAGTTGGTGGTTCG |
| | 805-148000878 (+) | AGCCCACCCAGGAACG |
| 59 | Asn_GTT_chr1: 149711 | GTCTCTGTGGCGCAATCGGCTAGCGCGTTTGGCTGTTAACTAAAAGGTTGGTGGTTCG |
| | 798-149711871 (−) | AACCCACCCAGAGGCG |
| 60 | Asn_GTT_chr1: 145979 | GTCTCTGTGGCGCAATCGGTTAGCGCGTTCGGCTGTTAACTGAAAGGTTAGTGGTTCG |
| | 034-145979107 (−) | AGCCCACCCGGGGACG |
| 61 | Asp_GTC_chr12: 98897 | TCCTCGTTAGTATAGTGGTTAGTATCCCCGCCTGTCACGCGGGAGACCGGGGTTCAATT |
| | 281-98897352 (+) | CCCCGACGGGGAG |
| 62 | Asp_GTC_chr1: 161410 | TCCTCGTTAGTATAGTGGTGAGTATCCCCGCCTGTCACGCGGGAGACCGGGGTTCGAT |
| | 615-161410686 (−) | TCCCCGACGGGGAG |
| 63 | Asp_GTC_chr6: 275512 | TCCTCGTTAGTATAGTGGTGAGTGTCCCCGTCTGTCACGCGGGAGACCGGGGTTCGATT |
| | 36-27551307 (−) | CCCCGACGGGGAG |
| 64 | Cys_GCA_chr7: 149007 | GGGGGCATAGCTCAGTGGTAGAGCATTTGACTGCAGATCAAGAGGTCCCTGGTTCAAA |
| | 281-149007352 (+) | TCCAGGTGCCCCCT |
| 65 | Cys_GCA_chr7: 149074 | GGGGGTATAGCTCAGGGGTAGAGCATTTGACTGCAGATCAAGAGGTCCCTGGTTCAAA |
| | 601-149074672 (−) | TCCAGGTGCCCCCC |
| 66 | Cys_GCA_chr7: 149112 | GGGGGTATAGCTTAGCGGTAGAGCATTTGACTGCAGATCAAGAGGTCCCCGGTTCAAA |
| | 229-149112300 (−) | TCCGGGTGCCCCCT |
| 67 | Cys_GCA_chr7: 149344 | GGGGGTATAGCTTAGGGGTAGAGCATTTGACTGCAGATCAAAAGGTCCCTGGTTCAAA |
| | 046-149344117 (−) | TCCAGGTGCCCCTT |
| 68 | Cys_GCA_chr7: 149052 | GGGGGTATAGCTCAGGGGTAGAGCATTTGACTGCAGATCAAGAGGTCCCCAGTTCAAA |
| | 766-149052837 (−) | TCTGGGTGCCCCCT |
| 69 | Cys_GCA_chr17: 37017 | GGGGGTATAGCTCAGGGGTAGAGCATTTGACTGCAGATCAAGAAGTCCCCGGTTCAAA |
| | 937-37018008 (−) | TCCGGGTGCCCCCT |
| 70 | Cys_GCA_chr7: 149281 | GGGGGTATAGCTCAGGGGTAGAGCATTTGACTGCAGATCAAGAGGTCTCTGGTTCAAA |
| | 816-149281887 (+) | TCCAGGTGCCCCCT |
| 71 | Cys_GCA_chr7: 149243 | GGGGGTATAGCTCAGGGGTAGAGCACTTGACTGCAGATCAAGAAGTCCTTGGTTCAAA |
| | 631-149243702 (+) | TCCAGGTGCCCCCT |
| 72 | Cys_GCA_chr7: 149388 | GGGGATATAGCTCAGGGGTAGAGCATTTGACTGCAGATCAAGAGGTCCCCGGTTCAAA |
| | 272-149388343 (−) | TCCGGGTGCCCCCC |
| 73 | Cys_GCA_chr7: 149072 | GGGGGTATAGTTCAGGGGTAGAGCATTTGACTGCAGATCAAGAGGTCCCTGGTTCAAA |
| | 850-149072921 (−) | TCCAGGTGCCCCCT |
| 74 | Cys_GCA_chr7: 149310 | GGGGGTATAGCTCAGGGGTAGAGCATTTGACTGCAAATCAAGAGGTCCCTGATTCAAA |
| | 156-149310227 (−) | TCCAGGTGCCCCCT |
| 75 | Cys_GCA_chr4: 124430 | GGGGGTATAGCTCAGTGGTAGAGCATTTGACTGCAGATCAAGAGGTCCCCGGTTCAAA |
| | 005-124430076 (−) | TCCGGGTGCCCCCT |
| 76 | Cys_GCA_chr7: 149295 | GGGCGTATAGCTCAGGGGTAGAGCATTTGACTGCAGATCAAGAGGTCCCCAGTTCAAA |
| | 046-149295117 (+) | TCTGGGTGCCCCCT |
| 77 | Cys_GCA_chr7: 149361 | GGGGGTATAGCTCACAGGTAGAGCATTTGACTGCAGATCAAGAGGTCCCCGGTTCAAA |
| | 915-149361986 (+) | TCTGGGTGCCCCCT |
| 78 | Cys_GCA_chr7: 149253 | GGGCGTATAGCTCAGGGGTAGAGCATTTGACTGCAGATCAAGAGGTCCCCAGTTCAAA |
| | 802-149253871 (+) | TCTGGGTGCCCA |
| 79 | Cys_GCA_chr7: 149292 | GGGGGTATAGCTCACAGGTAGAGCATTTGACTGCAGATCAAGAGGTCCCCGGTTCAAA |
| | 305-149292376 (−) | TCCGGTTACTCCCT |
| 80 | Cys_GCA_chr7: 149286 | GGGGGTATAGCTCAGGGGTAGAGCACTTGACTGCAGATCAAGAGGTCCCTGGTTCAAA |
| | 164-149286235 (−) | TCCAGGTGCCCCCT |
| 81 | Cys_GCA_chr17: 37025 | GGGGGTATAGCTCAGTGGTAGAGCATTTGACTGCAGATCAAGAGGTCCCTGGTTCAAA |
| | 545-37025616 (−) | TCCGGGTGCCCCCT |
| 82 | Cys_GCA_chr15: 80036 | GGGGGTATAGCTCAGTGGGTAGAGCATTTGACTGCAGATCAAGAGGTCCCCGGTTCAA |
| | 997-80037069 (+) | ATCCGGGTGCCCCCT |
| 83 | Cys_GCA_chr3: 131947 | GGGGGTGTAGCTCAGTGGTAGAGCATTTGACTGCAGATCAAGAGGTCCCTGGTTCAAA |
| | 944-131948015 (−) | TCCAGGTGCCCCCT |
| 84 | Cys_GCA_chr1: 939818 | GGGGGTATAGCTCAGGTGGTAGAGCATTTGACTGCAGATCAAGAGGTCCCCGGTTCAA |
| | 34-93981906 (−) | ATCCGGGTGCCCCCT |
| 85 | Cys_GCA_chr14: 73429 | GGGGGTATAGCTCAGGGGTAGAGCATTTGACTGCAGATCAAGAGGTCCCCGGTTCAAA |
| | 679-73429750 (+) | TCCGGGTGCCCCCT |
| 86 | Cys_GCA_chr3: 131950 | GGGGGTATAGCTCAGGGGTAGAGCATTTGACTGCAGATCAAGAGGTCCCTGGTTCAAA |
| | 642-131950713 (−) | TCCAGGTGCCCCCT |
| 87 | Gln_CTG_chr6: 188364 | GGTTCCATGGTGTAATGGTTAGCACTCTGGACTCTGAATCCAGCGATCCGAGTTCAAA |
| | 02-18836473 (+) | TCTCGGTGGAACCT |
| 88 | Gln_CTG_chr6: 275155 | GGTTCCATGGTGTAATGGTTAGCACTCTGGACTCTGAATCCAGCGATCCGAGTTCAAG |
| | 31-27515602 (−) | TCTCGGTGGAACCT |
| 89 | Gln_CTG_chr1: 145963 | GGTTCCATGGTGTAATGGTGAGCACTCTGGACTCTGAATCCAGCGATCCGAGTTCGAG |
| | 304-145963375 (+) | TCTCGGTGGAACCT |
| 90 | Gln_CTG_chr1: 147737 | GGTTCCATGGTGTAATGGTAAGCACTCTGGACTCTGAATCCAGCGATCCGAGTTCGAG |
| | 382-147737453 (−) | TCTCGGTGGAACCT |
| 91 | Gln_CTG_chr6: 272632 | GGTTCCATGGTGTAATGGTTAGCACTCTGGACTCTGAATCCGGTAATCCGAGTTCAAAT |
| | 12-27263283 (+) | CTCGGTGGAACCT |
| 92 | Gln_CTG_chr6: 277591 | GGCCCCATGGTGTAATGGTCAGCACTCTGGACTCTGAATCCAGCGATCCGAGTTCAAA |
| | 35-27759206 (−) | TCTCGGTGGGACCC |
| 93 | Gln_CTG_chr1: 147800 | GGTTCCATGGTGTAATGGTAAGCACTCTGGACTCTGAATCCAGCCATCTGAGTTCGAG |
| | 937-147801008 (+) | TCTCTGTGGAACCT |
| 94 | Gln_TTG_chr17: 47269 | GGTCCCATGGTGTAATGGTTAGCACTCTGGACTTTGAATCCAGCGATCCGAGTTCAAA |
| | 890-47269961 (+) | TCTCGGTGGGACCT |
| 95 | Gln_TTG_chr6: 285571 | GGTCCCATGGTGTAATGGTTAGCACTCTGGACTTTGAATCCAGCAATCCGAGTTCGAA |
| | 56-28557227 (+) | TCTCGGTGGGACCT |
| 96 | Gln_TTG_chr6: 263114 | GGCCCCATGGTGTAATGGTTAGCACTCTGGACTTTGAATCCAGCGATCCGAGTTCAAA |
| | 24-26311495 (−) | TCTCGGTGGGACCT |
| 97 | Gln_TTG_chr6: 145503 | GGTCCCATGGTGTAATGGTTAGCACTCTGGGCTTTGAATCCAGCAATCCGAGTTCGAA |
| | 859-145503930 (+) | TCTTGGTGGGACCT |
| 98 | Glu_CTC_chr1: 145399 | TCCCTGGTGGTCTAGTGGTTAGGATTCGGCGCTCTCACCGCCGCGGCCCGGGTTCGATT |
| | 233-145399304 (−) | CCCGGTCAGGGAA |
| 99 | Glu_CTC_chr1: 249168 | TCCCTGGTGGTCTAGTGGTTAGGATTCGGCGCTCTCACCGCCGCGGCCCGGGTTCGATT |
| | 447-249168518 (+) | CCCGGTCAGGAAA |
| 100 | Glu_TTC_chr2: 131094 | TCCCATATGGTCTAGCGGTTAGGATTCCTGGTTTTCACCCAGGTGGCCCGGGTTCGACT |
| | 701-131094772 (−) | CCCGGTATGGGAA |
| 101 | Glu_TTC_chr13: 45492 | TCCCACATGGTCTAGCGGTTAGGATTCCTGGTTTTCACCCAGGCGGCCCGGGTTCGACT |
| | 062-45492133 (−) | CCCGGTGTGGGAA |
| 102 | Glu_TTC_chr1: 171990 | TCCCTGGTGGTCTAGTGGCTAGGATTCGGCGCTTTCACCGCCGCGGCCCGGGTTCGATT |
| | 78-17199149 (+) | CCCGGCCAGGGAA |
| 103 | Glu_TTC_chr1: 168617 | TCCCTGGTGGTCTAGTGGCTAGGATTCGGCGCTTTCACCGCCGCGGCCCGGGTTCGATT |
| | 74-16861845 (−) | CCCGGTCAGGGAA |
| 104 | Gly_CCC_chr1: 168724 | GCATTGGTGGTTCAGTGGTAGAATTCTCGCCTCCCACGCGGGAGACCCGGGTTCAATT |
| | 34-16872504 (−) | CCCGGCCAATGCA |
| 105 | Gly_CCC_chr2: 704761 | GCGCCGCTGGTGTAGTGGTATCATGCAAGATTCCCATTCTTGCGACCCGGGTTCGATTC |
| | 23-70476193 (−) | CCGGGCGGCGCA |
| 106 | Gly_CCC_chr17: 19764 | GCATTGGTGGTTCAATGGTAGAATTCTCGCCTCCCACGCAGGAGACCCAGGTTCGATT |
| | 175-19764245 (+) | CCTGGCCAATGCA |
| 107 | Gly_GCC_chr1: 161413 | GCATGGGTGGTTCAGTGGTAGAATTCTCGCCTGCCACGCGGGAGGCCCGGGTTCGATT |
| | 094-161413164 (+) | CCCGGCCCATGCA |
| 108 | Gly_GCC_chr1: 161493 | GCATTGGTGGTTCAGTGGTAGAATTCTCGCCTGCCACGCGGGAGGCCCGGGTTCGATT |
| | 637-161493707 (−) | CCCGGCCAATGCA |
| 109 | Gly_GCC_chr16: 70812 | GCATTGGTGGTTCAGTGGTAGAATTCTCGCCTGCCACGCGGGAGGCCCGGGTTTGATT |
| | 114-70812184 (−) | CCCGGCCAGTGCA |
| 110 | Gly_GCC_chr1: 161450 | GCATAGGTGGTTCAGTGGTAGAATTCTTGCCTGCCACGCAGGAGGCCCAGGTTTGATT |
| | 356-161450426 (+) | CCTGGCCCATGCA |
| 111 | Gly_GCC_chr16: 70822 | GCATTGGTGGTTCAGTGGTAGAATTCTCGCCTGCCATGCGGGCGGCCGGGCTTCGATT |
| | 597-70822667 (+) | CCTGGCCAATGCA |
| 112 | Gly_TCC_chr19: 47240 | GCGTTGGTGGTATAGTGGTTAGCATAGCTGCCTTCCAAGCAGTTGACCCGGGTTCGATT |
| | 82-4724153 (+) | CCCGGCCAACGCA |
| 113 | Gly_TCC_chr1: 145397 | GCGTTGGTGGTATAGTGGTGAGCATAGCTGCCTTCCAAGCAGTTGACCCGGGTTCGAT |
| | 864-145397935 (−) | TCCCGGCCAACGCA |
| 114 | Gly_TCC_chr17: 81248 | GCGTTGGTGGTATAGTGGTAAGCATAGCTGCCTTCCAAGCAGTTGACCCGGGTTCGAT |
| | 66-8124937 (+) | TCCCGGCCAACGCA |
| 115 | Gly_TCC_chr1: 161409 | GCGTTGGTGGTATAGTGGTGAGCATAGTTGCCTTCCAAGCAGTTGACCCGGGCTCGAT |
| | 961-161410032 (−) | TCCCGCCCAACGCA |
| 116 | His_GTG_chr1: 145396 | GCCGTGATCGTATAGTGGTTAGTACTCTGCGTTGTGGCCGCAGCAACCTCGGTTCGAAT |
| | 881-145396952 (−) | CCGAGTCACGGCA |
| 117 | His_GTG_chr1: 149155 | GCCATGATCGTATAGTGGTTAGTACTCTGCGCTGTGGCCGCAGCAACCTCGGTTCGAA |
| | 828-149155899 (−) | TCCGAGTCACGGCA |
| 118 | Ile_AAT_chr6: 5814925 | GGCCGGTTAGCTCAGTTGGTTAGAGCGTGGCGCTAATAACGCCAAGGTCGCGGGTTCG |
| | 4-58149327 (+) | ATCCCCGTACGGGCCA |
| 119 | Ile_AAT_chr6: 2765596 | GGCCGGTTAGCTCAGTTGGTTAGAGCGTGGTGCTAATAACGCCAAGGTCGCGGGTTCG |
| | 7-27656040 (+) | ATCCCCGTACTGGCCA |
| 120 | Ile_AAT_chr6: 2724299 | GGCTGGTTAGCTCAGTTGGTTAGAGCGTGGTGCTAATAACGCCAAGGTCGCGGGTTCG |
| | 0-27243063 (−) | ATCCCCGTACTGGCCA |
| 121 | Ile_AAT_chr17: 813030 | GGCCGGTTAGCTCAGTTGGTTAGAGCGTGGTGCTAATAACGCCAAGGTCGCGGGTTCG |
| | 9-8130382 (−) | AACCCCGTACGGGCCA |
| 122 | Ile_AAT_chr6: 2655435 | GGCCGGTTAGCTCAGTTGGTTAGAGCGTGGTGCTAATAACGCCAAGGTCGCGGGTTCG |
| | 0-26554423 (+) | ATCCCCGTACGGGCCA |
| 123 | Ile_AAT_chr6: 2674525 | GGCCGGTTAGCTCAGTTGGTTAGAGCGTGGTGCTAATAACGCTAAGGTCGCGGGTTCG |
| | 5-26745328 (−) | ATCCCCGTACTGGCCA |
| 124 | Ile_AAT_chr6: 2672122 | GGCCGGTTAGCTCAGTTGGTCAGAGCGTGGTGCTAATAACGCCAAGGTCGCGGGTTCG |
| | 1-26721294 (−) | ATCCCCGTACGGGCCA |
| 125 | Ile_AAT_chr6: 2763636 | GGCCGGTTAGCTCAGTCGGCTAGAGCGTGGTGCTAATAACGCCAAGGTCGCGGGTTCG |
| | 2-27636435 (+) | ATCCCCGTACGGGCCA |
| 126 | Ile_AAT_chr6: 2724173 | GGCTGGTTAGTTCAGTTGGTTAGAGCGTGGTGCTAATAACGCCAAGGTCGTGGGTTCG |
| | 9-27241812 (+) | ATCCCCATATCGGCCA |
| 127 | Ile_GAT_chrX: 375641 | GGCCGGTTAGCTCAGTTGGTAAGAGCGTGGTGCTGATAACACCAAGGTCGCGGGCTCG |
| | 8-3756491 (−) | ACTCCCGCACCGGCCA |
| 128 | Ile_TAT_chr19: 399028 | GCTCCAGTGGCGCAATCGGTTAGCGCGCGGTACTTATATGACAGTGCGAGCGGAGCAA |
| | 08-39902900 (−) | TGCCGAGGTTGTGAGTTCGATCCTCACCTGGAGCA |
| 129 | Ile_TAT_chr2: 4303767 | GCTCCAGTGGCGCAATCGGTTAGCGCGCGGTACTTATACAGCAGTACATGCAGAGCAA |
| | 6-43037768 (+) | TGCCGAGGTTGTGAGTTCGAGCCTCACCTGGAGCA |
| 130 | Ile_TAT_chr6: 2698812 | GCTCCAGTGGCGCAATCGGTTAGCGCGCGGTACTTATATGGCAGTATGTGTGCGAGTG |
| | 5-26988218 (+) | ATGCCGAGGTTGTGAGTTCGAGCCTCACCTGGAGCA |
| 131 | Ile_TAT_chr6: 2759920 | GCTCCAGTGGCGCAATCGGTTAGCGCGCGGTACTTATACAACAGTATATGTGCGGGTG |
| | 0-27599293 (+) | ATGCCGAGGTTGTGAGTTCGAGCCTCACCTGGAGCA |
| 132 | Ile_TAT_chr6: 2850536 | GCTCCAGTGGCGCAATCGGTTAGCGCGCGGTACTTATAAGACAGTGCACCTGTGAGCA |
| | 7-28505460 (+) | ATGCCGAGGTTGTGAGTTCAAGCCTCACCTGGAGCA |
| 133 | Leu_AAG_chr5: 18052 | GGTAGCGTGGCCGAGCGGTCTAAGGCGCTGGATTAAGGCTCCAGTCTCTTCGGAGGCG |
| | 4474-180524555 (−) | TGGGTTCGAATCCCACCGCTGCCA |
| 134 | Leu_AAG_chr5: 18061 | GGTAGCGTGGCCGAGCGGTCTAAGGCGCTGGATTAAGGCTCCAGTCTCTTCGGGGGCG |
| | 4701-180614782 (+) | TGGGTTCGAATCCCACCGCTGCCA |
| 135 | Leu_AAG_chr6: 28956 | GGTAGCGTGGCCGAGCGGTCTAAGGCGCTGGATTAAGGCTCCAGTCTCTTCGGGGGCG |
| | 779-28956860 (+) | TGGGTTCAAATCCCACCGCTGCCA |
| 136 | Leu_AAG_chr6: 28446 | GGTAGCGTGGCCGAGTGGTCTAAGACGCTGGATTAAGGCTCCAGTCTCTTCGGGGGCG |
| | 400-28446481 (−) | TGGGTTTGAATCCCACCGCTGCCA |
| 137 | Leu_CAA_chr6: 288640 | GTCAGGATGGCCGAGTGGTCTAAGGCGCCAGACTCAAGCTAAGCTTCCTCCGCGGTGG |
| | 00-28864105 (−) | GGATTCTGGTCTCCAATGGAGGCGTGGGTTCGAATCCCACTTCTGACA |
| 138 | Leu_CAA_chr6: 289088 | GTCAGGATGGCCGAGTGGTCTAAGGCGCCAGACTCAAGCTTGGCTTCCTCGTGTTGAG |
| | 30-28908934 (+) | GATTCTGGTCTCCAATGGAGGCGTGGGTTCGAATCCCACTTCTGACA |
| 139 | Leu_CAA_chr6: 275734 | GTCAGGATGGCCGAGTGGTCTAAGGCGCCAGACTCAAGCTTACTGCTTCCTGTGTTCG |
| | 17-27573524 (−) | GGTCTTCTGGTCTCCGTATGGAGGCGTGGGTTCGAATCCCACTTCTGACA |
| 140 | Leu_CAA_chr6: 275703 | GTCAGGATGGCCGAGTGGTCTAAGGCGCCAGACTCAAGTTGCTACTTCCCAGGTTTGG |
| | 48-27570454 (−) | GGCTTCTGGTCTCCGCATGGAGGCGTGGGTTCGAATCCCACTTCTGACA |
| 141 | Leu_CAA_chr1: 249168 | GTCAGGATGGCCGAGTGGTCTAAGGCGCCAGACTCAAGGTAAGCACCTTGCCTGCGGG |
| | 054-249168159 (+) | CTTTCTGGTCTCCGGATGGAGGCGTGGGTTCGAATCCCACTTCTGACA |
| 142 | Leu_CAA_chr11: 92967 | GCCTCCTTAGTGCAGTAGGTAGCGCATCAGTCTCAAAATCTGAATGGTCCTGAGTTCA |
| | 90-9296863 (+) | AGCCTCAGAGGGGGCA |
| 143 | Leu_CAA_chr1: 161581 | GTCAGGATGGCCGAGCAGTCTTAAGGCGCTGCGTTCAAATCGCACCCTCCGCTGGAGG |
| | 736-161581819 (−) | CGTGGGTTCGAATCCCACTTTTGACA |
| 144 | Leu_CAG_chr1: 161411 | GTCAGGATGGCCGAGCGGTCTAAGGCGCTGCGTTCAGGTCGCAGTCTCCCCTGGAGGC |
| | 323-161411405 (+) | GTGGGTTCGAATCCCACTCCTGACA |
| 145 | Leu_CAG_chr16: 57333 | GTCAGGATGGCCGAGCGGTCTAAGGCGCTGCGTTCAGGTCGCAGTCTCCCCTGGAGGC |
| | 863-57333945 (+) | GTGGGTTCGAATCCCACTTCTGACA |
| 146 | Leu_TAA_chr6: 144537 | ACCAGGATGGCCGAGTGGTTAAGGCGTTGGACTTAAGATCCAATGGACATATGTCCGC |
| | 684-144537766 (+) | GTGGGTTCGAACCCCACTCCTGGTA |
| 147 | Leu_TAA_chr6: 276888 | ACCGGGATGGCCGAGTGGTTAAGGCGTTGGACTTAAGATCCAATGGGCTGGTGCCCGC |
| | 98-27688980 (−) | GTGGGTTCGAACCCCACTCTCGGTA |
| 148 | Leu_TAA_chr1l: 59319 | ACCAGAATGGCCGAGTGGTTAAGGCGTTGGACTTAAGATCCAATGGATTCATATCCGC |
| | 228-59319310 (+) | GTGGGTTCGAACCCCACTTCTGGTA |
| 149 | Leu_TAA_chr6: 271983 | ACCGGGATGGCTGAGTGGTTAAGGCGTTGGACTTAAGATCCAATGGACAGGTGTCCGC |
| | 34-27198416 (−) | GTGGGTTCGAGCCCCACTCCCGGTA |
| 150 | Leu_TAG_chr17: 80236 | GGTAGCGTGGCCGAGCGGTCTAAGGCGCTGGATTTAGGCTCCAGTCTCTTCGGAGGCG |
| | 32-8023713 (−) | TGGGTTCGAATCCCACCGCTGCCA |
| 151 | Leu_TAG_chr14: 21093 | GGTAGTGTGGCCGAGCGGTCTAAGGCGCTGGATTTAGGCTCCAGTCTCTTCGGGGGCG |
| | 529-21093610 (+) | TGGGTTCGAATCCCACCACTGCCA |
| 152 | Leu_TAG_chr16: 22207 | GGTAGCGTGGCCGAGTGGTCTAAGGCGCTGGATTTAGGCTCCAGTCATTTCGATGGCG |
| | 032-22207113 (−) | TGGGTTCGAATCCCACCGCTGCCA |
| 153 | Lys_CTT_chr14: 58706 | GCCCGGCTAGCTCAGTCGGTAGAGCATGGGACTCTTAATCCCAGGGTCGTGGGTTCGA |
| | 613-58706685 (−) | GCCCCACGTTGGGCG |
| 154 | Lys_CTT_chr19: 36066 | GCCCAGCTAGCTCAGTCGGTAGAGCATAAGACTCTTAATCTCAGGGTTGTGGATTCGT |
| | 750-36066822 (+) | GCCCCATGCTGGGTG |
| 155 | Lys_CTT_chr19: 52425 | GCAGCTAGCTCAGTCGGTAGAGCATGAGACTCTTAATCTCAGGGTCATGGGTTCGTGC |
| | 393-52425466 (−) | CCCATGTTGGGTGCCA |
| 156 | Lys_CTT_chr1: 145395 | GCCCGGCTAGCTCAGTCGGTAGAGCATGAGACTCTTAATCTCAGGGTCGTGGGTTCGA |
| | 522-145395594 (−) | GCCCCACGTTGGGCG |
| 157 | Lys_CTT_chr16: 32074 | GCCCGGCTAGCTCAGTCGGTAGAGCATGAGACCCTTAATCTCAGGGTCGTGGGTTCGA |
| | 06-3207478 (−) | GCCCCACGTTGGGCG |
| 158 | Lys_CTT_chr16: 32415 | GCCCGGCTAGCTCAGTCGGTAGAGCATGGGACTCTTAATCTCAGGGTCGTGGGTTCGA |
| | 01-3241573 (+) | GCCCCACGTTGGGCG |
| 159 | Lys_CTT_chr16: 32305 | GCCCGGCTAGCTCAGTCGATAGAGCATGAGACTCTTAATCTCAGGGTCGTGGGTTCGA |
| | 55-3230627 (−) | GCCGCACGTTGGGCG |
| 160 | Lys_CTT_chr1: 554235 | GCCCAGCTAGCTCAGTCGGTAGAGCATGAGACTCTTAATCTCAGGGTCATGGGTTTGA |
| | 42-55423614 (−) | GCCCCACGTTTGGTG |
| 161 | Lys_CTT_chr16: 32149 | GCCTGGCTAGCTCAGTCGGCAAAGCATGAGACTCTTAATCTCAGGGTCGTGGGCTCGA |
| | 39-3215011 (+) | GCTCCATGTTGGGCG |
| 162 | Lys_CTT_chr5: 261985 | GCCCGACTACCTCAGTCGGTGGAGCATGGGACTCTTCATCCCAGGGTTGTGGGTTCGA |
| | 39-26198611 (−) | GCCCCACATTGGGCA |
| 163 | Lys_TTT_chr16: 73512 | GCCTGGATAGCTCAGTTGGTAGAGCATCAGACTTTTAATCTGAGGGTCCAGGGTTCAA |
| | 216-73512288 (−) | GTCCCTGTTCAGGCA |
| 164 | Lys_TTT_chr12: 27843 | ACCCAGATAGCTCAGTCAGTAGAGCATCAGACTTTTAATCTGAGGGTCCAAGGTTCAT |
| | 306-27843378 (+) | GTCCCTTTTTGGGTG |
| 165 | Lys_TTT_chr11: 12243 | GCCTGGATAGCTCAGTTGGTAGAGCATCAGACTTTTAATCTGAGGGTCCAGGGTTCAA |
| | 0655-122430727 (+) | GTCCCTGTTCAGGCG |
| 166 | Lys_TTT_chr1: 204475 | GCCCGGATAGCTCAGTCGGTAGAGCATCAGACTTTTAATCTGAGGGTCCAGGGTTCAA |
| | 655-204475727 (+) | GTCCCTGTTCGGGCG |
| 167 | Lys_TTT_chr6: 275595 | GCCTGGATAGCTCAGTCGGTAGAGCATCAGACTTTTAATCTGAGGGTCCAGGGTTCAA |
| | 93-27559665 (−) | GTCCCTGTTCAGGCG |
| 168 | Lys_TTT_chr11: 59323 | GCCCGGATAGCTCAGTCGGTAGAGCATCAGACTTTTAATCTGAGGGTCCGGGGTTCAA |
| | 902-59323974 (+) | GTCCCTGTTCGGGCG |
| 169 | Lys_TTT_chr6: 273027 | GCCTGGGTAGCTCAGTCGGTAGAGCATCAGACTTTTAATCTGAGGGTCCAGGGTTCAA |
| | 69-27302841 (−) | GTCCCTGTCCAGGCG |
| 170 | Lys_TTT_chr6: 287155 | GCCTGGATAGCTCAGTTGGTAGAACATCAGACTTTTAATCTGACGGTGCAGGGTTCAA |
| | 21-28715593 (+) | GTCCCTGTTCAGGCG |
| 171 | Met_CAT_chr8: 124169 | GCCTCGTTAGCGCAGTAGGTAGCGCGTCAGTCTCATAATCTGAAGGTCGTGAGTTCGA |
| | 470-124169542 (−) | TCCTCACACGGGGCA |
| 172 | Met_CAT_chr16: 71460 | GCCCTCTTAGCGCAGTGGGCAGCGCGTCAGTCTCATAATCTGAAGGTCCTGAGTTCGA |
| | 396-71460468 (+) | GCCTCAGAGAGGGCA |
| 173 | Met_CAT_chr6: 289123 | GCCTCCTTAGCGCAGTAGGCAGCGCGTCAGTCTCATAATCTGAAGGTCCTGAGTTCGA |
| | 52-28912424 (+) | ACCTCAGAGGGGGCA |
| 174 | Met_CAT_chr6: 267355 | GCCCTCTTAGCGCAGCGGGCAGCGCGTCAGTCTCATAATCTGAAGGTCCTGAGTTCGA |
| | 74-26735646 (−) | GCCTCAGAGAGGGCA |
| 175 | Met_CAT_chr6: 267017 | GCCCTCTTAGCGCAGCTGGCAGCGCGTCAGTCTCATAATCTGAAGGTCCTGAGTTCAA |
| | 12-26701784 (+) | GCCTCAGAGAGGGCA |
| 176 | Met_CAT_chr16: 87417 | GCCTCGTTAGCGCAGTAGGCAGCGCGTCAGTCTCATAATCTGAAGGTCGTGAGTTCGA |
| | 628-87417700 (−) | GCCTCACACGGGGCA |
| 177 | Met_CAT_chr6: 581684 | GCCCTCTTAGTGCAGCTGGCAGCGCGTCAGTTTCATAATCTGAAAGTCCTGAGTTCAA |
| | 92-58168564 (−) | GCCTCAGAGAGGGCA |
| 178 | Phe_GAA_chr6: 287584 | GCCGAAATAGCTCAGTTGGGAGAGCGTTAGACTGAAGATCTAAAGGTCCCTGGTTCGA |
| | 99-28758571 (−) | TCCCGGGTTTCGGCA |
| 179 | Phe_GAA_chr11: 59333 | GCCGAAATAGCTCAGTTGGGAGAGCGTTAGACTGAAGATCTAAAGGTCCCTGGTTCAA |
| | 853-59333925 (−) | TCCCGGGTTTCGGCA |
| 180 | Phe_GAA_chr6: 287756 | GCCGAGATAGCTCAGTTGGGAGAGCGTTAGACTGAAGATCTAAAGGTCCCTGGTTCAA |
| | 10-28775682 (−) | TCCCGGGTTTCGGCA |
| 181 | Phe_GAA_chr6: 287910 | GCCGAAATAGCTCAGTTGGGAGAGCGTTAGACCGAAGATCTTAAAGGTCCCTGGTTCA |
| | 93-28791166 (−) | ATCCCGGGTTTCGGCA |
| 182 | Phe_GAA_chr6: 287313 | GCTGAAATAGCTCAGTTGGGAGAGCGTTAGACTGAAGATCTTAAAGTTCCCTGGTTCA |
| | 74-28731447 (−) | ACCCTGGGTTTCAGCC |
| 183 | Pro_AGG_chr16: 32419 | GGCTCGTTGGTCTAGGGGTATGATTCTCGCTTAGGATGCGAGAGGTCCCGGGTTCAAA |
| | 89-3242060 (+) | TCCCGGACGAGCCC |
| 184 | Pro_AGG_chr1: 167684 | GGCTCGTTGGTCTAGGGGTATGATTCTCGCTTAGGGTGCGAGAGGTCCCGGGTTCAAA |
| | 725-167684796 (−) | TCCCGGACGAGCCC |
| 185 | Pro_CGG_chr1: 167683 | GGCTCGTTGGTCTAGGGGTATGATTCTCGCTTCGGGTGCGAGAGGTCCCGGGTTCAAA |
| | 962-167684033 (+) | TCCCGGACGAGCCC |
| 186 | Pro_CGG_chr6: 270595 | GGCTCGTTGGTCTAGGGGTATGATTCTCGCTTCGGGTGTGAGAGGTCCCGGGTTCAAA |
| | 21-27059592 (+) | TCCCGGACGAGCCC |
| 187 | Pro_TGG_chr14: 21101 | GGCTCGTTGGTCTAGTGGTATGATTCTCGCTTTGGGTGCGAGAGGTCCCGGGTTCAAAT |
| | 165-21101236 (+) | CCCGGACGAGCCC |
| 188 | Pro_TGG_chr11: 75946 | GGCTCGTTGGTCTAGGGGTATGATTCTCGGTTTGGGTCCGAGAGGTCCCGGGTTCAAA |
| | 869-75946940 (−) | TCCCGGACGAGCCC |
| 189 | Pro_TGG_chr5: 180615 | GGCTCGTTGGTCTAGGGGTATGATTCTCGCTTTGGGTGCGAGAGGTCCCGGGTTCAAA |
| | 854-180615925 (−) | TCCCGGACGAGCCC |
| 190 | SeCTCA_chr19: 45981 | GCCCGGATGATCCTCAGTGGTCTGGGGTGCAGGCTTCAAACCTGTAGCTGTCTAGCGA |
| | 859-45981945 (−) | CAGAGTGGTTCAATTCCACCTTTCGGGCG |
| 191 | SeCTCA_chr22: 44546 | GCTCGGATGATCCTCAGTGGTCTGGGGTGCAGGCTTCAAACCTGTAGCTGTCTAGTGA |
| | 537-44546620 (+) | CAGAGTGGTTCAATTCCACCTTTGTA |
| 192 | Ser_AGA_chr6: 275095 | GTAGTCGTGGCCGAGTGGTTAAGGCGATGGACTAGAAATCCATTGGGGTTTCCCCGCG |
| | 54-27509635 (−) | CAGGTTCGAATCCTGCCGACTACG |
| 193 | Ser_AGA_chr6: 263278 | GTAGTCGTGGCCGAGTGGTTAAGGCGATGGACTAGAAATCCATTGGGGTCTCCCCGCG |
| | 17-26327898 (+) | CAGGTTCGAATCCTGCCGACTACG |
| 194 | Ser_AGA_chr6: 274999 | GTAGTCGTGGCCGAGTGGTTAAGGCGATGGACTAGAAATCCATTGGGGTTTCCCCACG |
| | 87-27500068 (+) | CAGGTTCGAATCCTGCCGACTACG |
| 195 | Ser_AGA_chr6: 275211 | GTAGTCGTGGCCGAGTGGTTAAGGTGATGGACTAGAAACCCATTGGGGTCTCCCCGCG |
| | 92-27521273 (−) | CAGGTTCGAATCCTGCCGACTACG |
| 196 | Ser_CGA_chr17: 80421 | GCTGTGATGGCCGAGTGGTTAAGGCGTTGGACTCGAAATCCAATGGGGTCTCCCCGCG |
| | 99-8042280 (−) | CAGGTTCGAATCCTGCTCACAGCG |
| 197 | Ser_CGA_chr6: 271776 | GCTGTGATGGCCGAGTGGTTAAGGCGTTGGACTCGAAATCCAATGGGGTCTCCCCGCG |
| | 28-27177709 (+) | CAGGTTCAAATCCTGCTCACAGCG |
| 198 | Ser_CGA_chr6: 276402 | GCTGTGATGGCCGAGTGGTTAAGGTGTTGGACTCGAAATCCAATGGGGGTTCCCCGCG |
| | 29-27640310 (−) | CAGGTTCAAATCCTGCTCACAGCG |
| 199 | Ser_CGA_chr12: 56584 | GTCACGGTGGCCGAGTGGTTAAGGCGTTGGACTCGAAATCCAATGGGGTTTCCCCGCA |
| | 148-56584229 (+) | CAGGTTCGAATCCTGTTCGTGACG |
| 200 | Ser_GCT_chr6: 270650 | GACGAGGTGGCCGAGTGGTTAAGGCGATGGACTGCTAATCCATTGTGCTCTGCACGCG |
| | 85-27065166 (+) | TGGGTTCGAATCCCACCCTCGTCG |
| 201 | Ser_GCT_chr6: 272657 | GACGAGGTGGCCGAGTGGTTAAGGCGATGGACTGCTAATCCATTGTGCTCTGCACGCG |
| | 75-27265856 (+) | TGGGTTCGAATCCCACCTTCGTCG |
| 202 | Ser_GCT_chr1l: 66115 | GACGAGGTGGCCGAGTGGTTAAGGCGATGGACTGCTAATCCATTGTGCTTTGCACGCG |
| | 591-66115672 (+) | TGGGTTCGAATCCCATCCTCGTCG |
| 203 | Ser_GCT_chr6: 285651 | GACGAGGTGGCCGAGTGGTTAAGGCGATGGACTGCTAATCCATTGTGCTCTGCACGCG |
| | 17-28565198 (−) | TGGGTTCGAATCCCATCCTCGTCG |
| 204 | Ser_GCT_chr6: 281808 | GACGAGGTGGCCGAGTGGTTAAGGCGATGGACTGCTAATCCATTGTGCTCTGCACACG |
| | 15-28180896 (+) | TGGGTTCGAATCCCATCCTCGTCG |
| 205 | Ser_GCT_chr6: 263057 | GGAGAGGCCTGGCCGAGTGGTTAAGGCGATGGACTGCTAATCCATTGTGCTCTGCACG |
| | 18-26305801 (−) | CGTGGGTTCGAATCCCATCCTCGTCG |
| 206 | Ser_TGA_chr10: 69524 | GCAGCGATGGCCGAGTGGTTAAGGCGTTGGACTTGAAATCCAATGGGGTCTCCCCGCG |
| | 261-69524342 (+) | CAGGTTCGAACCCTGCTCGCTGCG |
| 207 | Ser_TGA_chr6: 275134 | GTAGTCGTGGCCGAGTGGTTAAGGCGATGGACTTGAAATCCATTGGGGTTTCCCCGCG |
| | 68-27513549 (+) | CAGGTTCGAATCCTGCCGACTACG |
| 208 | Ser_TGA_chr6: 263128 | GTAGTCGTGGCCGAGTGGTTAAGGCGATGGACTTGAAATCCATTGGGGTCTCCCCGCG |
| | 24-26312905 (−) | CAGGTTCGAATCCTGCCGACTACG |
| 209 | Ser_TGA_chr6: 274736 | GTAGTCGTGGCCGAGTGGTTAAGGCGATGGACTTGAAATCCATTGGGGTTTCCCCGCG |
| | 07-27473688 (−) | CAGGTTCGAATCCTGTCGGCTACG |
| 210 | Thr_AGT_chr17: 80904 | GGCGCCGTGGCTTAGTTGGTTAAAGCGCCTGTCTAGTAAACAGGAGATCCTGGGTTCG |
| | 78-8090551 (+) | AATCCCAGCGGTGCCT |
| 211 | Thr_AGT_chr6: 265331 | GGCTCCGTGGCTTAGCTGGTTAAAGCGCCTGTCTAGTAAACAGGAGATCCTGGGTTCG |
| | 45-26533218 (−) | AATCCCAGCGGGGCCT |
| 212 | Thr_AGT_chr6: 286937 | GGCTCCGTAGCTTAGTTGGTTAAAGCGCCTGTCTAGTAAACAGGAGATCCTGGGTTCG |
| | 95-28693868 (+) | ACTCCCAGCGGGGCCT |
| 213 | Thr_AGT_chr6: 276944 | GGCTTCGTGGCTTAGCTGGTTAAAGCGCCTGTCTAGTAAACAGGAGATCCTGGGTTCG |
| | 73-27694546 (+) | AATCCCAGCGAGGCCT |
| 214 | Thr_AGT_chr17: 80427 | GGCGCCGTGGCTTAGCTGGTTAAAGCGCCTGTCTAGTAAACAGGAGATCCTGGGTTCG |
| | 70-8042843 (−) | AATCCCAGCGGTGCCT |
| 215 | Thr_AGT_chr6: 271300 | GGCCCTGTGGCTTAGCTGGTCAAAGCGCCTGTCTAGTAAACAGGAGATCCTGGGTTCG |
| | 50-27130123 (+) | AATCCCAGCGGGGCCT |
| 216 | Thr_CGT_chr6: 284567 | GGCTCTATGGCTTAGTTGGTTAAAGCGCCTGTCTCGTAAACAGGAGATCCTGGGTTCG |
| | 70-28456843 (−) | ACTCCCAGTGGGGCCT |
| 217 | Thr_CGT_chr16: 14379 | GGCGCGGTGGCCAAGTGGTAAGGCGTCGGTCTCGTAAACCGAAGATCACGGGTTCGA |
| | 750-14379821 (+) | ACCCCGTCCGTGCCT |
| 218 | Thr_CGT_chr6: 286159 | GGCTCTGTGGCTTAGTTGGCTAAAGCGCCTGTCTCGTAAACAGGAGATCCTGGGTTCG |
| | 84-28616057 (−) | AATCCCAGCGGGGCCT |
| 219 | Thr_CGT_chr17: 29877 | GGCGCGGTGGCCAAGTGGTAAGGCGTCGGTCTCGTAAACCGAAGATCGCGGGTTCGA |
| | 093-29877164 (+) | ACCCCGTCCGTGCCT |
| 220 | Thr_CGT_chr6: 275861 | GGCCCTGTAGCTCAGCGGTTGGAGCGCTGGTCTCGTAAACCTAGGGGTCGTGAGTTCA |
| | 35-27586208 (+) | AATCTCACCAGGGCCT |
| 221 | Thr_TGT_chr6: 284423 | GGCTCTATGGCTTAGTTGGTTAAAGCGCCTGTCTTGTAAACAGGAGATCCTGGGTTCG |
| | 29-28442402 (−) | AATCCCAGTAGAGCCT |
| 222 | Thr_TGT_chr1: 222638 | GGCTCCATAGCTCAGTGGTTAGAGCACTGGTCTTGTAAACCAGGGGTCGCGAGTTCGA |
| | 347-222638419 (+) | TCCTCGCTGGGGCCT |
| 223 | Thr_TGT_chr14: 21081 | GGCTCCATAGCTCAGGGGTTAGAGCGCTGGTCTTGTAAACCAGGGGTCGCGAGTTCAA |
| | 949-21082021 (−) | TTCTCGCTGGGGCCT |
| 224 | Thr_TGT_chr14: 21099 | GGCTCCATAGCTCAGGGGTTAGAGCACTGGTCTTGTAAACCAGGGGTCGCGAGTTCAA |
| | 319-21099391 (−) | ATCTCGCTGGGGCCT |
| 225 | Thr_TGT_chr14: 21149 | GGCCCTATAGCTCAGGGGTTAGAGCACTGGTCTTGTAAACCAGGGGTCGCGAGTTCAA |
| | 849-21149921 (+) | ATCTCGCTGGGGCCT |
| 226 | Thr_TGT_chr5: 180618 | GGCTCCATAGCTCAGGGGTTAGAGCACTGGTCTTGTAAACCAGGGTCGCGAGTTCAAA |
| | 687-180618758 (−) | TCTCGCTGGGGCCT |
| 227 | Trp_CCA_chr17: 81241 | GGCCTCGTGGCGCAACGGTAGCGCGTCTGACTCCAGATCAGAAGGTTGCGTGTTCAAA |
| | 87-8124258 (−) | TCACGTCGGGGTCA |
| 228 | Trp_CCA_chr17: 19411 | GACCTCGTGGCGCAATGGTAGCGCGTCTGACTCCAGATCAGAAGGTTGCGTGTTCAAG |
| | 494-19411565 (+) | TCACGTCGGGGTCA |
| 229 | Trp_CCA_chr6: 263193 | GACCTCGTGGCGCAACGGTAGCGCGTCTGACTCCAGATCAGAAGGTTGCGTGTTCAAA |
| | 30-26319401 (−) | TCACGTCGGGGTCA |
| 230 | Trp_CCA_chr12: 98898 | GACCTCGTGGCGCAACGGTAGCGCGTCTGACTCCAGATCAGAAGGCTGCGTGTTCGAA |
| | 030-98898101 (+) | TCACGTCGGGGTCA |
| 231 | Trp_CCA_chr7: 990673 | GACCTCGTGGCGCAACGGCAGCGCGTCTGACTCCAGATCAGAAGGTTGCGTGTTCAAA |
| | 07-99067378 (+) | TCACGTCGGGGTCA |
| 232 | Tyr_ATA_chr2: 219110 | CCTTCAATAGTTCAGCTGGTAGAGCAGAGGACTATAGCTACTTCCTCAGTAGGAGACG |
| | 549-219110641 (+) | TCCTTAGGTTGCTGGTTCGATTCCAGCTTGAAGGA |
| 233 | Tyr_GTA_chr6: 265690 | CCTTCGATAGCTCAGTTGGTAGAGCGGAGGACTGTAGTTGGCTGTGTCCTTAGACATC |
| | 86-26569176 (+) | CTTAGGTCGCTGGTTCGAATCCGGCTCGAAGGA |
| 234 | Tyr_GTA_chr2: 272736 | CCTTCGATAGCTCAGTTGGTAGAGCGGAGGACTGTAGTGGATAGGGCGTGGCAATCCT |
| | 50-27273738 (+) | TAGGTCGCTGGTTCGATTCCGGCTCGAAGGA |
| 235 | Tyr_GTA_chr6: 265773 | CCTTCGATAGCTCAGTTGGTAGAGCGGAGGACTGTAGGCTCATTAAGCAAGGTATCCT |
| | 32-26577420 (+) | TAGGTCGCTGGTTCGAATCCGGCTCGGAGGA |
| 236 | Tyr_GTA_chr14: 21125 | CCTTCGATAGCTCAGCTGGTAGAGCGGAGGACTGTAGATTGTATAGACATTTGCGGAC |
| | 623-21125716 (−) | ATCCTTAGGTCGCTGGTTCGATTCCAGCTCGAAGGA |
| 237 | Tyr_GTA_chr8: 670256 | CCTTCGATAGCTCAGCTGGTAGAGCGGAGGACTGTAGCTACTTCCTCAGCAGGAGACA |
| | 02-67025694 (+) | TCCTTAGGTCGCTGGTTCGATTCCGGCTCGAAGGA |
| 238 | Tyr_GTA_chr8: 670262 | CCTTCGATAGCTCAGCTGGTAGAGCGGAGGACTGTAGGCGCGCGCCCGTGGCCATCCT |
| | 23-67026311 (+) | TAGGTCGCTGGTTCGATTCCGGCTCGAAGGA |
| 239 | Tyr_GTA_chr14: 21121 | CCTTCGATAGCTCAGCTGGTAGAGCGGAGGACTGTAGCCTGTAGAAACATTTGTGGAC |
| | 258-21121351 (−) | ATCCTTAGGTCGCTGGTTCGATTCCGGCTCGAAGGA |
| 240 | Tyr_GTA_chr14: 21131 | CCTTCGATAGCTCAGCTGGTAGAGCGGAGGACTGTAGATTGTACAGACATTTGCGGAC |
| | 351-21131444 (−) | ATCCTTAGGTCGCTGGTTCGATTCCGGCTCGAAGGA |
| 241 | Tyr_GTA_chr14: 21151 | CCTTCGATAGCTCAGCTGGTAGAGCGGAGGACTGTAGTACTTAATGTGTGGTCATCCTT |
| | 432-21151520 (+) | AGGTCGCTGGTTCGATTCCGGCTCGAAGGA |
| 242 | Tyr_GTA_chr6: 265951 | CCTTCGATAGCTCAGCTGGTAGAGCGGAGGACTGTAGGGGTTTGAATGTGGTCATCCT |
| | 02-26595190 (+) | TAGGTCGCTGGTTCGAATCCGGCTCGGAGGA |
| 243 | Tyr_GTA_chr14: 21128 | CCTTCGATAGCTCAGCTGGTAGAGCGGAGGACTGTAGACTGCGGAAACGTTTGTGGAC |
| | 117-21128210 (−) | ATCCTTAGGTCGCTGGTTCAATTCCGGCTCGAAGGA |
| 244 | Tyr_GTA_chr6: 265757 | CTTTCGATAGCTCAGTTGGTAGAGCGGAGGACTGTAGGTTCATTAAACTAAGGCATCC |
| | 98-26575887 (+) | TTAGGTCGCTGGTTCGAATCCGGCTCGAAGGA |
| 245 | Tyr_GTA_chr8: 666095 | TCTTCAATAGCTCAGCTGGTAGAGCGGAGGACTGTAGGTGCACGCCCGTGGCCATTCT |
| | 32-66609619 (−) | TAGGTGCTGGTTTGATTCCGACTTGGAGAG |
| 246 | Val_AAC_chr3: 169490 | GTTTCCGTAGTGTAGTGGTTATCACGTTCGCCTAACACGCGAAAGGTCCCCGGTTCGA |
| | 018-169490090 (+) | AACCGGGCGGAAACA |
| 247 | Val_AAC_chr5: 180615 | GTTTCCGTAGTGTAGTGGTCATCACGTTCGCCTAACACGCGAAAGGTCCCCGGTTCGA |
| | 416-180615488 (−) | AACCGGGCGGAAACA |
| 248 | Val_AAC_chr6: 276187 | GTTTCCGTAGTGTAGTGGTTATCACGTTCGCCTAACACGCGAAAGGTCCCTGGATCAA |
| | 07-27618779 (−) | AACCAGGCGGAAACA |
| 249 | Val_AAC_chr6: 276488 | GTTTCCGTAGTGTAGTGGTTATCACGTTCGCCTAACACGCGAAAGGTCCGCGGTTCGA |
| | 85-27648957 (−) | AACCGGGCGGAAACA |
| 250 | Val_AAC_chr6: 272032 | GTTTCCGTAGTGTAGTGGTTATCACGTTTGCCTAACACGCGAAAGGTCCCCGGTTCGAA |
| | 88-27203360 (+) | ACCGGGCAGAAACA |
| 251 | Val_AAC_chr6: 287032 | GGGGGTGTAGCTCAGTGGTAGAGCGTATGCTTAACATTCATGAGGCTCTGGGTTCGAT |
| | 06-28703277 (−) | CCCCAGCACTTCCA |
| 252 | Val_CAC_chr1: 161369 | GTTTCCGTAGTGTAGTGGTTATCACGTTCGCCTCACACGCGAAAGGTCCCCGGTTCGAA |
| | 490-161369562 (−) | ACCGGGCGGAAACA |
| 253 | Val_CAC_chr6: 272480 | GCTTCTGTAGTGTAGTGGTTATCACGTTCGCCTCACACGCGAAAGGTCCCCGGTTCGAA |
| | 49-27248121 (−) | ACCGGGCAGAAGCA |
| 254 | Val_CAC_chr19: 47246 | GTTTCCGTAGTGTAGCGGTTATCACATTCGCCTCACACGCGAAAGGTCCCCGGTTCGAT |
| | 47-4724719 (−) | CCCGGGCGGAAACA |
| 255 | Val_CAC_chr1: 149298 | GTTTCCGTAGTGTAGTGGTTATCACGTTCGCCTCACACGCGAAAGGTCCCCGGTTCGAA |
| | 555-149298627 (−) | ACTGGGCGGAAACA |
| 256 | Val_CAC_chr1: 149684 | GTTTCCGTAGTGTAGTGGTTATCACGTTCGCCTCACACGCGTAAAGGTCCCCGGTTCGA |
| | 088-149684161 (−) | AACCGGGCGGAAACA |
| 257 | Val_CAC_chr6: 271738 | GTTTCCGTAGTGGAGTGGTTATCACGTTCGCCTCACACGCGAAAGGTCCCCGGTTTGA |
| | 67-27173939 (−) | AACCAGGCGGAAACA |
| 258 | Val_TAC_chr11: 59318 | GGTTCCATAGTGTAGTGGTTATCACGTCTGCTTTACACGCAGAAGGTCCTGGGTTCGAG |
| | 102-59318174 (−) | CCCCAGTGGAACCA |
| 259 | Val_TAC_chr11: 59318 | GGTTCCATAGTGTAGCGGTTATCACGTCTGCTTTACACGCAGAAGGTCCTGGGTTCGA |
| | 460-59318532 (−) | GCCCCAGTGGAACCA |
| 260 | Val_TAC_chr10: 58956 | GGTTCCATAGTGTAGTGGTTATCACATCTGCTTTACACGCAGAAGGTCCTGGGTTCAAG |
| | 74-5895746 (−) | CCCCAGTGGAACCA |
| 261 | Val_TAC_chr6: 272584 | GTTTCCGTGGTGTAGTGGTTATCACATTCGCCTTACACGCGAAAGGTCCTCGGGTCGAA |
| | 05-27258477 (+) | ACCGAGCGGAAACA |
| 262 | iMet_CAT_chr1: 15364 | AGCAGAGTGGCGCAGCGGAAGCGTGCTGGGCCCATAACCCAGAGGTCGATGGATCGA |
| | 3726-153643797 (+) | AACCATCCTCTGCTA |
| 263 | iMet_CAT_chr6: 27745 | AGCAGAGTGGCGCAGCGGAAGCGTGCTGGGCCCATAACCCAGAGGTCGATGGATCTA |
| | 664-27745735 (+) | AACCATCCTCTGCTA |
| 264 | Glu_TTC_chr1: 168617 | TCCCTGGTGGTCTAGTGGCTAGGATTCGGCGCTTTCACCGCCGCGGCCCGGGTTCGATT |
| | 73-16861845 (−) | CCCGGTCAGGGAAT |
| 265 | Gly_CCC_chr1: 170047 | GCGTTGGTGGTTTAGTGGTAGAATTCTCGCCTCCCATGCGGGAGACCCGGGTTCAATTC |
| | 65-17004836 (−) | CCGGCCACTGCAC |
| 266 | Gly_CCC_chr1: 170537 | GGCCTTGGTGGTGCAGTGGTAGAATTCTCGCCTCCCACGTGGGAGACCCGGGTTCAAT |
| | 79-17053850 (+) | TCCCGGCCAATGCA |
| 267 | Glu_TTC_chr1: 171990 | GTCCCTGGTGGTCTAGTGGCTAGGATTCGGCGCTTTCACCGCCGCGGCCCGGGTTCGAT |
| | 77-17199149 (+) | TCCCGGCCAGGGAA |
| 268 | Asn_GTT_chr1: 172161 | TGTCTCTGTGGCGCAATCGGTTAGCGCGTTCGGCTGTTAACCGAAAGATTGGTGGTTCG |
| | 71-17216245 (+) | AGCCCACCCAGGGACG |
| 269 | Arg_TCT_chr1: 943131 | TGGCTCCGTGGCGCAATGGATAGCGCATTGGACTTCTAGAGGCTGAAGGCATTCAAAG |
| | 28-94313213 (+) | GTTCCGGGTTCGAGTCCCGGCGGAGTCG |
| 270 | Lys_CTT_chr1: 145395 | GCCCGGCTAGCTCAGTCGGTAGAGCATGAGACTCTTAATCTCAGGGTCGTGGGTTCGA |
| | 521-145395594 (−) | GCCCCACGTTGGGCGC |
| 271 | His_GTG_chr1: 145396 | GCCGTGATCGTATAGTGGTTAGTACTCTGCGTTGTGGCCGCAGCAACCTCGGTTCGAAT |
| | 880-145396952 (−) | CCGAGTCACGGCAG |
| 272 | Gly_TCC_chr1: 145397 | GCGTTGGTGGTATAGTGGTGAGCATAGCTGCCTTCCAAGCAGTTGACCCGGGTTCGAT |
| | 863-145397935 (−) | TCCCGGCCAACGCAG |
| 273 | Glu_CTC_chr1: 145399 | TCCCTGGTGGTCTAGTGGTTAGGATTCGGCGCTCTCACCGCCGCGGCCCGGGTTCGATT |
| | 232-145399304 (−) | CCCGGTCAGGGAAA |
| 274 | Gln_CTG_chr1: 145963 | AGGTTCCATGGTGTAATGGTGAGCACTCTGGACTCTGAATCCAGCGATCCGAGTTCGA |
| | 303-145963375 (+) | GTCTCGGTGGAACCT |
| 275 | Asn_GTT_chr1: 148000 | TGTCTCTGTGGCGTAGTCGGTTAGCGCGTTCGGCTGTTAACCGAAAAGTTGGTGGTTCG |
| | 804-148000878 (+) | AGCCCACCCAGGAACG |
| 276 | Asn_GTT_chr1: 148248 | TGTCTCTGTGGCGCAATCGGTTAGCGCGTTCGGCTGTTAACCGAAAGGTTGGTGGTTCG |
| | 114-148248188 (+) | AGCCCACCCAGGGACG |
| 277 | Asn_GTT_chr1: 148598 | GTCTCTGTGGCGCAATCGGTTAGCGCATTCGGCTGTTAACCGAAAGGTTGGTGGTTCG |
| | 313-148598387 (−) | AGCCCACCCAGGGACGC |
| 278 | Asn_GTT_chr1: 149230 | GTCTCTGTGGCGCAATGGGTTAGCGCGTTCGGCTGTTAACCGAAAGGTTGGTGGTTCG |
| | 569-149230643 (−) | AGCCCATCCAGGGACGC |
| 279 | Val_CAC_chr1: 149294 | GCACTGGTGGTTCAGTGGTAGAATTCTCGCCTCACACGCGGGACACCCGGGTTCAATT |
| | 665-149294736 (−) | CCCGGTCAAGGCAA |
| 280 | Val_CAC_chr1: 149298 | GTTTCCGTAGTGTAGTGGTTATCACGTTCGCCTCACACGCGAAAGGTCCCCGGTTCGAA |
| | 554-149298627 (−) | ACTGGGCGGAAACAG |
| 281 | Gly_CCC_chr1: 149680 | GCACTGGTGGTTCAGTGGTAGAATTCTCGCCTCCCACGCGGGAGACCCGGGTTTAATT |
| | 209-149680280 (−) | CCCGGTCAAGATAA |
| 282 | Val_CAC_chr1: 149684 | GTTTCCGTAGTGTAGTGGTTATCACGTTCGCCTCACACGCGTAAAGGTCCCCGGTTCGA |
| | 087-149684161 (−) | AACCGGGCGGAAACAT |
| 283 | Met_CAT_chr1: 153643 | TAGCAGAGTGGCGCAGCGGAAGCGTGCTGGGCCCATAACCCAGAGGTCGATGGATCG |
| | 725-153643797 (+) | AAACCATCCTCTGCTA |
| 284 | Val_CAC_chr1: 161369 | GTTTCCGTAGTGTAGTGGTTATCACGTTCGCCTCACACGCGAAAGGTCCCCGGTTCGAA |
| | 489-161369562 (−) | ACCGGGCGGAAACAA |
| 285 | Asp_GTC_chr1: 161410 | TCCTCGTTAGTATAGTGGTGAGTATCCCCGCCTGTCACGCGGGAGACCGGGGTTCGAT |
| | 614-161410686 (−) | TCCCCGACGGGGAGG |
| 286 | Gly_GCC_chr1: 161413 | TGCATGGGTGGTTCAGTGGTAGAATTCTCGCCTGCCACGCGGGAGGCCCGGGTTCGAT |
| | 093-161413164 (+) | TCCCGGCCCATGCA |
| 287 | Glu_CTC_chr1: 161417 | TCCCTGGTGGTCTAGTGGTTAGGATTCGGCGCTCTCACCGCCGCGGCCCGGGTTCGATT |
| | 017-161417089 (−) | CCCGGTCAGGGAAG |
| 288 | Asp_GTC_chr1: 161492 | ATCCTTGTTACTATAGTGGTGAGTATCTCTGCCTGTCATGCGTGAGAGAGGGGGTCGAT |
| | 934-161493006 (+) | TCCCCGACGGGGAG |
| 289 | Gly_GCC_chr1: 161493 | GCATTGGTGGTTCAGTGGTAGAATTCTCGCCTGCCACGCGGGAGGCCCGGGTTCGATT |
| | 636-161493707 (−) | CCCGGCCAATGCAC |
| 290 | Leu_CAG_chr1: 161500 | GTCAGGATGGCCGAGCGGTCTAAGGCGCTGCGTTCAGGTCGCAGTCTCCCCTGGAGGC |
| | 131-161500214 (−) | GTGGGTTCGAATCCCACTCCTGACAA |
| 291 | Gly_TCC_chr1: 161500 | CGCGTTGGTGGTATAGTGGTGAGCATAGCTGCCTTCCAAGCAGTTGACCCGGGTTCGA |
| | 902-161500974 (+) | TTCCCGGCCAACGCA |
| 292 | Asn_GTT_chr1: 161510 | CGTCTCTGTGGCGCAATCGGTTAGCGCGTTCGGCTGTTAACCGAAAGGTTGGTGGTTC |
| | 030-161510104 (+) | GATCCCACCCAGGGACG |
| 293 | Glu_TTC_chr1: 161582 | CGCGTTGGTGGTGTAGTGGTGAGCACAGCTGCCTTTCAAGCAGTTAACGCGGGTTCGA |
| | 507-161582579 (+) | TTCCCGGGTAACGAA |
| 294 | Pro_CGG_chr1: 167683 | CGGCTCGTTGGTCTAGGGGTATGATTCTCGCTTCGGGTGCGAGAGGTCCCGGGTTCAA |
| | 961-167684033 (+) | ATCCCGGACGAGCCC |
| 295 | Pro_AGG_chr1: 167684 | GGCTCGTTGGTCTAGGGGTATGATTCTCGCTTAGGGTGCGAGAGGTCCCGGGTTCAAA |
| | 724-167684796 (−) | TCCCGGACGAGCCCT |
| 296 | Lys_TTT_chr1: 204475 | CGCCCGGATAGCTCAGTCGGTAGAGCATCAGACTTTTAATCTGAGGGTCCAGGGTTCA |
| | 654-204475727 (+) | AGTCCCTGTTCGGGCG |
| 297 | Lys_TTT_chr1: 204476 | GCCCGGATAGCTCAGTCGGTAGAGCATCAGACTTTTAATCTGAGGGTCCAGGGTTCAA |
| | 157-204476230 (−) | GTCCCTGTTCGGGCGT |
| 298 | Leu_CAA_chr1: 249168 | TGTCAGGATGGCCGAGTGGTCTAAGGCGCCAGACTCAAGGTAAGCACCTTGCCTGCGG |
| | 053-249168159 (+) | GCTTTCTGGTCTCCGGATGGAGGCGTGGGTTCGAATCCCACTTCTGACA |
| 299 | Glu_CTC_chr1: 249168 | TTCCCTGGTGGTCTAGTGGTTAGGATTCGGCGCTCTCACCGCCGCGGCCCGGGTTCGAT |
| | 446-249168518 (+) | TCCCGGTCAGGAAA |
| 300 | Tyr_GTA_chr2: 272736 | GCCTTCGATAGCTCAGTTGGTAGAGCGGAGGACTGTAGTGGATAGGGCGTGGCAATCC |
| | 49-27273738 (+) | TTAGGTCGCTGGTTCGATTCCGGCTCGAAGGA |
| 301 | Ala_AGC_chr2: 272740 | CGGGGGATTAGCTCAAATGGTAGAGCGCTCGCTTAGCATGCGAGAGGTAGCGGGATC |
| | 81-27274154 (+) | GATGCCCGCATCCTCCA |
| 302 | Ile_TAT_chr2: 4303767 | AGCTCCAGTGGCGCAATCGGTTAGCGCGCGGTACTTATACAGCAGTACATGCAGAGCA |
| | 5-43037768 (+) | ATGCCGAGGTTGTGAGTTCGAGCCTCACCTGGAGCA |
| 303 | Gly_CCC_chr2: 704761 | GCGCCGCTGGTGTAGTGGTATCATGCAAGATTCCCATTCTTGCGACCCGGGTTCGATTC |
| | 22-70476193 (−) | CCGGGCGGCGCAT |
| 304 | Glu_TTC_chr2: 131094 | TCCCATATGGTCTAGCGGTTAGGATTCCTGGTTTTCACCCAGGTGGCCCGGGTTCGACT |
| | 700-131094772 (−) | CCCGGTATGGGAAC |
| 305 | Ala_CGC_chr2: 157257 | GGGGGATGTAGCTCAGTGGTAGAGCGCGCGCTTCGCATGTGTGAGGTCCCGGGTTCAA |
| | 280-157257352 (+) | TCCCCGGCATCTCCA |
| 306 | Gly_GCC_chr2: 157257 | GCATTGGTGGTTCAGTGGTAGAATTCTCGCCTGCCACGCGGGAGGCCCGGGTTCGATT |
| | 658-157257729 (−) | CCCGGCCAATGCAA |
| 307 | Arg_ACG_chr3: 457304 | GGGCCAGTGGCGCAATGGATAACGCGTCTGACTACGGATCAGAAGATTCTAGGTTCGA |
| | 90-45730563 (−) | CTCCTGGCTGGCTCGC |
| 308 | Val_AAC_chr3: 169490 | GGTTTCCGTAGTGTAGTGGTTATCACGTTCGCCTAACACGCGAAAGGTCCCCGGTTCG |
| | 017-169490090 (+) | AAACCGGGCGGAAACA |
| 309 | Val_AAC_chr5: 180596 | AGTTTCCGTAGTGTAGTGGTTATCACGTTCGCCTAACACGCGAAAGGTCCCCGGTTCG |
| | 609-180596682 (+) | AAACCGGGCGGAAACA |
| 310 | Leu_AAG_chr5: 18061 | AGGTAGCGTGGCCGAGCGGTCTAAGGCGCTGGATTAAGGCTCCAGTCTCTTCGGGGGC |
| | 4700-180614782 (+) | GTGGGTTCGAATCCCACCGCTGCCA |
| 311 | Val_AAC_chr5: 180615 | GTTTCCGTAGTGTAGTGGTCATCACGTTCGCCTAACACGCGAAAGGTCCCCGGTTCGA |
| | 415-180615488 (−) | AACCGGGCGGAAACAT |
| 312 | Pro_TGG_chr5: 180615 | GGCTCGTTGGTCTAGGGGTATGATTCTCGCTTTGGGTGCGAGAGGTCCCGGGTTCAAA |
| | 853-180615925 (−) | TCCCGGACGAGCCCA |
| 313 | Thr_TGT_chr5: 180618 | GGCTCCATAGCTCAGGGGTTAGAGCACTGGTCTTGTAAACCAGGGTCGCGAGTTCAAA |
| | 686-180618758 (−) | TCTCGCTGGGGCCTG |
| 314 | Ala_TGC_chr5: 180633 | TGGGGATGTAGCTCAGTGGTAGAGCGCATGCTTTGCATGTATGAGGCCCCGGGTTCGA |
| | 867-180633939 (+) | TCCCCGGCATCTCCA |
| 315 | Lys_CTT_chr5: 180634 | CGCCCGGCTAGCTCAGTCGGTAGAGCATGAGACTCTTAATCTCAGGGTCGTGGGTTCG |
| | 754-180634827 (+) | AGCCCCACGTTGGGCG |
| 316 | Val_AAC_chr5: 180645 | GTTTCCGTAGTGTAGTGGTTATCACGTTCGCCTAACACGCGAAAGGTCCCCGGTTCGA |
| | 269-180645342 (−) | AACCGGGCGGAAACAA |
| 317 | Lys_CTT_chr5: 180648 | GCCCGGCTAGCTCAGTCGGTAGAGCATGAGACTCTTAATCTCAGGGTCGTGGGTTCGA |
| | 978-180649051 (−) | GCCCCACGTTGGGCGT |
| 318 | Val_CAC_chr5: 180649 | GTTTCCGTAGTGTAGTGGTTATCACGTTCGCCTCACACGCGAAAGGTCCCCGGTTCGAA |
| | 394-180649467 (−) | ACCGGGCGGAAACAC |
| 319 | Met_CAT_chr6: 262867 | CAGCAGAGTGGCGCAGCGGAAGCGTGCTGGGCCCATAACCCAGAGGTCGATGGATCG |
| | 53-26286825 (+) | AAACCATCCTCTGCTA |
| 320 | Ser_GCT_chr6: 263057 | GGAGAGGCCTGGCCGAGTGGTTAAGGCGATGGACTGCTAATCCATTGTGCTCTGCACG |
| | 17-26305801 (−) | CGTGGGTTCGAATCCCATCCTCGTCGC |
| 321 | Gln_TTG_chr6: 263114 | GGCCCCATGGTGTAATGGTTAGCACTCTGGACTTTGAATCCAGCGATCCGAGTTCAAA |
| | 23-26311495 (−) | TCTCGGTGGGACCTG |
| 322 | Gln_TTG_chr6: 263119 | GGCCCCATGGTGTAATGGTTAGCACTCTGGACTTTGAATCCAGCGATCCGAGTTCAAA |
| | 74-26312046 (−) | TCTCGGTGGGACCTA |
| 323 | Ser_TGA_chr6: 263128 | GTAGTCGTGGCCGAGTGGTTAAGGCGATGGACTTGAAATCCATTGGGGTCTCCCCGCG |
| | 23-26312905 (−) | CAGGTTCGAATCCTGCCGACTACGG |
| 324 | Met_CAT_chr6: 263133 | AGCAGAGTGGCGCAGCGGAAGCGTGCTGGGCCCATAACCCAGAGGTCGATGGATCGA |
| | 51-26313423 (−) | AACCATCCTCTGCTAT |
| 325 | Arg_TCG_chr6: 263230 | GGACCACGTGGCCTAATGGATAAGGCGTCTGACTTCGGATCAGAAGATTGAGGGTTCG |
| | 45-26323118 (+) | AATCCCTCCGTGGTTA |
| 326 | Ser_AGA_chr6: 263278 | TGTAGTCGTGGCCGAGTGGTTAAGGCGATGGACTAGAAATCCATTGGGGTCTCCCCGC |
| | 16-26327898 (+) | GCAGGTTCGAATCCTGCCGACTACG |
| 327 | Met_CAT_chr6: 263305 | AGCAGAGTGGCGCAGCGGAAGCGTGCTGGGCCCATAACCCAGAGGTCGATGGATCGA |
| | 28-26330600 (−) | AACCATCCTCTGCTAG |
| 328 | Leu_CAG_chr6: 265214 | CGTCAGGATGGCCGAGCGGTCTAAGGCGCTGCGTTCAGGTCGCAGTCTCCCCTGGAGG |
| | 35-26521518 (+) | CGTGGGTTCGAATCCCACTCCTGACA |
| 329 | Thr_AGT_chr6: 265331 | GGCTCCGTGGCTTAGCTGGTTAAAGCGCCTGTCTAGTAAACAGGAGATCCTGGGTTCG |
| | 44-26533218 (−) | AATCCCAGCGGGGCCTG |
| 330 | Arg_ACG_chr6: 265377 | AGGGCCAGTGGCGCAATGGATAACGCGTCTGACTACGGATCAGAAGATTCCAGGTTCG |
| | 25-26537798 (+) | ACTCCTGGCTGGCTCG |
| 331 | Val_CAC_chr6: 265382 | GGTTTCCGTAGTGTAGTGGTTATCACGTTCGCCTCACACGCGAAAGGTCCCCGGTTCGA |
| | 81-26538354 (+) | AACCGGGCGGAAACA |
| 332 | Ala_CGC_chr6: 265537 | AGGGGATGTAGCTCAGTGGTAGAGCGCATGCTTCGCATGTATGAGGTCCCGGGTTCGA |
| | 30-26553802 (+) | TCCCCGGCATCTCCA |
| 333 | Ile_AAT_chr6: 2655434 | TGGCCGGTTAGCTCAGTTGGTTAGAGCGTGGTGCTAATAACGCCAAGGTCGCGGGTTC |
| | 9-26554423 (+) | GATCCCCGTACGGGCCA |
| 334 | Pro_AGG_chr6: 265554 | CGGCTCGTTGGTCTAGGGGTATGATTCTCGCTTAGGGTGCGAGAGGTCCCGGGTTCAA |
| | 97-26555569 (+) | ATCCCGGACGAGCCC |
| 335 | Lys_CTT_chr6: 265567 | AGCCCGGCTAGCTCAGTCGGTAGAGCATGAGACTCTTAATCTCAGGGTCGTGGGTTCG |
| | 73-26556846 (+) | AGCCCCACGTTGGGCG |
| 336 | Tyr_GTA_chr6: 265690 | TCCTTCGATAGCTCAGTTGGTAGAGCGGAGGACTGTAGTTGGCTGTGTCCTTAGACATC |
| | 85-26569176 (+) | CTTAGGTCGCTGGTTCGAATCCGGCTCGAAGGA |
| 337 | Ala_AGC_chr6: 265720 | GGGGAATTAGCTCAAATGGTAGAGCGCTCGCTTAGCATGCGAGAGGTAGCGGGATCG |
| | 91-26572164 (−) | ATGCCCGCATTCTCCAG |
| 338 | Met_CAT_chr6: 267664 | CGCCCTCTTAGCGCAGCGGGCAGCGCGTCAGTCTCATAATCTGAAGGTCCTGAGTTCG |
| | 43-26766516 (+) | AGCCTCAGAGAGGGCA |
| 339 | Ile_TAT_chr6: 2698812 | TGCTCCAGTGGCGCAATCGGTTAGCGCGCGGTACTTATATGGCAGTATGTGTGCGAGT |
| | 4-26988218 (+) | GATGCCGAGGTTGTGAGTTCGAGCCTCACCTGGAGCA |
| 340 | His_GTG_chr6: 271259 | TGCCGTGATCGTATAGTGGTTAGTACTCTGCGTTGTGGCCGCAGCAACCTCGGTTCGAA |
| | 05-27125977 (+) | TCCGAGTCACGGCA |
| 341 | Ile_AAT_chr6: 2714499 | GGCCGGTTAGCTCAGTTGGTTAGAGCGTGGTGCTAATAACGCCAAGGTCGCGGGTTCG |
| | 3-27145067 (−) | ATCCCCGTACGGGCCAC |
| 342 | Val_AAC_chr6: 272032 | AGTTTCCGTAGTGTAGTGGTTATCACGTTTGCCTAACACGCGAAAGGTCCCCGGTTCGA |
| | 87-27203360 (+) | AACCGGGCAGAAACA |
| 343 | Val_CAC_chr6: 272480 | GCTTCTGTAGTGTAGTGGTTATCACGTTCGCCTCACACGCGAAAGGTCCCCGGTTCGAA |
| | 48-27248121 (−) | ACCGGGCAGAAGCAA |
| 344 | Asp_GTC_chr6: 274474 | TTCCTCGTTAGTATAGTGGTGAGTATCCCCGCCTGTCACGCGGGAGACCGGGGTTCGA |
| | 52-27447524 (+) | TTCCCCGACGGGGAG |
| 345 | Ser_TGA_chr6: 274736 | GTAGTCGTGGCCGAGTGGTTAAGGCGATGGACTTGAAATCCATTGGGGTTTCCCCGCG |
| | 06-27473688 (−) | CAGGTTCGAATCCTGTCGGCTACGG |
| 346 | Gln_CTG_chr6: 274873 | AGGTTCCATGGTGTAATGGTTAGCACTCTGGACTCTGAATCCAGCGATCCGAGTTCAA |
| | 07-27487379 (+) | ATCTCGGTGGAACCT |
| 347 | Asp_GTC_chr6: 275512 | TCCTCGTTAGTATAGTGGTGAGTGTCCCCGTCTGTCACGCGGGAGACCGGGGTTCGATT |
| | 35-27551307 (−) | CCCCGACGGGGAGA |
| 348 | Val_AAC_chr6: 276187 | GTTTCCGTAGTGTAGTGGTTATCACGTTCGCCTAACACGCGAAAGGTCCCTGGATCAA |
| | 06-27618779 (−) | AACCAGGCGGAAACAA |
| 349 | Ile_AAT_chr6: 2765596 | CGGCCGGTTAGCTCAGTTGGTTAGAGCGTGGTGCTAATAACGCCAAGGTCGCGGGTTC |
| | 6-27656040 (+) | GATCCCCGTACTGGCCA |
| 350 | Gln_CTG_chr6: 277591 | GGCCCCATGGTGTAATGGTCAGCACTCTGGACTCTGAATCCAGCGATCCGAGTTCAAA |
| | 34-27759206 (−) | TCTCGGTGGGACCCA |
| 351 | Gln_TTG_chr6: 277636 | GGCCCCATGGTGTAATGGTTAGCACTCTGGACTTTGAATCCAGCGATCCGAGTTCAAA |
| | 39-27763711 (−) | TCTCGGTGGGACCTT |
| 352 | Ala_AGC_chr6: 285749 | TGGGGGTGTAGCTCAGTGGTAGAGCGCGTGCTTAGCATGTACGAGGTCCCGGGTTCAA |
| | 32-28575004 (+) | TCCCCGGCACCTCCA |
| 353 | Ala_AGC_chr6: 286260 | GGGGATGTAGCTCAGTGGTAGAGCGCATGCTTAGCATGCATGAGGTCCCGGGTTCGAT |
| | 13-28626085 (−) | CCCCAGCATCTCCAG |
| 354 | Ala_CGC_chr6: 286970 | AGGGGGTGTAGCTCAGTGGTAGAGCGCGTGCTTCGCATGTACGAGGCCCCGGGTTCGA |
| | 91-28697163 (+) | CCCCCGGCTCCTCCA |
| 355 | Ala_AGC_chr6: 288062 | GGGGGTGTAGCTCAGTGGTAGAGCGCGTGCTTAGCATGCACGAGGCCCCGGGTTCAAT |
| | 20-28806292 (−) | CCCCGGCACCTCCAT |
| 356 | Ala_AGC_chr6: 288314 | GGGGGTGTAGCTCAGTGGTAGAGCGCGTGCTTAGCATGCACGAGGCCCCGGGTTCAAT |
| | 61-28831533 (−) | CCCCGGCACCTCCAG |
| 357 | Leu_CAA_chr6: 288639 | GTCAGGATGGCCGAGTGGTCTAAGGCGCCAGACTCAAGCTAAGCTTCCTCCGCGGTGG |
| | 99-28864105 (−) | GGATTCTGGTCTCCAATGGAGGCGTGGGTTCGAATCCCACTTCTGACAC |
| 358 | Leu_CAA_chr6: 289088 | TGTCAGGATGGCCGAGTGGTCTAAGGCGCCAGACTCAAGCTTGGCTTCCTCGTGTTGA |
| | 29-28908934 (+) | GGATTCTGGTCTCCAATGGAGGCGTGGGTTCGAATCCCACTTCTGACA |
| 359 | Gln_CTG_chr6: 289093 | GGTTCCATGGTGTAATGGTTAGCACTCTGGACTCTGAATCCAGCGATCCGAGTTCAAA |
| | 77-28909449 (−) | TCTCGGTGGAACCTT |
| 360 | Leu_AAG_chr6: 28911 | GGTAGCGTGGCCGAGCGGTCTAAGGCGCTGGATTAAGGCTCCAGTCTCTTCGGGGGCG |
| | 398-28911480 (−) | TGGGTTCGAATCCCACCGCTGCCAG |
| 361 | Met_CAT_chr6: 289123 | TGCCTCCTTAGCGCAGTAGGCAGCGCGTCAGTCTCATAATCTGAAGGTCCTGAGTTCG |
| | 51-28912424 (+) | AACCTCAGAGGGGGCA |
| 362 | Lys_TTT_chr6: 289188 | AGCCCGGATAGCTCAGTCGGTAGAGCATCAGACTTTTAATCTGAGGGTCCAGGGTTCA |
| | 05-28918878 (+) | AGTCCCTGTTCGGGCG |
| 363 | Met_CAT_chr6: 289210 | GCCTCCTTAGCGCAGTAGGCAGCGCGTCAGTCTCATAATCTGAAGGTCCTGAGTTCGA |
| | 41-28921114 (−) | ACCTCAGAGGGGGCAG |
| 364 | Glu_CTC_chr6: 289499 | TTCCCTGGTGGTCTAGTGGTTAGGATTCGGCGCTCTCACCGCCGCGGCCCGGGTTCGAT |
| | 75-28950047 (+) | TCCCGGTCAGGGAA |
| 365 | Leu_TAA_chr6: 144537 | CACCAGGATGGCCGAGTGGTTAAGGCGTTGGACTTAAGATCCAATGGACATATGTCCG |
| | 683-144537766 (+) | CGTGGGTTCGAACCCCACTCCTGGTA |
| 366 | Pro_AGG_chr7: 128423 | TGGCTCGTTGGTCTAGGGGTATGATTCTCGCTTAGGGTGCGAGAGGTCCCGGGTTCAA |
| | 503-128423575 (+) | ATCCCGGACGAGCCC |
| 367 | Arg_CCT_chr7: 139025 | AGCCCCAGTGGCCTAATGGATAAGGCATTGGCCTCCTAAGCCAGGGATTGTGGGTTCG |
| | 445-139025518 (+) | AGTCCCATCTGGGGTG |
| 368 | Cys_GCA_chr7: 149388 | GGGGATATAGCTCAGGGGTAGAGCATTTGACTGCAGATCAAGAGGTCCCCGGTTCAAA |
| | 271-149388343 (−) | TCCGGGTGCCCCCCC |
| 369 | Tyr_GTA_chr8: 670256 | CCCTTCGATAGCTCAGCTGGTAGAGCGGAGGACTGTAGCTACTTCCTCAGCAGGAGAC |
| | 01-67025694 (+) | ATCCTTAGGTCGCTGGTTCGATTCCGGCTCGAAGGA |
| 370 | Tyr_GTA_chr8: 670262 | CCCTTCGATAGCTCAGCTGGTAGAGCGGAGGACTGTAGGCGCGCGCCCGTGGCCATCC |
| | 22-67026311 (+) | TTAGGTCGCTGGTTCGATTCCGGCTCGAAGGA |
| 371 | Ala_AGC_chr8: 670264 | TGGGGGATTAGCTCAAATGGTAGAGCGCTCGCTTAGCATGCGAGAGGTAGCGGGATC |
| | 23-67026496 (+) | GATGCCCGCATCCTCCA |
| 372 | Ser_AGA_chr8: 962818 | GTAGTCGTGGCCGAGTGGTTAAGGCGATGGACTAGAAATCCATTGGGGTCTCCCCGCG |
| | 84-96281966 (−) | CAGGTTCGAATCCTGCCGACTACGG |
| 373 | Met_CAT_chr8: 124169 | GCCTCGTTAGCGCAGTAGGTAGCGCGTCAGTCTCATAATCTGAAGGTCGTGAGTTCGA |
| | 469-124169542 (−) | TCCTCACACGGGGCAC |
| 374 | Arg_TCT_chr9: 131102 | GGCTCTGTGGCGCAATGGATAGCGCATTGGACTTCTAGCTGAGCCTAGTGTGGTCATT |
| | 354-131102445 (−) | CAAAGGTTGTGGGTTCGAGTCCCACCAGAGTCGA |
| 375 | Asn_GTT_chr10: 22518 | GTCTCTGTGGCGCAATCGGTTAGCGCGTTCGGCTGTTAACCGAAAGGTTGGTGGTTCG |
| | 437-22518511 (−) | AGCCCACCCAGGGACGC |
| 376 | Ser_TGA_chr10: 69524 | GGCAGCGATGGCCGAGTGGTTAAGGCGTTGGACTTGAAATCCAATGGGGTCTCCCCGC |
| | 260-69524342 (+) | GCAGGTTCGAACCCTGCTCGCTGCG |
| 377 | Val_TAC_chr11: 59318 | GGTTCCATAGTGTAGTGGTTATCACGTCTGCTTTACACGCAGAAGGTCCTGGGTTCGAG |
| | 101-59318174 (−) | CCCCAGTGGAACCAT |
| 378 | Val_TAC_chr11: 59318 | GGTTCCATAGTGTAGCGGTTATCACGTCTGCTTTACACGCAGAAGGTCCTGGGTTCGA |
| | 459-59318532 (−) | GCCCCAGTGGAACCAC |
| 379 | Arg_TCT_chr11: 59318 | TGGCTCTGTGGCGCAATGGATAGCGCATTGGACTTCTAGATAGTTAGAGAAATTCAAA |
| | 766-59318852 (+) | GGTTGTGGGTTCGAGTCCCACCAGAGTCG |
| 380 | Leu_TAA_chr11: 59319 | TACCAGAATGGCCGAGTGGTTAAGGCGTTGGACTTAAGATCCAATGGATTCATATCCG |
| | 227-59319310 (+) | CGTGGGTTCGAACCCCACTTCTGGTA |
| 381 | Lys_TTT_chr11: 59323 | GGCCCGGATAGCTCAGTCGGTAGAGCATCAGACTTTTAATCTGAGGGTCCGGGGTTCA |
| | 901-59323974 (+) | AGTCCCTGTTCGGGCG |
| 382 | Phe_GAA_chr11: 59324 | GCCGAAATAGCTCAGTTGGGAGAGCGTTAGACTGAAGATCTAAAGGTCCCTGGTTCGA |
| | 969-59325042 (−) | TCCCGGGTTTCGGCAG |
| 383 | Lys_TTT_chr11: 59327 | GCCCGGATAGCTCAGTCGGTAGAGCATCAGACTTTTAATCTGAGGGTCCAGGGTTCAA |
| | 807-59327880 (−) | GTCCCTGTTCGGGCGG |
| 384 | Phe_GAA_chr11: 59333 | GCCGAAATAGCTCAGTTGGGAGAGCGTTAGACTGAAGATCTAAAGGTCCCTGGTTCAA |
| | 852-59333925 (−) | TCCCGGGTTTCGGCAG |
| 385 | Ser_GCT_chr11: 66115 | GGACGAGGTGGCCGAGTGGTTAAGGCGATGGACTGCTAATCCATTGTGCTTTGCACGC |
| | 590-66115672 (+) | GTGGGTTCGAATCCCATCCTCGTCG |
| 386 | Pro_TGG_chr11: 75946 | GGCTCGTTGGTCTAGGGGTATGATTCTCGGTTTGGGTCCGAGAGGTCCCGGGTTCAAA |
| | 868-75946940 (−) | TCCCGGACGAGCCCC |
| 387 | Ser_CGA_chr12: 56584 | AGTCACGGTGGCCGAGTGGTTAAGGCGTTGGACTCGAAATCCAATGGGGTTTCCCCGC |
| | 147-56584229 (+) | ACAGGTTCGAATCCTGTTCGTGACG |
| 388 | Asp_GTC_chr12: 98897 | CTCCTCGTTAGTATAGTGGTTAGTATCCCCGCCTGTCACGCGGGAGACCGGGGTTCAAT |
| | 280-98897352 (+) | TCCCCGACGGGGAG |
| 389 | Trp_CCA_chr12: 98898 | GGACCTCGTGGCGCAACGGTAGCGCGTCTGACTCCAGATCAGAAGGCTGCGTGTTCGA |
| | 029-98898101 (+) | ATCACGTCGGGGTCA |
| 390 | Ala_TGC_chr12: 12540 | GGGGATGTAGCTCAGTGGTAGAGCGCATGCTTTGCATGTATGAGGCCCCGGGTTCGAT |
| | 6300-125406372 (−) | CCCCGGCATCTCCAT |
| 391 | Phe_GAA_chr12: 12541 | GCCGAAATAGCTCAGTTGGGAGAGCGTTAGACTGAAGATCTAAAGGTCCCTGGTTCGA |
| | 2388-125412461 (−) | TCCCGGGTTTCGGCAC |
| 392 | Ala_TGC_chr12: 12542 | AGGGGATGTAGCTCAGTGGTAGAGCGCATGCTTTGCACGTATGAGGCCCCGGGTTCAA |
| | 4511-125424583 (+) | TCCCCGGCATCTCCA |
| 393 | Asn_GTT_chr13: 31248 | GTCTCTGTGGCGCAATCGGTTAGCGCGTTCGGCTGTTAACCGAAAGGTTGGTGGTTCG |
| | 100-31248174 (−) | AGCCCACCCAGGGACGG |
| 394 | Glu_TTC_chr13: 45492 | TCCCACATGGTCTAGCGGTTAGGATTCCTGGTTTTCACCCAGGCGGCCCGGGTTCGACT |
| | 061-45492133 (−) | CCCGGTGTGGGAAC |
| 395 | Thr_TGT_chr14: 21081 | GGCTCCATAGCTCAGGGGTTAGAGCGCTGGTCTTGTAAACCAGGGGTCGCGAGTTCAA |
| | 948-21082021 (−) | TTCTCGCTGGGGCCTG |
| 396 | Leu_TAG_chr14: 21093 | TGGTAGTGTGGCCGAGCGGTCTAAGGCGCTGGATTTAGGCTCCAGTCTCTTCGGGGGC |
| | 528-21093610 (+) | GTGGGTTCGAATCCCACCACTGCCA |
| 397 | Thr_TGT_chr14: 21099 | GGCTCCATAGCTCAGGGGTTAGAGCACTGGTCTTGTAAACCAGGGGTCGCGAGTTCAA |
| | 318-21099391 (−) | ATCTCGCTGGGGCCTC |
| 398 | Pro_TGG_chr14: 21101 | TGGCTCGTTGGTCTAGTGGTATGATTCTCGCTTTGGGTGCGAGAGGTCCCGGGTTCAAA |
| | 164-21101236 (+) | TCCCGGACGAGCCC |
| 399 | Tyr_GTA_chr14: 21131 | CCTTCGATAGCTCAGCTGGTAGAGCGGAGGACTGTAGATTGTACAGACATTTGCGGAC |
| | 350-21131444 (−) | ATCCTTAGGTCGCTGGTTCGATTCCGGCTCGAAGGAA |
| 400 | Thr_TGT_chr14: 21149 | AGGCCCTATAGCTCAGGGGTTAGAGCACTGGTCTTGTAAACCAGGGGTCGCGAGTTCA |
| | 848-21149921 (+) | AATCTCGCTGGGGCCT |
| 401 | Tyr_GTA_chr14: 21151 | TCCTTCGATAGCTCAGCTGGTAGAGCGGAGGACTGTAGTACTTAATGTGTGGTCATCCT |
| | 431-21151520 (+) | TAGGTCGCTGGTTCGATTCCGGCTCGAAGGA |
| 402 | Pro_TGG_chr14: 21152 | TGGCTCGTTGGTCTAGGGGTATGATTCTCGCTTTGGGTGCGAGAGGTCCCGGGTTCAA |
| | 174-21152246 (+) | ATCCCGGACGAGCCC |
| 403 | Lys_CTT_chr14: 58706 | GCCCGGCTAGCTCAGTCGGTAGAGCATGGGACTCTTAATCCCAGGGTCGTGGGTTCGA |
| | 612-58706685 (−) | GCCCCACGTTGGGCGC |
| 404 | Ile_AAT_chr14: 102783 | CGGCCGGTTAGCTCAGTTGGTTAGAGCGTGGTGCTAATAACGCCAAGGTCGCGGGTTC |
| | 428-102783502 (+) | GATCCCCGTACGGGCCA |
| 405 | Glu_TTC_chr15: 26327 | TCCCACATGGTCTAGCGGTTAGGATTCCTGGTTTTCACCCAGGCGGCCCGGGTTCGACT |
| | 380-26327452 (−) | CCCGGTGTGGGAAT |
| 406 | Ser_GCT_chr15: 40886 | GACGAGGTGGCCGAGTGGTTAAGGCGATGGACTGCTAATCCATTGTGCTCTGCACGCG |
| | 022-40886104 (−) | TGGGTTCGAATCCCATCCTCGTCGA |
| 407 | His_GTG_chr15: 45490 | GCCGTGATCGTATAGTGGTTAGTACTCTGCGTTGTGGCCGCAGCAACCTCGGTTCGAAT |
| | 803-45490875 (−) | CCGAGTCACGGCAT |
| 408 | His_GTG_chr15: 45493 | CGCCGTGATCGTATAGTGGTTAGTACTCTGCGTTGTGGCCGCAGCAACCTCGGTTCGA |
| | 348-45493420 (+) | ATCCGAGTCACGGCA |
| 409 | Gln_CTG_chr15: 66161 | GGTTCCATGGTGTAATGGTTAGCACTCTGGACTCTGAATCCAGCGATCCGAGTTCAAA |
| | 399-66161471 (−) | TCTCGGTGGAACCTG |
| 410 | Lys_CTT_chr15: 79152 | TGCCCGGCTAGCTCAGTCGGTAGAGCATGGGACTCTTAATCCCAGGGTCGTGGGTTCG |
| | 903-79152976 (+) | AGCCCCACGTTGGGCG |
| 411 | Arg_TCG_chr15: 89878 | GGGCCGCGTGGCCTAATGGATAAGGCGTCTGACTTCGGATCAGAAGATTGCAGGTTCG |
| | 303-89878376 (+) | AGTCCTGCCGCGGTCG |
| 412 | Gly_CCC_chr16: 68673 | GCGCCGCTGGTGTAGTGGTATCATGCAAGATTCCCATTCTTGCGACCCGGGTTCGATTC |
| | 5-686806 (−) | CCGGGCGGCGCAC |
| 413 | Arg_CCG_chr16: 32006 | GGGCCGCGTGGCCTAATGGATAAGGCGTCTGATTCCGGATCAGAAGATTGAGGGTTCG |
| | 74-3200747 (+) | AGTCCCTTCGTGGTCG |
| 414 | Arg_CCT_chr16: 32029 | CGCCCCGGTGGCCTAATGGATAAGGCATTGGCCTCCTAAGCCAGGGATTGTGGGTTCG |
| | 00-3202973 (+) | AGTCCCACCCGGGGTA |
| 415 | Lys_CTT_chr16: 32074 | GCCCGGCTAGCTCAGTCGGTAGAGCATGAGACCCTTAATCTCAGGGTCGTGGGTTCGA |
| | 05-3207478 (−) | GCCCCACGTTGGGCGT |
| 416 | Thr_CGT_chr16: 14379 | AGGCGCGGTGGCCAAGTGGTAAGGCGTCGGTCTCGTAAACCGAAGATCACGGGTTCG |
| | 749-14379821 (+) | AACCCCGTCCGTGCCT |
| 417 | Leu_TAG_chr16: 22207 | GGTAGCGTGGCCGAGTGGTCTAAGGCGCTGGATTTAGGCTCCAGTCATTTCGATGGCG |
| | 031-22207113 (−) | TGGGTTCGAATCCCACCGCTGCCAC |
| 418 | Leu_AAG_chr16: 2230 | GGGTAGCGTGGCCGAGCGGTCTAAGGCGCTGGATTAAGGCTCCAGTCTCTTCGGGGGC |
| | 8460-22308542 (+) | GTGGGTTCGAATCCCACCGCTGCCA |
| 419 | Leu_CAG_chr16: 57333 | AGTCAGGATGGCCGAGCGGTCTAAGGCGCTGCGTTCAGGTCGCAGTCTCCCCTGGAGG |
| | 862-57333945 (+) | CGTGGGTTCGAATCCCACTTCTGACA |
| 420 | Leu_CAG_chr16: 57334 | GTCAGGATGGCCGAGCGGTCTAAGGCGCTGCGTTCAGGTCGCAGTCTCCCCTGGAGGC |
| | 391-57334474 (−) | GTGGGTTCGAATCCCACTTCTGACAG |
| 421 | Met_CAT_chr16: 87417 | GCCTCGTTAGCGCAGTAGGCAGCGCGTCAGTCTCATAATCTGAAGGTCGTGAGTTCGA |
| | 627-87417700 (−) | GCCTCACACGGGGCAG |
| 422 | Leu_TAG_chr17: 80236 | GGTAGCGTGGCCGAGCGGTCTAAGGCGCTGGATTTAGGCTCCAGTCTCTTCGGAGGCG |
| | 31-8023713 (−) | TGGGTTCGAATCCCACCGCTGCCAG |
| 423 | Arg_TCT_chr17: 80242 | TGGCTCTGTGGCGCAATGGATAGCGCATTGGACTTCTAGTGACGAATAGAGCAATTCA |
| | 42-8024330 (+) | AAGGTTGTGGGTTCGAATCCCACCAGAGTCG |
| 424 | Gly_GCC_chr17: 80290 | CGCATTGGTGGTTCAGTGGTAGAATTCTCGCCTGCCACGCGGGAGGCCCGGGTTCGAT |
| | 63-8029134 (+) | TCCCGGCCAATGCA |
| 425 | Ser_CGA_chr17: 80421 | GCTGTGATGGCCGAGTGGTTAAGGCGTTGGACTCGAAATCCAATGGGGTCTCCCCGCG |
| | 98-8042280 (−) | CAGGTTCGAATCCTGCTCACAGCGT |
| 426 | Thr_AGT_chr17: 80427 | GGCGCCGTGGCTTAGCTGGTTAAAGCGCCTGTCTAGTAAACAGGAGATCCTGGGTTCG |
| | 69-8042843 (−) | AATCCCAGCGGTGCCTG |
| 427 | Trp_CCA_chr17: 80896 | CGACCTCGTGGCGCAACGGTAGCGCGTCTGACTCCAGATCAGAAGGTTGCGTGTTCAA |
| | 75-8089747 (+) | ATCACGTCGGGGTCA |
| 428 | Ser_GCT_chr17: 80901 | AGACGAGGTGGCCGAGTGGTTAAGGCGATGGACTGCTAATCCATTGTGCTCTGCACGC |
| | 83-8090265 (+) | GTGGGTTCGAATCCCATCCTCGTCG |
| 429 | Thr_AGT_chr17: 80904 | CGGCGCCGTGGCTTAGTTGGTTAAAGCGCCTGTCTAGTAAACAGGAGATCCTGGGTTC |
| | 77-8090551 (+) | GAATCCCAGCGGTGCCT |
| 430 | Trp_CCA_chr17: 81241 | GGCCTCGTGGCGCAACGGTAGCGCGTCTGACTCCAGATCAGAAGGTTGCGTGTTCAAA |
| | 86-8124258 (−) | TCACGTCGGGGTCAA |
| 431 | Gly_TCC_chr17: 81248 | AGCGTTGGTGGTATAGTGGTAAGCATAGCTGCCTTCCAAGCAGTTGACCCGGGTTCGA |
| | 65-8124937 (+) | TTCCCGGCCAACGCA |
| 432 | Asp_GTC_chr17: 81255 | TCCTCGTTAGTATAGTGGTGAGTATCCCCGCCTGTCACGCGGGAGACCGGGGTTCGAT |
| | 55-8125627 (−) | TCCCCGACGGGGAGA |
| 433 | Pro_CGG_chr17: 81261 | GGCTCGTTGGTCTAGGGGTATGATTCTCGCTTCGGGTGCGAGAGGTCCCGGGTTCAAA |
| | 50-8126222 (−) | TCCCGGACGAGCCCT |
| 434 | Thr_AGT_chr17: 81295 | GGCGCCGTGGCTTAGTTGGTTAAAGCGCCTGTCTAGTAAACAGGAGATCCTGGGTTCG |
| | 52-8129626 (−) | AATCCCAGCGGTGCCTT |
| 435 | Ser_AGA_chr17: 81299 | GTAGTCGTGGCCGAGTGGTTAAGGCGATGGACTAGAAATCCATTGGGGTCTCCCCGCG |
| | 27-8130009 (−) | CAGGTTCGAATCCTGCCGACTACGT |
| 436 | Trp_CCA_chr17: 19411 | TGACCTCGTGGCGCAATGGTAGCGCGTCTGACTCCAGATCAGAAGGTTGCGTGTTCAA |
| | 493-19411565 (+) | GTCACGTCGGGGTCA |
| 437 | Thr_CGT_chr17: 29877 | AGGCGCGGTGGCCAAGTGGTAAGGCGTCGGTCTCGTAAACCGAAGATCGCGGGTTCG |
| | 092-29877164 (+) | AACCCCGTCCGTGCCT |
| 438 | Cys_GCA_chr17: 37023 | AGGGGGTATAGCTCAGTGGTAGAGCATTTGACTGCAGATCAAGAGGTCCCCGGTTCAA |
| | 897-37023969 (+) | ATCCGGGTGCCCCCT |
| 439 | Cys_GCA_chr17: 37025 | GGGGGTATAGCTCAGTGGTAGAGCATTTGACTGCAGATCAAGAGGTCCCTGGTTCAAA |
| | 544-37025616 (−) | TCCGGGTGCCCCCTC |
| 440 | Cys_GCA_chr17: 37309 | GGGGGTATAGCTCAGTGGTAGAGCATTTGACTGCAGATCAAGAGGTCCCCGGTTCAAA |
| | 986-37310058 (−) | TCCGGGTGCCCCCTC |
| 441 | Gln_TTG_chr17: 47269 | AGGTCCCATGGTGTAATGGTTAGCACTCTGGACTTTGAATCCAGCGATCCGAGTTCAA |
| | 889-47269961 (+) | ATCTCGGTGGGACCT |
| 442 | Arg_CCG_chr17: 66016 | GACCCAGTGGCCTAATGGATAAGGCATCAGCCTCCGGAGCTGGGGATTGTGGGTTCGA |
| | 012-66016085 (−) | GTCCCATCTGGGTCGC |
| 443 | Arg_CCT_chr17: 73030 | AGCCCCAGTGGCCTAATGGATAAGGCACTGGCCTCCTAAGCCAGGGATTGTGGGTTCG |
| | 000-73030073 (+) | AGTCCCACCTGGGGTA |
| 444 | Arg_CCT_chr17: 73030 | GCCCCAGTGGCCTAATGGATAAGGCACTGGCCTCCTAAGCCAGGGATTGTGGGTTCGA |
| | 525-73030598 (−) | GTCCCACCTGGGGTGT |
| 445 | Arg_TCG_chr17: 73031 | AGACCGCGTGGCCTAATGGATAAGGCGTCTGACTTCGGATCAGAAGATTGAGGGTTCG |
| | 207-73031280 (+) | AGTCCCTTCGTGGTCG |
| 446 | Asn_GTT_chr19: 13835 | CGTCTCTGTGGCGCAATCGGTTAGCGCGTTCGGCTGTTAACCGAAAGGTTGGTGGTTC |
| | 61-1383635 (+) | GAGCCCACCCAGGGACG |
| 447 | Gly_TCC_chr19: 47240 | GGCGTTGGTGGTATAGTGGTTAGCATAGCTGCCTTCCAAGCAGTTGACCCGGGTTCGA |
| | 81-4724153 (+) | TTCCCGGCCAACGCA |
| 448 | Val_CAC_chr19: 47246 | GTTTCCGTAGTGTAGCGGTTATCACATTCGCCTCACACGCGAAAGGTCCCCGGTTCGAT |
| | 46-4724719 (−) | CCCGGGCGGAAACAG |
| 449 | Thr_AGT_chr19: 33667 | TGGCGCCGTGGCTTAGTTGGTTAAAGCGCCTGTCTAGTAAACAGGAGATCCTGGGTTC |
| | 962-33668036 (+) | GAATCCCAGCGGTGCCT |
| 450 | Ile_TAT_chr19: 399028 | GCTCCAGTGGCGCAATCGGTTAGCGCGCGGTACTTATATGACAGTGCGAGCGGAGCAA |
| | 07-39902900 (−) | TGCCGAGGTTGTGAGTTCGATCCTCACCTGGAGCAC |
| 451 | Gly_GCC_chr21: 18827 | GCATGGGTGGTTCAGTGGTAGAATTCTCGCCTGCCACGCGGGAGGCCCGGGTTCGATT |
| | 106-18827177 (−) | CCCGGCCCATGCAG |

In an embodiment, a TREM, e.g., an exogenous TREM, comprises 1, 2, 3, or 4 of the following properties:

(a) differs by at least one nucleotide or one post transcriptional modification from the closest sequence tRNA in a reference cell, e.g., a cell into which the exogenous nucleic acid is introduced;

(b) has been introduced into a cell other than the cell in which it was transcribed;

(c) is present in a cell other than one in which it naturally occurs; or

(d) has an expression profile, e.g., level or distribution, that is non-wildtype, e.g., it is expressed at a higher level than wildtype.

In an embodiment, the expression profile can be mediated by a change introduced into a nucleic acid that modulates expression, or by addition of an agent that modulates expression of the RNA molecule.

In an embodiment, a TREM, e.g., an exogenous TREM comprises (a), (b), (c) and (d).

In an embodiment, a TREM, e.g., an exogenous TREM comprises (a), (b) and (c).

In an embodiment, a TREM, e.g., an exogenous TREM comprises (a), (b) and (d).

In an embodiment, a TREM, e.g., an exogenous TREM comprises (a), (c) and (d).

In an embodiment, a TREM, e.g., an exogenous TREM comprises (b), (c) and (d).

In an embodiment, a TREM, e.g., an exogenous TREM comprises (a) and (d).

In an embodiment, a TREM, e.g., an exogenous TREM comprises (c) and (d).

### TREM Fragments

In an embodiment, a TREM comprises a fragment (sometimes referred to herein as a TREM fragment), e.g., a fragment of a RNA encoded by a deoxyribonucleic acid sequence disclosed in Table 2. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., Dicer, Angiogenin, RNaseP, RNaseZ, Rny1, or PrrC.

In an embodiment, a TREM fragment can be produced in vivo, ex vivo or in vitro. In an embodiment, a TREM fragment is produced in vivo, in the host cell. In an embodiment, a TREM fragment is produced ex vivo. In an embodiment, a TREM fragment is produced in vitro, e.g., as described in Example 12. In an embodiment, the TREM fragment is produced by fragmenting an expressed TREM after production of the TREM by the cell, e.g., a TREM produced by the host cell is fragmented after release or purification from the host cell, e.g., the TREM is fragmented ex vivo or in vitro.

Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 2, e.g., any one of SEQ ID NOs: 1-451 disclosed in Table 2. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 2, e.g., any one of SEQ ID NOs: 1-451 disclosed in Table 2. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 2, e.g., any one of SEQ ID NOs: 1-451 disclosed in Table 2.

In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 2, e.g., any one of SEQ ID NOs: 1-451 disclosed in Table 2. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 2, e.g., any one of SEQ ID NOs: 1-451 disclosed in Table 2. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 2, e.g., any one of SEQ ID NOs: 1-451 disclosed in Table 2.

In an embodiment, a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

In an embodiment, a TREM fragment comprises a TREM structure, domain, or activity, e.g., as described herein above. In an embodiment, a TREM fragment comprises adaptor function, e.g., as described herein. In an embodiment, a TREM fragment comprises cognate adaptor function, e.g., as described herein. In an embodiment, a TREM fragment comprises non-cognate adaptor function, e.g., as described herein. In an embodiment, a TREM fragment comprises regulatory function, e.g., as described herein.

In an embodiment, a TREM fragment comprises translation inhibition function, e.g., displacement of an initiation factor, e.g., eIF4G.

In an embodiment, a TREM fragment comprises epigenetic function, e.g., epigenetic inheritance of a disorder, e.g., a metabolic disorder. In some embodiments, an epigenetic inheritance function can have a generational impact, e.g., as compared to somatic epigenetic regulation.

In an embodiment, a TREM fragment comprises retroviral regulation function, e.g., regulation of retroviral reverse transcription, e.g., HERV regulation.

In an embodiment, a TREM fragment comprises gene silencing function, e.g., by binding to AGO and/or PIWI.

In an embodiment, a TREM fragment comprises neuroprotectant function, e.g., by the sequestration of a translation initiation factor, e.g., in stress granules, to promote, e.g., motor neuron survival under cellular stress.

In an embodiment, a TREM fragment comprises anti-cancer function, e.g., by preventing cancer progression through the binding and/or sequestration of, e.g., metastatic transcript-stabilizing proteins.

In an embodiment, a TREM fragment comprises cell survival function, e.g., increased cell survival, by binding to, e.g., cytochrome c and/or cyt c ribonucleoprotein complex.

In an embodiment, a TREM fragment comprises ribosome biogenesis function, e.g., a TREM fragment can regulate ribosome biogenesis by, e.g., regulation of, e.g., binding to, an mRNA coding for ribosomal proteins.

### TREM Modifications

A TREM described herein can comprise a moiety, often referred to herein as a modification, e.g., a moiety described in Table 3. While the term modification as used herein should not generally be construed to be the product of any particular process, in embodiments, the formation of a modification can be mediated by an enzyme in Table 3. In embodiments, the modification is formed post-transcriptionally. In embodiments, the modification is formed co-transcriptionally. In an embodiment, the modification occurs in vivo, e.g., in the host cell.

In an embodiment, the modification is a modification listed in any of rows 1-62 of Table 3. In an embodiment, the modification is a modification listed in any of rows 1-62 of Table 3, and the formation of the modification is mediated by an enzyme in Table 3. In an embodiment the modification is selected from a row in Table 3 and the formation of the modification is mediated by an enzyme from the same row in Table 3.

TABLE 3
List of tRNA modifications and associated enzymes.

| | Short | | |
|---|---|---|---|
| | Name | Modification | Enzyme list |
| 1 | mlAm | 1,2′-O-dimethyladenosine | METTL3 |
| 2 | imG | wyosine | Trm5, Tywl, Tyw2, Tyw3, and Tyw4 |
| 3 | m5s2U | 5-methyl-2-thiouridine | TrmU |
| 4 | m6t6A | N6-methyl-N6-threonylcarbamoyladenosine | TRMO, Trm0 |
| 5 | QtRNA | queuosine | TGTase |
| 6 | OHyW | hydroxywybutosine | Trm5, TYW1, TYW2, TYW3, TYW4 |
| 7 | io6A | N6-(cis-hydroxyisopentenyl)adenosine | TRIT1 |
| 8 | Gr(p) | 2′-O-ribosylguanosine (phosphate) | |
| 9 | ho5U | 5-hydroxyuridine | |
| 10 | ncm5Um | 5-carbamoylmethy1-2′-O-methyluridine | ELP1, ELP2, ELP3, ELP4, ELP5, ELP6, |
| | | | KTI111, KTI112, KTI113, Uba4, Urm1, Tum1, |
| | | | Ncs6, Ncs2, Trm9, Sit4, Isu1, Isu2, Sap185, |
| | | | Sap190 |
| 11 | OHyW* | hydroxywybutosine | wybutosine hydroxylases |
| 12 | acp3U | 3-(3-amino-3-carboxypropyl)uridine | |
| 13 | mcm5s2 | 5 -methoxycarbonylmethyl-2-thiouridine | ALKBH8, Ncs6, Trm9, Ncs2, TrmU, |
| | U | | CTU1,CTU2, ELP1, ELP2, ELP3, ELP4, ELP5, |
| | | | ELP6 |
| 14 | m5U | 5-methyluridine | Trm2 |
| 15 | D | dihydrouridine | DUS1, DUS2, DUS3, DUS4 |
| 16 | mcm5U | 5-methoxycarbonylmethy1-2′-O- | ELP1, ELP2, ELP3, ELP4, ELP5, ELP6, Trm9, |
| | m | methyluridine | ALKBH-MT, ? |
| 17 | m5C | 5-methylcytidine | Dnmt2, Dnmt2, EfmM, Nop2, Rcm1, RlmI, |
| | | | RlmO,RsmB, RsmF, Trm4, nsun2 |
| 18 | ac4C | N4-acetylcytidine | NAT10, Rra1, TmcA |
| 19 | m1A | 1-methyladenosine | Bmt2, KamB, NpmA, Rrp8, TRMT10C, |
| | | | Trm61, TrmI, TrmK,Trmt61A,Trmt61B |
| 20 | tm5U | 5-taurinomethyluridine | MTU1 |
| 21 | m1G | 1-methylguanosine | AviRa, RImA(I), RlmA(II), TRM5, TRMT10A, |
| | | | TRMT10B,TRMT10C, Taw22, Trm10, Trm5, |
| | | | Trmb, TrmD |
| 22 | Cm | 2-O-methylcytidine | |
| 23 | m1I | 1-methylinosine | |
| 24 | Ar(p) | 2′O-ribosyladenosine (phosphate) | |
| 25 | galQtRN | galactosyl-queuosine | |
| | A | | |
| 26 | mcm5U | 5-methoxycarbonylmethyluridine | ALKBH8, Trm9, ELP1, ELP2, ELP3, ELP4, |
| | | | ELP5, ELP6 |
| 27 | m1Y | 1-methylpseudouridine | |
| 28 | Gm | 2'O-methylguanosine | MRM1, Mrm1, Nop1, RNMTL1, RlmB, Spb 1, |
| | | | Trm3, Trm7, TrmH |
| 29 | manQtR | mannosyl-queuosine | Man/Gal-Q-transferase |
| | NA | | |
| 30 | yW | wybutosine | TYW1 ,2, 3, 4 |
| 31 | f5C | 5-formylcytidine | MTU1 |
| 32 | tm5s2U | 5-taurinomethy1-2-thiouridine | TrmU |
| 33 | m2, 2G | N2, N2-dimethylguanosine | Trm1 |
| 34 | chm5U | 5-carboxyhydroxymethyluridine | |
| 35 | s2U | 2-thiouridine | MnmA, Mtul, Ncs2, Ncs6, TrmU |
| 36 | mnm5s2 | 5-methylaminomethy1-2-thiouridine | MnmCD, MnmD, MnmA, Mtu1, TrmU |
| | U | | |
| 37 | m6A | N6-methyladenosine | ErmAM, ErmBC, ErmC′, Ime4, METTL14, |
| | | | METTL3, RlmF, RlmJ, RsmA, TrmM |
| 38 | mchm5U | 5-(carboxyhydroxymethyl) uridine methyl | ALKBH8 |
| | | ester | |
| 39 | m2G | N2-methylguanosine | Trm112, Trm11 |
| 40 | cmnm5U | 5-carboxymethylaminomethyluridine | tRNA (cytidine(34)-2′-O)-methyltransferase |
| 41 | Ym | 2′O-methylpseudouridine | NEP1 |
| 42 | f5 Cm | 5-formy1-2′-O-methylcytidine | |
| 43 | ncm5U | 5-carbamoylmethyluridine | ELP1, ELP2, ELP3, ELP4, ELP5, ELP6 |
| 44 | I | inosine | Tad1, Tad2, Tad3, TadA |
| 45 | g6A | N6-glycinylcarbamoyladenosine | METTL8 |
| 46 | cmnm5s | 5-carboxymethylaminomethy1-2-thiouridine | MnmA, Mtu1, TrmU, MnmE, MnmG, Mss1, |
| | 2U | | Mto1 |
| 47 | Um | 2′O-methyluridine | AviRb, MRM2, Mrm2, Nop1, RlmE, Spb1, |
| | | | Trm44, TrmJ, TrmL, aTrm56 |
| 48 | Y | pseudouridine | Cbf5, Pus1, Pus10, Pus2, Pus3, Pus4, Pus5, |
| | | | Pus6, Pus7, Pus8, Pus9, RluA, RluB, RluC, |
| | | | RluD, RluE, RluF, TruA, TruB, TruC, TruD |
| 49 | ms2i6A | 2-methylthio-N6-isopentenyladenosine | MiaA |
| 50 | m3C | 3-methylcytidine | Trm140, METTL2 and METTL6 |
| 51 | o2yW | peroxywybutosine | TRM5, TYWL, TYW2, TYW3, TYW4, TYW5, |
| | | | TRM4 |
| 52 | m5Um | 5,2′O-dimethyluridine | |
| 53 | ms2t6A | 2-methylthio-N6- | Yrdc/Sua5, MtaB/e-MtaB, SAM, ″S″ |
| | | threonylcarbamoyladenosine | |
| 54 | i6A | N6-isopentenyladenosine | MiaA, Mod5 |
| 55 | ms2io6A | 2-methylthio-N6-(cis-hydroxyisopentenyl) | MiaE |
| | | adenosine | |
| 56 | Am | 2_-O-methyladenosine | (2′-O-methyladenosine-N6-)-methyltransferase |
| 57 | m7G | 7-me thylguanosine | Abd1, ArmA, Bud23, RlmKL, RmtB, RsmG, |
| | | | Sgm, TRMB, Trm8, TrmB, WDR4 |
| 58 | t6A | N6-threonylcarbamoyladenosine | Bud32, Gon7, Cgi121 |
| 59 | | N1-methylguanine | Trm10 |
| 60 | | N7-methylguanine | Trm8, Trm82 |
| 61 | | 2′-O methylribose | Trm3, Trm13, Trm44, Trm7, Trm732, Rtt10 |
| 62 | | Ribose 2′-O-ribosyl phosphate | Rit1 |

### TREM Fusion

In an embodiment, a TREM disclosed herein comprises an additional moiety, e.g., a fusion moiety. In an embodiment, the fusion moiety can be used for purification, to alter folding of the TREM, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM. In an embodiment, the fusion moiety can be encoded by the same or different nucleic acid molecule that encodes the TREM.

### Method of Making TREMs

A TREM can be made according to any of the methods known in the art. For example, a TREM can be made using a synthetic method, e.g., synthesized using solid state synthesis or liquid phase synthesis. As another example, a TREM can be made using in vitro transcription (IVT) methods. As yet another example, a TREM can be made by expressing a vector encoding a TREM in a cell.

In vitro methods for synthesizing oligonucleotides are known in the art and can be used to make a TREM disclosed herein. For example, a chemical synthesis method of making a TREM is disclosed in Example 27. An example of an in vitro transcription method for making a TREM is disclosed in Example 28.

Additional methods for making synthetic oligonucleotides via 5′-Silyl-2′-Orthoester (2′-ACE) Chemistry are disclosed in Hartsel S A et al., (2005) Oligonucleotide Synthesis, 033-050, the entire contents of which are hereby incorporated by reference, and can be used to make a TREM disclosed herein.

Methods for designing and constructing expression vectors and modifying a host cell for production of a target (e.g., a TREM or an enzyme disclosed herein) use techniques known in the art. For example, a cell is genetically modified to express an exogenous TREM using cultured mammalian cells (e.g., cultured human cells), insect cells, yeast, bacteria, or other cells under the control of appropriate promoters. Generally, recombinant methods may be used. See, in general, Pharmaceutical Biotechnology: Fundamentals and Applications, Springer (2013); Green and Sambrook (Eds.), Molecular Cloning: A Laboratory Manual (Fourth Edition), Cold Spring Harbor Laboratory Press (2012). For example, mammalian expression vectors may comprise non-transcribed elements such as an origin of replication, a suitable promoter and enhancer, and other 5′ or 3′ flanking non-transcribed sequences. DNA sequences derived from the SV40 viral genome, for example, SV40 origin, early promoter, enhancer, splice, and polyadenylation sites may be used to provide the other genetic elements required for expression of a heterologous DNA sequence.

In an embodiment, a method of making a TREM or composition comprising a TREM disclosed herein comprises use of a host cell, e.g., a modified host cell, expressing a TREM.

The modified host cell is cultured under conditions that allow for expression of the TREM. In an embodiment, the culture conditions can be modulated to increase expression of the TREM. The method of making a TREM further comprises purifying the expressed TREM from the host cell culture to produce a composition comprising a TREM. In an embodiment the TREM is a TREM fragment, e.g., a fragment of a tRNA encoded by a deoxyribonucleic acid sequence disclosed in Table 2. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., RNase A, Dicer, Angiogenin, RNaseP, RNaseZ, Rny1 or PrrC.

In an embodiment, a method of making a TREM described herein comprises contacting (e.g., transducing or transfecting) a host cell (e.g., as described herein, e.g., a modified host cell) with an exogenous nucleic acid described herein, e.g., a DNA or RNA, encoding a TREM under conditions sufficient to express the TREM. In an embodiment, the exogenous nucleic acid comprises an RNA (or DNA encoding an RNA) that comprises a ribonucleic acid (RNA) sequence of an RNA encoded by a DNA sequence disclosed in Table 2. In an embodiment, the exogenous nucleic acid comprises an RNA sequence (or DNA encoding an RNA sequence) that is at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 2. In an embodiment, the exogenous nucleic acid comprises an RNA sequence (or DNA encoding an RNA sequence) that comprises at least 30 consecutive nucleotides of a ribonucleic acid (RNA) sequence encoded by a deoxyribonucleic acid (DNA) sequence disclosed in Table 2. In an embodiment, the exogenous nucleic acid comprises an RNA sequence (or DNA encoding an RNA sequence) that comprises at least 30 consecutive nucleotides of an RNA sequence at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 2.

In an embodiment, the host cell is transduced with a virus (e.g., a lentivirus, adenovirus or retrovirus) expressing a TREM, e.g., as described in Example 8.

The expressed TREM can be purified from the host cell or host cell culture to produce a composition comprising a TREM, e.g., as described herein. Purification of the TREM can be performed by affinity purification, e.g., as described in the MACS Isolation of specific tRNA molecules protocol, or other methods known in the art. In an embodiment, a TREM is purified by a method described in Example 7.

In an embodiment, a method of making a TREM, e.g., a composition comprising a TREM, comprises contacting a TREM with a reagent, e.g., a capture reagent comprising a nucleic acid sequence complimentary with a TREM. A single capture reagent or a plurality of capture reagents can be used to make a TREM, e.g., a composition comprising a TREM. When a single capture reagent is used, the capture reagent can have at least 50%, 55%, 60%, 65%, 70%, 75%, 80%, 85%, 90%, or 95% complimentary sequence with the TREM. When a plurality of capture reagents is used, a composition of TREMs having a plurality of different TREMs can be made. In an embodiment, the capture reagent can be conjugated to an agent, e.g., biotin.

In an embodiment, the method comprises denaturing the TREM, e.g., prior to hybridization with the capture reagent. In an embodiment, the method comprises, renaturing the TREM, after hybridization and/or release from the capture reagent.

In an embodiment, a method of making a TREM, e.g., a composition comprising a TREM, comprises contacting a TREM with a reagent, e.g., a separation reagent, e.g., a chromatography reagent. In an embodiment, a chromatography reagent includes a column chromatography reagent, a planar chromatography reagent, a displacement chromatography reagent, a gas chromatography reagent, a liquid chromatography reagent, an affinity chromatography reagent, an ion-exchange chromatography reagent, or a size-exclusion chromatography reagent.

In an embodiment, a TREM made by any of the methods described herein can be: (i) charged with an amino acid, e.g., a cognate amino acid; (ii) charged with a non-cognate amino acid (e.g., a mischarged TREM (mTREM); or (iii) not charged with an amino acid, e.g., an uncharged TREM (uTREM).

In an embodiment, a TREM made by any of the methods described herein is an uncharged TREM (uTREM). In an embodiment, a method of making a uTREM comprises culturing the host cell in media that has a limited amount of one or more nutrients, e.g., the media is nutrient starved.

In an embodiment, a charged TREM, e.g., a TREM charged with a cognate AA or a non-cognate AA, can be uncharged, e.g., by dissociating the AA, e.g., by incubating the TREM at a high temperature.

### Exogenous Nucleic Acid Encoding a TREM or a TREM Fragment

In an embodiment, an exogenous nucleic acid, e.g., a DNA or RNA, encoding a TREM comprises a nucleic acid sequence comprising a nucleic acid sequence of one or a plurality of RNA sequences encoded by a DNA sequence disclosed in Table 2, e.g., any one of SEQ ID NOs: 1-451 as disclosed in Table 2. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, or 99% identical to an RNA sequence encoded by a DNA sequence disclosed in Table 2, e.g., any one of SEQ ID NOs: 1-451 as disclosed in Table 2.

In an embodiment, an exogenous nucleic acid, e.g., a DNA or RNA, encoding a TREM comprises the nucleic acid sequence of an RNA sequence encoded by a DNA sequence disclosed in Table 2, e.g., any one of SEQ ID NOs: 1-451 as disclosed in Table 2. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, 99%, or 100% identical to a plurality of RNA sequences encoded by a DNA sequence disclosed in Table 2, e.g., any one of SEQ ID NOs: 1-451 as disclosed in Table 2. In an embodiment, an exogenous nucleic acid encoding a TREM comprises an RNA sequence encoded by a DNA sequence at least 60%, 65%, 70%, 75%, 80%, 85%, 90%, 95%, 96%, 97%, 98%, or 99% identical to a DNA sequence disclosed in Table 2, e.g., any one of SEQ ID NOs: 1-451 as disclosed in Table 2.

In an embodiment, an exogenous nucleic acid, e.g., a DNA or RNA, encoding a TREM comprises an RNA sequence of one or a plurality of TREM fragments, e.g., a fragment of an RNA encoded by a DNA sequence disclosed in Table 2, e.g., as described herein, e.g., a fragment of any one of SEQ ID NOs: 1-451 as disclosed in Table 2. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence of an RNA encoded by a DNA sequence provided in Table 2, e.g., any one of SEQ ID NOs: 1-451 as disclosed in Table 2. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence at least 60%, 65%, 70%, 75%, 80%, 85%, 90%, 95%, 96%, 97%, 98%, or 99% identical to an RNA encoded by a DNA sequence provided in Table 2. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence encoded by a DNA sequence at least 60%, 65%, 70%, 75%, 80%, 85%, 90%, 95%, 96%, 97%, 98%, or 99% identical to a DNA sequence provided in Table 2, e.g., any one of SEQ ID NOs: 1-451 as disclosed in Table 2.

In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence disclosed in Table 2 e.g., any one of SEQ ID NOs: 1-451 as disclosed in Table 2. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 2 e.g., any one of SEQ ID NOs: 1-451 as disclosed in Table 2. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 2 e.g., any one of SEQ ID NOs: 1-451 as disclosed in Table 2.

In an embodiment, the exogenous nucleic acid comprises a DNA, which upon transcription, expresses a TREM.

In an embodiment, the exogenous nucleic acid comprises an RNA, which upon reverse transcription, results in a DNA which can be transcribed to provide the TREM.

In an embodiment, the exogenous nucleic acid encoding a TREM comprises: (i) a control region sequence; (ii) a sequence encoding a modified TREM; (iii) a sequence encoding more than one TREM; or (iv) a sequence other than a tRNAMET sequence.

In an embodiment, the exogenous nucleic acid encoding a TREM comprises a promoter sequence. In an embodiment, the exogenous nucleic acid comprises an RNA Polymerase III (Pol III) recognition sequence, e.g., a Pol III binding sequence. In an embodiment, the promoter sequence comprises a U6 promoter sequence or fragment thereof. In an embodiment, the nucleic acid sequence comprises a promoter sequence that comprises a mutation, e.g., a promoter-up mutation, e.g., a mutation that increases transcription initiation, e.g., a mutation that increases TFIIIB binding. In an embodiment, the nucleic acid sequence comprises a promoter sequence which increases Pol III binding and results in increased tRNA production, e.g., TREM production.

Also disclosed herein is a plasmid comprising an exogenous nucleic acid encoding a TREM. In an embodiment, the plasmid comprises a promoter sequence, e.g., as described herein.

### Composition Comprising a TREM

In an embodiment, a composition comprising a TREM, e.g., a pharmaceutical composition comprising a TREM, comprises a pharmaceutically acceptable excipient. Exemplary excipients include those provided in the FDA Inactive Ingredient Database (https://www.accessdata.fda.gov/scripts/cder/iig/index.Cfm).

In an embodiment, a composition comprising a TREM, e.g., a pharmaceutical composition comprising a TREM, comprises at least 1, 2, 3, 4, 5, 6, 7, 8, 9, 10, 15, 20, 30, 40, 50, 60, 70, 80, 90, 100 or 150 grams of TREM. In an embodiment, a composition comprising a TREM, e.g., a pharmaceutical composition comprising a TREM, comprises at least 1, 2, 3, 4, 5, 6, 7, 8, 9, 10, 15, 20, 30, 40, 50 or 100 milligrams of TREM.

In an embodiment, a composition comprising a TREM, e.g., a pharmaceutical composition comprising a TREM, is at least 10, 20, 30, 40, 50, 60, 70, 80, 90, 95 or 99% dry weight TREMs.

In an embodiment, a composition comprising a TREM produced by any of the methods of making disclosed herein can be charged with an amino acid using an in vitro charging reaction as disclosed in Example 12, or as known in the art.

In an embodiment, a composition comprising a TREM comprises at least 1×10⁶ TREM molecules, at least 1×10⁷ TREM molecules, at least 1×10⁸ TREM molecules or at least 1×10⁹ TREM molecules.

### TREM Purification

A composition comprising a TREM, e.g., a pharmaceutical composition comprising a TREM, may be purified from host cells by nucleotide purification techniques. In one embodiment, a composition comprising a TREM is purified by affinity purification, e.g., as described in the MACS Isolation of specific tRNA molecules protocol, or by a method described in Example 7. In one embodiment, a composition comprising a TREM is purified by liquid chromatography, e.g., reverse-phase ion-pair chromatography (IP-RP), ion-exchange chromatography (IE), affinity chromatography (AC), size-exclusion chromatography (SEC), and combinations thereof. See, e.g., Baronti et al. Analytical and Bioanalytical Chemistry (2018) 410:3239-3252.

### TREM Quality Control and Production Assessment

A TREM or a composition comprising a TREM, e.g., a pharmaceutical composition comprising a TREM, produced by any of the methods disclosed herein can be assessed for a characteristic associated with the TREM or the TREM preparation, such as purity, host cell protein or DNA content, endotoxin level, sterility, TREM concentration, TREM structure, or functional activity of the TREM. Any of the above-mentioned characteristics can be evaluated by providing a value for the characteristic, e.g., by evaluating or testing the TREM, the composition comprising a TREM, or an intermediate in the production of the composition comprising a TREM. The value can also be compared with a standard or a reference value. Responsive to the evaluation, the composition comprising a TREM can be classified, e.g., as ready for release, meets production standard for human trials, complies with ISO standards, complies with cGMP standards, or complies with other pharmaceutical standards. Responsive to the evaluation, the composition comprising a TREM can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the composition comprising a TREM. For example, the composition comprising a TREM can be modulated, processed or re-processed to (i) increase the purity of the composition comprising a TREM; (ii) decrease the amount of HCP in the composition; (iii) decrease the amount of DNA in the composition; (iv) decrease the amount of fragments in the composition; (v) decrease the amount of endotoxins in the composition; (vi) increase the in vitro translation activity of the composition; (vii) increase the TREM concentration of the composition; or (viii) inactivate or remove any viral contaminants present in the composition, e.g., by reducing the pH of the composition or by filtration.

In an embodiment, the TREM (e.g., the composition comprising a TREM or an intermediate in the production of the composition comprising a TREM) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., the composition comprising a TREM or an intermediate in the production of the composition comprising a TREM) has a host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, the TREM (e.g., the composition comprising a TREM or an intermediate in the production of the composition comprising a TREM) has a host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the composition.

In an embodiment, the TREM (e.g., the composition comprising a TREM or an intermediate in the production of the composition comprising a TREM) has a DNA content, e.g., host cell DNA content, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, the TREM (e.g., the composition comprising a TREM or an intermediate in the production of the composition comprising a TREM) has less than 0.1%, 1%, 2%, 3%, 4%, 5%, 6%, 7%, 8%, 9%, 10%, 15%, 20%, 25% TREM fragments.

In an embodiment, the TREM (e.g., the composition comprising a TREM or an intermediate in the production of the composition comprising a TREM) has low levels or absence of endotoxins, e.g., as measured by the Limulus amebocyte lysate (LAL) test;

In an embodiment, the TREM (e.g., the composition comprising a TREM or an intermediate in the production of the composition comprising a TREM) has in-vitro translation activity, e.g., as measured by an assay described in Example 15.

In an embodiment, the TREM (e.g., the composition comprising a TREM or an intermediate in the production of the composition comprising a TREM) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM (e.g., the composition comprising a TREM or an intermediate in the production of the composition comprising a TREM) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM (e.g., the composition comprising a TREM or an intermediate in the production of the composition comprising a TREM) has an absence of, or an undetectable level of a viral contaminant, e.g., no viral contaminants. In an embodiment, a viral contaminant, e.g., any residual virus, present in the composition is inactivated or removed. In an embodiment, a viral contaminant, e.g., any residual virus, is inactivated, e.g., by reducing the pH of the composition. In an embodiment, a viral contaminant, e.g., any residual virus, is removed, e.g., by filtration or other methods known in the field.

### TREM Administration

A composition comprising a TREM or a pharmaceutical composition comprising a TREM described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

In an embodiment, a composition comprising a TREM or a pharmaceutical composition comprising a TREM disclosed herein is administered to a subject having a symptom or disorder disclosed herein. In an embodiment, a composition comprising a TREM or a pharmaceutical composition comprising a TREM disclosed herein is administered to prevent or treat the symptom or disorder. In an embodiment, administration of the composition comprising a TREM or a pharmaceutical composition comprising a TREM results in treatment or prevention of the symptom or disorder. In an embodiment, administration of the composition comprising a TREM or a pharmaceutical composition comprising a TREM modulates a tRNA pool in the subject, e.g., resulting in treatment of the symptom or disorder. In an embodiment, the disorder is chosen from Table 1.

In an embodiment, a composition comprising a TREM or a pharmaceutical composition comprising a TREM disclosed herein is administered to a cell from a subject having a symptom or disorder disclosed herein. In an embodiment, administration of the composition comprising a TREM or the pharmaceutical composition comprising a TREM modulates a tRNA pool in the cell from the subject. In an embodiment, the composition comprising a TREM or pharmaceutical composition comprising a TREM can be administered to the cell in vivo, in vitro or ex vivo. In an embodiment, the subject has a disorder chosen from Table 1.

In an embodiment, a composition comprising a TREM or a pharmaceutical composition comprising a TREM disclosed herein is administered to a tissue in a subject having a symptom or disorder disclosed herein. In an embodiment, administration of the composition comprising a TREM or pharmaceutical composition comprising a TREM modulates a tRNA pool in the tissue in the subject. In an embodiment, the subject has a disorder chosen from Table 1.

### Vectors and Carriers

In some embodiments the TREM, composition comprising a TREM or pharmaceutical composition comprising a TREM described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

### Carriers

A TREM, a composition comprising a TREM or a pharmaceutical composition comprising a TREM described herein may comprise, may be formulated with, or may be delivered in, a carrier.

### Viral Vectors

The carrier may be a viral vector (e.g., a viral vector comprising a sequence encoding a TREM). The viral vector may be administered to a cell or to a subject (e.g., a human subject or animal model) to deliver a TREM, a composition comprising a TREM or a pharmaceutical composition comprising a TREM. A viral vector may be systemically or locally administered (e.g., injected).

Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a composition comprising a TREM or a pharmaceutical composition comprising a TREM described herein can be administered to a cell in a vesicle or other membrane-based carrier.

In embodiments, a TREM, composition comprising a TREM or pharmaceutical composition comprising a TREM described herein is administered in or via a cell, vesicle or other membrane-based carrier. In one embodiment, the TREM, composition comprising a TREM or pharmaceutical composition comprising a TREM can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for a TREM, composition comprising a TREM or pharmaceutical composition comprising a TREM described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for a TREM, or composition comprising a TREM, or a pharmaceutical composition comprising a TREM described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, composition comprising a TREM or a pharmaceutical composition comprising a TREM described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a TREM, a composition comprising a TREM, or a pharmaceutical composition comprising a TREM described herein.

Virosomes and virus-like particles (VLPs) can also be used as carriers to deliver a TREM, a composition comprising a TREM, or a pharmaceutical composition comprising a TREM described herein to targeted cells.

Plant nanovesicles, e.g., as described in WO2011097480A1, WO2013070324A1, or WO2017004526A1 can also be used as carriers to deliver the TREM, composition comprising a TREM, or pharmaceutical composition comprising a TREM described herein.

Delivery without a Carrier

A TREM, a composition comprising a TREM, or a pharmaceutical composition comprising a TREM described herein can be administered to a cell without a carrier, e.g., via naked delivery of the TREM, composition comprising a TREM, or pharmaceutical composition comprising a TREM.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a composition comprising a TREM, or a pharmaceutical composition comprising a TREM described herein is delivered to a cell without a carrier, e.g., via naked delivery. In some embodiments, the delivery without a carrier, e.g., naked delivery, comprises delivery with a moiety, e.g., a targeting peptide.

### Use of TREMs

A composition comprising a TREM (e.g., a pharmaceutical composition comprising a TREM as described herein) can be used to modulate a tRNA pool in a cell or subject, e.g., as described herein. In embodiments, a composition comprising a TREM (e.g., a pharmaceutical composition comprising a TREM) described herein is contacted with a cell or tissue, or administered to a subject in need thereof, in an amount and for a time sufficient to modulate (increase or decrease) the tRNA pool. In embodiments, the tRNA pool comprises a first tRNA moiety and an additional tRNA moiety, e.g., a second tRNA moiety. In an embodiment, a tRNA moiety comprises an endogenous tRNA and/or a TREM.

In an embodiment, a composition comprising a TREM (e.g., a pharmaceutical composition comprising a TREM as described herein) can be used to treat a subject having an endogenous ORF comprising a codon comprising a synonymous mutation (a synonymous mutation codon or SMC). In an embodiment, the subject has a disorder disclosed in Table 1.

A composition comprising a TREM (e.g., a pharmaceutical composition comprising a TREM as described herein) can also be used to modulate a function in a cell, tissue or subject. In embodiments, a composition comprising a TREM (e.g., a pharmaceutical composition comprising a TREM) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein localization; protein folding; protein stability; protein transduction; or protein compartmentalization.

A parameter may be modulated, e.g., by at least 5% (e.g., at least 10%, 15%, 20%, 25%, 30%, 40%. 50%. 60%. 70%, 80%, 90%, 100%, 150%, 200% or more) compared to a reference tissue, cell or subject (e.g., a healthy, wild-type or control cell, tissue or subject).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

Table of Contents for Examples
Manufacture and use of TREMs

| | |
|---|---|
| Example 1 | Manufacture of a TREM in a mammalian production host cell from |
| | transient transfection |
| Example 2 | Manufacture of a TREM in a mammalian production host cell from |
| | stable cell lines |
| Example 3 | Manufacture of a TREM in a mammalian production host cell from |
| | stable cell lines-2 |
| Example 4 | Delivery of TREMs to mammalian cells |
| Example 5 | Increased cell growth in mammalian cells with TREM |
| Example 6 | TREM translational activity assay in Human Cell Extract Cell-Free |
| | Protein Synthesis (hCFPS) lysate |

Manufacture and preparation of TREMs

| | |
|---|---|
| Example 7 | Manufacture of TREM in a mammalian production host cell, and use |
| | thereof to modulate a cellular function |
| Example 8 | Manufacture of TREM in a mammalian production host cell, and use |
| | thereof to modulate a cellular function-2 |
| Example 9 | Manufacture of TREM in a modified mammalian production host cell |
| | expressing an oncogene |
| Example 10 | Preparation of a TREM production host cell modified to inhibit a |
| | repressor of tRNA synthesis |
| Example 11 | Manufacture of TREMs in modified mammalian production host cell |
| | overexpressing an oncogene and a tRNA modifying enzyme |

Production of TREMs

| | |
|---|---|
| Example 12 | Production of mischarged TREMs |
| Example 13 | Production of TREM fragments (in vitro) |
| Example 14 | Production of TREM fragments in a cell expression system |

Assays to analyze TREM activity

| | |
|---|---|
| Example 15 | TREM translational activity assay |
| Example 16 | Assay for modulation of cell state |
| Example 17 | Assay for the activity of an uncharged TREM to modulate autophagy |
| Example 18 | Assay for activity of a mischarged TREM (mTREM) |

Identifying SMC-containing proteins that would benefit from TREM administration

| | |
|---|---|
| Example 19 | Identification of disease-associated SMC that could be ameliorated by |
| | TREM modulation |
| Example 20 | PNPL3A SMC |
| Example 21 | TERT SMC |
| Example 22 | ACHE SMC |
| Example 23 | CFTR SMC |
| Example 24 | MAP3K1 SMC |

Production of TREMs

| | |
|---|---|
| Example 25 | Production of a candidate TREM complementary to the SMC through |
| | mammalian cell purification |
| Example 26 | Production of a candidate TREM complementary to the SMC through |
| | bacterial cell purification |
| Example 27 | Production of a candidate TREM complementary to the SMC through |
| | chemical synthesis |
| Example 28 | Production of a candidate TREM complementary to the SMC through in |
| | vitro transcription |

Modulation of tRNA pool

| | |
|---|---|
| Example 29 | Modulation of a tRNA pool through TREM administration to a cell |
| Example 30 | Modulation of a tRNA pool through TREM administration to a cell |
| | using liposome |
| Example 31 | Modulation of a tRNA pool through delivery of TREM-encoding |
| | plasmid to a cell |
| Example 32 | Modulation of a tRNA pool through delivery of a TREM-encoding viral |
| | vector to a cell |

Method of making a cell line with an SMC of interest to test effects of TREM administration

| | |
|---|---|
| Example 33 | System to test the effects of TREM administration on an SMC |
| | containing ORF |

Assays to test TREM administration effects on characteristics of SMC-containing protein

| | |
|---|---|
| Example 34 | Determining that administration of a TREM affects protein expression |
| | levels of SMC-containing ORF |
| Example 35 | TREM administration changes protein translation rate of SMC- |
| | containing ORF |
| Example 36 | Determining that modulation of a TREM complementary to the SMC |
| | changes the function of the protein derived from the SMC-containing ORF |
| Example 37 | Determining that modulation of a TREM complementary to an SMC |
| | changes the localization of the protein derived from the SMC-containing ORF |
| Example 38 | Determining that modulation of a TREM complementary to an SMC |
| | changes folding of the protein translated from the SMC-containing ORF |
| Example 39 | Determining that modulation of a TREM complementary to an SMC |
| | alters the cellular phenotype resulting from translation of the SMC- |
| | containing ORF |
| Example 40 | Modulation of TREM to ameliorate a disease state resulting from |
| | translation of the SMC-containing ORF |

### Example 1: Manufacture of a TREM in a Mammalian Production Host Cell from Transient Transfection

This example describes the manufacture of a TREM produced in mammalian host cells which transiently express a TREM.

### Plasmid Generation

To generate a plasmid comprising a sequence encoding a TREM, in this example, iMet-CAT TREM, a DNA fragment containing one copy of the sequence AGCAGAGTGGCGCAGCGGAAGCGTGCTGGGCCCATAACCCAGAGGTCGATGGATCG AAACCATCCTCTGCTA (SEQ ID NO: 262) was synthesized and cloned into the pLKO.1-puro-mCherry backbone plasmid with a U6 promoter following the manufacturer's instructions and standard molecular cloning techniques.

### Transfection

Three (3) μg of plasmid described above was used to transfect a T175 flask of HEK293T cells plated at 80% confluency using 9 uL of lipofectamine RNAiMax reagents according to the manufacturer's instructions. Cells were harvested at 48 hours post-transfection for purification.

### Purification Using a Small RNA Isolation Kit

The iMet-overexpressing cells were lysed. To generate a small RNA (sRNA) fraction, a small RNA isolation kit, such as the Qiagen miRNeasy kit, was used to separate RNAs smaller than 200 nucleotides from the rest of the total RNA pool in the lysate, per manufacturer's instructions. To further exclude larger RNAs, a LiCl precipitation was performed to remove remaining large RNAs in the sRNA fraction. Finally, the sRNA fraction was added to a G50 column to remove RNAs smaller than 10 nucleotides from the sRNA fraction and for buffer exchange.

To isolate the TREM from the sRNA fraction, a probe binding method was used. A biotinylated capture probe corresponding to a DNA probe or a 2′-OMe nucleic acid that is complementary to a unique region of the target TREM being purified, in this example, a probe conjugated to biotin at the 5′ end with the sequence TAGCAGAGGATGGTTTCGATCCATCA (SEQ ID NO: 455), was used to bind and purify the iMet-CAT-TREM. The sRNA fraction was incubated with annealing buffer and the biotinylated capture probe at 90° C. for 4-5 minutes and cooled at a rate of 0.1° C./s to 25° C.

The admixture was then incubated with binding buffer and streptavidin-conjugated RNase-free magnetic beads for 15 minutes to enable binding of the DNA-TREM complexes to the beads. The mixture was then added to a magnetic field separator rack and washed 2-3 times with wash buffer. The TREM retained on the beads was eluted by adding elution buffer with or without a DNase enzyme to ensure complete removal of the DNA capture probe and then admixed with a pharmaceutically acceptable excipient to make a test TREM product.

### Example 2: Manufacture of a TREM in a Mammalian Production Host Cell from Stable Cell Lines

This example describes the manufacture of a TREM produced in mammalian host cells stably expressing a TREM.

### Preparation of TREM Expressing Lentivirus

To prepare a TREM expressing lentivirus in a 10 mm dish, packaging cells, such as HEK293T cells (293T cells (ATCC® CRL-3216™), were forward transfected with 9 μg of a plasmid comprising a sequence encoding a TREM as described in Example 1, and 9 μg ViraPower lentiviral packaging mix using TransIT-LT1 transfection reagents according to the manufacturer's instructions.

After 18 hours, the media was replaced with fresh antibiotic-free high-FBS (30% FBS) media and 24 hours later, the media containing the virus was harvested and stored at 4° C. Another 15 mL of high-FBS media was added to the plate and harvested 24 hours later. Both virus-containing media harvests were pooled and filtered through a 0.45-micron filter. The viral copy number was assessed using the Lenti-X qRT-PCR Titration Kit according to the manufacturer's protocol.

Transduction of Host Cells with TREM Expressing Lentivirus

To transduce the cells with TREM expressing lentivirus, the lentivirus-containing media was diluted with complete cell media at a 1:4 ratio, in the presence of 10 μg/mL polybrene, and added to the cells. In this example 293T cells were used. The plate was spun for 2 hours at 1000×g to spin infect the cells. After 18 hours, the media was replaced to allow the cells to recover. Forty-eight hours after transduction, puromycin (at 2 μg/mL) antibiotic selection was performed for 5-7 days alongside a population of untransduced control cells.

The TREMs were isolated, purified, and formulated as described in Example 1 to result in a TREM preparation.

### Purification Using Phenol Chloroform Extraction

The total RNA pool from cells was recovered from cells by guanidinium thiocyanate-phenol-chloroform extraction and concentrated by ethanol precipitation as described in J. Sambrook and D. Russell (2001) Molecular Cloning: A Laboratory Manual, vol. 2, Cold Spring Harbor Laboratory Press, New York, N.Y., USA, 3rd edition2. The total tRNA pool in the precipitate was then separated from larger nucleic acids (including rRNA and DNA) by precipitation under high lithium salt conditions as described in Cathala, G. et al., DNA, 1983; 2(4):329-35. The elution fraction containing the TREM was further purified through probe binding.

The TREM fraction was incubated with annealing buffer and the biotinylated capture probe corresponding to a DNA probe or a 2′-OMe nucleic acid that is complementary to a unique region of the target TREM being purified. In this example, a probe conjugated to biotin at the 5′ end with the sequence TAGCAGAGGATGGTTTCGATCCATCA (SEQ ID NO: 455), was used to purify the TREM comprising iMet-CAT. The mixture was incubated at 90° C. for 4-5 minutes and cooled at a rate of 0.1° C./s to 25° C.

The admixture was then incubated with binding buffer and streptavidin-conjugated RNase-free magnetic beads for 15 minutes to enable binding of the DNA-TREM complexes to the beads. The mixture was then added to a magnetic field separator rack and washed 2-3 times. The TREM retained on the beads were eluted by adding elution buffer with or without a DNase enzyme to ensure complete removal the DNA capture probe and then admixed with a pharmaceutically acceptable excipient to make a test TREM product.

### Example 3: Manufacture of a TREM in a Mammalian Production Host Cell from Stable Cell Lines −2

This example describes the manufacture of a TREM from crude cell lysate, produced from mammalian host cells.

### Generation of Stable Cells Expressing TREM

In this example, a plasmid comprising a sequence encoding a TREM is generated as described in Example 1 or 2. Preparation of TREM expressing lentivirus and transduction of host cells with TREM-expressing lentivirus was performed as described in Example 2.

Purification from Crude Cell Lysate

The TREM-overexpressing cells, in this example the iMet-CAT-TREM overexpressing cells, were lysed and the lysed material was incubated with annealing buffer and the biotinylated capture probe corresponding to a DNA probe or a 2′-OMe nucleic acid that is complementary to a unique region of the target TREM being purified. In this example, a probe conjugated to biotin at the 5′ end with the sequence TAGCAGAGGATGGTTTCGATCCATCA (SEQ ID NO: 455), was used to purify the TREM comprising iMet-CAT. The mixture was incubated at 90° C. for 4-5 minutes and cooled at a rate of 0.1° C./s to 25° C.

The admixture was then incubated with binding buffer and streptavidin-conjugated RNase-free magnetic beads for 15 minutes to enable binding of the DNA-TREM complexes to the beads. The mixture was then added to a magnetic field separator rack and washed 2-3 times. The TREM retained on the beads were eluted by adding elution buffer with or without a DNase enzyme to ensure complete removal the DNA capture probe and then admixed with a pharmaceutically acceptable excipient to make a test TREM product.

### Example 4: Delivery of TREMs to Mammalian Cells

This example describes the delivery of a TREM to mammalian cells.

To ensure proper folding, the TREM was heated at 85° C. for 2 minutes and then snap cooled at 4° C. for 5 minutes. To deliver the TREM to mammalian cells, 100 nM of two TREM preparations labeled with Cy3 at different positions (Cy3-iMET-1 and Cy3-iMET-2) were transfected in U2OS (U-2 OS (ATCC® HTB-96™)), H1299 (NCI-H1299 (ATCC® CRL-5803™)), and HeLa (HeLa (ATCC® CCL-2™)) cells using RNAiMax reagents according to the manufacturer's instructions. After 18 hours, the transfection media was removed and replaced with fresh complete media (U2OS: McCoy's 5A, 10% FBS, 1% PenStrep; H1299: RPMI1640, 10% FBS, 1% PenStrep; HeLa: EMEM, 10% FBS, 1% PenStrep).

To observe TREM delivery to cells, the cells were monitored in a live cell analysis system. In this example, the IncuCyte (from Essen Bioscience) was used to monitor cells. The cells were monitored for 4 days (20×, red 550 ms).

Cy3 fluorescence signal was readily detected from cells that had been delivered the Cy3-labeled TREMs. The Cy3 fluorescence signal was observed for over 48 hours from the cells in which the TREMs had been delivered. Detection of Cy-3 fluorescence from the cells confirmed delivery of the Cy3-labeled TREM to the cells.

### Example 5: Increased Cell Growth in Mammalian Cells with TREM

This example describes increased cell growth of a mammalian cell upon TREM delivery.

To ensure proper folding, the iMet TREM was heated at 85° C. for 2 minutes and then snap cooled at 4° C. for 5 minutes. To deliver the iMet TREM to mammalian cells, 100 nM of Cy3-labeled iMet TREM was transfected in U2OS (U-2 OS (ATCC® HTB-96™)), H1299 (NCI-H1299 (ATCC® CRL-5803™)), and HeLa (HeLa (ATCC® CCL-2™)) cells using RNAiMax reagents according to the manufacturer's instructions. As a control, a Cy3-labeled non targeted control siRNA was delivered to cells. After 18 hours, the transfection media was removed and replaced with fresh complete media (U2OS: McCoy's 5A, 10% FBS, 1% PenStrep; H1299: RPMI1640, 10% FBS, 1% PenStrep; HeLa: EMEM, 10% FBS, 1% PenStrep). To observe changes in cell growth, the cells were monitored in a live cell analysis system, in this example in the IncuCyte (from Essen Bioscience), for 4 days (20×, phase contrast).

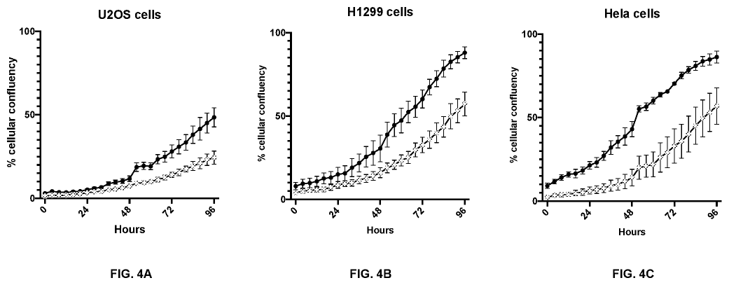

Delivery of iMet TREM to U2OS cells (FIG. 4A), H1299 (FIG. 4B) or Hela cells (FIG. 4C) led to a substantial increase in cell growth in all of the cell lines that were tested. The increase in cell growth was compared to cell growth observed with delivery of a Cy3-labeled non-targeted control (Cy3-NTC). The data demonstrates that delivery of a TREM to cells results in increased proliferation and growth.

### Example 6: TREM Translational Activity Assay in Human Cell Extract Cell-Free Protein Synthesis (hCFPS) Lysate

This example describes a TREM mediated increase in translational activity in a cell-free lysate system.

### Preparing Human Cell Extracts

HEK293T cells were grown to ˜80% confluency in 40×150 mm culture dishes. The cells were harvested, washed in PBS, resuspended 1:1 in ice-cold hypotonic lysis buffer (20 mM HEPES pH 7.6, 10 mM KAc, 1.5 mM MgAc, 5 mM DTT and 5× complete EDTA-free proteinase inhibitor cocktail) and incubated on ice for 30 minutes. Cells were lysed using a Dounce homogenizer or by passing the lysate through a 27G needle, until >95% of the cells were disrupted. The lysate was centrifuged at 14,000 g for 10 mins at 4° C., the supernatant was collected and diluted with the hypotonic lysis buffer to get a ˜15 mg/ml protein solution.

Transcribing mRNAs

mRNA transcription templates were designed to have a T7 polymerase promoter, a beta-globin 3′UTR, a nanoLuc ORF, and a short artificial 3′UTR. The templates were PCR amplified and used to transcribe capped and poly-adenylated mRNAs with a HiScribe T7 ARCA mRNA kit with tailing (New England Biolabs) following the manufacturer's recommended protocol.

Performing the TREM Translational Activity Assay in hCFPS Lysate

Translation reactions were set up in translation buffer (16 mM HEPES pH 7.6, 2.2 mM MgAc, 60 mM KCl, 0.02 mM complete amino acid mix, 1 mM ATP, 0.5 mM GTP, 20 mM creatine phosphate, 0.1 μg/μL creatine kinase, 0.1 mM spermidine, 2 U/μl RiboLock RNase Inhibitor) with 35% HEK293T lysate, 0.02 μM capped and poly-adenylated nanoLuc mRNA and 2 μM cell-purified TREM (purified according to Example 2). The reactions were performed in 10 μl triplicates at 37° C. for 30 minutes. For the control reactions, one control reaction was performed with no TREM addition to the reaction and one control reaction was performed with no mRNA addition to the reaction. Then, the NanoLuc activity was detected by mixing each reaction with 40 μl of room temperature Nano-Glo Luciferase assay system (Promega) and reading the luminescence in a plate reader.

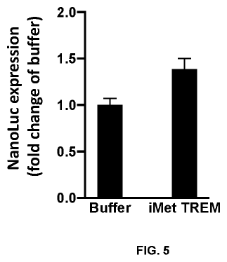

As shown in FIG. 5, the iMET TREM reaction resulted in about a 1.5 fold increase in NanoLuc expression as compared to the control reaction (buffer). The data shows that delivery of the TREM results in an increase in nanoLuc mRNA translation as reflected by an increase in luminescence.

### Example 7: Manufacture of TREM in a Mammalian Production Host Cell, and Use Thereof to Modulate a Cellular Function

This example describes the manufacturing of a TREM produced in mammalian host cells.

### Plasmid Generation

To generate a plasmid comprising a TREM which comprises a tRNA gene, in this example, tRNAiMet, a DNA fragment containing the tRNA gene (chr6.tRNA-iMet(CAT) with genomic location 6p22.2 and sequence AGCAGAGTGGCGCAGCGGAAGCGTGCTGGGCCCATAACCCAGAGGTCGATGGATCG AAACCATCCTCTGCTA (SEQ ID NO: 262)) is PCR-amplified from human genomic DNA using the following primer pairs: 5′-TGAGTTGGCAACCTGTGGTA (SEQ ID NO: 452) and 5′-TTGGGTGTCCATGAAAATCA (SEQ ID NO: 453). This fragment is cloned into the pLKO.1 puro backbone plasmid with a U6 promoter (or any other RNA polymerase III recruiting promoter) following the manufacturer's instructions.

### Transfection

1 mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR).

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe or a 2′-OMe nucleic acid that is complementary to a unique region of the target TREM being purified, in this example, a probe conjugated to biotin at the 3′ end with the sequence UAGCAGAGGAUGGUUUCGAUCCAUCA (SEQ ID NO: 454), is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Use

One microgram of the test TREM preparation and a control agent are contacted by transfection, electroporation or liposomal delivery, with a cultured cell line, such as a HEP-3B or HEK293T, a tissue or a subject, for a time sufficient for the TREM preparation to modulate a translation level or activity of the cell, relative to the control agent.

### Example 8: Manufacture of TREM in a Mammalian Production Host Cell, and Use Thereof to Modulate a Cellular Function

This example describes the manufacturing of a TREM produced in mammalian host cells.

### Plasmid Generation

To generate a plasmid comprising a TREM which comprises a tRNA gene, in this example, tRNA-iMet-CAT, a DNA fragment containing at least one copy of the tRNA gene with the sequence AGCAGAGTGGCGCAGCGGAAGCGTGCTGGGCCCATAACCCAGAGGTCGATGGATCG AAACCATCCTCTGCTA (SEQ ID NO: 262) is synthesized and cloned into the pLKO.1 puro backbone plasmid with a U6 promoter (or any other RNA polymerase III recruiting promoter) following the manufacturer's instructions and standard molecular cloning techniques.

### Transfection

1 mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by Northern blot, or by quantitative PCR (q-PCR) or Nanopore sequencing.

### Purification

At the optimized harvest timepoint, the cells are lysed and separation from the lysate of RNAs smaller than 200 nucleotides is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe or a 2′-OMe nucleic acid that is complementary to a unique region of the target TREM being purified. In this example, a probe with the sequence 5′biotin-TAGCAGAGGATGGTTTCGATCCATCA (SEQ ID NO: 455) is used to purify the TREM comprising tRNA-iMet (CAT). The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. Alternatively, the beads are washed three times and the final wash is examined by UV spectroscopy to measure the amount of nucleic acid present in the final wash. The TREM retained on the beads are eluted three times using RNase-free water which can be pre-heated to 80° C., and then admixed with a pharmaceutically acceptable excipient to make a test TREM product.

### Use

One microgram of the test TREM preparation and a control agent are contacted by transfection, electroporation or liposomal delivery, with a cultured cell line, such as HeLa, HEP-3B or HEK293T, a tissue or a subject, for a time sufficient for the TREM preparation to modulate a translation level or activity of the cell, relative to the control agent.

### Example 9: Manufacture of TREMs in Modified Mammalian Production Host Cell Expressing an Oncogene

This example describes the manufacturing of a TREM in mammalian host cells modified to overexpress myc.

### Plasmid Generation and Host Cell Modification

To make the production host cells for this example, HeLa cells (ATCC® CCL-2™) or HEP-3B cells (ATCC® HB-8064™) are transfected with a plasmid containing the gene sequence coding for the c-myc oncogene protein (e.g., pcDNA3-cmyc (Addgene plasmid #16011)) using routine molecular biology techniques. The resulting cell line is referred to herein as HeLamyc+ host cells or HEP-3Bmyc+ host cells.

### Preparation of TREM Expressing Lentivirus

To prepare a TREM expressing lentivirus, HEK293T cells are co-transfected with 3 μg of each packaging vector (pRSV-Rev, pCMV-VSVG-G and pCgpV) and 9 μg of the plasmid comprising a TREM as described in Example 7, using Lipofectamine 2000 according to manufacturer's instructions. After 24 hours, the media is replaced with fresh antibiotic-free media and after 48 hours, virus-containing supernatant is collected and centrifuged for 10 min at 2000 rpm before being filtered through a 0.45 μm filter.

Transduction of Host Cells with TREM Expressing Lentivirus

2 mL of virus prepared as described above is used to transduce 100,000 HeLamyc+ host cells or HEP-3Bmyc+ host cells, in the presence of 8 μg/mL polybrene. Forty-eight hours after transduction, puromycin (at 2 μg/mL) antibiotic selection is performed for 2-7 days alongside a population of untransduced control cells.

The TREMs are isolated, purified, and formulated as described in Example 7 or 8 to result in a composition comprising a TREM or preparation comprising a TREM.

### Example 10: Preparation of a TREM Production Host Cell Modified to Inhibit a Repressor of tRNA Synthesis

This example describes the preparation of Hek293Maf−/TRM1 cells for the production of a TREM.

Maf1 is a repressor of tRNA synthesis. A Maf1 knockout HEK293T cell line is generated using standard CRISPR/Cas knockout techniques, e.g., a CRISPR/Cas system can be designed to introduce a frameshift mutation in a coding exon of Maf1 to reduce the expression of Maf1 or knockout Maf1 expression, to generate a Hek293Maf− cell line that has reduced expression level and/or activity of Maf1. This cell line is then transfected with an expression plasmid for modifying enzyme Trm1 (tRNA (guanine26-N2)-dimethyltransferase) such as pCMV6-XL4-Trm1, and selected with a selection marker, e.g., neomycin, to generate a stable cell line overexpressing Trm1 (Hek293Maf−/TRM1 cells).

Hek293Maf−/TRM1 cells can be used as production host cells for the preparation of a TREM as described in any of Examples 7-9.

### Example 11: Manufacture of TREM in Modified Mammalian Production Host Cell Overexpressing an Oncogene and a tRNA Modifying Enzyme

This Example describes the manufacturing of a TREM in mammalian host cells modified to overexpress Myc and Trm1.

### Plasmid Generation

In this example, a plasmid comprising a TREM is generated as described in Example 7 or 8.

### Host Cell Modification, Transduction and Purification

A human cell line, such as HEK293T, stably overexpressing Myc oncogene is generated by transduction of retrovirus expressing the myc oncogene from the pBABEpuro-c-myc^{T58A} plasmid into HEK293T cells. To generate myc-expressing retrovirus, HEK293T cells are transfected using the calcium phosphate method with the human c-myc retroviral vector, pBABEpuro-c-myc^{T58A} and the packaging vector, ψ2 vector. After 6 hours, transfection media is removed and replaced with fresh media. After a 24-hour incubation, media is collected and filtered through a 0.45 um filter. For the retroviral infection, HEK293T cells are infected with retrovirus and polybrene (8 ug/ml) using spin infection at 18° C. for 1 hour at 2500 rpm. After 24 hours, the cell culture medium is replaced with fresh medium and 24 hours later, the cells are selected with 2 μg/mL puromycin. Once cells stably overexpressing the oncogene myc are established, they are transfected with a Trm1 plasmid, such as the pCMV6-XL4-Trm1 plasmid, and selected with a selection marker, in this case with neomycin, to generate a stable cell line overexpressing Trm1, in addition to Myc. In parallel, lentivirus to overexpress TREM is generated as described in Example 9 with HEK293T cells and PLKO.1-tRNA vectors.

1×10⁵ cells overexpressing Myc and Trm1 are transduced with the TREM virus in the presence of 8 μg/mL polybrene. Media is replaced 24 hours later. Forty-eight hours after transduction, antibiotic selection is performed with 2 μg/mL puromycin for 2-7 days alongside a population of untransduced control cells. The TREMs are isolated, purified and formulated using the method described in Example 7 or 8 to produce a TREM preparation.

### Example 12: Production of a Mischarged TREM

This example describes the production of a TREM charged with an amino acid that does not correspond to its natural anticodon.

A TREM is produced as described in any of Examples 7-11. The TREM product is charged with a heterologous amino acid using an in vitro charging reaction known in the art (see, e.g., Walker & Fredrick (2008) Methods (San Diego, Calif.) 44(2):81-6). Briefly, the purified TREM, for example a TREM comprising tRNA-Val(GTG), is placed in a buffer with the heterologous amino acid of interest (for example glutamic acid), and the corresponding aminoacyl-tRNA synthetase (for example a Valyl-tRNA synthetase mutated to enhance tRNA mischarging), to induce TREM charging. To isolate the aminoacyl-TREM, the in vitro charging reaction is passed through a spin column and the concentration based on the A260 absorbance is determined as is the extent of aminoacylation using acid gel electrophoresis. Aminoacylated TREM can also be isolated by binding to His6-tagged (SEQ ID NO: 456) EF-Tu, followed by affinity chromatography on Ni-NTA agarose, phenol-chloroform extraction and subsequent precipitation of the nucleic acids as described in Rezgui et al., 2013, PNAS 110:12289-12294.

### Example 13: Production of a TREM Fragment (In Vitro)

This example describes the production of a TREM fragment in vitro, from a TREM manufactured in mammalian host cells.

A TREM is made as described in any Example above. An enzymatic cleavage assay with enzymes known to generate tRNA fragments, such as RNase A or angiogenin, is used to produce fragments for administration to a cell, tissue or subject.

Briefly, a TREM manufactured as describe above is incubated in one of: 0.1M Hepes/NaOH, pH 7.4 with 10 nM final concentration of RNase A for 10 min at 30° C., or 0.1M MES, 0.1M NaCl, pH 6.0, with an effective amount of angiogenin, and BSA for 6 hours at 37° C.

To isolate a target TREM fragment after enzymatic treatment, a sequence affinity purification procedure is performed, as described above.

### Example 14: Production of a TREM Fragment in a Cell Expression System

This example describes the production of a TREM fragment in a cell expression system.

A cell line stably overexpressing a TREM is generated as described in any of Examples 7-9 or 11. Hek293T cells overexpressing the TREM are treated with 0.5 pg/ml recombinant angiogenin for 90 min before total RNA is extracted with Trizol. Size selection of RNAs smaller than 200 nucleotides is performed using a small RNA isolation kit per manufacturer's instructions. Streptavidin-conjugated RNase-free magnetic beads are incubated at room temperature for 30 min with 200 mM of biotinylated oligonucleotides corresponding to a probe or a DNA probe that is complementary to a unique region of the tRNA half being purified. The beads are washed and heated for 10 min at 75° C. The size-selected RNA eluate is also heated for 10 min at 75° C. and then mixed with the beads. The TREM-bead mixture is incubated at room temperature for 3 hours to allow binding of the TREMs to the bead-bound DNA probe. The beads are then washed until the wash solution at 260 nm is close to zero (0). Alternatively, the beads are washed three times and the final wash is examined by UV spectroscopy to measure the amount of nucleic acid present in the final wash. The TREM retained on the beads are eluted 3 times using RNase-free water pre-heated to 80° C. or elution buffer pre-heated to 80° C.

### Example 15: TREM Translational Activity Assays

This example describes assays to evaluate the ability of a TREM to be incorporated into a nascent polypeptide chain.

### Translation of the FLAG-AA-his Peptide Sequence

A test TREM is assayed in an in-vitro translation reaction with an mRNA encoding the peptide FLAG-XXX-His6× (“His6×” disclosed as SEQ ID NO: 456), where XXX are 3 consecutive codons corresponding to the test TREM anticodon.

A tRNA-depleted rabbit reticulocyte lysate (Jackson et al. 2001. RNA 7:765-773) is incubated 1 hour at 30° C. with 10-25 ug/mL of the test TREM in addition to 10-25 ug/mL of the tRNAs required for the FLAG and His tag translation. In this example, the TREM used is tRNA-Ile-GAT, therefore the peptide used is FLAG-LLL-His6× (“His6×” disclosed as SEQ ID NO: 456) and the tRNAs added are tRNA-Ile-GAT, in addition to the following, which are added for translate the peptide FLAG and HIS tags: tRNA-Asp-GAC, tRNA-Tyr-TAC, tRNA-Lys-AAA, tRNA-Lys-AAAG, tRNA-Asp-GAT, tRNA-His-CAT. To determine if the test TREM is functionally able to be incorporated into a nascent peptide, an ELISA capture assay is performed. Briefly, an immobilized anti-His6λ antibody (“His6×” disclosed as SEQ ID NO: 456) is used to capture the FLAG-LLL-His6× peptide (“His6×” disclosed as SEQ ID NO: 456) from the reaction mixture. The reaction mixture is then washed off and the peptide is detected with an enzyme-conjugated anti-FLAG antibody, which reacts to a substrate in the ELISA detection step. If the TREM produced is functional, the FLAG-LLL-His6 peptide (“His6” disclosed as SEQ ID NO: 456) is produced and detection occurs by the ELISA capture assay. The methods described in this example can be adopted for use to evaluate the functionality of the TREM.

### Translational Suppression Assay

This assay describes a test TREM having translational adaptor molecule function by rescuing a suppression mutation and allowing the full protein to be translated. The test TREM, in this example tRNA-Ile-GAT, is produced such that it contains the sequence of the tRNA-Ile-GAT body but with the anticodon sequence corresponding to CUA instead of GAT. HeLa cells are co-transfected with 50 ng of TREM and with 200 ng of a DNA plasmid encoding a mutant GFP containing a UAG stop codon at the S29 position as described in Geslain et al. 2010. J Mol Biol. 396:821-831. HeLa cells transfected with the GFP plasmid alone serve as a negative control. After 24 hours, cells are collected and analyzed for fluorescence recovery by flow cytometry. The fluorescence is read out with an emission peak at 509 nm (excitation at 395 nm). The methods described in this example can be adopted for use to evaluate the functionality of the TREM, or if the TREM can rescue the stop mutation in the GFP molecule and can produce the full-length fluorescent protein.

### In Vitro Translational Assay

This assay describes a test TREM having translational adaptor molecule function by successfully being incorporated into a nascent polypeptide chain in an in vitro translation reaction. First, a rabbit reticulocyte lysate that is depleted of the endogenous tRNA using an antisense or complimentary oligonucleotide which (i) targets the sequence between the anticodon and variable loop; or (ii) binds the region between the anticodon and variable loop is generated (see, e.g., Cui et al. 2018. Nucleic Acids Res. 46(12):6387-6400). 10-25 ug/mL of the test TREM is added in addition to 2 ug/uL of a GFP-encoding mRNA to the depleted lysate. A non-depleted lysate with the GFP mRNA and with or without test TREM added are used as a positive control. A depleted lysate with the GFP mRNA but without the test TREM added is used as a negative control. The progress of GFP mRNA translation is monitored by fluorescence increase on a microplate reader at 37° C. for 3-5 h using λₑₓ485/λₑₘ528. The methods described in this example can be adopted for use to evaluate if the test TREM can complement the depleted lysate and is thus likely functional.

### Example 16: Assay for Modulation of Cell State

This example describes an assay for detecting activity of a TREM in modulating cell status, e.g., cell death.

TREM fragments are produced as described in Example 13.1 uM of TREM fragments are transfected into HEK293T cells with Lipofectamine 3000 and incubated for 1-6 hours in hour-long intervals followed by cell lysis. Cell lysates are analyzed by Western blotting and blots are probed with antibodies against total and cleaved caspase 3 and 9 as readouts of apoptosis. To measure cellular viability, cells are washed and fixed with 4% paraformaldehyde in PBS for 15 minutes at room temperature. Fixed and washed cells are then treated with 0.1% Triton X-100 for 10 minutes at room temperature and washed with PBS three times. Finally, cells are treated with TUNEL assay reaction mixture at 37° C. for 1 hour in the dark. Samples are analyzed by flow cytometry.

### Example 17: Assay for the Activity of an Uncharged TREM to Modulate Autophagy

This example describes an assay to test an uncharged TREM for ability to modulate, e.g., induce, autophagy, e.g., the ability to activate GCN2-dependent stress response (starvation) pathway signaling, inhibit mTOR or activate autophagy.

A test uncharged TREM (uTREM) preparation is delivered to HEK293T or HeLa cells through transfection or liposomal delivery. Once the uTREM is delivered, a time course is performed ranging from 30 minutes to 6 hours with hour-long interval time points. Cells are then trypsinized, washed and lysed. The same procedure is executed with a charged control TREM as well as random RNA oligos as controls. Cell lysates are analyzed by Western blotting and blots are probed with antibodies against known readouts of GCN2 pathway activation, mTOR pathway inhibition or autophagy induction, including but not limited to phospho-eIF2a, ATF4, phospho-ULK1, phospho-4EBP1, phospho-eIF2a, phospho-Akt and phospho-p70S6K. A total protein loading control, such as GAPDH, actin or tubulin, as well as the non-modified (i.e. non-phosphorylated) signaling protein, i.e. using eIF2a as a control for phospho-eIF2a, are probed as loading controls. The methods described in this example can be adopted for use to evaluate activation of GCN2 starvation signaling pathway, autophagy pathway and/or inhibition of the mTOR pathway upon TREM delivery.

### Example 18: Assay for Activity of a Mischarged TREM (mTREM)

This example describes an assay to test the functionality of a mTREM produced in a cell system using plasmid transfection followed by in vitro mischarging.

In this example, an mTREM can translate a mutant mRNA into a wild type (WT) protein by incorporation of the WT amino acid in the protein despite an mRNA containing a mutated codon. GFP mRNA molecules with either a T2031 or E222G mutation, which prevent GFP excitation at the 470 nm and 390 nm wavelengths, respectively, are used for this example. GFP mutants which prevent GFP fluorescence could also be used as reporter proteins in this assay. Briefly, an in vitro translation assay is used, using a rabbit reticulocyte lysate containing the GFP E222G mutated mRNA (GAG→GGG mutation) and an excess of the mTREM, in this case tRNA-Glu-CCC. As a negative control, no mischarged TREM is added to the reaction. The methods described in this example can be adopted for use to evaluate the functionality of the mTREM.

### Example 19: Identification of Disease-Associated SMC that could be Ameliorated by TREM Modulation

This example describes SMC-containing protein target selection for TREM-based therapy. SMCs can be understood as mutations that are informationally silent, they change the codon sequence to a synonymous codon but may have an effect on a translational or post-translational property. The selection method was segmented into three progressive selection steps (1) SMC identification, (2) examination of tRNA frequency and (3) annotation of disease relevance. These steps are described in further detail below.

### SMC Identification

A curated inclusive list of all known SNPs was utilized as a starting point for SMC selection. In this example, the db SNP NCBI mutation database (https://www.ncbi.nlm.nih.gov/ and FTP site ftp://ftp.ncbi.nih.gov/snp/organisms/) was filtered to select for a SMC, also known as synonymous SNPs (i.e. single nucleotide changes in the coding sequence not causing a change in the amino acid). Briefly, the mutated sequences were aligned to the human genome (here GRCh38p7) and the SNPS were classified into variant and mutation types, such as: non-coding-variant or coding-variant; and synonymous or non-synonymous mutations. Those classified as coding variants with synonymous mutations were designated as SMCs and taken forward into the next selection.

Examination of tRNA Frequency

For each SMC, the corresponding tRNA to each wildtype and mutated codon (SMC) was identified. The abundance of the tRNA for each of the wildtype and mutated codon (SMC) was determined from tRNA-sequencing data. In this example, the tRNA-seq previously determined from HEK293T cells (Zheng et al., Nature Methods 12, 835-837 (2015)) was utilized. SNPs that have differences, e.g., large differences, such as >10× change, in the tRNA abundance are prioritized into the next selection.

### Determining Disease Relevance

SNP IDs were mapped to a collection of known disease associated SNPs to determine which SNPs have disease correlation. In this example we utilize the GWAS (Genome Wide Associate Studies) (https://www.ebi.ac.uk/gwas/) or a similar resource to determine which SNPs have known disease correlations. Those with therapeutically relevant disease correlations (e.g., oncogenic, or relevant to a neurological disorder) were taken forward to the next step.

### Final Selection

The filtered list of SMCs contains SMCs in coding regions that: (1) do not alter the coding sequence of an amino acid; (2) have difference, e.g., large difference, in tRNA population; and (3) have disease relevancy. In this example, the final selection is done based upon a disease of interest, e.g., pancreatic cancer. The BCAR1 gene is, e.g., known to be associated with pancreatic cancer, and has a SNP (r57190458) that causes a change from codon CUC to CUU. This coding sequence change results in a corresponding change in incorporated TREMs. In some embodiments, the mutated incorporated TREMs has, e.g., about a 100× fold decrease in abundance making it a potential target for upregulation and/or amelioration of the disease phenotype.

### Example 20: PNPL3A SMC

The method of Example 19 was used to identify an SMC in the PNPL3A gene. The PNPL3A gene has a rs738408 polymorphism that was identified as a predisposing factor for nonalcoholic fatty liver disease, fibrosis and elevation of serum alanine transaminase in the human. The rs738408 polymorphism is a SMC as it is located in an ORF and changes the codon from CCC to CCU. Both the CCC and CCU codons code for the proline amino acid, resulting in an identical polypeptide sequence at that position of the chain as that of the wildtype PNPL3A ORF. This polypeptide chain is the adiponutrin protein.

### Example 21: TERT SMC

The method of Example 19 was used to identify an SMC in the TERT gene. The TERT gene has a rs2736098 polymorphism that was identified as a susceptibility factor for pancreatic cancer and non-small cell lung carcinoma in the human. The rs2736098 polymorphism is a SMC as it is located in an ORF and changes the codon from GCG to GCA. Both the GCG and GCA codons code for the alanine amino acid, resulting in an identical polypeptide sequence at that position of the chain as that of the wildtype TERT ORF. This polypeptide chain is the telomerase reverse transcriptase protein.

### Example 22: ACHE SMC

The method of Example 19 was used to identify an SMC in the ACHE gene. The ACHE gene has a rs7636 polymorphism that was identified as a susceptibility factor for Type 2 Diabetes in Asian populations. The rs7636 polymorphism is a SMC as it is located in an ORF and changes the codon from CCC to CCT. Both the CCC and CCT codons code for the proline amino acid, resulting in an identical polypeptide sequence at that position of the chain as that of the wildtype ACHE ORF. This polypeptide chain is the acetylcholinesterase (AChE) protein, which is the primary enzyme responsible for the hydrolytic metabolism of the neurotransmitter acetylcholine (ACh) into choline and acetate.

### Example 23: CFTR SMC

The method of Example 19 was used to identify an SMC in the CFTR gene. The CFTR gene has a rs1042077 polymorphism that is present in patients with CFTR-related disorders. The rs1042077 polymorphism is a SMC as it is located in an ORF and changes the codon from ACT to ACG. Both the ACT and ACG codons code for the threonine amino acid, resulting in an identical polypeptide sequence at that position of the chain as that of the wildtype CFTR ORF. This polypeptide chain is the cystic fibrosis transmembrane conductance regulator (CFTR).

### Example 24: MAP3K1 SMC

The method of Example 19 was used to identify an SMC in the MAP3K1 gene. The MAP3K1 gene has a rs2229882 polymorphism that was identified as a susceptibility factor for the early onset of breast cancer. The rs2229882 polymorphism is a SMC as it is located in an ORF and changes the codon from ACC to ACT. Both the ACC and ACT codons code for the threonine amino acid, resulting in an identical polypeptide sequence at that position of the chain as that of the wildtype MAP3K1 ORF. This polypeptide chain is the Mitogen-Activated Protein Kinase Kinase Kinase 1 (MAP3K1), which is serine/threonine kinase that regulates the ERK and JNK MAPK pathways as well as the transcription factor NF-kappa-B pathway.

### Example 25: Production of a Candidate TREM Complementary to the SMC Through Mammalian Cell Purification

This example describes the production of a TREM in mammalian host cells.

### Plasmid Generation

To generate a plasmid comprising a TREM which comprises a tRNA gene, in this example, tRNA-Ser-AGA, a DNA fragment containing at least one copy of the tRNA gene with the sequence GTAGTCGTGGCCGAGTGGTTAAGGCGATGGACTAGAAATCCATTGGGGTTTCCCCGC GCAGGTTCGAATCCTGCCGACTACG (SEQ ID NO: 192) is synthesized and cloned into the pLKO.1 puro backbone plasmid with a U6 promoter (or any other RNA polymerase III recruiting promoter) following the manufacturer's instructions and standard molecular cloning techniques.

### Transfection

One (1) mg of plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for TREM expression as determined by a quantitative method such as Northern blot, quantitative PCR (q-PCR) or Nanopore sequencing.

### Purification

At the optimized harvest timepoint, the cells are lysed, and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is incubated with annealing buffer and the biotinylated capture probe corresponding to a DNA probe that is complementary to a unique region of the target TREM being purified, in this example, a probe with the sequence 3′ biotin-CCAATGGATTTCTATCCATCGCCTTAACCACTCGGCCACGACTACAAAA (SEQ ID NO: 457) is used to purify the TREM comprising tRNA-Ser-AGA. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 26: Production of a Candidate TREM Complementary to the SMC Through Bacterial Cell Purification

This example describes the production of a TREM in bacterial host cells.

### Plasmid Generation

To generate a plasmid to produce a TREM in bacteria, a tRNA gene, in this example, a DNA fragment containing at least one copy of the tRNA-Lys-UUU gene with the sequence GCCCGGATAGCTCAGTCGGTAGAGCATCAGACTTTTAATCTGAGGGTCCAGGGTTCA AGTCCCTGTTCGGGCG (SEQ ID NO: 166) is synthesized and cloned into a bacterial tRNA expression vector as previously described in Ponchon et al., Nat Protoc 4, 947-959 (2009).

### Transformation

1×10⁹ bacteria grown from TREM expression plasmid transformed competent bacteria will be harvested at different cell density points, in this example OD(600)=0.5, OD(600)=0.7, OD(600)=0.9 to determine the optimal point of TREM expression as determined by a quantitative method such as Northern blot, quantitative PCR (q-PCR) or Nanopore sequencing.

### Purification

At the optimized harvest cell density point, the TREM is purified as previously described in Cayama et al., Nucleic Acids Research. 28 (12), e64 (2000). Briefly, short RNAs (e.g., tRNAs) are recovered from cells by phenol extraction and concentrated by ethanol precipitation. The total tRNA in the precipitate is then separated from larger nucleic acids (including rRNA and DNA) under high salt conditions by a stepwise isopropanol precipitation. The elution fraction containing the TREM is further purified through probe binding. The TREM fraction is incubated with annealing buffer and the biotinylated capture probe corresponding to a DNA probe that is complementary to a unique region of the target TREM being purified, in this example, a probe conjugated to biotin at the 3′ end with the sequence CAGAUUAAAAGUCUG (SEQ ID NO: 458), is used to purify the TREM comprising tRNA-Lys-UUU. The mixture is incubated at 90° C. for 2-3 minutes and quickly cooled down to 45° C. and incubated overnight at 45° C. The admixture is then incubated with binding buffer previously heated to 45° C. and streptavidin-conjugated RNase-free magnetic beads for 3 hours to allow binding of the DNA-tRNA complexes to the beads. The mixture is then added to a pre-equilibrated column in a magnetic field separator rack and washed 4 times. The TREM retained on the beads are eluted three times by adding elution buffer pre-heated to 80° C. and then admixed with a pharmaceutically acceptable excipient to make a test TREM product.

### Example 27: Production of a Candidate TREM Complementary to the SMC Through Chemical Synthesis

This example describes production of a TREM using chemical synthesis.

The TREM, in this example, tRNA-Thr-CGT, is chemically synthesized with the sequence GGCUCUAUGGCUUAGUUGGUUAAAGCGCCUGUCUCGUAAACAGGAGAUCCUGGG UUCGACUCCCAGUGGGGCCUCAA (SEQ ID NO: 459). This TREM is produced by solid-phase chemical synthesis using phosphoroamedite chemistry as previously described, for example as in Zlatev et. al. (2012) Current Protocols, 50 (1), 1.28.1-1.28.16. Briefly, protected RNA phorphoroamedites are sequentially added in a desired order to a growing chain immobilized on a solid support (e.g. controlled pore glass). Each cycle of addition has multiple steps, including: (i) deblocking the DMT group protecting the 5′-hydroxyl of the growing chain, (ii) coupling the growing chain to an incoming phosphoramidite building block, (iii) capping any chain molecules still featuring a 5′-hydroxyl, i.e. those that failed to couple with the desired incoming building block, and (iv) oxidation of the newly formed tricoordinated phosphite triester linkage. After the final building block has been coupled and oxidized, the chain is cleaved from the solid support and all protecting groups except for the DMT group protecting the 5′-hydroxyl are removed. The chain is then purified by RP-HPLC (e.g., DMT-on purification) and the fraction containing the chain is subjected to deprotection of the DMT group under acidic conditions, affording the final TREM. The TREM will feature a 5′-phosphate and a 3′-OH. The TREM is then admixed with a pharmaceutically acceptable excipient to make a test TREM product.

If the TREM needs to be charged, the TREM produced by the chemical synthesis reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with threonyl-tRNA synthetase and threonine, respectively, and then phenol extracted, filtered using a Nuc-trap column, and ethanol precipitated. The TREM is then admixed with a pharmaceutically acceptable excipient to make a test TREM product.

### Example 28: Production of a Candidate TREM Complementary to the SMC Through In Vitro Transcription

This example describes production of a TREM using in vitro transcription (IVT).

The TREM, in this example, tRNA-Leu-CAA, is produced using in vitro transcription with the sequence GUCAGGAUGGCCGAGUGGUCUAAGGCGCCAGACUCAAGUUCUGGUCUCCGUAUG GAGGCGUGGGUUCGAAUCCCACUUCUGACA (SEQ ID NO: 460) as previously described in Pestova et al., RNA 7 (10): 1496-505 (2001). Briefly, a DNA plasmid containing a bacteriophage T7 promoter followed by the tRNA-Leu-CAA gene sequence is linearized and transcribed in vitro with T7 RNA polymerase at 37° C. for 45 min and then phenol extracted, filtered using a Nuc-trap column, and ethanol precipitated. The TREM is then admixed with a pharmaceutically acceptable excipient to make a test TREM product.

If the TREM needs to be charged, the TREM produced by the IVT reaction is then aminoacylated in vitro using aminoacyl tRNA synthetase, as previously described in Stanley, Methods Enzymol 29:530-547 (1974). Briefly, the TREM is incubated for 30 min at 37° C. with its synthetase and its cognate amino, in this example, with leucyl-tRNA synthetase and leucine, respectively, and then phenol extracted, filtered using a Nuc-trap column, and ethanol precipitated. The TREM is then admixed with a pharmaceutically acceptable excipient to make a test TREM product.

### Example 29: Modulation of a tRNA Pool Through TREM Administration to a Cell

This example describes administration of a TREM to a cell to modulate tRNA pools in the cell.

TREMs produced as in Examples 25-28 are delivered to a cell through electroporation, as previously described in Nature Methods 3, 67-68 (2006). Briefly, 10⁶-10⁷ cells, in this example the human epithelial MCF10A cells, are transferred in an electroporation cuvette and mixed gently after the addition of 1-30 ug of TREM, in this example tRNA-Thr-CGT with the sequence GGCUCUAUGGCUUAGUUGGUUAAAGCGCCUGUCUCGUAAACAGGAGAUCCUGGG UUCGACUCCCAGUGGGGCCUCAA (SEQ ID NO: 459). The cuvette is transferred to the electroporator and the device is discharged (a voltage of 200-350V is used). Place cuvette on ice and transfer the electroporated cells to a culture dish with complete medium and transfer to an incubator for 24-48 hrs.

Once delivered, the change in tRNA pools can be quantified by methods such as Nanopore sequencing, tRNA-sequencing, Northern blotting or quantitative RT-PCR. In this example, the tRNA pool changes are monitored using Oxford Nanopore direct RNA sequencing, as previously described in Sadaoka et al., Nature Communications (2019) 10, 754.

Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 30 minutes. The solution is neutralized by the addition of an equal volume of 100 mM Na-acetate/acetic acid (pH 4.8) and 100 mM NaCl, followed by ethanol precipitation. Deacylated sRNA is dissolved in water, and its integrity verified by agarose gel electrophoresis. Deacylated sRNA is then polyadenylated using yeast poly(A) tailing kit per manufacturer's instructions to generate a sRNA polyadenylated pool. Following polyadenylation, a reverse transcription reaction is performed to generate cDNA using SuperScript III Reverse Transcriptase (Thermo Fisher Scientific) or a thermostable group II intron RT (TGIRT, InGex LLC) that is less sensitive to RNA structure and modifications. A sequencing adapter is ligated onto the cDNA mixture by incubating the cDNA mixture with RNA adapter, T4 ligase and ligation buffer following the standard protocol for Oxford Nanopore. Nanopore sequencing is then performed on the libraries and the sequences are mapped to a genomic database, in this example to the genomic tRNA database, GtRNAdb. The methods described in this example can be adopted for use to evaluate the tRNA pool in the cells administered with a TREM compared to those not administered with a TREM.

### Example 30: Modulation of a tRNA Pool Through TREM Administration to a Cell Using Liposome

This example describes administration of a TREM to a cell using liposome vesicles to modulate tRNA pools in the cell.

TREMs produced as in Examples 25-28 are delivered to a cell in a vesicle or other lipid-based carrier, such as liposomes or lipid nanoparticles. In this example, a liposome kit (from Sigma or other vendor) is used to prepare liposomes containing the TREM, in this example tRNA-Thr-CGT with the sequence GGCUCUAUGGCUUAGUUGGUUAAAGCGCCUGUCUCGUAAACAGGAGAUCCUGGG UUCGACUCCCAGUGGGGCCUCAA (SEQ ID NO: 459) following manufacturer's directions. The human cell line, HEK293T, is used in this example. Cells are seeded to obtain 70-80% confluency the day of the transfection. The media is replaced 30 minutes prior to the transfection with serum-free media after which the liposomes are added to the cell media.

Once delivered, the change in tRNA pools can be quantified by methods such as Nanopore sequencing, tRNA-sequencing (Zheng et al., Nature Methods 12, 835-837 (2015)), Northern blotting or quantitative RT-PCR. In this example, the tRNA pool changes are monitored using tRNA-sequencing. Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is treated with a demethylase mixture to remove m¹A, m¹G and m³C modifications located at the Watson-Crick face. Following demethylation of the tRNA pool, a cDNA library is generated from the tRNAs using a thermostable group II intron RT (TGIRT) that is less sensitive to tRNA structure. This reverse transcriptase adds RNA-sequencing adaptors to the tRNAs by template-switching without requiring RNA ligation. Illumina sequencing is then performed on the libraries generated from the tRNAs and the sequencing reads are mapped to a genomic database, in this example to the genomic tRNA database, GtRNAdb. The methods described in this example can be adopted for use to evaluate the tRNA pool in the cells administered with a TREM compared to those not administered with a TREM.

### Example 31: Modulation of a tRNA Pool Through Delivery of TREM-Encoding Plasmid to a Cell

This example describes delivery of TREM-encoding plasmid to a cell to modulate tRNA pools in the cell.

A TREM is expressed in cells through delivery of a TREM-encoding plasmid using a vesicle-based carrier. To express a TREM in human cells, a plasmid is created, which contains a tRNA gene, in this example, tRNA-Gly-GCC, with the sequence GCATTGGTGGTTCAGTGGTAGAATTCTCGCCTGCCACGCGGGAGGCCCGGGTTCGAT TCCCGGCCAATGCA (SEQ ID NO: 108). The plasmid is generated using seamless assembly of DNA fragments, in this example using NEBuilder HiFi Assembly Master Mix, where a linearized mammalian expression vector of interest, in this example pLKO.1-puro-turboGFP linearized by PpuMI enzyme restriction, is fused with a DNA fragment that contains the tRNA gene. The DNA fragment in this example includes the following elements in 5′ to 3′ order: a 25 nucleotide-long sequence from the 3′ end of the vector linearization site, a U6 promoter, the tRNA sequence, a RNA polymerase III termination signal, a 25 nucleotide-long sequence from the 5′ end of the vector linearization site.

Once the plasmid is made, the human cell line, in this example HEK293T, is transfected with TREM-encoding plasmid, using Lipofactamine 3000 following manufacturer's directions. Once delivered, the change in tRNA pools can be quantified by methods such as Nanopore sequencing, tRNA-sequencing (Zheng et al., Nature Methods 12, 835-837 (2015)), Northern blotting or quantitative RT-PCR. In this example, the tRNA pool changes are monitored using tRNA-sequencing. Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is treated with a demethylase mixture to remove m¹A, m¹G and m³C modifications located at the Watson-Crick face. Following demethylation of the tRNA pool, a cDNA library is generated from the tRNAs using a thermostable group II intron RT (TGIRT) that is less sensitive to tRNA structure. This reverse transcriptase adds RNA-sequencing adaptors to the tRNAs by template-switching without requiring RNA ligation. Illumina sequencing is then performed on the libraries generated from the tRNAs and the sequencing reads are mapped to a genomic database, in this example to the genomic tRNA database, GtRNAdb. The methods described in this example can be adopted for use to evaluate the tRNA pool in the cells administered with a TREM compared to those not administered with a TREM.

### Example 32: Modulation of a tRNA Pool Through Delivery of a TREM-Encoding Viral Vector to a Cell

This example describes delivery of a TREM-encoding viral vector to a cell to modulate tRNA pools in the cell.

A TREM is expressed in cells through delivery of a TREM-encoding viral vector. In this example, a lentivirus packaging and delivery system encoding a TREM is used. Briefly, the TREM-encoding viral vector is built by first generating a plasmid comprising a TREM, in this example, tRNA-Gly-GCC, with the sequence GCATTGGTGGTTCAGTGGTAGAATTCTCGCCTGCCACGCGGGAGGCCCGGGTTCGAT TCCCGGCCAATGCA (SEQ ID NO: 108). The plasmid is generated using seamless assembly of DNA fragments where the pLKO.1-puro-turboGFP linearized vector is ligated to a DNA fragment containing the tRNA sequence as described in Example 31. To prepare a TREM expressing lentivirus, HEK293T cells are co-transfected with 3 μg of each packaging vector (pRSV-Rev, pCMV-VSVG-G and pCgpV) and 9 μg of the plasmid comprising a TREM, using Lipofectamine 3000 according to manufacturer's instructions. After 24 hours, the media is replaced with fresh antibiotic-free media and after 48 hours, virus-containing supernatant is collected and centrifuged for 10 min at 2000 rpm before being filtered through a 0.45 μm filter.

The cell of interest is then infected with the virus. In this example, 2 mL of virus prepared is used to transduce 100,000 HeLa cells, in the presence of 8 μg/mL polybrene. Forty-eight hours after transduction, puromycin (at 2 μg/mL) antibiotic selection is performed for 2-7 days alongside a population of untransduced control cells to select for cells that integrated the TREM in their genome for expression.

The change in tRNA pools can be quantified by methods such as Nanopore sequencing, tRNA-sequencing (Zheng et al., Nature Methods 12, 835-837 (2015)), Northern blotting or quantitative RT-PCR. In this example, the tRNA pool changes are monitored using quantitative RT-PCR (Korniy et al., Nucleic Acids Research (2019), gkz202). Briefly, the TREM-transfected cells are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is treated with a demethylase mixture to remove m¹A, m¹G and m³C modifications located at the Watson-Crick face. Following demethylation, the pool is reverse transcribed into cDNA using stem-loop adapters complimentary to the 3′-ends of the tRNAs of interest. In this step, reverse transcription (RT) is performed using the Superscript III first strand synthesis system (ThermoFisher Scientific). Quantitative PCR is then performed using the QuantiTect SYBR Green Kit (Qiagen) according to the manufacturer's protocol with forward primers complimentary to the region of cDNA encoded by the tRNAs of interest and a universal primer complimentary to the stem-loop adapter appended during RT. The methods described in this example can be adopted for use to evaluate the levels of glycine specifying molecule that can pair with the CGT codon in the cells administered with a TREM compared to those not administered with a TREM.

### Example 33: System to Test the Effects of TREM Administration on an SMC Containing ORF

This example describes a system, in this example a cell line, that expresses an SMC-containing ORF to study the effects of TREM administration.

To study the effects of TREM administration on a SMC-containing ORF, in this example on the rs2229882 polymorphism of the MAP3K1 gene, an established cell line, in this example human breast epithelial cells, such as MCF10A or 184A1 cells, are genomically edited by CRISPR-Cas to knock out the expression of the endogenous gene of interest, in this example the MAP3K1 gene. MAP3K1 knockout cells were generated using the CRISPR-Cas9 system to insert 1 bp in a coding exon of MAP3K1 to cause a frameshift mutation as previously described (for example, Bauer et al., J. Vis. Exp., (95), doi:10.3791/52118 (2015)). Briefly, an online design tool that predicts the most effective guide RNA to use for genome editing, for example, https://portals.broadinstitue.org/gpp/public/analysis-tools/sgran-design, is used to select a high-score guide RNA (gRNA) containing a 20-base pair (bp) target sequence that minimizes genomic matches to reduce the risk of off-target site cleavage. In this example, the targeting sequence is CAGTGTGTGAAGACGGCTGC (SEQ ID NO: 461). The targeting sequence is cloned into pSpCas9 (BB) plasmid (pX330) (Addgene plasmid ID 42230). HEK293T cells are transiently transfected with the CRISPR/Cas9 construct targeting MAP3K1 and a puromycin expression construct for clone selection. The next day, cells are selected with puromycin for 2 days and subcloned to form single colonies. MAP3K1 KO clones are identified by PCR screen. The obtained clones are validated by qPCR and immunoblot using an antibody against MAP3K1.

Once created, this cell line is used to overexpress the WT or SMC-containing mRNA through transient plasmid transfection or through stable lentivirus transduction methods. The TREM of interest is then administered to each cell line and its effect on the SMC-containing ORF compared to the WT ORF is assessed using assays such as the ones described in Examples 19-24.

### Example 34: Determining that Administration of a TREM Affects Protein Expression Levels of SMC-Containing ORF

This example describes administration of a TREM to alter expression levels of an SMC-containing ORF.

To create a system in which to study the effects of TREM administration on protein expression levels of SMC-containing protein, in this example, from the PNPL3A gene coding for adiponutrin, a plasmid containing the PNPL3A rs738408 ORF sequence is transfected in the normal human hepatocyte cell line THLE-3, edited by CRISPR/Cas to contain a frameshift mutation in a coding exon of PNPLA3 to knock out endogenous PNPLA3 (THLE-3_PNPLA3KO cells). As a control, an aliquot of THLE-3_PNPLA3KO cells are transfected with a plasmid containing the wildtype PNPL3A ORF sequence.

A TREM is delivered to the THLE-3_PNPLA3KO cells containing the rs738408 ORF sequence as well as to the THLE-3_PNPLA3KO cells containing the wildtype PNPL3A ORF sequence. In this example, the TREM contains a proline isoacceptor containing an AGG anticodon, that base pairs to the CCT codon, i.e. with the sequence GGCUCGUUGGUCUAGGGGUAUGAUUCUCGCUUAGGGUGCGAGAGGUCCCGGGUU CAAAUCCCGGACGAGCCC (SEQ ID NO: 462). A time course is performed ranging from 30 minutes to 6 hours with hour-long interval time points. At each time point, cells are trypsinized, washed and lysed. Cell lysates are analyzed by Western blotting and blots are probed with antibodies against the adiponutrin protein. A total protein loading control, such as GAPDH, actin or tubulin, is also probed as a loading control.

The methods described in this example can be adopted for use to evaluate the expression levels of the adiponutrin protein in rs738408 ORF containing cells.

### Example 35: TREM Administration to Change Protein Translation Rate of SMC-Containing ORF

This example describes administration of a TREM to alter the rate of protein translation of an SMC-containing ORF.

To monitor the effects of TREM addition on translation elongation rates, an in vitro translation system, in this example the RRL system from Promega, is used in which the fluorescence change over time of a reporter gene, in this example GFP, is a surrogate for translation rates. First, a rabbit reticulocyte lysate that is depleted of the endogenous tRNA using an antisense oligonucleotide targeting the sequence between the anticodon and variable loop is generated (see, e.g., Cui et al. 2018. Nucleic Acids Res. 46(12):6387-6400). In this example, a TREM comprising an alanine isoacceptor containing an UGC anticodon, that base pairs to the GCA codon, i.e. with the sequence GGGGAUGUAGCUCAGUGGUAGAGCGCAUGCUUUGCAUGUAUGAGGUCCCGGGUU CGAUCCCCGGCAUCUCCA (SEQ ID NO: 463) is added to the in vitro translation assay lysate in addition to 0.1-0.5 ug/uL of mRNA coding for the wildtype TERT ORF fused to the GFP ORF by a linker or an mRNA coding for the rs2736098 TERT ORF fused to the GFP ORF by a linker. The progress of GFP mRNA translation is monitored by fluorescence increase on a microplate reader at 37° C. using λₑₓ485/λₑₘ528 with data points collected every 30 seconds over a period of 1 hour. The amount of fluorescence change over time is plotted to determine the rate of translation elongation of the wildtype ORF compared to the rs2736098 ORF with and without TREM addition. The methods described in this example can be adopted for use to evaluate the translation rate of the rs2736098 ORF and the wildtype ORF in the presence or absence of TREM.

### Example 36: Determining that Modulation of a TREM Complementary to the SMC Changes the Function of the Protein Derived from the SMC-Containing ORF

This example describes administration of a TREM to change the function of an SMC-containing ORF.

Using an in vitro translation (IVT) system (such as the RRL system from Promega), the wildtype and SMC containing mRNAs are translated in the presence and absence of a TREM. In this example the SMC-containing gene is AChE, coding for the acetylcholinesterase protein, and the TREM contains a proline isoacceptor containing an AGG anticodon, that base pairs to the CCU codon, i.e. with the sequence GGCUCGUUGGUCUAGGGGUAUGAUCUCGCUUAGGGUGCGAGAGGUCCCGGGUUC AAAUCCCGGACGAGCCC (SEQ ID NO: 464).

To determine if addition of the TREM changes the functional activity of the SMC-containing protein, in this example the acetylcholinesterase protein, a functional assay that uses DTNB to quantify the thiocholine produced from the hydrolysis of acetylthiocholine by AChE is used. Briefly, the translation reactions are incubated at room temperature for 10-30 minutes with the kit AChE reaction mixture, after which the absorption intensity of DTNB adduct at OD 410 nm is used to measure the amount of thiocholine formed, which is proportional to the AChE activity. The methods described in this example can be adopted for use to evaluate AChE activity of the protein resulting from the translation of the rs7636 AChE mRNA or the wildtype AChE mRNA.

### Example 37: Determining that Modulation of a TREM Complementary to an SMC Changes the Localization of the Protein Derived from the SMC-Containing ORF

This example describes administration of a TREM to alter the localization of an SMC-containing ORF.

To create a system in which to study the effects of TREM administration on protein localization of an SMC-containing ORF, a plasmid containing the CFTR rs1042077 ORF sequence tagged with a reporter, such as GFP or myc, is transfected in the human lung epithelial cell line MRC-5. As a control, a plasmid containing the wildtype CFTR ORF sequence tagged with a reporter is also transfected in parallel in MRC-5 cells.

To determine if TREM addition changes the localization of CFTR, the cells are seeded on coverslips and 24 hours later are transfected with a TREM complementary to the CFTR SMC or a control TREM. In this example the TREM complementary to the CFTR SMC comprises a threonine isoacceptor containing an CGU anticodon, that base pairs to the ACG codon, i.e. with the sequence GGCUCUGUGGCUUAGUUGGCUAAAGCGCCUGUCUCGUAAACAGGAGAUCCUGGG UUCGAAUCCCAGCGGGGCCU (SEQ ID NO: 465). The control TREM consists of either a scrambled sequence or the threonine sequence where the 5′ end of the TREM has been changed to prevent charging. After 24 hours, the cells are fixed, stained for CFTR and its reporter and visualized under a microscope. The methods described in this example can be adopted for use to evaluate the localization of wildtype CFTR and rs1042077 CFTR.

### Example 38: Determining that Modulation of a TREM Complementary to an SMC Changes Folding of the Protein Translated from the SMC-Containing ORF

This example describes administration of a TREM to alter the folding of an SMC-containing ORF.

### Plasmid Generation and Transfection

To identify SMCs that result in protein misfolding, the SMC-ORF containing protein, in this example the rs7190458 BCAR1 ORF is synthesized and cloned into a plasmid containing a CMV promoter (or any other mammalian promoter) and a purification tag, in this example a FLAG tag (DYKDDDDK epitope (SEQ ID NO: 466)), following the manufacturer's instructions and standard molecular cloning techniques. Here, the pFLAG-CMV-1 plasmid is used. The plasmid is transfected in the human HeLa cell line. A TREM, in this example comprising a leucine isoacceptor containing an UUG anticodon, that base pairs to the CUU codon, i.e. with the sequence GGUAGCGUGGCCGAGCGGUCUAAGGCGCUGGAUUAAGGCUCCAGUCUCUUCGGA GGCGUGGGUUCGAAUCCCACCGCUGCCA (SEQ ID NO: 467) is also transfected into the HeLa cells. As a control, the BCAR1 KO cells are transfected with the SMC BCAR1 ORF containing plasmid alone and separately with a plasmid containing the wildtype BCAR1 ORF sequence.

### Purification

At the optimized harvest timepoint, in this example 72 hours post-transfection, the cells are lysed, and centrifuged at 12,000×g for 10 minutes. The supernatant is loaded under gravity flow onto a pre-packed and equilibrated anti-flag packed M2-agarose column. The column is washed with 10-20 column volumes of TBS (Tris HCl, NaCl) or with a salt containing buffer. To elute the FLAG-tagged protein from the beads, the beads are incubated with FLAG-tag peptide. The eluate is run on an SDS-PAGE gel for purity quality control. This purification is performed on cells transfected with the WT BCAR1 ORF and the SMC BCAR1 ORF in the presence and absence of a TREM.

### Initial Examination of Protein Folding

To examine the effects of protein folding, the stability of the purified proteins derived from the WT and SMC containing ORFs are monitored using thermal melting. In this example, Differential Scanning Fluorimetry (DSF) with a fluorescent dye (Sypro Orange), which measures the changes of binding of the intercalator dye to the unfolding protein is used. Alterations in protein folding results in variations of the thermal melting curves. Using this methodology, the SMC ORF-derived protein with and without TREM addition is compared to the control wildtype BCAR1. The methods described in this example can be adopted for use to evaluate the thermal melting curve of proteins derived from SMC-containing ORFs.

### Example 39: Determining that Modulation of a TREM Complementary to an SMC Alters the Cellular Phenotype Resulting from Translation of the SMC-Containing ORF

This example describes administration of a TREM to alter the cellular phenotype of an SMC-containing ORF.

To create a system in which to study the effects of TREM administration on cellular processes, in this example on cell migration, a plasmid containing the SMC-containing ORF, in this example the rs7190458 BCAR1 ORF sequence, is transfected in the human pancreatic cancer cell line PANC-1, in which BCAR1 has been knocked out using CRISPR/Cas. As a control, the PANC-1 BCAR1 KO cells are transfected with a plasmid containing the WT BCAR1 ORF sequence.

A TREM, in this example comprising a leucine isoacceptor containing an UUG anticodon, that base pairs to the CUU codon, i.e. with the sequence GGUAGCGUGGCCGAGCGGUCUAAGGCGCUGGAUUAAGGCUCCAGUCUCUUCGGA GGCGUGGGUUCGAAUCCCACCGCUGCCA (SEQ ID NO: 467) is delivered to the PANC-1 cells. Delivery of a control TREM containing either a scrambled sequence or the leucine sequence where the 5′ end of the TREM has been changed to prevent charging are used as a control. The cells are grown to 80% confluency in a monolayer and scratched with a new 1 ml pipette tip across the center of the well. The cells are rinsed twice to remove floating cells and the media is replenished. After 48 hours, the cells are fixed and stained with crystal violet. The stained monolayer is photographed, and the gap distance quantified. The methods described in this example can be adopted for use to evaluate the migratory phenotype of cells administered a TREM.

### Example 40: Modulation of TREM to Ameliorate a Disease State Resulting from Translation of the SMC-Containing ORF

This example describes increasing TREM levels to ameliorate a disease state resulting from an SMC-containing ORF.

To create a system in which to study the effects of TREM administration on disease state, in this example on breast cancer onset, a plasmid containing the SMC-containing ORF, in this example the rs2229882 MAP3K1 ORF sequence, is transfected in the human non-transformed breast cell line MCF10A, in which MAP3K1 has been knocked out using CRISPR/Cas. As a control, the MCF10A MAP3K1 KO cells are transfected with a plasmid containing the wildtype MAP3K1 ORF sequence.

A TREM, in this example comprising a threonine isoacceptor containing an AGU anticodon, that base pairs to the ACU codon, i.e. with the sequence GGCGCCGUGGCUUAGUUGGUUAAAGCGCCUGUCUAGUAAACAGGAGAUCCUGGG UUCGAAUCCCAGCGGUGCCU (SEQ ID NO: 468) is delivered to the MCF10A cells. Delivery of a control TREM containing either a scrambled sequence or the threonine sequence where the 5′ end of the TREM has been changed to prevent charging are used as a control. The cells are monitored for increased MAPK signaling by Western blotting using antibodies against the phosphorylation state of the ERK and JNK kinases. A total protein loading control, such as GAPDH, actin or tubulin, as well as the non-modified (i.e. non-phosphorylated) signaling protein, i.e. using ERK as a control for phospho-ERK, are probed as loading controls. The cells are also monitored for cell proliferation and invasion using standard proliferation and transwell invasion assays. To monitor breast cancer progression, the cells are injected subcutaneously or in the mammary fat pad of SCID mice and tumor volume is monitored daily using calipers to measure the length, width and height of the tumor(s). The methods described in this example can be adopted for use to evaluate tumor phenotype.

What is claimed is:

1. A method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of (i) and (ii) in the cell wherein (i) is a tRNA moiety having an anticodon that pairs with the codon of the ORF having the first sequence (first tRNA moiety) and (ii) is an isoacceptor tRNA moiety having an anticodon that pairs with a codon other than the codon having the first sequence (second tRNA moiety);
contacting the cell with a composition comprising a TREM, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety in the cell,
thereby modulating the tRNA pool in the cell.

2. A method of modulating a tRNA pool in a subject having an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the subject wherein (i) is a tRNA moiety having an anticodon that pairs with the codon of the ORF having the first sequence (first tRNA moiety) and (ii) is an isoacceptor tRNA moiety having an anticodon that pairs with a codon other than the codon having the first sequence (second tRNA moiety) in the subject;
contacting the subject with a composition comprising a TREM, wherein the TREM has an anticodon that pairs with: (a) the codon having the first sequence; or (b) the codon other than the codon having the first sequence, in an amount and time sufficient to modulate the relative amounts of the first tRNA moiety and the second tRNA moiety in the subject,
thereby modulating the tRNA pool in the subject.

3. A method of evaluating a tRNA pool in a cell having an endogenous ORF, which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the cell wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the cell.

4. A method of evaluating a tRNA pool in a subject having an endogenous ORF, which ORF comprises a codon having a first sequence, comprising acquiring, e.g., directly or indirectly acquiring, knowledge of the abundance of one or both of (i) and (ii), e.g., acquiring knowledge of the relative amounts of (i) and (ii) in the subject wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell, thereby evaluating the tRNA pool in the subject.

5. The method of any one of claims 1-4, comprising acquiring knowledge of (i).

6. The method of any one of claims 1-4, comprising acquiring knowledge of (ii).

7. The method of any one of claims 1-4, comprising acquiring knowledge of (i) and (ii).

8. The method of any one of claim 1-5 or 7, wherein acquiring knowledge of (i) comprises acquiring a value for the abundance, e.g., relative amount, of (i).

9. The method of any one of claim 1-4 or 6-7, wherein acquiring knowledge of (ii) comprises acquiring a value for the abundance, e.g., relative amount, of (ii).

10. The method of claim 8 or 9, wherein responsive to said value the method comprises contacting the cell or subject with a composition comprising a TREM, wherein the TREM has an anticodon that pairs with: (a) the codon having the first sequence; or (b) the codon other than the codon having the first sequence, in an amount and for a time sufficient to modulate the relative amounts of the first tRNA moiety and the second tRNA moiety.

11. The method of any one of claim 1-2 or 5-10, wherein the composition comprising a TREM is a pharmaceutical composition comprising a TREM or a GMP-grade composition comprising a TREM.

12. The method of any one of claim 1-2 or 5-11, wherein the TREM does not comprise an anticodon that pairs with a stop codon.

13. A method of modulating a tRNA pool in a subject, or a cell, comprising an endogenous open reading frame (ORF) comprising a codon comprising a synonymous mutation (a synonymous mutation codon or SMC), comprising:
providing a composition comprising a TREM, wherein the TREM comprises an isoacceptor tRNA moiety comprising an anticodon sequence that pairs with the SMC (the TREM);
contacting the subject with the composition comprising the TREM, or in the case of a cell, contacting the cell with the TREM from a composition comprising the TREM, in an amount and/or for a time sufficient to modulate the tRNA pool in the subject, or in the cell,
thereby modulating the tRNA pool in the subject or the cell.

14. The method of claim 13, wherein prior to contacting with the composition comprising a TREM, the subject or the cell comprises a first tRNA moiety having an anticodon that pairs with the SMC (the first tRNA moiety), and a second tRNA moiety having an anticodon that pairs with a codon other than the SMC (the second tRNA moiety).

15. A method of treating a subject having an endogenous open reading frame (ORF) which comprises a codon having a first sequence, comprising:
providing a composition comprising a TREM, wherein the TREM comprises an isoacceptor tRNA moiety having: an anticodon that pairs with the codon of the ORF having the first sequence; or an anticodon that pairs with a codon other than the codon having the first sequence,
contacting the subject with the composition comprising the TREM in an amount and/or for a time sufficient to treat the subject,
thereby treating the subject.

16. A method of treating a subject having an endogenous ORF comprising a codon comprising a synonymous mutation (a synonymous mutation codon or SMC), comprising:
providing a composition comprising a TREM, wherein the TREM comprises an isoacceptor tRNA moiety having an anticodon that pairs with the SMC (the TREM);
contacting the subject with the composition comprising a TREM in an amount and for a time sufficient to treat the subject,
thereby treating the subject.

17. A method of treating a subject having an endogenous ORF comprising a codon comprising a synonymous mutation (a synonymous mutation codon or SMC), comprising:
(i) acquiring, e.g., directly or indirectly acquiring, a value for the SMC status of the subject, wherein said value comprises a measure of the presence or absence of SMC in a sample from the subject, and identifying the subject as having a SMC; and
(ii) responsive to said value, administering a composition comprising a TREM, wherein the TREM comprises an isoacceptor tRNA moiety having an anticodon that pairs with the SMC, to the subject,
thereby treating the subject.

18. A method of treating a subject having an endogenous ORF comprising a codon having a first sequence, comprising:
(i) acquiring, e.g., directly or indirectly acquiring, a value for the status of the codon having the first sequence in the subject, wherein said value comprises a measure of the presence or absence of the codon having the first sequence in a sample from the subject; and identifying the subject as having the codon having the first sequence; and
(ii) responsive to said value, administering a composition comprising a TREM, wherein the TREM comprises an isoacceptor tRNA moiety having an anticodon that pairs with the codon having the first sequence, to the subject,
thereby treating the subject.

19. A method of evaluating a subject having an endogenous open reading frame (ORF) comprising a codon having a first sequence, comprising:
acquiring, e.g., directly or indirectly acquiring, a value for the status of the codon having the first sequence in the subject, wherein said value comprises a measure of the presence or absence of the codon having the first sequence in a sample from the subject; and
identifying the subject as having a codon having the first sequence,
thereby evaluating the subject.

20. A method of evaluating a subject having an endogenous ORF comprising a codon comprising a synonymous mutation (a synonymous mutation codon or SMC), comprising:
acquiring, e.g., directly or indirectly acquiring, a value for the SMC status of the subject, wherein said value comprises a measure of the presence or absence of SMC in a sample from the subject; and
identifying the subject as having a SMC,
thereby evaluating the subject.

21. The method of any one of claims 2-20, wherein the subject has or is identified as having a disorder or symptom chosen from Table 1.

22. The method of any one of claim 1, or 3-20, wherein the cell is associated with a disorder or symptom chosen from Table 1.

23. The method of any one of the preceding claims, wherein (a) the ORF codon having the first sequence; or (b) the SMC; in the absence of contact with the composition comprising a TREM, is associated with a phenotype, e.g., an unwanted phenotype, e.g., a disorder or symptom, e.g., a disorder or symptom chosen from Table 1.

24. The method of any one of claims 21-23, wherein the disorder or symptom is chosen from a disease group provided in Table 1, e.g., cardiovascular, dermatology, endocrine, immunology, neurology, oncology, ophthalmology, or respiratory.

25. The method of any one of claims 21-23, wherein the disorder is cardiac hypertrophy.

26. The method of any one of claims 21-23, wherein the disorder is coronary artery disease.

27. The method of any one of claims 21-23, wherein the disorder is hypertension.

28. The method of any one of claims 21-23, wherein the disorder or symptom is an obesity-related trait.

29. The method of any one of claims 21-23, wherein the disorder is type-1 diabetes.

30. The method of any one of claims 21-23, wherein the disorder is type-2 diabetes.

31. The method of any one of claims 21-23, wherein the disorder is psoriasis.

32. The method of any one of claims 21-23, wherein the disorder is endometriosis.

33. The method of any one of claims 21-23, wherein the disorder is a chronic inflammatory disease, e.g., ankylosing spondylitis, Crohn's disease, psoriasis, primary sclerosing cholangitis, ulcerative colitis, or pleiotropy.

34. The method of any one of claims 21-23, wherein the disorder is Crohn's disease.

35. The method of any one of claims 21-23, wherein the disorder is Grave's disease.

36. The method of any one of claims 21-23, wherein the disorder is Alzheimer's disease, e.g., age onset Alzheimer's disease or familial Alzheimer's disease.

37. The method of any one of claims 21-23, wherein the disorder is a major depressive disorder.

38. The method of any one of claims 21-23, wherein the disorder is migraine.

39. The method of any one of claims 21-23, wherein the disorder is Parkinson's disease.

40. The method of any one of claims 21-23, wherein the disorder is schizophrenia.

41. The method of any one of claims 21-23, wherein the disorder or symptom is adverse response to chemotherapy, e.g., neutropenia or leukopenia.

42. The method of any one of claims 21-23, wherein the disorder is breast cancer, e.g., early onset breast cancer.

43. The method of any one of claims 21-23, wherein the disorder is ovarian cancer.

44. The method of any one of claims 21-23, wherein the disorder is colorectal cancer.

45. The method of any one of claims 21-23, wherein the disorder is carboplatin disposition in epithelial ovarian cancer.

46. The method of any one of claims 21-23, wherein the disorder is Clostridium difficile infection in multiple myeloma.

47. The method of any one of claims 21-23, wherein the disorder is endometrial cancer, e.g., with endometrioid histology.

48. The method of any one of claims 21-23, wherein the disorder is esophageal squamous cell cancer.

49. The method of any one of claims 21-23, wherein the disorder is glioblastoma.

50. The method of any one of claims 21-23, wherein the disorder is lung cancer.

51. The method of any one of claims 21-23, wherein the disorder or symptom is Macrophage Migration Inhibitory Factor levels.

52. The method of any one of claims 21-23, wherein the disorder is oral cavity and pharyngeal cancer.

53. The method of any one of claims 21-23, wherein the disorder is pancreatic cancer.

54. The method of any one of claims 21-23, wherein the disorder is myopia.

55. The method of any one of claims 21-23, wherein the disorder is COPD.

56. The method of any one of claims 21-23, wherein the disorder is asthma.

57. The method of any one of the preceding claims, wherein the ORF codon having the first sequence; or the SMC is situated in a transcript provided in Table 1.

58. The method of any one of the preceding claims, wherein the ORF codon having the first sequence; or the SMC comprises a codon provided in Table 1, e.g., a codon listed in the “Codon From/To” column of Table 1, e.g., the second codon listed in said column in Table 1.

59. The method of any one of the preceding claims, wherein the first tRNA moiety comprises an endogenous tRNA and a TREM.

60. The method of any one of the preceding claims, wherein the second tRNA moiety comprises an endogenous tRNA and a TREM.

61. The method of any one of claim 1-2 or 10-60, wherein the composition comprising a TREM is made by a method described herein, e.g., using a synthetic method (e.g., synthesized using solid state synthesis or liquid phase synthesis); using in vitro transcription (IVT), or by expressing a vector encoding a TREM in a cell.

62. The method of any one of the preceding claims, wherein the ORF or the SMC containing ORF encodes a polypeptide.

63. The method of any one of the preceding claims, wherein the ORF or the SMC containing ORF is a chromosomal ORF or a mitochondrial ORF.

64. The method of any one of claim 1-2 or 10-63, wherein the composition comprising a TREM is a pharmaceutical composition comprising a TREM.

65. The method of any one of claim 1-2 or 10-64, wherein the composition comprising a TREM comprises a pharmaceutical excipient.

66. The method of any one of claim 1-2 or 10-65, wherein the composition comprising a TREM is administered with a delivery agent, e.g., a liposome, a polymer (e.g., a polymer conjugate), a particle, a microsphere, microparticle, or a nanoparticle.

67. The method of any one of claim 1-2 or 10-65, wherein the composition comprising a TREM is administered without a carrier, e.g., via naked delivery of the TREM.

68. The method of any one of claim 1-2 or 10-67, wherein the TREM comprises cognate adaptor function, and optionally wherein the TREM mediates acceptance and incorporation of an amino acid associated in nature with the anti-codon of the TREM in the initiation or elongation of a peptide chain.

69. The method of any one of claim 1-2 or 10-68, wherein the TREM comprises an RNA sequence at least 80% identical to an RNA encoded by a DNA sequence listed in Table 2, or a fragment or functional fragment thereof.

70. The method of any one of claim 1-2 or 10-69, wherein the TREM comprises an RNA sequence encoded by a DNA sequence listed in Table 2, or a fragment thereof.

71. The method of any one of claim 1-2 or 10-70, wherein the TREM comprises an RNA sequence at least XX % identical to an RNA sequence encoded by a DNA sequence listed in Table 2, or a fragment thereof, wherein XX is selected from 80, 85, 90, 95, 96, 97, 98, or 99.


SEQUENCE LISTING (468 sequences)

SEQ ID NO: 1 - DNA, 72 bp, Homo sapiens
gggggtatagctcagtggtagagcgcgtgcttagcatgcacgaggtcctgggttcgatccccagtacctcca

SEQ ID NO: 2 - DNA, 73 bp, Homo sapiens
ggggaattagctcaagtggtagagcgcttgcttagcacgcaagaggtagtgggatcgatgcccacattctcca

SEQ ID NO: 3 - DNA, 73 bp, Homo sapiens
ggggaattagctcaaatggtagagcgctcgcttagcatgcgagaggtagcgggatcgatgcccgcattctcca

SEQ ID NO: 4 - DNA, 73 bp, Homo sapiens
ggggaattagctcaagtggtagagcgcttgcttagcatgcaagaggtagtgggatcgatgcccacattctcca

SEQ ID NO: 5 - DNA, 73 bp, Homo sapiens
ggggaattagctcaagcggtagagcgcttgcttagcatgcaagaggtagtgggatcgatgcccacattctcca

SEQ ID NO: 6 - DNA, 73 bp, Homo sapiens
ggggaattagctcaagtggtagagcgcttgcttagcatgcaagaggtagtgggatcaatgcccacattctcca

SEQ ID NO: 7 - DNA, 73 bp, Homo sapiens
ggggaattagctcaagtggtagagcgctcgcttagcatgcgagaggtagtgggatcgatgcccgcattctcca

SEQ ID NO: 8 - DNA, 73 bp, Homo sapiens
ggggaattagcccaagtggtagagcgcttgcttagcatgcaagaggtagtgggatcgatgcccacattctcca

SEQ ID NO: 9 - DNA, 72 bp, Homo sapiens
gggggtgtagctcagtggtagagcgcgtgcttagcatgcacgaggccccgggttcaatccccggcacctcca

SEQ ID NO: 10 - DNA, 72 bp, Homo sapiens
gggggtgtagctcagtggtagagcgcgtgcttagcatgtacgaggtcccgggttcaatccccggcacctcca

SEQ ID NO: 11 - DNA, 72 bp, Homo sapiens
ggggatgtagctcagtggtagagcgcatgcttagcatgcatgaggtcccgggttcgatccccagcatctcca

SEQ ID NO: 12 - DNA, 72 bp, Homo sapiens
gggggtgtagctcagtggtagagcgcgtgcttagcatgcacgaggccctgggttcaatccccagcacctcca

SEQ ID NO: 13 - DNA, 72 bp, Homo sapiens
gggggtatagctcagcggtagagcgcgtgcttagcatgcacgaggtcctgggttcaatccccaatacctcca

SEQ ID NO: 14 - DNA, 72 bp, Homo sapiens
gggggtgtagctcagtggtagagcgcgtgcttagcatgcacgaggccccgggttcaatccctggcacctcca

SEQ ID NO: 15 - DNA, 73 bp, Homo sapiens
gggggattagctcaaatggtagagcgctcgcttagcatgcgagaggtagcgggatcgatgcccgcatcctcca

SEQ ID NO: 16 - DNA, 73 bp, Homo sapiens
ggggaattagctcaggcggtagagcgctcgcttagcatgcgagaggtagcgggatcgacgcccgcattctcca

SEQ ID NO: 17 - DNA, 72 bp, Homo sapiens
ggggatgtagctcagtggtagagcgcatgcttcgcatgtatgaggtcccgggttcgatccccggcatctcca

SEQ ID NO: 18 - DNA, 72 bp, Homo sapiens
ggggatgtagctcagtggtagagcgcatgcttcgcatgtatgaggccccgggttcgatccccggcatctcca

SEQ ID NO: 19 - DNA, 72 bp, Homo sapiens
ggggatgtagctcagtggtagagcgcgcgcttcgcatgtgtgaggtcccgggttcaatccccggcatctcca

SEQ ID NO: 20 - DNA, 72 bp, Homo sapiens
gggggtgtagctcagtggtagagcgcgtgcttcgcatgtacgaggccccgggttcgacccccggctcctcca

SEQ ID NO: 21 - DNA, 72 bp, Homo sapiens
gggggtgtagctcagtggtagagcgcatgctttgcatgtatgaggtcccgggttcgatccccggcacctcca

SEQ ID NO: 22 - DNA, 72 bp, Homo sapiens
ggggatgtagctcagtggtagagcgcatgctttgcatgtatgaggtcccgggttcgatccccggcatctcca

SEQ ID NO: 23 - DNA, 72 bp, Homo sapiens
ggggatgtagctcagtggtagagcgcatgctttgcatgtatgaggccccgggttcgatccccggcatctcca

SEQ ID NO: 24 - DNA, 72 bp, Homo sapiens
ggggatgtagctcagtggtagagcgcatgctttgcacgtatgaggccccgggttcaatccccggcatctcca

SEQ ID NO: 25 - DNA, 72 bp, Homo sapiens
gggggtgtagctcagtggtagagcgcatgctttgcatgtatgaggcctcgggttcgatccccgacacctcca

SEQ ID NO: 26 - DNA, 72 bp, Homo sapiens
gggggtgtagctcagtggtagagcacatgctttgcatgtgtgaggccccgggttcgatccccggcacctcca

SEQ ID NO: 27 - DNA, 71 bp, Homo sapiens
gggggtgtagctcagtggtagagcgcatgctttgcatgtatgaggcctcggttcgatccccgacacctcca

SEQ ID NO: 28 - DNA, 73 bp, Homo sapiens
gggccagtggcgcaatggataacgcgtctgactacggatcagaagattccaggttcgactcctggctggctcg

SEQ ID NO: 29 - DNA, 73 bp, Homo sapiens
gggccagtggcgcaatggataacgcgtctgactacggatcagaagattctaggttcgactcctggctggctcg

SEQ ID NO: 30 - DNA, 73 bp, Homo sapiens
ggccgcgtggcctaatggataaggcgtctgattccggatcagaagattgagggttcgagtcccttcgtggtcg

SEQ ID NO: 31 - DNA, 73 bp, Homo sapiens
gacccagtggcctaatggataaggcatcagcctccggagctggggattgtgggttcgagtcccatctgggtcg

SEQ ID NO: 32 - DNA, 73 bp, Homo sapiens
gccccagtggcctaatggataaggcactggcctcctaagccagggattgtgggttcgagtcccacctggggta

SEQ ID NO: 33 - DNA, 73 bp, Homo sapiens
gccccagtggcctaatggataaggcactggcctcctaagccagggattgtgggttcgagtcccacctggggtg

SEQ ID NO: 34 - DNA, 73 bp, Homo sapiens
gccccggtggcctaatggataaggcattggcctcctaagccagggattgtgggttcgagtcccacccggggta

SEQ ID NO: 35 - DNA, 73 bp, Homo sapiens
gccccagtggcctaatggataaggcattggcctcctaagccagggattgtgggttcgagtcccatctggggtg

SEQ ID NO: 36 - DNA, 73 bp, Homo sapiens
gccccagtggcctgatggataaggtactggcctcctaagccagggattgtgggttcgagttccacctggggta

SEQ ID NO: 37 - DNA, 73 bp, Homo sapiens
ggccgcgtggcctaatggataaggcgtctgacttcggatcagaagattgcaggttcgagtcctgccgcggtcg

SEQ ID NO: 38 - DNA, 73 bp, Homo sapiens
gaccacgtggcctaatggataaggcgtctgacttcggatcagaagattgagggttcgaatccctccgtggtta

SEQ ID NO: 39 - DNA, 73 bp, Homo sapiens
gaccgcgtggcctaatggataaggcgtctgacttcggatcagaagattgagggttcgagtcccttcgtggtcg

SEQ ID NO: 40 - DNA, 73 bp, Homo sapiens
gaccacgtggcctaatggataaggcgtctgacttcggatcagaagattgagggttcgaatcccttcgtggtta

SEQ ID NO: 41 - DNA, 73 bp, Homo sapiens
gaccacgtggcctaatggataaggcgtctgacttcggatcagaagattgagggttcgaatcccttcgtggttg

SEQ ID NO: 42 - DNA, 73 bp, Homo sapiens
ggccgtgtggcctaatggataaggcgtctgacttcggatcaaaagattgcaggtttgagttctgccacggtcg

SEQ ID NO: 43 - DNA, 85 bp, Homo sapiens
ggctccgtggcgcaatggatagcgcattggacttctagaggctgaaggcattcaaaggttccgggttcgagtcccggcggagtcg

SEQ ID NO: 44 - DNA, 88 bp, Homo sapiens
ggctctgtggcgcaatggatagcgcattggacttctagtgacgaatagagcaattcaaaggttgtgggttcgaatcccaccagagtcg

SEQ ID NO: 45 - DNA, 91 bp, Homo sapiens
ggctctgtggcgcaatggatagcgcattggacttctagctgagcctagtgtggtcattcaaaggttgtgggttcgagtcccaccagagtcg

SEQ ID NO: 46 - DNA, 86 bp, Homo sapiens
ggctctgtggcgcaatggatagcgcattggacttctagatagttagagaaattcaaaggttgtgggttcgagtcccaccagagtcg

SEQ ID NO: 47 - DNA, 74 bp, Homo sapiens
gtctctgtggcgcaatggacgagcgcgctggacttctaatccagaggttccgggttcgagtcccggcagagatg

SEQ ID NO: 48 - DNA, 87 bp, Homo sapiens
ggctctgtggcgcaatggatagcgcattggacttctagcctaaatcaagagattcaaaggttgcgggttcgagtccctccagagtcg

SEQ ID NO: 49 - DNA, 74 bp, Homo sapiens
gtctctgtggcgcaatcggttagcgcgttcggctgttaaccgaaaggttggtggttcgatcccacccagggacg

SEQ ID NO: 50 - DNA, 74 bp, Homo sapiens
gtctctgtggcgcaatcggctagcgcgtttggctgttaactaaaaggttggcggttcgaacccacccagaggcg

SEQ ID NO: 51 - DNA, 74 bp, Homo sapiens
gtctctgtggtgcaatcggttagcgcgttccgctgttaaccgaaagcttggtggttcgagcccacccagggatg

SEQ ID NO: 52 - DNA, 74 bp, Homo sapiens
gtctctgtggcgcaatcggctagcgcgtttggctgttaactaaaaagttggtggttcgaacacacccagaggcg

SEQ ID NO: 53 - DNA, 74 bp, Homo sapiens
gtctctgtggcgcaatcggttagcgcgttcggctgttaaccgaaaggttggtggttcgagcccacccagggacg

SEQ ID NO: 54 - DNA, 74 bp, Homo sapiens
gtctctgtggcgcaatcggttagcgcattcggctgttaaccgaaaggttggtggttcgagcccacccagggacg

SEQ ID NO: 55 - DNA, 74 bp, Homo sapiens
gtctctgtggcgcaatcggttagcgcgttcggctgttaaccgaaagattggtggttcgagcccacccagggacg

SEQ ID NO: 56 - DNA, 74 bp, Homo sapiens
gtctctgtggcgcaatcggttagcgcgttcggctgttaactgaaaggttggtggttcgagcccacccagggacg

SEQ ID NO: 57 - DNA, 74 bp, Homo sapiens
gtctctgtggcgcaatgggttagcgcgttcggctgttaaccgaaaggttggtggttcgagcccatccagggacg

SEQ ID NO: 58 - DNA, 74 bp, Homo sapiens
gtctctgtggcgtagtcggttagcgcgttcggctgttaaccgaaaagttggtggttcgagcccacccaggaacg

SEQ ID NO: 59 - DNA, 74 bp, Homo sapiens
gtctctgtggcgcaatcggctagcgcgtttggctgttaactaaaaggttggtggttcgaacccacccagaggcg

SEQ ID NO: 60 - DNA, 74 bp, Homo sapiens
gtctctgtggcgcaatcggttagcgcgttcggctgttaactgaaaggttagtggttcgagcccacccggggacg

SEQ ID NO: 61 - DNA, 72 bp, Homo sapiens
tcctcgttagtatagtggttagtatccccgcctgtcacgcgggagaccggggttcaattccccgacggggag

SEQ ID NO: 62 - DNA, 72 bp, Homo sapiens
tcctcgttagtatagtggtgagtatccccgcctgtcacgcgggagaccggggttcgattccccgacggggag

SEQ ID NO: 63 - DNA, 72 bp, Homo sapiens
tcctcgttagtatagtggtgagtgtccccgtctgtcacgcgggagaccggggttcgattccccgacggggag

SEQ ID NO: 64 - DNA, 72 bp, Homo sapiens
gggggcatagctcagtggtagagcatttgactgcagatcaagaggtccctggttcaaatccaggtgccccct

SEQ ID NO: 65 - DNA, 72 bp, Homo sapiens
gggggtatagctcaggggtagagcatttgactgcagatcaagaggtccctggttcaaatccaggtgcccccc

SEQ ID NO: 66 - DNA, 72 bp, Homo sapiens
gggggtatagcttagcggtagagcatttgactgcagatcaagaggtccccggttcaaatccgggtgccccct

SEQ ID NO: 67 - DNA, 72 bp, Homo sapiens
gggggtatagcttaggggtagagcatttgactgcagatcaaaaggtccctggttcaaatccaggtgcccctt

SEQ ID NO: 68 - DNA, 72 bp, Homo sapiens
gggggtatagctcaggggtagagcatttgactgcagatcaagaggtccccagttcaaatctgggtgccccct

SEQ ID NO: 69 - DNA, 72 bp, Homo sapiens
gggggtatagctcaggggtagagcatttgactgcagatcaagaagtccccggttcaaatccgggtgccccct

SEQ ID NO: 70 - DNA, 72 bp, Homo sapiens
gggggtatagctcaggggtagagcatttgactgcagatcaagaggtctctggttcaaatccaggtgccccct

SEQ ID NO: 71 - DNA, 72 bp, Homo sapiens
gggggtatagctcaggggtagagcacttgactgcagatcaagaagtccttggttcaaatccaggtgccccct

SEQ ID NO: 72 - DNA, 72 bp, Homo sapiens
ggggatatagctcaggggtagagcatttgactgcagatcaagaggtccccggttcaaatccgggtgcccccc

SEQ ID NO: 73 - DNA, 72 bp, Homo sapiens
gggggtatagttcaggggtagagcatttgactgcagatcaagaggtccctggttcaaatccaggtgccccct

SEQ ID NO: 74 - DNA, 72 bp, Homo sapiens
gggggtatagctcaggggtagagcatttgactgcaaatcaagaggtccctgattcaaatccaggtgccccct

SEQ ID NO: 75 - DNA, 72 bp, Homo sapiens
gggggtatagctcagtggtagagcatttgactgcagatcaagaggtccccggttcaaatccgggtgccccct

SEQ ID NO: 76 - DNA, 72 bp, Homo sapiens
gggcgtatagctcaggggtagagcatttgactgcagatcaagaggtccccagttcaaatctgggtgccccct

SEQ ID NO: 77 - DNA, 72 bp, Homo sapiens
gggggtatagctcacaggtagagcatttgactgcagatcaagaggtccccggttcaaatctgggtgccccct

SEQ ID NO: 78 - DNA, 70 bp, Homo sapiens
gggcgtatagctcaggggtagagcatttgactgcagatcaagaggtccccagttcaaatctgggtgccca

SEQ ID NO: 79 - DNA, 72 bp, Homo sapiens
gggggtatagctcacaggtagagcatttgactgcagatcaagaggtccccggttcaaatccggttactccct

SEQ ID NO: 80 - DNA, 72 bp, Homo sapiens
gggggtatagctcaggggtagagcacttgactgcagatcaagaggtccctggttcaaatccaggtgccccct

SEQ ID NO: 81 - DNA, 72 bp, Homo sapiens
gggggtatagctcagtggtagagcatttgactgcagatcaagaggtccctggttcaaatccgggtgccccct

SEQ ID NO: 82 - DNA, 73 bp, Homo sapiens
gggggtatagctcagtgggtagagcatttgactgcagatcaagaggtccccggttcaaatccgggtgccccct

SEQ ID NO: 83 - DNA, 72 bp, Homo sapiens
gggggtgtagctcagtggtagagcatttgactgcagatcaagaggtccctggttcaaatccaggtgccccct

SEQ ID NO: 84 - DNA, 73 bp, Homo sapiens
gggggtatagctcaggtggtagagcatttgactgcagatcaagaggtccccggttcaaatccgggtgccccct

SEQ ID NO: 85 - DNA, 72 bp, Homo sapiens
gggggtatagctcaggggtagagcatttgactgcagatcaagaggtccccggttcaaatccgggtgccccct

SEQ ID NO: 86 - DNA, 72 bp, Homo sapiens
gggggtatagctcaggggtagagcatttgactgcagatcaagaggtccctggttcaaatccaggtgccccct

SEQ ID NO: 87 - DNA, 72 bp, Homo sapiens
ggttccatggtgtaatggttagcactctggactctgaatccagcgatccgagttcaaatctcggtggaacct

SEQ ID NO: 88 - DNA, 72 bp, Homo sapiens
ggttccatggtgtaatggttagcactctggactctgaatccagcgatccgagttcaagtctcggtggaacct

SEQ ID NO: 89 - DNA, 72 bp, Homo sapiens
ggttccatggtgtaatggtgagcactctggactctgaatccagcgatccgagttcgagtctcggtggaacct

SEQ ID NO: 90 - DNA, 72 bp, Homo sapiens
ggttccatggtgtaatggtaagcactctggactctgaatccagcgatccgagttcgagtctcggtggaacct

SEQ ID NO: 91 - DNA, 72 bp, Homo sapiens
ggttccatggtgtaatggttagcactctggactctgaatccggtaatccgagttcaaatctcggtggaacct

SEQ ID NO: 92 - DNA, 72 bp, Homo sapiens
ggccccatggtgtaatggtcagcactctggactctgaatccagcgatccgagttcaaatctcggtgggaccc

SEQ ID NO: 93 - DNA, 72 bp, Homo sapiens
ggttccatggtgtaatggtaagcactctggactctgaatccagccatctgagttcgagtctctgtggaacct

SEQ ID NO: 94 - DNA, 72 bp, Homo sapiens
ggtcccatggtgtaatggttagcactctggactttgaatccagcgatccgagttcaaatctcggtgggacct

SEQ ID NO: 95 - DNA, 72 bp, Homo sapiens
ggtcccatggtgtaatggttagcactctggactttgaatccagcaatccgagttcgaatctcggtgggacct

SEQ ID NO: 96 - DNA, 72 bp, Homo sapiens
ggccccatggtgtaatggttagcactctggactttgaatccagcgatccgagttcaaatctcggtgggacct

SEQ ID NO: 97 - DNA, 72 bp, Homo sapiens
ggtcccatggtgtaatggttagcactctgggctttgaatccagcaatccgagttcgaatcttggtgggacct

SEQ ID NO: 98 - DNA, 72 bp, Homo sapiens
tccctggtggtctagtggttaggattcggcgctctcaccgccgcggcccgggttcgattcccggtcagggaa

SEQ ID NO: 99 - DNA, 72 bp, Homo sapiens
tccctggtggtctagtggttaggattcggcgctctcaccgccgcggcccgggttcgattcccggtcaggaaa

SEQ ID NO: 100 - DNA, 72 bp, Homo sapiens
tcccatatggtctagcggttaggattcctggttttcacccaggtggcccgggttcgactcccggtatgggaa

SEQ ID NO: 101 - DNA, 72 bp, Homo sapiens
tcccacatggtctagcggttaggattcctggttttcacccaggcggcccgggttcgactcccggtgtgggaa

SEQ ID NO: 102 - DNA, 72 bp, Homo sapiens
tccctggtggtctagtggctaggattcggcgctttcaccgccgcggcccgggttcgattcccggccagggaa

SEQ ID NO: 103 - DNA, 72 bp, Homo sapiens
tccctggtggtctagtggctaggattcggcgctttcaccgccgcggcccgggttcgattcccggtcagggaa

SEQ ID NO: 104 - DNA, 71 bp, Homo sapiens
gcattggtggttcagtggtagaattctcgcctcccacgcgggagacccgggttcaattcccggccaatgca

SEQ ID NO: 105 - DNA, 71 bp, Homo sapiens
gcgccgctggtgtagtggtatcatgcaagattcccattcttgcgacccgggttcgattcccgggcggcgca

SEQ ID NO: 106 - DNA, 71 bp, Homo sapiens
gcattggtggttcaatggtagaattctcgcctcccacgcaggagacccaggttcgattcctggccaatgca

SEQ ID NO: 107 - DNA, 71 bp, Homo sapiens
gcatgggtggttcagtggtagaattctcgcctgccacgcgggaggcccgggttcgattcccggcccatgca

SEQ ID NO: 108 - DNA, 71 bp, Homo sapiens
gcattggtggttcagtggtagaattctcgcctgccacgcgggaggcccgggttcgattcccggccaatgca

SEQ ID NO: 109 - DNA, 71 bp, Homo sapiens
gcattggtggttcagtggtagaattctcgcctgccacgcgggaggcccgggtttgattcccggccagtgca

SEQ ID NO: 110 - DNA, 71 bp, Homo sapiens
gcataggtggttcagtggtagaattcttgcctgccacgcaggaggcccaggtttgattcctggcccatgca

SEQ ID NO: 111 - DNA, 71 bp, Homo sapiens
gcattggtggttcagtggtagaattctcgcctgccatgcgggcggccgggcttcgattcctggccaatgca

SEQ ID NO: 112 - DNA, 72 bp, Homo sapiens
gcgttggtggtatagtggttagcatagctgccttccaagcagttgacccgggttcgattcccggccaacgca

SEQ ID NO: 113 - DNA, 72 bp, Homo sapiens
gcgttggtggtatagtggtgagcatagctgccttccaagcagttgacccgggttcgattcccggccaacgca

SEQ ID NO: 114 - DNA, 72 bp, Homo sapiens
gcgttggtggtatagtggtaagcatagctgccttccaagcagttgacccgggttcgattcccggccaacgca

SEQ ID NO: 115 - DNA, 72 bp, Homo sapiens
gcgttggtggtatagtggtgagcatagttgccttccaagcagttgacccgggctcgattcccgcccaacgca

SEQ ID NO: 116 - DNA, 72 bp, Homo sapiens
gccgtgatcgtatagtggttagtactctgcgttgtggccgcagcaacctcggttcgaatccgagtcacggca

SEQ ID NO: 117 - DNA, 72 bp, Homo sapiens
gccatgatcgtatagtggttagtactctgcgctgtggccgcagcaacctcggttcgaatccgagtcacggca

SEQ ID NO: 118 - DNA, 74 bp, Homo sapiens
ggccggttagctcagttggttagagcgtggcgctaataacgccaaggtcgcgggttcgatccccgtacgggcca

SEQ ID NO: 119 - DNA, 74 bp, Homo sapiens
ggccggttagctcagttggttagagcgtggtgctaataacgccaaggtcgcgggttcgatccccgtactggcca

SEQ ID NO: 120 - DNA, 74 bp, Homo sapiens
ggctggttagctcagttggttagagcgtggtgctaataacgccaaggtcgcgggttcgatccccgtactggcca

SEQ ID NO: 121 - DNA, 74 bp, Homo sapiens
ggccggttagctcagttggttagagcgtggtgctaataacgccaaggtcgcgggttcgaaccccgtacgggcca

SEQ ID NO: 122 - DNA, 74 bp, Homo sapiens
ggccggttagctcagttggttagagcgtggtgctaataacgccaaggtcgcgggttcgatccccgtacgggcca

SEQ ID NO: 123 - DNA, 74 bp, Homo sapiens
ggccggttagctcagttggttagagcgtggtgctaataacgctaaggtcgcgggttcgatccccgtactggcca

SEQ ID NO: 124 - DNA, 74 bp, Homo sapiens
ggccggttagctcagttggtcagagcgtggtgctaataacgccaaggtcgcgggttcgatccccgtacgggcca

SEQ ID NO: 125 - DNA, 74 bp, Homo sapiens
ggccggttagctcagtcggctagagcgtggtgctaataacgccaaggtcgcgggttcgatccccgtacgggcca

SEQ ID NO: 126 - DNA, 74 bp, Homo sapiens
ggctggttagttcagttggttagagcgtggtgctaataacgccaaggtcgtgggttcgatccccatatcggcca

SEQ ID NO: 127 - DNA, 74 bp, Homo sapiens
ggccggttagctcagttggtaagagcgtggtgctgataacaccaaggtcgcgggctcgactcccgcaccggcca

SEQ ID NO: 128 - DNA, 93 bp, Homo sapiens
gctccagtggcgcaatcggttagcgcgcggtacttatatgacagtgcgagcggagcaatgccgaggttgtgagttcgatcctcacctggagca

SEQ ID NO: 129 - DNA, 93 bp, Homo sapiens
gctccagtggcgcaatcggttagcgcgcggtacttatacagcagtacatgcagagcaatgccgaggttgtgagttcgagcctcacctggagca

SEQ ID NO: 130 - DNA, 94 bp, Homo sapiens
gctccagtggcgcaatcggttagcgcgcggtacttatatggcagtatgtgtgcgagtgatgccgaggttgtgagttcgagcctcacctggagca

SEQ ID NO: 131 - DNA, 94 bp, Homo sapiens
gctccagtggcgcaatcggttagcgcgcggtacttatacaacagtatatgtgcgggtgatgccgaggttgtgagttcgagcctcacctggagca

SEQ ID NO: 132 - DNA, 94 bp, Homo sapiens
gctccagtggcgcaatcggttagcgcgcggtacttataagacagtgcacctgtgagcaatgccgaggttgtgagttcaagcctcacctggagca

SEQ ID NO: 133 - DNA, 82 bp, Homo sapiens
ggtagcgtggccgagcggtctaaggcgctggattaaggctccagtctcttcggaggcgtgggttcgaatcccaccgctgcca

SEQ ID NO: 134 - DNA, 82 bp, Homo sapiens
ggtagcgtggccgagcggtctaaggcgctggattaaggctccagtctcttcgggggcgtgggttcgaatcccaccgctgcca

SEQ ID NO: 135 - DNA, 82 bp, Homo sapiens
ggtagcgtggccgagcggtctaaggcgctggattaaggctccagtctcttcgggggcgtgggttcaaatcccaccgctgcca

SEQ ID NO: 136 - DNA, 82 bp, Homo sapiens
ggtagcgtggccgagtggtctaagacgctggattaaggctccagtctcttcgggggcgtgggtttgaatcccaccgctgcca

SEQ ID NO: 137 - DNA, 106 bp, Homo sapiens
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, Homo sapiens
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, Homo sapiens
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, Homo sapiens
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, Homo sapiens
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, Homo sapiens
gcctccttagtgcagtaggtagcgcatcagtctcaaaatctgaatggtcctgagttcaagcctcagagggggca

SEQ ID NO: 143 - DNA, 84 bp, Homo sapiens
gtcaggatggccgagcagtcttaaggcgctgcgttcaaatcgcaccctccgctggaggcgtgggttcgaatcccacttttgaca

SEQ ID NO: 144 - DNA, 83 bp, Homo sapiens
gtcaggatggccgagcggtctaaggcgctgcgttcaggtcgcagtctcccctggaggcgtgggttcgaatcccactcctgaca

SEQ ID NO: 145 - DNA, 83 bp, Homo sapiens
gtcaggatggccgagcggtctaaggcgctgcgttcaggtcgcagtctcccctggaggcgtgggttcgaatcccacttctgaca

SEQ ID NO: 146 - DNA, 83 bp, Homo sapiens
accaggatggccgagtggttaaggcgttggacttaagatccaatggacatatgtccgcgtgggttcgaaccccactcctggta

SEQ ID NO: 147 - DNA, 83 bp, Homo sapiens
accgggatggccgagtggttaaggcgttggacttaagatccaatgggctggtgcccgcgtgggttcgaaccccactctcggta

SEQ ID NO: 148 - DNA, 83 bp, Homo sapiens
accagaatggccgagtggttaaggcgttggacttaagatccaatggattcatatccgcgtgggttcgaaccccacttctggta

SEQ ID NO: 149 - DNA, 83 bp, Homo sapiens
accgggatggctgagtggttaaggcgttggacttaagatccaatggacaggtgtccgcgtgggttcgagccccactcccggta

SEQ ID NO: 150 - DNA, 82 bp, Homo sapiens
ggtagcgtggccgagcggtctaaggcgctggatttaggctccagtctcttcggaggcgtgggttcgaatcccaccgctgcca

SEQ ID NO: 151 - DNA, 82 bp, Homo sapiens
ggtagtgtggccgagcggtctaaggcgctggatttaggctccagtctcttcgggggcgtgggttcgaatcccaccactgcca

SEQ ID NO: 152 - DNA, 82 bp, Homo sapiens
ggtagcgtggccgagtggtctaaggcgctggatttaggctccagtcatttcgatggcgtgggttcgaatcccaccgctgcca

SEQ ID NO: 153 - DNA, 73 bp, Homo sapiens
gcccggctagctcagtcggtagagcatgggactcttaatcccagggtcgtgggttcgagccccacgttgggcg

SEQ ID NO: 154 - DNA, 73 bp, Homo sapiens
gcccagctagctcagtcggtagagcataagactcttaatctcagggttgtggattcgtgccccatgctgggtg

SEQ ID NO: 155 - DNA, 74 bp, Homo sapiens
gcagctagctcagtcggtagagcatgagactcttaatctcagggtcatgggttcgtgccccatgttgggtgcca

SEQ ID NO: 156 - DNA, 73 bp, Homo sapiens
gcccggctagctcagtcggtagagcatgagactcttaatctcagggtcgtgggttcgagccccacgttgggcg

SEQ ID NO: 157 - DNA, 73 bp, Homo sapiens
gcccggctagctcagtcggtagagcatgagacccttaatctcagggtcgtgggttcgagccccacgttgggcg

SEQ ID NO: 158 - DNA, 73 bp, Homo sapiens
gcccggctagctcagtcggtagagcatgggactcttaatctcagggtcgtgggttcgagccccacgttgggcg

SEQ ID NO: 159 - DNA, 73 bp, Homo sapiens
gcccggctagctcagtcgatagagcatgagactcttaatctcagggtcgtgggttcgagccgcacgttgggcg

SEQ ID NO: 160 - DNA, 73 bp, Homo sapiens
gcccagctagctcagtcggtagagcatgagactcttaatctcagggtcatgggtttgagccccacgtttggtg

SEQ ID NO: 161 - DNA, 73 bp, Homo sapiens
gcctggctagctcagtcggcaaagcatgagactcttaatctcagggtcgtgggctcgagctccatgttgggcg

SEQ ID NO: 162 - DNA, 73 bp, Homo sapiens
gcccgactacctcagtcggtggagcatgggactcttcatcccagggttgtgggttcgagccccacattgggca

SEQ ID NO: 163 - DNA, 73 bp, Homo sapiens
gcctggatagctcagttggtagagcatcagacttttaatctgagggtccagggttcaagtccctgttcaggca

SEQ ID NO: 164 - DNA, 73 bp, Homo sapiens
acccagatagctcagtcagtagagcatcagacttttaatctgagggtccaaggttcatgtccctttttgggtg

SEQ ID NO: 165 - DNA, 73 bp, Homo sapiens
gcctggatagctcagttggtagagcatcagacttttaatctgagggtccagggttcaagtccctgttcaggcg

SEQ ID NO: 166 - DNA, 73 bp, Homo sapiens
gcccggatagctcagtcggtagagcatcagacttttaatctgagggtccagggttcaagtccctgttcgggcg

SEQ ID NO: 167 - DNA, 73 bp, Homo sapiens
gcctggatagctcagtcggtagagcatcagacttttaatctgagggtccagggttcaagtccctgttcaggcg

SEQ ID NO: 168 - DNA, 73 bp, Homo sapiens
gcccggatagctcagtcggtagagcatcagacttttaatctgagggtccggggttcaagtccctgttcgggcg

SEQ ID NO: 169 - DNA, 73 bp, Homo sapiens
gcctgggtagctcagtcggtagagcatcagacttttaatctgagggtccagggttcaagtccctgtccaggcg

SEQ ID NO: 170 - DNA, 73 bp, Homo sapiens
gcctggatagctcagttggtagaacatcagacttttaatctgacggtgcagggttcaagtccctgttcaggcg

SEQ ID NO: 171 - DNA, 73 bp, Homo sapiens
gcctcgttagcgcagtaggtagcgcgtcagtctcataatctgaaggtcgtgagttcgatcctcacacggggca

SEQ ID NO: 172 - DNA, 73 bp, Homo sapiens
gccctcttagcgcagtgggcagcgcgtcagtctcataatctgaaggtcctgagttcgagcctcagagagggca

SEQ ID NO: 173 - DNA, 73 bp, Homo sapiens
gcctccttagcgcagtaggcagcgcgtcagtctcataatctgaaggtcctgagttcgaacctcagagggggca

SEQ ID NO: 174 - DNA, 73 bp, Homo sapiens
gccctcttagcgcagcgggcagcgcgtcagtctcataatctgaaggtcctgagttcgagcctcagagagggca

SEQ ID NO: 175 - DNA, 73 bp, Homo sapiens
gccctcttagcgcagctggcagcgcgtcagtctcataatctgaaggtcctgagttcaagcctcagagagggca

SEQ ID NO: 176 - DNA, 73 bp, Homo sapiens
gcctcgttagcgcagtaggcagcgcgtcagtctcataatctgaaggtcgtgagttcgagcctcacacggggca

SEQ ID NO: 177 - DNA, 73 bp, Homo sapiens
gccctcttagtgcagctggcagcgcgtcagtttcataatctgaaagtcctgagttcaagcctcagagagggca

SEQ ID NO: 178 - DNA, 73 bp, Homo sapiens
gccgaaatagctcagttgggagagcgttagactgaagatctaaaggtccctggttcgatcccgggtttcggca

SEQ ID NO: 179 - DNA, 73 bp, Homo sapiens
gccgaaatagctcagttgggagagcgttagactgaagatctaaaggtccctggttcaatcccgggtttcggca

SEQ ID NO: 180 - DNA, 73 bp, Homo sapiens
gccgagatagctcagttgggagagcgttagactgaagatctaaaggtccctggttcaatcccgggtttcggca

SEQ ID NO: 181 - DNA, 74 bp, Homo sapiens
gccgaaatagctcagttgggagagcgttagaccgaagatcttaaaggtccctggttcaatcccgggtttcggca

SEQ ID NO: 182 - DNA, 74 bp, Homo sapiens
gctgaaatagctcagttgggagagcgttagactgaagatcttaaagttccctggttcaaccctgggtttcagcc

SEQ ID NO: 183 - DNA, 72 bp, Homo sapiens
ggctcgttggtctaggggtatgattctcgcttaggatgcgagaggtcccgggttcaaatcccggacgagccc

SEQ ID NO: 184 - DNA, 72 bp, Homo sapiens
ggctcgttggtctaggggtatgattctcgcttagggtgcgagaggtcccgggttcaaatcccggacgagccc

SEQ ID NO: 185 - DNA, 72 bp, Homo sapiens
ggctcgttggtctaggggtatgattctcgcttcgggtgcgagaggtcccgggttcaaatcccggacgagccc

SEQ ID NO: 186 - DNA, 72 bp, Homo sapiens
ggctcgttggtctaggggtatgattctcgcttcgggtgtgagaggtcccgggttcaaatcccggacgagccc

SEQ ID NO: 187 - DNA, 72 bp, Homo sapiens
ggctcgttggtctagtggtatgattctcgctttgggtgcgagaggtcccgggttcaaatcccggacgagccc

SEQ ID NO: 188 - DNA, 72 bp, Homo sapiens
ggctcgttggtctaggggtatgattctcggtttgggtccgagaggtcccgggttcaaatcccggacgagccc

SEQ ID NO: 189 - DNA, 72 bp, Homo sapiens
ggctcgttggtctaggggtatgattctcgctttgggtgcgagaggtcccgggttcaaatcccggacgagccc

SEQ ID NO: 190 - DNA, 87 bp, Homo sapiens
gcccggatgatcctcagtggtctggggtgcaggcttcaaacctgtagctgtctagcgacagagtggttcaattccacctttcgggcg

SEQ ID NO: 191 - DNA, 84 bp, Homo sapiens
gctcggatgatcctcagtggtctggggtgcaggcttcaaacctgtagctgtctagtgacagagtggttcaattccacctttgta

SEQ ID NO: 192 - DNA, 82 bp, Homo sapiens
gtagtcgtggccgagtggttaaggcgatggactagaaatccattggggtttccccgcgcaggttcgaatcctgccgactacg

SEQ ID NO: 193 - DNA, 82 bp, Homo sapiens
gtagtcgtggccgagtggttaaggcgatggactagaaatccattggggtctccccgcgcaggttcgaatcctgccgactacg

SEQ ID NO: 194 - DNA, 82 bp, Homo sapiens
gtagtcgtggccgagtggttaaggcgatggactagaaatccattggggtttccccacgcaggttcgaatcctgccgactacg

SEQ ID NO: 195 - DNA, 82 bp, Homo sapiens
gtagtcgtggccgagtggttaaggtgatggactagaaacccattggggtctccccgcgcaggttcgaatcctgccgactacg

SEQ ID NO: 196 - DNA, 82 bp, Homo sapiens
gctgtgatggccgagtggttaaggcgttggactcgaaatccaatggggtctccccgcgcaggttcgaatcctgctcacagcg

SEQ ID NO: 197 - DNA, 82 bp, Homo sapiens
gctgtgatggccgagtggttaaggcgttggactcgaaatccaatggggtctccccgcgcaggttcaaatcctgctcacagcg

SEQ ID NO: 198 - DNA, 82 bp, Homo sapiens
gctgtgatggccgagtggttaaggtgttggactcgaaatccaatgggggttccccgcgcaggttcaaatcctgctcacagcg

SEQ ID NO: 199 - DNA, 82 bp, Homo sapiens
gtcacggtggccgagtggttaaggcgttggactcgaaatccaatggggtttccccgcacaggttcgaatcctgttcgtgacg

SEQ ID NO: 200 - DNA, 82 bp, Homo sapiens
gacgaggtggccgagtggttaaggcgatggactgctaatccattgtgctctgcacgcgtgggttcgaatcccaccctcgtcg

SEQ ID NO: 201 - DNA, 82 bp, Homo sapiens
gacgaggtggccgagtggttaaggcgatggactgctaatccattgtgctctgcacgcgtgggttcgaatcccaccttcgtcg

SEQ ID NO: 202 - DNA, 82 bp, Homo sapiens
gacgaggtggccgagtggttaaggcgatggactgctaatccattgtgctttgcacgcgtgggttcgaatcccatcctcgtcg

SEQ ID NO: 203 - DNA, 82 bp, Homo sapiens
gacgaggtggccgagtggttaaggcgatggactgctaatccattgtgctctgcacgcgtgggttcgaatcccatcctcgtcg

SEQ ID NO: 204 - DNA, 82 bp, Homo sapiens
gacgaggtggccgagtggttaaggcgatggactgctaatccattgtgctctgcacacgtgggttcgaatcccatcctcgtcg

SEQ ID NO: 205 - DNA, 84 bp, Homo sapiens
ggagaggcctggccgagtggttaaggcgatggactgctaatccattgtgctctgcacgcgtgggttcgaatcccatcctcgtcg

SEQ ID NO: 206 - DNA, 82 bp, Homo sapiens
gcagcgatggccgagtggttaaggcgttggacttgaaatccaatggggtctccccgcgcaggttcgaaccctgctcgctgcg

SEQ ID NO: 207 - DNA, 82 bp, Homo sapiens
gtagtcgtggccgagtggttaaggcgatggacttgaaatccattggggtttccccgcgcaggttcgaatcctgccgactacg

SEQ ID NO: 208 - DNA, 82 bp, Homo sapiens
gtagtcgtggccgagtggttaaggcgatggacttgaaatccattggggtctccccgcgcaggttcgaatcctgccgactacg

SEQ ID NO: 209 - DNA, 82 bp, Homo sapiens
gtagtcgtggccgagtggttaaggcgatggacttgaaatccattggggtttccccgcgcaggttcgaatcctgtcggctacg

SEQ ID NO: 210 - DNA, 74 bp, Homo sapiens
ggcgccgtggcttagttggttaaagcgcctgtctagtaaacaggagatcctgggttcgaatcccagcggtgcct

SEQ ID NO: 211 - DNA, 74 bp, Homo sapiens
ggctccgtggcttagctggttaaagcgcctgtctagtaaacaggagatcctgggttcgaatcccagcggggcct

SEQ ID NO: 212 - DNA, 74 bp, Homo sapiens
ggctccgtagcttagttggttaaagcgcctgtctagtaaacaggagatcctgggttcgactcccagcggggcct

SEQ ID NO: 213 - DNA, 74 bp, Homo sapiens
ggcttcgtggcttagctggttaaagcgcctgtctagtaaacaggagatcctgggttcgaatcccagcgaggcct

SEQ ID NO: 214 - DNA, 74 bp, Homo sapiens
ggcgccgtggcttagctggttaaagcgcctgtctagtaaacaggagatcctgggttcgaatcccagcggtgcct

SEQ ID NO: 215 - DNA, 74 bp, Homo sapiens
ggccctgtggcttagctggtcaaagcgcctgtctagtaaacaggagatcctgggttcgaatcccagcggggcct

SEQ ID NO: 216 - DNA, 74 bp, Homo sapiens
ggctctatggcttagttggttaaagcgcctgtctcgtaaacaggagatcctgggttcgactcccagtggggcct

SEQ ID NO: 217 - DNA, 72 bp, Homo sapiens
ggcgcggtggccaagtggtaaggcgtcggtctcgtaaaccgaagatcacgggttcgaaccccgtccgtgcct

SEQ ID NO: 218 - DNA, 74 bp, Homo sapiens
ggctctgtggcttagttggctaaagcgcctgtctcgtaaacaggagatcctgggttcgaatcccagcggggcct

SEQ ID NO: 219 - DNA, 72 bp, Homo sapiens
ggcgcggtggccaagtggtaaggcgtcggtctcgtaaaccgaagatcgcgggttcgaaccccgtccgtgcct

SEQ ID NO: 220 - DNA, 74 bp, Homo sapiens
ggccctgtagctcagcggttggagcgctggtctcgtaaacctaggggtcgtgagttcaaatctcaccagggcct

SEQ ID NO: 221 - DNA, 74 bp, Homo sapiens
ggctctatggcttagttggttaaagcgcctgtcttgtaaacaggagatcctgggttcgaatcccagtagagcct

SEQ ID NO: 222 - DNA, 73 bp, Homo sapiens
ggctccatagctcagtggttagagcactggtcttgtaaaccaggggtcgcgagttcgatcctcgctggggcct

SEQ ID NO: 223 - DNA, 73 bp, Homo sapiens
ggctccatagctcaggggttagagcgctggtcttgtaaaccaggggtcgcgagttcaattctcgctggggcct

SEQ ID NO: 224 - DNA, 73 bp, Homo sapiens
ggctccatagctcaggggttagagcactggtcttgtaaaccaggggtcgcgagttcaaatctcgctggggcct

SEQ ID NO: 225 - DNA, 73 bp, Homo sapiens
ggccctatagctcaggggttagagcactggtcttgtaaaccaggggtcgcgagttcaaatctcgctggggcct

SEQ ID NO: 226 - DNA, 72 bp, Homo sapiens
ggctccatagctcaggggttagagcactggtcttgtaaaccagggtcgcgagttcaaatctcgctggggcct

SEQ ID NO: 227 - DNA, 72 bp, Homo sapiens
ggcctcgtggcgcaacggtagcgcgtctgactccagatcagaaggttgcgtgttcaaatcacgtcggggtca

SEQ ID NO: 228 - DNA, 72 bp, Homo sapiens
gacctcgtggcgcaatggtagcgcgtctgactccagatcagaaggttgcgtgttcaagtcacgtcggggtca

SEQ ID NO: 229 - DNA, 72 bp, Homo sapiens
gacctcgtggcgcaacggtagcgcgtctgactccagatcagaaggttgcgtgttcaaatcacgtcggggtca

SEQ ID NO: 230 - DNA, 72 bp, Homo sapiens
gacctcgtggcgcaacggtagcgcgtctgactccagatcagaaggctgcgtgttcgaatcacgtcggggtca

SEQ ID NO: 231 - DNA, 72 bp, Homo sapiens
gacctcgtggcgcaacggcagcgcgtctgactccagatcagaaggttgcgtgttcaaatcacgtcggggtca

SEQ ID NO: 232 - DNA, 93 bp, Homo sapiens
ccttcaatagttcagctggtagagcagaggactatagctacttcctcagtaggagacgtccttaggttgctggttcgattccagcttgaagga

SEQ ID NO: 233 - DNA, 91 bp, Homo sapiens
ccttcgatagctcagttggtagagcggaggactgtagttggctgtgtccttagacatccttaggtcgctggttcgaatccggctcgaagga

SEQ ID NO: 234 - DNA, 89 bp, Homo sapiens
ccttcgatagctcagttggtagagcggaggactgtagtggatagggcgtggcaatccttaggtcgctggttcgattccggctcgaagga

SEQ ID NO: 235 - DNA, 89 bp, Homo sapiens
ccttcgatagctcagttggtagagcggaggactgtaggctcattaagcaaggtatccttaggtcgctggttcgaatccggctcggagga

SEQ ID NO: 236 - DNA, 94 bp, Homo sapiens
ccttcgatagctcagctggtagagcggaggactgtagattgtatagacatttgcggacatccttaggtcgctggttcgattccagctcgaagga

SEQ ID NO: 237 - DNA, 93 bp, Homo sapiens
ccttcgatagctcagctggtagagcggaggactgtagctacttcctcagcaggagacatccttaggtcgctggttcgattccggctcgaagga

SEQ ID NO: 238 - DNA, 89 bp, Homo sapiens
ccttcgatagctcagctggtagagcggaggactgtaggcgcgcgcccgtggccatccttaggtcgctggttcgattccggctcgaagga

SEQ ID NO: 239 - DNA, 94 bp, Homo sapiens
ccttcgatagctcagctggtagagcggaggactgtagcctgtagaaacatttgtggacatccttaggtcgctggttcgattccggctcgaagga

SEQ ID NO: 240 - DNA, 94 bp, Homo sapiens
ccttcgatagctcagctggtagagcggaggactgtagattgtacagacatttgcggacatccttaggtcgctggttcgattccggctcgaagga

SEQ ID NO: 241 - DNA, 89 bp, Homo sapiens
ccttcgatagctcagctggtagagcggaggactgtagtacttaatgtgtggtcatccttaggtcgctggttcgattccggctcgaagga

SEQ ID NO: 242 - DNA, 89 bp, Homo sapiens
ccttcgatagctcagctggtagagcggaggactgtaggggtttgaatgtggtcatccttaggtcgctggttcgaatccggctcggagga

SEQ ID NO: 243 - DNA, 94 bp, Homo sapiens
ccttcgatagctcagctggtagagcggaggactgtagactgcggaaacgtttgtggacatccttaggtcgctggttcaattccggctcgaagga

SEQ ID NO: 244 - DNA, 90 bp, Homo sapiens
ctttcgatagctcagttggtagagcggaggactgtaggttcattaaactaaggcatccttaggtcgctggttcgaatccggctcgaagga

SEQ ID NO: 245 - DNA, 88 bp, Homo sapiens
tcttcaatagctcagctggtagagcggaggactgtaggtgcacgcccgtggccattcttaggtgctggtttgattccgacttggagag

SEQ ID NO: 246 - DNA, 73 bp, Homo sapiens
gtttccgtagtgtagtggttatcacgttcgcctaacacgcgaaaggtccccggttcgaaaccgggcggaaaca

SEQ ID NO: 247 - DNA, 73 bp, Homo sapiens
gtttccgtagtgtagtggtcatcacgttcgcctaacacgcgaaaggtccccggttcgaaaccgggcggaaaca

SEQ ID NO: 248 - DNA, 73 bp, Homo sapiens
gtttccgtagtgtagtggttatcacgttcgcctaacacgcgaaaggtccctggatcaaaaccaggcggaaaca

SEQ ID NO: 249 - DNA, 73 bp, Homo sapiens
gtttccgtagtgtagtggttatcacgttcgcctaacacgcgaaaggtccgcggttcgaaaccgggcggaaaca

SEQ ID NO: 250 - DNA, 73 bp, Homo sapiens
gtttccgtagtgtagtggttatcacgtttgcctaacacgcgaaaggtccccggttcgaaaccgggcagaaaca

SEQ ID NO: 251 - DNA, 72 bp, Homo sapiens
gggggtgtagctcagtggtagagcgtatgcttaacattcatgaggctctgggttcgatccccagcacttcca

SEQ ID NO: 252 - DNA, 73 bp, Homo sapiens
gtttccgtagtgtagtggttatcacgttcgcctcacacgcgaaaggtccccggttcgaaaccgggcggaaaca

SEQ ID NO: 253 - DNA, 73 bp, Homo sapiens
gcttctgtagtgtagtggttatcacgttcgcctcacacgcgaaaggtccccggttcgaaaccgggcagaagca

SEQ ID NO: 254 - DNA, 73 bp, Homo sapiens
gtttccgtagtgtagcggttatcacattcgcctcacacgcgaaaggtccccggttcgatcccgggcggaaaca

SEQ ID NO: 255 - DNA, 73 bp, Homo sapiens
gtttccgtagtgtagtggttatcacgttcgcctcacacgcgaaaggtccccggttcgaaactgggcggaaaca

SEQ ID NO: 256 - DNA, 74 bp, Homo sapiens
gtttccgtagtgtagtggttatcacgttcgcctcacacgcgtaaaggtccccggttcgaaaccgggcggaaaca

SEQ ID NO: 257 - DNA, 73 bp, Homo sapiens
gtttccgtagtggagtggttatcacgttcgcctcacacgcgaaaggtccccggtttgaaaccaggcggaaaca

SEQ ID NO: 258 - DNA, 73 bp, Homo sapiens
ggttccatagtgtagtggttatcacgtctgctttacacgcagaaggtcctgggttcgagccccagtggaacca

SEQ ID NO: 259 - DNA, 73 bp, Homo sapiens
ggttccatagtgtagcggttatcacgtctgctttacacgcagaaggtcctgggttcgagccccagtggaacca

SEQ ID NO: 260 - DNA, 73 bp, Homo sapiens
ggttccatagtgtagtggttatcacatctgctttacacgcagaaggtcctgggttcaagccccagtggaacca

SEQ ID NO: 261 - DNA, 73 bp, Homo sapiens
gtttccgtggtgtagtggttatcacattcgccttacacgcgaaaggtcctcgggtcgaaaccgagcggaaaca

SEQ ID NO: 262 - DNA, 72 bp, Homo sapiens
agcagagtggcgcagcggaagcgtgctgggcccataacccagaggtcgatggatcgaaaccatcctctgcta

SEQ ID NO: 263 - DNA, 72 bp, Homo sapiens
agcagagtggcgcagcggaagcgtgctgggcccataacccagaggtcgatggatctaaaccatcctctgcta

SEQ ID NO: 264 - DNA, 73 bp, Homo sapiens
tccctggtggtctagtggctaggattcggcgctttcaccgccgcggcccgggttcgattcccggtcagggaat

SEQ ID NO: 265 - DNA, 72 bp, Homo sapiens
gcgttggtggtttagtggtagaattctcgcctcccatgcgggagacccgggttcaattcccggccactgcac

SEQ ID NO: 266 - DNA, 72 bp, Homo sapiens
ggccttggtggtgcagtggtagaattctcgcctcccacgtgggagacccgggttcaattcccggccaatgca

SEQ ID NO: 267 - DNA, 73 bp, Homo sapiens
gtccctggtggtctagtggctaggattcggcgctttcaccgccgcggcccgggttcgattcccggccagggaa

SEQ ID NO: 268 - DNA, 75 bp, Homo sapiens
tgtctctgtggcgcaatcggttagcgcgttcggctgttaaccgaaagattggtggttcgagcccacccagggacg

SEQ ID NO: 269 - DNA, 86 bp, Homo sapiens
tggctccgtggcgcaatggatagcgcattggacttctagaggctgaaggcattcaaaggttccgggttcgagtcccggcggagtcg

SEQ ID NO: 270 - DNA, 74 bp, Homo sapiens
gcccggctagctcagtcggtagagcatgagactcttaatctcagggtcgtgggttcgagccccacgttgggcgc

SEQ ID NO: 271 - DNA, 73 bp, Homo sapiens
gccgtgatcgtatagtggttagtactctgcgttgtggccgcagcaacctcggttcgaatccgagtcacggcag

SEQ ID NO: 272 - DNA, 73 bp, Homo sapiens
gcgttggtggtatagtggtgagcatagctgccttccaagcagttgacccgggttcgattcccggccaacgcag

SEQ ID NO: 273 - DNA, 73 bp, Homo sapiens
tccctggtggtctagtggttaggattcggcgctctcaccgccgcggcccgggttcgattcccggtcagggaaa

SEQ ID NO: 274 - DNA, 73 bp, Homo sapiens
aggttccatggtgtaatggtgagcactctggactctgaatccagcgatccgagttcgagtctcggtggaacct

SEQ ID NO: 275 - DNA, 75 bp, Homo sapiens
tgtctctgtggcgtagtcggttagcgcgttcggctgttaaccgaaaagttggtggttcgagcccacccaggaacg

SEQ ID NO: 276 - DNA, 75 bp, Homo sapiens
tgtctctgtggcgcaatcggttagcgcgttcggctgttaaccgaaaggttggtggttcgagcccacccagggacg

SEQ ID NO: 277 - DNA, 75 bp, Homo sapiens
gtctctgtggcgcaatcggttagcgcattcggctgttaaccgaaaggttggtggttcgagcccacccagggacgc

SEQ ID NO: 278 - DNA, 75 bp, Homo sapiens
gtctctgtggcgcaatgggttagcgcgttcggctgttaaccgaaaggttggtggttcgagcccatccagggacgc

SEQ ID NO: 279 - DNA, 72 bp, Homo sapiens
gcactggtggttcagtggtagaattctcgcctcacacgcgggacacccgggttcaattcccggtcaaggcaa

SEQ ID NO: 280 - DNA, 74 bp, Homo sapiens
gtttccgtagtgtagtggttatcacgttcgcctcacacgcgaaaggtccccggttcgaaactgggcggaaacag

SEQ ID NO: 281 - DNA, 72 bp, Homo sapiens
gcactggtggttcagtggtagaattctcgcctcccacgcgggagacccgggtttaattcccggtcaagataa

SEQ ID NO: 282 - DNA, 75 bp, Homo sapiens
gtttccgtagtgtagtggttatcacgttcgcctcacacgcgtaaaggtccccggttcgaaaccgggcggaaacat

SEQ ID NO: 283 - DNA, 73 bp, Homo sapiens
tagcagagtggcgcagcggaagcgtgctgggcccataacccagaggtcgatggatcgaaaccatcctctgcta

SEQ ID NO: 284 - DNA, 74 bp, Homo sapiens
gtttccgtagtgtagtggttatcacgttcgcctcacacgcgaaaggtccccggttcgaaaccgggcggaaacaa

SEQ ID NO: 285 - DNA, 73 bp, Homo sapiens
tcctcgttagtatagtggtgagtatccccgcctgtcacgcgggagaccggggttcgattccccgacggggagg

SEQ ID NO: 286 - DNA, 72 bp, Homo sapiens
tgcatgggtggttcagtggtagaattctcgcctgccacgcgggaggcccgggttcgattcccggcccatgca

SEQ ID NO: 287 - DNA, 73 bp, Homo sapiens
tccctggtggtctagtggttaggattcggcgctctcaccgccgcggcccgggttcgattcccggtcagggaag

SEQ ID NO: 288 - DNA, 73 bp, Homo sapiens
atccttgttactatagtggtgagtatctctgcctgtcatgcgtgagagagggggtcgattccccgacggggag

SEQ ID NO: 289 - DNA, 72 bp, Homo sapiens
gcattggtggttcagtggtagaattctcgcctgccacgcgggaggcccgggttcgattcccggccaatgcac

SEQ ID NO: 290 - DNA, 84 bp, Homo sapiens
gtcaggatggccgagcggtctaaggcgctgcgttcaggtcgcagtctcccctggaggcgtgggttcgaatcccactcctgacaa

SEQ ID NO: 291 - DNA, 73 bp, Homo sapiens
cgcgttggtggtatagtggtgagcatagctgccttccaagcagttgacccgggttcgattcccggccaacgca

SEQ ID NO: 292 - DNA, 75 bp, Homo sapiens
cgtctctgtggcgcaatcggttagcgcgttcggctgttaaccgaaaggttggtggttcgatcccacccagggacg

SEQ ID NO: 293 - DNA, 73 bp, Homo sapiens
cgcgttggtggtgtagtggtgagcacagctgcctttcaagcagttaacgcgggttcgattcccgggtaacgaa

SEQ ID NO: 294 - DNA, 73 bp, Homo sapiens
cggctcgttggtctaggggtatgattctcgcttcgggtgcgagaggtcccgggttcaaatcccggacgagccc

SEQ ID NO: 295 - DNA, 73 bp, Homo sapiens
ggctcgttggtctaggggtatgattctcgcttagggtgcgagaggtcccgggttcaaatcccggacgagccct

SEQ ID NO: 296 - DNA, 74 bp, Homo sapiens
cgcccggatagctcagtcggtagagcatcagacttttaatctgagggtccagggttcaagtccctgttcgggcg

SEQ ID NO: 297 - DNA, 74 bp, Homo sapiens
gcccggatagctcagtcggtagagcatcagacttttaatctgagggtccagggttcaagtccctgttcgggcgt

SEQ ID NO: 298 - DNA, 107 bp, Homo sapiens
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, Homo sapiens
ttccctggtggtctagtggttaggattcggcgctctcaccgccgcggcccgggttcgattcccggtcaggaaa

SEQ ID NO: 300 - DNA, 90 bp, Homo sapiens
gccttcgatagctcagttggtagagcggaggactgtagtggatagggcgtggcaatccttaggtcgctggttcgattccggctcgaagga

SEQ ID NO: 301 - DNA, 74 bp, Homo sapiens
cgggggattagctcaaatggtagagcgctcgcttagcatgcgagaggtagcgggatcgatgcccgcatcctcca

SEQ ID NO: 302 - DNA, 94 bp, Homo sapiens
agctccagtggcgcaatcggttagcgcgcggtacttatacagcagtacatgcagagcaatgccgaggttgtgagttcgagcctcacctggagca

SEQ ID NO: 303 - DNA, 72 bp, Homo sapiens
gcgccgctggtgtagtggtatcatgcaagattcccattcttgcgacccgggttcgattcccgggcggcgcat

SEQ ID NO: 304 - DNA, 73 bp, Homo sapiens
tcccatatggtctagcggttaggattcctggttttcacccaggtggcccgggttcgactcccggtatgggaac

SEQ ID NO: 305 - DNA, 73 bp, Homo sapiens
gggggatgtagctcagtggtagagcgcgcgcttcgcatgtgtgaggtcccgggttcaatccccggcatctcca

SEQ ID NO: 306 - DNA, 72 bp, Homo sapiens
gcattggtggttcagtggtagaattctcgcctgccacgcgggaggcccgggttcgattcccggccaatgcaa

SEQ ID NO: 307 - DNA, 74 bp, Homo sapiens
gggccagtggcgcaatggataacgcgtctgactacggatcagaagattctaggttcgactcctggctggctcgc

SEQ ID NO: 308 - DNA, 74 bp, Homo sapiens
ggtttccgtagtgtagtggttatcacgttcgcctaacacgcgaaaggtccccggttcgaaaccgggcggaaaca

SEQ ID NO: 309 - DNA, 74 bp, Homo sapiens
agtttccgtagtgtagtggttatcacgttcgcctaacacgcgaaaggtccccggttcgaaaccgggcggaaaca

SEQ ID NO: 310 - DNA, 83 bp, Homo sapiens
aggtagcgtggccgagcggtctaaggcgctggattaaggctccagtctcttcgggggcgtgggttcgaatcccaccgctgcca

SEQ ID NO: 311 - DNA, 74 bp, Homo sapiens
gtttccgtagtgtagtggtcatcacgttcgcctaacacgcgaaaggtccccggttcgaaaccgggcggaaacat

SEQ ID NO: 312 - DNA, 73 bp, Homo sapiens
ggctcgttggtctaggggtatgattctcgctttgggtgcgagaggtcccgggttcaaatcccggacgagccca

SEQ ID NO: 313 - DNA, 73 bp, Homo sapiens
ggctccatagctcaggggttagagcactggtcttgtaaaccagggtcgcgagttcaaatctcgctggggcctg

SEQ ID NO: 314 - DNA, 73 bp, Homo sapiens
tggggatgtagctcagtggtagagcgcatgctttgcatgtatgaggccccgggttcgatccccggcatctcca

SEQ ID NO: 315 - DNA, 74 bp, Homo sapiens
cgcccggctagctcagtcggtagagcatgagactcttaatctcagggtcgtgggttcgagccccacgttgggcg

SEQ ID NO: 316 - DNA, 74 bp, Homo sapiens
gtttccgtagtgtagtggttatcacgttcgcctaacacgcgaaaggtccccggttcgaaaccgggcggaaacaa

SEQ ID NO: 317 - DNA, 74 bp, Homo sapiens
gcccggctagctcagtcggtagagcatgagactcttaatctcagggtcgtgggttcgagccccacgttgggcgt

SEQ ID NO: 318 - DNA, 74 bp, Homo sapiens
gtttccgtagtgtagtggttatcacgttcgcctcacacgcgaaaggtccccggttcgaaaccgggcggaaacac

SEQ ID NO: 319 - DNA, 73 bp, Homo sapiens
cagcagagtggcgcagcggaagcgtgctgggcccataacccagaggtcgatggatcgaaaccatcctctgcta

SEQ ID NO: 320 - DNA, 85 bp, Homo sapiens
ggagaggcctggccgagtggttaaggcgatggactgctaatccattgtgctctgcacgcgtgggttcgaatcccatcctcgtcgc

SEQ ID NO: 321 - DNA, 73 bp, Homo sapiens
ggccccatggtgtaatggttagcactctggactttgaatccagcgatccgagttcaaatctcggtgggacctg

SEQ ID NO: 322 - DNA, 73 bp, Homo sapiens
ggccccatggtgtaatggttagcactctggactttgaatccagcgatccgagttcaaatctcggtgggaccta

SEQ ID NO: 323 - DNA, 83 bp, Homo sapiens
gtagtcgtggccgagtggttaaggcgatggacttgaaatccattggggtctccccgcgcaggttcgaatcctgccgactacgg

SEQ ID NO: 324 - DNA, 73 bp, Homo sapiens
agcagagtggcgcagcggaagcgtgctgggcccataacccagaggtcgatggatcgaaaccatcctctgctat

SEQ ID NO: 325 - DNA, 74 bp, Homo sapiens
ggaccacgtggcctaatggataaggcgtctgacttcggatcagaagattgagggttcgaatccctccgtggtta

SEQ ID NO: 326 - DNA, 83 bp, Homo sapiens
tgtagtcgtggccgagtggttaaggcgatggactagaaatccattggggtctccccgcgcaggttcgaatcctgccgactacg

SEQ ID NO: 327 - DNA, 73 bp, Homo sapiens
agcagagtggcgcagcggaagcgtgctgggcccataacccagaggtcgatggatcgaaaccatcctctgctag

SEQ ID NO: 328 - DNA, 84 bp, Homo sapiens
cgtcaggatggccgagcggtctaaggcgctgcgttcaggtcgcagtctcccctggaggcgtgggttcgaatcccactcctgaca

SEQ ID NO: 329 - DNA, 75 bp, Homo sapiens
ggctccgtggcttagctggttaaagcgcctgtctagtaaacaggagatcctgggttcgaatcccagcggggcctg

SEQ ID NO: 330 - DNA, 74 bp, Homo sapiens
agggccagtggcgcaatggataacgcgtctgactacggatcagaagattccaggttcgactcctggctggctcg

SEQ ID NO: 331 - DNA, 74 bp, Homo sapiens
ggtttccgtagtgtagtggttatcacgttcgcctcacacgcgaaaggtccccggttcgaaaccgggcggaaaca

SEQ ID NO: 332 - DNA, 73 bp, Homo sapiens
aggggatgtagctcagtggtagagcgcatgcttcgcatgtatgaggtcccgggttcgatccccggcatctcca

SEQ ID NO: 333 - DNA, 75 bp, Homo sapiens
tggccggttagctcagttggttagagcgtggtgctaataacgccaaggtcgcgggttcgatccccgtacgggcca

SEQ ID NO: 334 - DNA, 73 bp, Homo sapiens
cggctcgttggtctaggggtatgattctcgcttagggtgcgagaggtcccgggttcaaatcccggacgagccc

SEQ ID NO: 335 - DNA, 74 bp, Homo sapiens
agcccggctagctcagtcggtagagcatgagactcttaatctcagggtcgtgggttcgagccccacgttgggcg

SEQ ID NO: 336 - DNA, 92 bp, Homo sapiens
tccttcgatagctcagttggtagagcggaggactgtagttggctgtgtccttagacatccttaggtcgctggttcgaatccggctcgaagga

SEQ ID NO: 337 - DNA, 74 bp, Homo sapiens
ggggaattagctcaaatggtagagcgctcgcttagcatgcgagaggtagcgggatcgatgcccgcattctccag

SEQ ID NO: 338 - DNA, 74 bp, Homo sapiens
cgccctcttagcgcagcgggcagcgcgtcagtctcataatctgaaggtcctgagttcgagcctcagagagggca

SEQ ID NO: 339 - DNA, 95 bp, Homo sapiens
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, Homo sapiens
tgccgtgatcgtatagtggttagtactctgcgttgtggccgcagcaacctcggttcgaatccgagtcacggca

SEQ ID NO: 341 - DNA, 75 bp, Homo sapiens
ggccggttagctcagttggttagagcgtggtgctaataacgccaaggtcgcgggttcgatccccgtacgggccac

SEQ ID NO: 342 - DNA, 74 bp, Homo sapiens
agtttccgtagtgtagtggttatcacgtttgcctaacacgcgaaaggtccccggttcgaaaccgggcagaaaca

SEQ ID NO: 343 - DNA, 74 bp, Homo sapiens
gcttctgtagtgtagtggttatcacgttcgcctcacacgcgaaaggtccccggttcgaaaccgggcagaagcaa

SEQ ID NO: 344 - DNA, 73 bp, Homo sapiens
ttcctcgttagtatagtggtgagtatccccgcctgtcacgcgggagaccggggttcgattccccgacggggag

SEQ ID NO: 345 - DNA, 83 bp, Homo sapiens
gtagtcgtggccgagtggttaaggcgatggacttgaaatccattggggtttccccgcgcaggttcgaatcctgtcggctacgg

SEQ ID NO: 346 - DNA, 73 bp, Homo sapiens
aggttccatggtgtaatggttagcactctggactctgaatccagcgatccgagttcaaatctcggtggaacct

SEQ ID NO: 347 - DNA, 73 bp, Homo sapiens
tcctcgttagtatagtggtgagtgtccccgtctgtcacgcgggagaccggggttcgattccccgacggggaga

SEQ ID NO: 348 - DNA, 74 bp, Homo sapiens
gtttccgtagtgtagtggttatcacgttcgcctaacacgcgaaaggtccctggatcaaaaccaggcggaaacaa

SEQ ID NO: 349 - DNA, 75 bp, Homo sapiens
cggccggttagctcagttggttagagcgtggtgctaataacgccaaggtcgcgggttcgatccccgtactggcca

SEQ ID NO: 350 - DNA, 73 bp, Homo sapiens
ggccccatggtgtaatggtcagcactctggactctgaatccagcgatccgagttcaaatctcggtgggaccca

SEQ ID NO: 351 - DNA, 73 bp, Homo sapiens
ggccccatggtgtaatggttagcactctggactttgaatccagcgatccgagttcaaatctcggtgggacctt

SEQ ID NO: 352 - DNA, 73 bp, Homo sapiens
tgggggtgtagctcagtggtagagcgcgtgcttagcatgtacgaggtcccgggttcaatccccggcacctcca

SEQ ID NO: 353 - DNA, 73 bp, Homo sapiens
ggggatgtagctcagtggtagagcgcatgcttagcatgcatgaggtcccgggttcgatccccagcatctccag

SEQ ID NO: 354 - DNA, 73 bp, Homo sapiens
agggggtgtagctcagtggtagagcgcgtgcttcgcatgtacgaggccccgggttcgacccccggctcctcca

SEQ ID NO: 355 - DNA, 73 bp, Homo sapiens
gggggtgtagctcagtggtagagcgcgtgcttagcatgcacgaggccccgggttcaatccccggcacctccat

SEQ ID NO: 356 - DNA, 73 bp, Homo sapiens
gggggtgtagctcagtggtagagcgcgtgcttagcatgcacgaggccccgggttcaatccccggcacctccag

SEQ ID NO: 357 - DNA, 107 bp, Homo sapiens
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, Homo sapiens
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, Homo sapiens
ggttccatggtgtaatggttagcactctggactctgaatccagcgatccgagttcaaatctcggtggaacctt

SEQ ID NO: 360 - DNA, 83 bp, Homo sapiens
ggtagcgtggccgagcggtctaaggcgctggattaaggctccagtctcttcgggggcgtgggttcgaatcccaccgctgccag

SEQ ID NO: 361 - DNA, 74 bp, Homo sapiens
tgcctccttagcgcagtaggcagcgcgtcagtctcataatctgaaggtcctgagttcgaacctcagagggggca

SEQ ID NO: 362 - DNA, 74 bp, Homo sapiens
agcccggatagctcagtcggtagagcatcagacttttaatctgagggtccagggttcaagtccctgttcgggcg

SEQ ID NO: 363 - DNA, 74 bp, Homo sapiens
gcctccttagcgcagtaggcagcgcgtcagtctcataatctgaaggtcctgagttcgaacctcagagggggcag

SEQ ID NO: 364 - DNA, 73 bp, Homo sapiens
ttccctggtggtctagtggttaggattcggcgctctcaccgccgcggcccgggttcgattcccggtcagggaa

SEQ ID NO: 365 - DNA, 84 bp, Homo sapiens
caccaggatggccgagtggttaaggcgttggacttaagatccaatggacatatgtccgcgtgggttcgaaccccactcctggta

SEQ ID NO: 366 - DNA, 73 bp, Homo sapiens
tggctcgttggtctaggggtatgattctcgcttagggtgcgagaggtcccgggttcaaatcccggacgagccc

SEQ ID NO: 367 - DNA, 74 bp, Homo sapiens
agccccagtggcctaatggataaggcattggcctcctaagccagggattgtgggttcgagtcccatctggggtg

SEQ ID NO: 368 - DNA, 73 bp, Homo sapiens
ggggatatagctcaggggtagagcatttgactgcagatcaagaggtccccggttcaaatccgggtgccccccc

SEQ ID NO: 369 - DNA, 94 bp, Homo sapiens
cccttcgatagctcagctggtagagcggaggactgtagctacttcctcagcaggagacatccttaggtcgctggttcgattccggctcgaagga

SEQ ID NO: 370 - DNA, 90 bp, Homo sapiens
cccttcgatagctcagctggtagagcggaggactgtaggcgcgcgcccgtggccatccttaggtcgctggttcgattccggctcgaagga

SEQ ID NO: 371 - DNA, 74 bp, Homo sapiens
tgggggattagctcaaatggtagagcgctcgcttagcatgcgagaggtagcgggatcgatgcccgcatcctcca

SEQ ID NO: 372 - DNA, 83 bp, Homo sapiens
gtagtcgtggccgagtggttaaggcgatggactagaaatccattggggtctccccgcgcaggttcgaatcctgccgactacgg

SEQ ID NO: 373 - DNA, 74 bp, Homo sapiens
gcctcgttagcgcagtaggtagcgcgtcagtctcataatctgaaggtcgtgagttcgatcctcacacggggcac

SEQ ID NO: 374 - DNA, 92 bp, Homo sapiens
ggctctgtggcgcaatggatagcgcattggacttctagctgagcctagtgtggtcattcaaaggttgtgggttcgagtcccaccagagtcga

SEQ ID NO: 375 - DNA, 75 bp, Homo sapiens
gtctctgtggcgcaatcggttagcgcgttcggctgttaaccgaaaggttggtggttcgagcccacccagggacgc

SEQ ID NO: 376 - DNA, 83 bp, Homo sapiens
ggcagcgatggccgagtggttaaggcgttggacttgaaatccaatggggtctccccgcgcaggttcgaaccctgctcgctgcg

SEQ ID NO: 377 - DNA, 74 bp, Homo sapiens
ggttccatagtgtagtggttatcacgtctgctttacacgcagaaggtcctgggttcgagccccagtggaaccat

SEQ ID NO: 378 - DNA, 74 bp, Homo sapiens
ggttccatagtgtagcggttatcacgtctgctttacacgcagaaggtcctgggttcgagccccagtggaaccac

SEQ ID NO: 379 - DNA, 87 bp, Homo sapiens
tggctctgtggcgcaatggatagcgcattggacttctagatagttagagaaattcaaaggttgtgggttcgagtcccaccagagtcg

SEQ ID NO: 380 - DNA, 84 bp, Homo sapiens
taccagaatggccgagtggttaaggcgttggacttaagatccaatggattcatatccgcgtgggttcgaaccccacttctggta

SEQ ID NO: 381 - DNA, 74 bp, Homo sapiens
ggcccggatagctcagtcggtagagcatcagacttttaatctgagggtccggggttcaagtccctgttcgggcg

SEQ ID NO: 382 - DNA, 74 bp, Homo sapiens
gccgaaatagctcagttgggagagcgttagactgaagatctaaaggtccctggttcgatcccgggtttcggcag

SEQ ID NO: 383 - DNA, 74 bp, Homo sapiens
gcccggatagctcagtcggtagagcatcagacttttaatctgagggtccagggttcaagtccctgttcgggcgg

SEQ ID NO: 384 - DNA, 74 bp, Homo sapiens
gccgaaatagctcagttgggagagcgttagactgaagatctaaaggtccctggttcaatcccgggtttcggcag

SEQ ID NO: 385 - DNA, 83 bp, Homo sapiens
ggacgaggtggccgagtggttaaggcgatggactgctaatccattgtgctttgcacgcgtgggttcgaatcccatcctcgtcg

SEQ ID NO: 386 - DNA, 73 bp, Homo sapiens
ggctcgttggtctaggggtatgattctcggtttgggtccgagaggtcccgggttcaaatcccggacgagcccc

SEQ ID NO: 387 - DNA, 83 bp, Homo sapiens
agtcacggtggccgagtggttaaggcgttggactcgaaatccaatggggtttccccgcacaggttcgaatcctgttcgtgacg

SEQ ID NO: 388 - DNA, 73 bp, Homo sapiens
ctcctcgttagtatagtggttagtatccccgcctgtcacgcgggagaccggggttcaattccccgacggggag

SEQ ID NO: 389 - DNA, 73 bp, Homo sapiens
ggacctcgtggcgcaacggtagcgcgtctgactccagatcagaaggctgcgtgttcgaatcacgtcggggtca

SEQ ID NO: 390 - DNA, 73 bp, Homo sapiens
ggggatgtagctcagtggtagagcgcatgctttgcatgtatgaggccccgggttcgatccccggcatctccat

SEQ ID NO: 391 - DNA, 74 bp, Homo sapiens
gccgaaatagctcagttgggagagcgttagactgaagatctaaaggtccctggttcgatcccgggtttcggcac

SEQ ID NO: 392 - DNA, 73 bp, Homo sapiens
aggggatgtagctcagtggtagagcgcatgctttgcacgtatgaggccccgggttcaatccccggcatctcca

SEQ ID NO: 393 - DNA, 75 bp, Homo sapiens
gtctctgtggcgcaatcggttagcgcgttcggctgttaaccgaaaggttggtggttcgagcccacccagggacgg

SEQ ID NO: 394 - DNA, 73 bp, Homo sapiens
tcccacatggtctagcggttaggattcctggttttcacccaggcggcccgggttcgactcccggtgtgggaac

SEQ ID NO: 395 - DNA, 74 bp, Homo sapiens
ggctccatagctcaggggttagagcgctggtcttgtaaaccaggggtcgcgagttcaattctcgctggggcctg

SEQ ID NO: 396 - DNA, 83 bp, Homo sapiens
tggtagtgtggccgagcggtctaaggcgctggatttaggctccagtctcttcgggggcgtgggttcgaatcccaccactgcca

SEQ ID NO: 397 - DNA, 74 bp, Homo sapiens
ggctccatagctcaggggttagagcactggtcttgtaaaccaggggtcgcgagttcaaatctcgctggggcctc

SEQ ID NO: 398 - DNA, 73 bp, Homo sapiens
tggctcgttggtctagtggtatgattctcgctttgggtgcgagaggtcccgggttcaaatcccggacgagccc

SEQ ID NO: 399 - DNA, 95 bp, Homo sapiens
ccttcgatagctcagctggtagagcggaggactgtagattgtacagacatttgcggacatccttaggtcgctggttcgattccggctcgaaggaa

SEQ ID NO: 400 - DNA, 74 bp, Homo sapiens
aggccctatagctcaggggttagagcactggtcttgtaaaccaggggtcgcgagttcaaatctcgctggggcct

SEQ ID NO: 401 - DNA, 90 bp, Homo sapiens
tccttcgatagctcagctggtagagcggaggactgtagtacttaatgtgtggtcatccttaggtcgctggttcgattccggctcgaagga

SEQ ID NO: 402 - DNA, 73 bp, Homo sapiens
tggctcgttggtctaggggtatgattctcgctttgggtgcgagaggtcccgggttcaaatcccggacgagccc

SEQ ID NO: 403 - DNA, 74 bp, Homo sapiens
gcccggctagctcagtcggtagagcatgggactcttaatcccagggtcgtgggttcgagccccacgttgggcgc

SEQ ID NO: 404 - DNA, 75 bp, Homo sapiens
cggccggttagctcagttggttagagcgtggtgctaataacgccaaggtcgcgggttcgatccccgtacgggcca

SEQ ID NO: 405 - DNA, 73 bp, Homo sapiens
tcccacatggtctagcggttaggattcctggttttcacccaggcggcccgggttcgactcccggtgtgggaat

SEQ ID NO: 406 - DNA, 83 bp, Homo sapiens
gacgaggtggccgagtggttaaggcgatggactgctaatccattgtgctctgcacgcgtgggttcgaatcccatcctcgtcga

SEQ ID NO: 407 - DNA, 73 bp, Homo sapiens
gccgtgatcgtatagtggttagtactctgcgttgtggccgcagcaacctcggttcgaatccgagtcacggcat

SEQ ID NO: 408 - DNA, 73 bp, Homo sapiens
cgccgtgatcgtatagtggttagtactctgcgttgtggccgcagcaacctcggttcgaatccgagtcacggca

SEQ ID NO: 409 - DNA, 73 bp, Homo sapiens
ggttccatggtgtaatggttagcactctggactctgaatccagcgatccgagttcaaatctcggtggaacctg

SEQ ID NO: 410 - DNA, 74 bp, Homo sapiens
tgcccggctagctcagtcggtagagcatgggactcttaatcccagggtcgtgggttcgagccccacgttgggcg

SEQ ID NO: 411 - DNA, 74 bp, Homo sapiens
gggccgcgtggcctaatggataaggcgtctgacttcggatcagaagattgcaggttcgagtcctgccgcggtcg

SEQ ID NO: 412 - DNA, 72 bp, Homo sapiens
gcgccgctggtgtagtggtatcatgcaagattcccattcttgcgacccgggttcgattcccgggcggcgcac

SEQ ID NO: 413 - DNA, 74 bp, Homo sapiens
gggccgcgtggcctaatggataaggcgtctgattccggatcagaagattgagggttcgagtcccttcgtggtcg

SEQ ID NO: 414 - DNA, 74 bp, Homo sapiens
cgccccggtggcctaatggataaggcattggcctcctaagccagggattgtgggttcgagtcccacccggggta

SEQ ID NO: 415 - DNA, 74 bp, Homo sapiens
gcccggctagctcagtcggtagagcatgagacccttaatctcagggtcgtgggttcgagccccacgttgggcgt

SEQ ID NO: 416 - DNA, 73 bp, Homo sapiens
aggcgcggtggccaagtggtaaggcgtcggtctcgtaaaccgaagatcacgggttcgaaccccgtccgtgcct

SEQ ID NO: 417 - DNA, 83 bp, Homo sapiens
ggtagcgtggccgagtggtctaaggcgctggatttaggctccagtcatttcgatggcgtgggttcgaatcccaccgctgccac

SEQ ID NO: 418 - DNA, 83 bp, Homo sapiens
gggtagcgtggccgagcggtctaaggcgctggattaaggctccagtctcttcgggggcgtgggttcgaatcccaccgctgcca

SEQ ID NO: 419 - DNA, 84 bp, Homo sapiens
agtcaggatggccgagcggtctaaggcgctgcgttcaggtcgcagtctcccctggaggcgtgggttcgaatcccacttctgaca

SEQ ID NO: 420 - DNA, 84 bp, Homo sapiens
gtcaggatggccgagcggtctaaggcgctgcgttcaggtcgcagtctcccctggaggcgtgggttcgaatcccacttctgacag

SEQ ID NO: 421 - DNA, 74 bp, Homo sapiens
gcctcgttagcgcagtaggcagcgcgtcagtctcataatctgaaggtcgtgagttcgagcctcacacggggcag

SEQ ID NO: 422 - DNA, 83 bp, Homo sapiens
ggtagcgtggccgagcggtctaaggcgctggatttaggctccagtctcttcggaggcgtgggttcgaatcccaccgctgccag

SEQ ID NO: 423 - DNA, 89 bp, Homo sapiens
tggctctgtggcgcaatggatagcgcattggacttctagtgacgaatagagcaattcaaaggttgtgggttcgaatcccaccagagtcg

SEQ ID NO: 424 - DNA, 72 bp, Homo sapiens
cgcattggtggttcagtggtagaattctcgcctgccacgcgggaggcccgggttcgattcccggccaatgca

SEQ ID NO: 425 - DNA, 83 bp, Homo sapiens
gctgtgatggccgagtggttaaggcgttggactcgaaatccaatggggtctccccgcgcaggttcgaatcctgctcacagcgt

SEQ ID NO: 426 - DNA, 75 bp, Homo sapiens
ggcgccgtggcttagctggttaaagcgcctgtctagtaaacaggagatcctgggttcgaatcccagcggtgcctg

SEQ ID NO: 427 - DNA, 73 bp, Homo sapiens
cgacctcgtggcgcaacggtagcgcgtctgactccagatcagaaggttgcgtgttcaaatcacgtcggggtca

SEQ ID NO: 428 - DNA, 83 bp, Homo sapiens
agacgaggtggccgagtggttaaggcgatggactgctaatccattgtgctctgcacgcgtgggttcgaatcccatcctcgtcg

SEQ ID NO: 429 - DNA, 75 bp, Homo sapiens
cggcgccgtggcttagttggttaaagcgcctgtctagtaaacaggagatcctgggttcgaatcccagcggtgcct

SEQ ID NO: 430 - DNA, 73 bp, Homo sapiens
ggcctcgtggcgcaacggtagcgcgtctgactccagatcagaaggttgcgtgttcaaatcacgtcggggtcaa

SEQ ID NO: 431 - DNA, 73 bp, Homo sapiens
agcgttggtggtatagtggtaagcatagctgccttccaagcagttgacccgggttcgattcccggccaacgca

SEQ ID NO: 432 - DNA, 73 bp, Homo sapiens
tcctcgttagtatagtggtgagtatccccgcctgtcacgcgggagaccggggttcgattccccgacggggaga

SEQ ID NO: 433 - DNA, 73 bp, Homo sapiens
ggctcgttggtctaggggtatgattctcgcttcgggtgcgagaggtcccgggttcaaatcccggacgagccct

SEQ ID NO: 434 - DNA, 75 bp, Homo sapiens
ggcgccgtggcttagttggttaaagcgcctgtctagtaaacaggagatcctgggttcgaatcccagcggtgcctt

SEQ ID NO: 435 - DNA, 83 bp, Homo sapiens
gtagtcgtggccgagtggttaaggcgatggactagaaatccattggggtctccccgcgcaggttcgaatcctgccgactacgt

SEQ ID NO: 436 - DNA, 73 bp, Homo sapiens
tgacctcgtggcgcaatggtagcgcgtctgactccagatcagaaggttgcgtgttcaagtcacgtcggggtca

SEQ ID NO: 437 - DNA, 73 bp, Homo sapiens
aggcgcggtggccaagtggtaaggcgtcggtctcgtaaaccgaagatcgcgggttcgaaccccgtccgtgcct

SEQ ID NO: 438 - DNA, 73 bp, Homo sapiens
agggggtatagctcagtggtagagcatttgactgcagatcaagaggtccccggttcaaatccgggtgccccct

SEQ ID NO: 439 - DNA, 73 bp, Homo sapiens
gggggtatagctcagtggtagagcatttgactgcagatcaagaggtccctggttcaaatccgggtgccccctc

SEQ ID NO: 440 - DNA, 73 bp, Homo sapiens
gggggtatagctcagtggtagagcatttgactgcagatcaagaggtccccggttcaaatccgggtgccccctc

SEQ ID NO: 441 - DNA, 73 bp, Homo sapiens
aggtcccatggtgtaatggttagcactctggactttgaatccagcgatccgagttcaaatctcggtgggacct

SEQ ID NO: 442 - DNA, 74 bp, Homo sapiens
gacccagtggcctaatggataaggcatcagcctccggagctggggattgtgggttcgagtcccatctgggtcgc

SEQ ID NO: 443 - DNA, 74 bp, Homo sapiens
agccccagtggcctaatggataaggcactggcctcctaagccagggattgtgggttcgagtcccacctggggta

SEQ ID NO: 444 - DNA, 74 bp, Homo sapiens
gccccagtggcctaatggataaggcactggcctcctaagccagggattgtgggttcgagtcccacctggggtgt

SEQ ID NO: 445 - DNA, 74 bp, Homo sapiens
agaccgcgtggcctaatggataaggcgtctgacttcggatcagaagattgagggttcgagtcccttcgtggtcg

SEQ ID NO: 446 - DNA, 75 bp, Homo sapiens
cgtctctgtggcgcaatcggttagcgcgttcggctgttaaccgaaaggttggtggttcgagcccacccagggacg

SEQ ID NO: 447 - DNA, 73 bp, Homo sapiens
ggcgttggtggtatagtggttagcatagctgccttccaagcagttgacccgggttcgattcccggccaacgca

SEQ ID NO: 448 - DNA, 74 bp, Homo sapiens
gtttccgtagtgtagcggttatcacattcgcctcacacgcgaaaggtccccggttcgatcccgggcggaaacag

SEQ ID NO: 449 - DNA, 75 bp, Homo sapiens
tggcgccgtggcttagttggttaaagcgcctgtctagtaaacaggagatcctgggttcgaatcccagcggtgcct

SEQ ID NO: 450 - DNA, 94 bp, Homo sapiens
gctccagtggcgcaatcggttagcgcgcggtacttatatgacagtgcgagcggagcaatgccgaggttgtgagttcgatcctcacctggagcac

SEQ ID NO: 451 - DNA, 72 bp, Homo sapiens
gcatgggtggttcagtggtagaattctcgcctgccacgcgggaggcccgggttcgattcccggcccatgcag

SEQ ID NO: 452 - DNA, 20 bp, Artificial Sequence
  source: /note=“Description of Artificial Sequence Synthetic primer”
tgagttggcaacctgtggta

SEQ ID NO: 453 - DNA, 20 bp, Artificial Sequence
  source: /note=“Description of Artificial Sequence Synthetic primer”
ttgggtgtccatgaaaatca

SEQ ID NO: 454 - RNA, 26 nt, Artificial Sequence
  source: /note=“Description of Artificial Sequence Synthetic probe”
uagcagaggaugguuucgauccauca

SEQ ID NO: 455 - DNA, 26 bp, Artificial Sequence
  source: /note=“Description of Artificial Sequence Synthetic probe”
tagcagaggatggtttcgatccatca

SEQ ID NO: 456 - protein, 6 aa, Artificial Sequence
  source: /note=“Description of Artificial Sequence Synthetic 6xHis tag”
HHHHHH

SEQ ID NO: 457 - DNA, 49 bp, Artificial Sequence
  source: /note=“Description of Artificial Sequence Synthetic probe”
ccaatggatttctatccatcgccttaaccactcggccacgactacaaaa

SEQ ID NO: 458 - RNA, 15 nt, Artificial Sequence
  source: /note=“Description of Artificial Sequence Synthetic probe”
cagauuaaaagucug

SEQ ID NO: 459 - RNA, 77 nt, Artificial Sequence
  source: /note=“Description of Artificial Sequence Synthetic oligonucleotide”
ggcucuauggcuuaguugguuaaagcgccugucucguaaacaggagauccuggguucgacucccaguggggccucaa

SEQ ID NO: 460 - RNA, 84 nt, Artificial Sequence
  source: /note=“Description of Artificial Sequence Synthetic oligonucleotide”
gucaggauggccgaguggucuaaggcgccagacucaaguucuggucuccguauggaggcguggguucgaaucccacuucugaca

SEQ ID NO: 461 - DNA, 20 bp, Unknown
  source: /note=“Description of Unknown Target sequence”
cagtgtgtgaagacggctgc

SEQ ID NO: 462 - RNA, 72 nt, Artificial Sequence
  source: /note=“Description of Artificial Sequence Synthetic oligonucleotide”
ggcucguuggucuagggguaugauucucgcuuagggugcgagaggucccggguucaaaucccggacgagccc

SEQ ID NO: 463 - RNA, 72 nt, Artificial Sequence
  source: /note=“Description of Artificial Sequence Synthetic oligonucleotide”
ggggauguagcucagugguagagcgcaugcuuugcauguaugaggucccggguucgauccccggcaucucca

SEQ ID NO: 464 - RNA, 71 nt, Artificial Sequence
  source: /note=“Description of Artificial Sequence Synthetic oligonucleotide”
ggcucguuggucuagggguaugaucucgcuuagggugcgagaggucccggguucaaaucccggacgagccc

SEQ ID NO: 465 - RNA, 74 nt, Artificial Sequence
  source: /note=“Description of Artificial Sequence Synthetic oligonucleotide”
ggcucuguggcuuaguuggcuaaagcgccugucucguaaacaggagauccuggguucgaaucccagcggggccu

SEQ ID NO: 466 - protein, 8 aa, Artificial Sequence
  source: /note=“Description of Artificial Sequence Synthetic peptide”
DYKDDDDK

SEQ ID NO: 467 - RNA, 82 nt, Artificial Sequence
  source: /note=“Description of Artificial Sequence Synthetic oligonucleotide”
gguagcguggccgagcggucuaaggcgcuggauuaaggcuccagucucuucggaggcguggguucgaaucccaccgcugcca

SEQ ID NO: 468 - RNA, 74 nt, Artificial Sequence
  source: /note=“Description of Artificial Sequence Synthetic oligonucleotide”
ggcgccguggcuuaguugguuaaagcgccugucuaguaaacaggagauccuggguucgaaucccagcggugccu